(19) United States
(12) Patent Application Publication
(10) Pub. No.: US 2022/0056451 A1
(43) Pub. Date: Feb. 24, 2022

(54) HBV BINDING OLIGONUCLEOTIDES AND METHODS OF USE

(71) Applicant: Aligos Therapeutics, Inc., South San Francisco, CA (US)

(72) Inventors: Jin Hong, South San Francisco, CA (US); Leonid Beigelman, South San Francisco, CA (US); Aneerban Bhattacharya, South San Francisco, CA (US); N. Tilani S. De Costa, South San Francisco, CA (US)

(21) Appl. No.: 17/385,208

(22) Filed: Jul. 26, 2021

Related U.S. Application Data
(60) Provisional application No. 63/056,883, filed on Jul. 27, 2020.
(60) Provisional application No. 63/197,181, filed on Jun. 4, 2021.

(51) Int. Cl. C12N 15/113; A61K 31/713; A61K 45/06; A61P 31/20

(52) U.S. Cl. CPC C12N 15/1131; A61K 31/713; A61K 45/06; A61P 31/20; C12N 2320/31; C12N 2310/3231; C12N 2310/315; C12N 2310/351; C12N 2310/314; C12N 2310/321

(57) ABSTRACT

Oligonucleotides that target hepatitis B virus (HBV) viral sequences, such as rcDNA, cccDNA, and HBV transcripts, are described herein. In addition, compositions and kits comprising such oligonucleotides are further described. Further disclosed herein are uses of such oligonucleotides and compositions to reduce rcDNA to cccDNA conversion, reduce cccDNA levels, and/or treat an HBV infection.

### CROSS-REFERENCE TO RELATED APPLICATIONS

This application claims priority to U.S. Provisional Application No. 63/056,883, filed Jul. 27, 2020, and U.S. Provisional Application No. 63/197,181, filed Jun. 4, 2021, the disclosures of which are hereby incorporated by reference in their entireties.

### BACKGROUND

About 240 million people are chronically infected with HBV worldwide and long-term risks such as cirrhosis and hepatocellular carcinoma (HCC) account for approximately 600,000 deaths annually. Current HBV therapies that do not eliminate covalently closed circular DNA (cccDNA) in the nucleus of infected cells may result in persistence and relapse of HBV infection. Thus, there is a need in the art to develop an HBV therapy that can eliminate or permanently silence HBV infection.

Disclosed herein are oligonucleotides that bind to hepatitis B virus (HBV) nucleic acid sequences, such as the rcDNA and cccDNA forms of the HBV genome and HBV transcripts. In addition, compositions and kits comprising such oligonucleotides and uses of such oligonucleotides and compositions to reduce rcDNA to cccDNA conversion, reduce cccDNA levels, silencing cccDNA transcription and/or treat an HBV infection are described herein.

### SUMMARY

Disclosed herein are oligonucleotides that are identical, complementary, hybridize, or bind to HBV viral target sequences, acting as steric blockers. In some embodiments, the oligonucleotide comprises a nucleotide sequence comprising 5 to 40 nucleotides, wherein one or more of the 5 to 40 nucleotides is a modified nucleoside, wherein at least 5 consecutive nucleotides of the 5 to 40 nucleotides is identical, complementary, hybridizes or binds to a viral target sequence, wherein the viral target sequence is within (a) a relaxed circular DNA (rcDNA) form of a hepatitis B virus (HBV) genome; (b) a covalently closed circular DNA (cccDNA) of the HBV genome; or (c) an HBV transcript.

In some embodiments, the viral target sequence is in a gap region of the rcDNA. In some embodiments, the viral target sequence comprises 5 to 40 nucleotides within a gap region of the rcDNA. In some embodiments, the gap region comprises positions 1 to 1600, 200 to 1600, 300 to 1600, 400 to 1600, 500 to 1600, 600 to 1600, 650 to 1600, 700 to 1600, 750 to 1600, 800 to 1600, 850 to 1600, 900 to 1600, 950 to 1600, 1000 to 1600, 1050 to 1600, 1100 to 1600, 1150 to 1600, 1200 to 1600, 1250 to 1600, 1300 to 1600, 1350 to 1600, 1400 to 1600, 1450 to 1600, 1500 to 1600, 1550 to 1600, or 1580 to 1600 of SEQ ID NO: 1 or a comparable region in SEQ ID NO: 2 or a sequence of any of HBV genotypes A-J.

In some embodiments, the viral target sequence is in a non-gap region of the rcDNA. In some embodiments, the viral target sequence comprises 5 to 40 nucleotides within a non-gap region of the rcDNA. In some embodiments, the non-gap region comprises positions 1601 to 3215, 1601 to 3100, 1601 to 2900, 1601 to 2800, 1601 to 2700, 1601 to 2600, 1601 to 2500, 1601 to 2400, 1601 to 2300, 1601 to 2250, 1601 to 2200, 1601 to 2150, 1601 to 2100, 1601 to 2050, 1601 to 2000, 1601 to 1950, 1601 to 1900, 1601 to 1850, 1601 to 1800, 1601 to 1750, or 1601 to 1700 of SEQ ID NO: 1 or a comparable region in SEQ ID NO: 2 or a sequence of any of HBV genotypes A-J.

In some embodiments, the viral target sequence is in the cccDNA. In some embodiments, the viral target sequence comprises 5 to 40 nucleotides within the cccDNA. In some embodiments, the viral target sequence comprises 5 to 40 nucleotides within positions 950-1050, 975-1025, 975-1010, 980-1010, 1150-1275, 1175-1275, 1180-1270, 1180-1250, 1180-1225, 1180-1210, 1225-1350, 1225-1325, 1225-1300, 1225-1290, 1250-1350, 1250-1325, 1250-1300, 1250-1290, 1375-1475, 1375-1450, 1375-1440, 1375-1430, 1390-1475, 1390-1450, 1390-1440, 1390-1430, 1500-1700, 1500-1650, 1500-1620, 1500-1595, 1510-1700, 1510-1650, 1510-1620, 1510-1595, 1515-1700, 1515-1650, 1515-1620, 1515-1590, or 2817-3050 of SEQ ID NO: 1 or a comparable region in SEQ ID NO: 2 or a sequence of any of HBV genotypes A-J.

In some embodiments, the nucleotide sequence is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical to 5 to 40 consecutive nucleotides starting at position 1181, 1255, 1256, 1257, 1258, 1259, 1260, 1261, 1263, 1264, 1265, 1266, 1267, 1268, 1393, 1394, 1410, 1411, 1412, 1515, 1516, 1517, 1523, 1527, 1528, 1529, 1530, 1531, 1577, or 2817 of SEQ ID NO: 1 or a comparable position in SEQ ID NO: 2 or a sequence of any of HBV genotypes A-J.

In some embodiments, the nucleotide sequence is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical to 5 to 40 consecutive nucleotides within positions 950-1050, 975-1025, 975-1010, 980-1010, 1150-1275, 1175-1275, 1180-1270, 1180-1250, 1180-1225, 1180-1210, 1225-1350, 1225-1325, 1225-1300, 1225-1290, 1250-1350, 1250-1325, 1250-1300, 1250-1290, 1375-1475, 1375-1450, 1375-1440, 1375-1430, 1390-1475, 1390-1450, 1390-1440, 1390-1430, 1500-1700, 1500-1650, 1500-1620, 1500-1595, 1510-1700, 1510-1650, 1510-1620, 1510-1595, 1515-1700, 1515-1650, 1515-1620, 1515-1590, or 2817-3050 of SEQ ID NO: 1 or a comparable region in SEQ ID NO: 2 or a sequence of any of HBV genotypes A-J.

In some embodiments, the nucleotide sequence is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% complementary to 5 to 40 consecutive nucleotides starting at position 1181, 1255, 1256, 1257, 1258, 1259, 1260, 1261, 1263, 1264, 1265, 1266, 1267, 1268, 1393, 1394, 1410, 1411, 1412, 1515, 1516, 1517, 1523, 1527, 1528, 1529, 1530, 1531, 1577, or 2817 of SEQ ID NO: 1 or a comparable position in SEQ ID NO: 2 or a sequence of any of HBV genotypes A-J.

In some embodiments, the nucleotide sequence is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% complementary to 5 to 40 consecutive nucleotides within positions 950-1050, 975-1025, 975-1010, 980-1010, 1150-1275, 1175-1275, 1180-1270, 1180-1250, 1180-1225, 1180-1210, 1225-1350, 1225-1325, 1225-1300, 1225-1290, 1250-1350, 1250-1325, 1250-1300, 1250-1290, 1375-1475, 1375-1450, 1375-1440, 1375-1430, 1390-1475, 1390-1450, 1390-1440, 1390-1430, 1500-1700, 1500-1650, 1500-1620, 1500-1595, 1510-1700, 1510-1650, 1510-1620, 1510-1595, 1515-1700, 1515-1650, 1515-1620, 1515-1590, or 2817-3050 of SEQ ID NO: 1 or a comparable region in SEQ ID NO: 2 or a sequence of any of HBV genotypes A-J.

In some embodiments, the nucleotide sequence hybridizes under high stringency conditions to 5 to 40 consecutive nucleotides starting at position 1181, 1255, 1256, 1257, 1258, 1259, 1260, 1261, 1263, 1264, 1265, 1266, 1267, 1268, 1393, 1394, 1410, 1411, 1412, 1515, 1516, 1517, 1523, 1527, 1528, 1529, 1530, 1531, 1577, or 2817 of SEQ ID NO: 1 or a comparable region in SEQ ID NO: 2 or a sequence of any of HBV genotypes A-J.

In some embodiments, the nucleotide sequence hybridizes under high stringency conditions to 5 to 40 consecutive nucleotides within positions 950-1050, 975-1025, 975-1010, 980-1010, 1150-1275, 1175-1275, 1180-1270, 1180-1250, 1180-1225, 1180-1210, 1225-1350, 1225-1325, 1225-1300, 1225-1290, 1250-1350, 1250-1325, 1250-1300, 1250-1290, 1375-1475, 1375-1450, 1375-1440, 1375-1430, 1390-1475, 1390-1450, 1390-1440, 1390-1430, 1500-1700, 1500-1650, 1500-1620, 1500-1595, 1510-1700, 1510-1650, 1510-1620, 1510-1595, 1515-1700, 1515-1650, 1515-1620, 1515-1590, or 2817-3050 of SEQ ID NO: 1 or a comparable region in SEQ ID NO: 2 or a sequence of any of HBV genotypes A-J.

In some embodiments, the nucleotide sequence preferentially hybridizes to 5 to 40 consecutive nucleotides starting at position 1181, 1255, 1256, 1257, 1258, 1259, 1260, 1261, 1263, 1264, 1265, 1266, 1267, 1268, 1393, 1394, 1410, 1411, 1412, 1515, 1516, 1517, 1523, 1527, 1528, 1529, 1530, 1531, 1577, or 2817 of SEQ ID NO: 1 as compared to other positions within SEQ ID NO: 1.

In some embodiments, the nucleotide sequence preferentially hybridizes to 5 to 40 consecutive nucleotides within positions 950-1050, 975-1025, 975-1010, 980-1010, 1150-1275, 1175-1275, 1180-1270, 1180-1250, 1180-1225, 1180-1210, 1225-1350, 1225-1325, 1225-1300, 1225-1290, 1250-1350, 1250-1325, 1250-1300, 1250-1290, 1375-1475, 1375-1450, 1375-1440, 1375-1430, 1390-1475, 1390-1450, 1390-1440, 1390-1430, 1500-1700, 1500-1650, 1500-1620, 1500-1595, 1510-1700, 1510-1650, 1510-1620, 1510-1595, 1515-1700, 1515-1650, 1515-1620, 1515-1590, or 2817-3050 of SEQ ID NO: 1 as compared to other positions within SEQ ID NO: 1.

In some embodiments, the viral target sequence is in an X region of the rcDNA. In some embodiments, the viral target sequence comprises 5 to 40 nucleotides within the X region. In some embodiments, the viral target sequence comprises 5 to 40 nucleotides within position 1374 to 1603, 1400 to 1603, 1450 to 1603, 1500 to 1603, or 1550 to 1603 of SEQ ID NO: 1 or a comparable region in SEQ ID NO: 2 or a sequence of any of HBV genotypes A-J. In some embodiments, the viral target sequence is in an S region of the rcDNA. In some embodiments, the viral target sequence comprises 5 to 40 nucleotides within the S region.

In some embodiments, the viral target sequence comprises 5 to 40 nucleotides within position 155 to 1373, 200 to 1373, 300 to 1373, 400 to 1373, 500 to 1373, 600 to 1373, 650 to 1373, 700 to 1373, 750 to 1373, or 800 to 1373 of SEQ ID NO: 1 or a comparable region in SEQ ID NO: 2 or a sequence of any of HBV genotypes A-J.

In some embodiments, the viral target sequence is in the HBV transcript. In some embodiments, the HBV transcript is selected from pre-genomic RNA (pgRNA), pre-Core RNA, pre-S1 RNA, pre-S2 RNA and X RNA.

In some embodiments, the viral target sequence is in pgRNA. In some embodiments, the viral target sequence comprises 5 to 40 nucleotides within the pgRNA.

In some embodiments, the viral target sequence is in pre-Core RNA. In some embodiments, the viral target sequence comprises 5 to 40 nucleotides within the pre-Core RNA.

In some embodiments, the viral target sequence is in pre-S1 RNA. In some embodiments, the viral target sequence comprises 5 to 40 nucleotides within the pre-S1 RNA.

In some embodiments, the viral target sequence is in pre-S2 RNA. In some embodiments, the viral target sequence comprises 5 to 40 nucleotides within the pre-S2 RNA.

In some embodiments, the viral target sequence is in X RNA. In some embodiments, the viral target sequence comprises 5 to 40 nucleotides within the X RNA.

In some embodiments, the nucleotide sequence comprises 10 to 25, 15 to 25, 14 to 24, 14 to 23, 14 to 22, or 15 to 22 nucleotides. In some embodiments, the nucleotide sequence comprises at least 5, 6, 7, 8, 9, 10, 11, 12, 13, 14, 15, 16, or 17 nucleotides. In some embodiments, the nucleotide sequence comprises less than or equal to 30, 29, 28, 27, 26, 25, 24, 23, 22, 21, or 20 nucleotides.

In some embodiments, at least 2, 3, 4, 5, 6, 7, 8, 9, 10, 11, 12, 13, 14, 15, 16, 17, 18, 19, 20, 21, 22, 23, 24 or more of the 5 to 40 nucleotides are modified nucleosides. In some embodiments, fewer than or equal to 25, 24, 23, 22, 21, 20, 19, 18, 17, 16, 15, 14, 13, 12, 10, 11, 10, 9, 8, 7, 6, 5, 4, 3, or 2 of the 5 to 40 nucleotides are modified nucleosides.

The oligonucleotide of any preceding claim, wherein at least 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, 97%, 99%, or 100% of the 5 to 40 nucleotides are modified nucleosides.

In some embodiments, less than or equal to 100%, 99%, 97%, 95%, 90%, 85%, 80%, 75%, 70%, 65%, 60%, 55%, 50%, 45%, 40%, 35%, 30%, 25%, or 20% of the 5 to 40 nucleotides are modified nucleosides.

In some embodiments, at least 1, 2, 3, 4, 5, 6, 7, 8, 9, 10, 11, 12, 13, 14, 15, 16, 17, 18, 19, 20, 21, 22, 23, 24 or more of the 5 to 40 nucleotides are independently selected from any of the modified nucleosides shown in Table 4. In some embodiments, fewer than or equal to 25, 24, 23, 22, 21, 20, 19, 18, 17, 16, 15, 14, 13, 12, 10, 11, 10, 9, 8, 7, 6, 5, 4, 3, or 2 of the 5 to 40 nucleotides are independently selected from any of the modified nucleosides shown in Table 4.

In some embodiments, at least 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, 97%, 99%, or 100% of the 5 to 40 nucleotides are independently selected from any of the modified nucleosides shown in Table 4. In some embodiments, less than or equal to 100%, 99%, 97%, 95%, 90%, 85%, 80%, 75%, 70%, 65%, 60%, 55%, 50%, 45%, 40%, 35%, 30%, 25%, or 20% of the 5 to 40 nucleotides are independently selected from any of the modified nucleosides shown in Table 4.

In some embodiments, the modified nucleoside is a locked nucleoside, a 2′-substituted nucleoside, or a methylated nucleoside. In some embodiments, at least 1, 2, 3, 4, 5, 6, 7, 8, 9, 10, 11, 12, 13, 14, 15, 16, 17, 18, 19, 20, 21, 22, 23, 24 or more of the 5 to 40 nucleotides are locked nucleosides. In some embodiments, fewer than or equal to 25, 24, 23, 22, 21, 20, 19, 18, 17, 16, 15, 14, 13, 12, 10, 11, 10, 9, 8, 7, 6, 5, 4, 3, or 2 of the 5 to 40 nucleotides are locked nucleosides.

In some embodiments, at least 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, 97%, 99%, or 100% of the 5 to 40 nucleotides are locked nucleosides. In some embodiments, less than or equal to 100%, 99%, 97%, 95%, 90%, 85%, 80%, 75%, 70%, 65%, 60%, 55%, 50%, 45%, 40%, 35%, 30%, 25%, or 20% of the 5 to 40 nucleotides are locked nucleosides.

In some embodiments, the locked nucleoside is selected from: LNA, scpBNA, AmNA (N—H), AmNA (N-Me), GuNA, GuNA (N—R) where R is selected from Me, Et, i-Pr, t-Bu and combinations thereof.

In some embodiments, at least 1, 2, 3, 4, 5, 6, 7, 8, 9, 10, 11, 12, 13, 14, 15, 16, 17, 18, 19, 20, 21, 22, 23, 24 or more of the 5 to 40 nucleotides are 2′-substituted nucleosides. In some embodiments, fewer than or equal to 25, 24, 23, 22, 21, 20, 19, 18, 17, 16, 15, 14, 13, 12, 10, 11, 10, 9, 8, 7, 6, 5, 4, 3, or 2 of the 5 to 40 nucleotides are 2′-substituted nucleosides.

In some embodiments, at least 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, 97%, 99%, or 100% of the 5 to 40 nucleotides are 2′-substituted nucleosides. In some embodiments, less than or equal to 100%, 99%, 97%, 95%, 90%, 85%, 80%, 75%, 70%, 65%, 60%, 55%, 50%, 45%, 40%, 35%, 30%, 25%, or 20% of the 5 to 40 nucleotides are 2′-substituted nucleosides.

In some embodiments, at least 1, 2, 3, 4, 5, 6, 7, 8, 9, 10, 11, 12, 13, 14, 15, 16, 17, 18, 19, 20, 21, 22, 23, 24 or more of the 5 to 40 nucleotides are 2′-O-methoxy-ethyl (2′-MOE) nucleosides. In some embodiments, fewer than or equal to 25, 24, 23, 22, 21, 20, 19, 18, 17, 16, 15, 14, 13, 12, 10, 11, 10, 9, 8, 7, 6, 5, 4, 3, or 2 of the 5 to 40 nucleotides are 2′-MOE nucleosides.

In some embodiments, at least 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, 97%, 99%, or 100% of the 5 to 40 nucleotides are 2′-MOE nucleosides. In some embodiments, less than or equal to 100%, 99%, 97%, 95%, 90%, 85%, 80%, 75%, 70%, 65%, 60%, 55%, 50%, 45%, 40%, 35%, 30%, 25%, or 20% of the 5 to 40 nucleotides are 2′-MOE nucleosides.

In some embodiments, at least 1, 2, 3, 4, 5, 6, 7, 8, 9, 10, 11, 12, 13, 14, 15, 16, 17, 18, 19, 20, 21, 22, 23, 24 or more of the 5 to 40 nucleotides are 2′-O-methyl nucleosides. In some embodiments, fewer than or equal to 25, 24, 23, 22, 21, 20, 19, 18, 17, 16, 15, 14, 13, 12, 10, 11, 10, 9, 8, 7, 6, 5, 4, 3, or 2 of the 5 to 40 nucleotides are 2′-O-methyl nucleosides.

In some embodiments, at least 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, 97%, 99%, or 100% of the 5 to 40 nucleotides are 2′-O-methyl nucleosides. In some embodiments, less than or equal to 100%, 99%, 97%, 95%, 90%, 85%, 80%, 75%, 70%, 65%, 60%, 55%, 50%, 45%, 40%, 35%, 30%, 25%, or 20% of the 5 to 40 nucleotides are 2′-O-methyl nucleosides.

In some embodiments, at least 1, 2, 3, 4, 5, 6, 7, 8, 9, 10, 11, 12, 13, 14, 15, or more of the 5 to 40 nucleotides are 5-methylcytosines ((5m)C). In some embodiments, fewer than or equal to 20, 19, 18, 17, 16, 15, 14, 13, 12, 10, 11, 10, 9, 8, 7, 6, 5, 4, 3, or 2 of the 5 to 40 nucleotides are (5m)C.

In some embodiments, at least 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50% of the 5 to 40 nucleotides are (5m)C. In some embodiments, less than or equal to 75%, 70%, 65%, 60%, 55%, 50%, 45%, 40%, 35%, 30%, 25%, 20%, 15%, or 10% of the 5 to 40 nucleotides are (5m)C.

In some embodiments, at least 1, 2, 3, 4, 5, 6, 7, 8, 9, 10, 11, 12, 13, 14, 15, 16, 17, 18, 19, 20, 21, 22, 23, 24 or more of the 5 to 40 nucleotides are deoxyribonucleosides. In some embodiments, fewer than or equal to 25, 24, 23, 22, 21, 20, 19, 18, 17, 16, 15, 14, 13, 12, 10, 11, 10, 9, 8, 7, 6, 5, 4, 3, or 2 of the 5 to 40 nucleotides are deoxyribonucleosides.

In some embodiments, at least 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, 97%, 99%, or 100% of the 5 to 40 nucleotides are deoxyribonucleosides. In some embodiments, less than or equal to 100%, 99%, 97%, 95%, 90%, 85%, 80%, 75%, 70%, 65%, 60%, 55%, 50%, 45%, 40%, 35%, 30%, 25%, or 20% of the 5 to 40 nucleotides are deoxyribonucleosides.

In some embodiments, at least 1, 2, 3, 4, 5, 6, 7, 8, 9, 10, 11, 12, 13, 14, 15, 16, 17, 18, 19, 20, 21, 22, 23, 24 or more of the 5 to 40 nucleotides are ribonucleosides. In some embodiments, fewer than or equal to 25, 24, 23, 22, 21, 20, 19, 18, 17, 16, 15, 14, 13, 12, 10, 11, 10, 9, 8, 7, 6, 5, 4, 3, or 2 of the 5 to 40 nucleotides are ribonucleosides.

In some embodiments, at least 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, 97%, 99%, or 100% of the 5 to 40 nucleotides are ribonucleosides. In some embodiments, less than or equal to 100%, 99%, 97%, 95%, 90%, 85%, 80%, 75%, 70%, 65%, 60%, 55%, 50%, 45%, 40%, 35%, 30%, 25%, or 20% of the 5 to 40 nucleotides are ribonucleosides.

In some embodiments, at least 1, 2, 3, 4, 5, 6, 7, 8, 9, 10, 11, 12, 13, 14, 15, 16, 17, 18, 19, 20, 21, 22, 23, 24 or more of the 5 to 40 nucleotides are purines. In some embodiments, fewer than or equal to 25, 24, 23, 22, 21, 20, 19, 18, 17, 16, 15, 14, 13, 12, 10, 11, 10, 9, 8, 7, 6, 5, 4, 3, or 2 of the 5 to 40 nucleotides are purines.

In some embodiments, at least 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, 97%, 99%, or 100% of the 5 to 40 nucleotides are purines. In some embodiments, less than or equal to 100%, 99%, 97%, 95%, 90%, 85%, 80%, 75%, 70%, 65%, 60%, 55%, 50%, 45%, 40%, 35%, 30%, 25%, or 20% of the 5 to 40 nucleotides are purines.

In some embodiments, at least 1, 2, 3, 4, 5, 6, 7, 8, 9, 10, 11, 12, 13, 14, 15, 16, 17, 18, 19, 20, 21, 22, 23, 24 or more of the 5 to 40 nucleotides are pyrimidines. In some embodiments, fewer than or equal to 25, 24, 23, 22, 21, 20, 19, 18, 17, 16, 15, 14, 13, 12, 10, 11, 10, 9, 8, 7, 6, 5, 4, 3, or 2 of the 5 to 40 nucleotides are pyrimidines.

In some embodiments, at least 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, 97%, 99%, or 100% of the 5 to 40 nucleotides are pyrimidines. In some embodiments, less than or equal to 100%, 99%, 97%, 95%, 90%, 85%, 80%, 75%, 70%, 65%, 60%, 55%, 50%, 45%, 40%, 35%, 30%, 25%, or 20% of the 5 to 40 nucleotides are pyrimidines.

In some embodiments, the nucleotide sequence comprises 15 or 16 nucleotides. In some embodiments, the nucleotide sequence comprises 15 nucleotides. In some embodiments, the nucleotide sequence comprises 16 nucleotides.

In some embodiments, the oligonucleotide comprises a nucleotide modification pattern of (XY)ₙ, wherein X represents a first class of modified nucleosides, and Y represents a second class of modified nucleosides, wherein X and Y are different, and n is a number between 1 to 15.

In some embodiments, the first class of modified nucleosides is selected from locked nucleosides and 2′-O-methyl nucleosides. In some embodiments, the first class of modified nucleosides is selected from locked nucleosides, 2′-MOE nucleosides, and 2′-O-methyl nucleosides.

In some embodiments, the second class of modified nucleosides is selected from locked nucleosides and 2′-O-methyl nucleosides. In some embodiments, the second class of modified nucleosides is selected from locked nucleosides and 2′-O-methyl nucleosides, and 2′-MOE nucleosides.

In some embodiments, at least 2, 3, or 4 consecutive nucleotides in the nucleotide modification pattern comprise at least 2, 3, or 4 different nucleobases. In some embodiments, at least 2, 3, or 4 consecutive nucleotides in the nucleotide modification pattern comprise the same nucleobase.

In some embodiments, the nucleotide sequence comprises 20, 21, or 22 nucleotides.

In some embodiments, at least 50%, 60%, 70%, 75%, 80%, 85%, 90%, 95%, 96%, 97%, 98%, 99%, or 100% of the 20, 21, or 22 nucleotides are 2′-O-methyl nucleosides. In some embodiments, at least 10, 11, 12, 13, 14, 15, 16, 17, 18, 19, 20. 21, or 22 of the 20, 21, or 22 nucleotides are 2′-O-methyl nucleosides

In some embodiments, the oligonucleotide has a melting temperature (Tm) for the complementary viral target sequence of between 50 to 90° C., 60 to 90° C., 65 to 90° C., 70 to 90° C., 75 to 90° C., 80 to 90° C., or 80 to 85° C.

In some embodiments, at least 2, 3, 4, 5, 6, 7, 8, 9, 10, 11, 12, 13, 14, 15, 16, 17, 18, 19, 20, 21, 22, 23, 24 or more of the 5 to 40 nucleotides are linked by phosphorothioate linkages. In some embodiments, fewer than or equal to 25, 24, 23, 22, 21, 20, 19, 18, 17, 16, 15, 14, 13, 12, 10, 11, 10, 9, 8, 7, 6, 5, 4, 3, or 2 of the 5 to 40 nucleotides are linked by phosphorothioate linkages.

In some embodiments, at least 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, 97%, 99%, or 100% of the 5 to 40 nucleotides are linked by phosphorothioate linkages. In some embodiments, less than or equal to 100%, 99%, 97%, 95%, 90%, 85%, 80%, 75%, 70%, 65%, 60%, 55%, 50%, 45%, 40%, 35%, 30%, 25%, or 20% of the 5 to 40 nucleotides are linked by phosphorothioate linkages.

In some embodiments, the oligonucleotide further comprises a tissue targeting conjugate. In some embodiments, the tissue targeting conjugate is attached to the oligonucleotide and targets the oligonucleotide to the liver. In some embodiments, the tissue targeting conjugate comprises a galactosamine. In some embodiments, the galactosamine is N-acetylgalactosamine (GalNAc) of Formula (I):

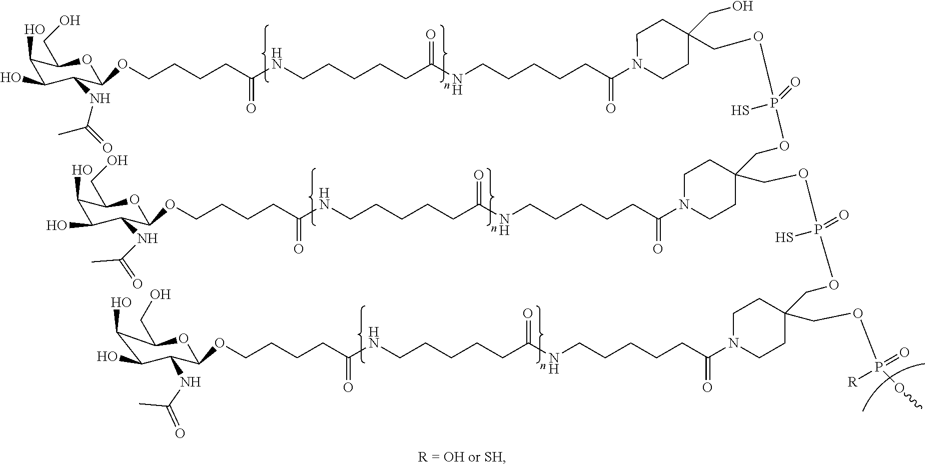

wherein each n is independently 1 or 2.

In some embodiments, the galactosamine is N-acetylgalactosamine (GalNAc) of Formula (II):

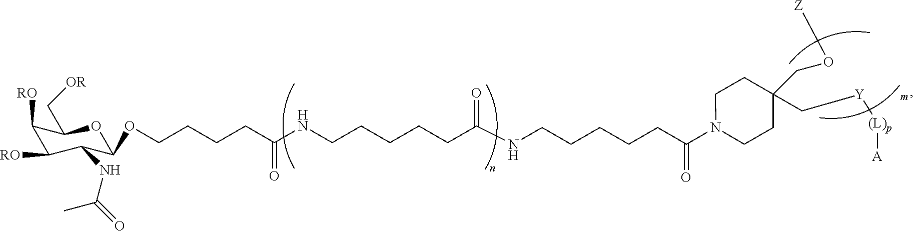

wherein
m is 1, 2, 3, 4, or 5;
each n is independently 1 or 2;
p is 0 or 1;
each R is independently H;
each Y is independently selected from —O—P(═O)(SH)—, —O—P(═O)(O)—, —O—P(═O)(OH)—, and —O—P(S)S—;
Z is H or a second protecting group;
either L is a linker or L and Y in combination are a linker; and
A is H, OH, a third protecting group, an activated group, or an oligonucleotide.

In some embodiments, the tissue targeting conjugate is attached to the 3′ end of the nucleotide sequence.

In some embodiments, the tissue targeting conjugate is attached to the 5′ end of the nucleotide sequence.

In some embodiments, the tissue targeting conjugate is attached to the nucleotide sequence via one or more linkages independently selected from a phosphodiester linkage, phosphorothioate linkage, or phosphorodithioate linkage.

In some embodiments, the tissue targeting conjugate is attached to the nucleotide sequence via a linker sequence, wherein the linker sequence comprises 1, 2, 3, 4, 5, 6, 7, 8, 9, 10, 11, 12, 13, 14, or 15 nucleotides.

In some embodiments, the linker sequence is located between the tissue targeting conjugate and the nucleotide sequence.

In some embodiments, the tissue targeting conjugate is attached to the linker sequence via one or more linkages independently selected from a phosphodiester linkage, phosphorothioate linkage, or phosphorodithioate linkage.

In some embodiments, the nucleotide sequence is selected from a sequence as shown in Tables 1-3.

In some embodiments, the nucleotide sequence comprises a sequence selected from the group consisting of SEQ ID NO: 78, 100, 161, and 171. In some embodiments, the nucleotide sequence is SEQ ID NO: 78. In some embodiments, the nucleotide sequence is SEQ ID NO: 100.

In some embodiments, the nucleotide sequence is SEQ ID NO: 161. In some embodiments, the nucleotide sequence is SEQ ID NO: 171.

In some embodiments, the oligonucleotide does not result in cleavage of the viral target sequence.

In some embodiments, the oligonucleotide reduces conversion of the rcDNA to cccDNA. In some embodiments, the oligonucleotide reduces conversion of the rcDNA to cccDNA by at least about 20%, 30%, 40%, 50%, 60%, 70%, 80%, 90%, 95%, or 100%.

In some embodiments, the oligonucleotide reduces the amount of cccDNA. In some embodiments, the oligonucleotide reduces the amount of cccDNA by at least about 20%, 30%, 40%, 50%, 60%, 70%, 80%, 90%, 95%, or 100%.

In some embodiments, the oligonucleotide results in degradation of cccDNA. In some embodiments, at least about 20%, 30%, 40%, 50%, 60%, 70%, 80%, 90%, 95%, or 100% of the cccDNA is degraded.

In some embodiments, the oligonucleotide reduces the viral titer. In some embodiments, the oligonucleotide reduces the viral titer by at least about 20%, 30%, 40%, 50%, 60%, 70%, 80%, 90%, 95%, or 100%.

In some embodiments, the oligonucleotide does not induce or activate RNAse H or RNA interference.

In some embodiments, the viral target sequence comprises at least a portion of the HBV genome of any one of HBV genotypes A-J. In some embodiments, the viral target sequence comprises at least a portion of the HBV genome of any one of HBV genotypes A-D.

In some embodiments, at least 6, 7, 8, 9, 10, 11, 12, 13, 14, 15, 16, 17, 18, 19, 20, 21, 22, 23, 24, or 25 of the 5 to 40 nucleotides is identical, complementary, hybridizes, or binds to the viral target sequence.

In some embodiments, at least 10 of the 5 to 40 nucleotides is identical, complementary, hybridizes, or binds to the viral target sequence.

In some embodiments, at least 15 of the 5 to 40 nucleotides is identical, complementary, hybridizes, or binds to the viral target sequence.

In some embodiments, at least 19 of the 5 to 40 nucleotides is identical, complementary, hybridizes, or binds to the viral target sequence.

Further disclosed herein is a composition comprising: (a) any of the oligonucleotides disclosed herein; and (b) a pharmaceutically acceptable carrier, excipient, diluent, or adjuvant.

Further disclosed herein is a composition comprising: (a) a first oligonucleotide comprising any of the oligonucleotides disclosed herein; and (b) a second oligonucleotide comprising any of the oligonucleotides disclosed herein, wherein the first and second oligonucleotide differ by at least one nucleotide.

Further disclosed herein is a composition comprising 2, 3, 4, 5, 6, 7, 8, 9 or more of any of the oligonucleotides disclosed herein, wherein the 2, 3, 4, 5, 6, 7, 8, 9 or more oligonucleotides differ by at least one nucleotide.

Further disclosed herein is a composition comprising: (a) any of the oligonucleotides disclosed herein; and (b) an anti-HBV drug.

Further disclosed herein is a composition comprising: (a) a first oligonucleotide comprising any of the oligonucleotides disclosed herein; (b) a second oligonucleotide comprising any of the oligonucleotides disclosed herein, wherein the first and second oligonucleotide differ by at least one nucleotide; and (c) an anti-HBV drug.

Further disclosed herein is a composition comprising (a) 2, 3, 4, 5, 6, 7, 8, 9 or more of any of the oligonucleotides disclosed herein, wherein the 2, 3, 4, 5, 6, 7, 8, 9 or more oligonucleotides differ by at least one nucleotide; and (b) an anti-HBV drug.

Further disclosed herein is a composition comprising: (a) any of the oligonucleotides disclosed in any of Tables 1-3; and (b) a pharmaceutically acceptable carrier, excipient, diluent, or adjuvant.

Further disclosed herein is a composition comprising: (a) a first oligonucleotide comprising any of the oligonucleotides disclosed in any of Tables 1-3; and (b) a second oligonucleotide comprising any of the oligonucleotides disclosed in any of Tables 1-3, wherein the first and second oligonucleotide differ by at least one nucleotide.

Further disclosed herein is a composition comprising 2, 3, 4, 5, 6, 7, 8, 9 or more of the oligonucleotides disclosed in any of Tables 1-3, wherein the 2, 3, 4, 5, 6, 7, 8, 9 or more oligonucleotides differ by at least one nucleotide.

Further disclosed herein is a composition comprising: (a) any of the oligonucleotides disclosed in any of Tables 1-3; and (b) an anti-HBV drug.

Further disclosed herein is a composition comprising: (a) a first oligonucleotide comprising any of the oligonucleotides disclosed in any of Tables 1-3; (b) a second oligonucleotide comprising any of the oligonucleotides disclosed in any of Tables 1-3, wherein the first and second oligonucleotide differ by at least one nucleotide; and (c) an anti-HBV drug.

Further disclosed herein is a composition comprising (a) 2, 3, 4, 5, 6, 7, 8, 9 or more of the oligonucleotides disclosed in any of Tables 1-3, wherein the 2, 3, 4, 5, 6, 7, 8, 9 or more oligonucleotides differ by at least one nucleotide; and (b) an anti-HBV drug.

In some embodiments, any of the compositions or kits disclosed herein comprise an anti-HBV drug. In some embodiments, the anti-HBV drug is selected from an oligonucleotide therapy, a capsid assembly modulator, a recombinant interferon, a nucleoside analog, and a nucleotide analog. In some embodiments, the anti-HBV drug is selected from the group consisting of include ALG-010133, ALG-000184, ALG-020572, ALG-125755, recombinant interferon alpha 2b, IFN-a, PEG-IFN-a-2a, lamivudine, telbivudine, adefovir dipivoxil, clevudine, entecavir, tenofovir alafenamide, tenofovir disoproxil, NVR3-778, BAY41-4109, JNJ-632, JNJ-3989 (ARO-HBV), RG6004, GSK3228836, REP-2139, REP-2165, AB-729, VIR-2218, DCR-HBVS (RG-6346), ALG-020572, ALG-125755, JNJ-6379, GLS4, ABI-H0731, JNJ-440, NZ-4, RG7907, EDP-514, AB-423, AB-506, ABI-H03733 and ABI-H2158. In some embodiments, the oligonucleotide therapy is selected from STOPS, siRNA, and ASO.

In some embodiments, any of the compositions disclosed herein further comprise a pharmaceutically acceptable carrier, excipient, diluent, or adjuvant.

Further disclosed herein is a kit comprising any of the oligonucleotides disclosed herein. In some embodiments, the kit comprises any of the oligonucleotides disclosed in any of Tables 1-3.

Further disclosed herein is a plasmid comprising any of the oligonucleotides disclosed herein. In some embodiments, the plasmid comprises any of the oligonucleotides disclosed in any of Tables 1-3.

Further disclosed herein is a viral vector comprising any of the oligonucleotides disclosed herein. Further disclosed herein is a viral vector comprising any of the oligonucleotides disclosed in any of Tables 1-3.

Further disclosed herein is a particle comprising any of the oligonucleotides disclosed herein. In some embodiments, the particle comprises any of the oligonucleotides disclosed in any of Tables 1-3.

Further disclosed herein are methods of reducing conversion of hepatitis B virus (HBV) relaxed circular DNA (rcDNA) to covalently closed circular DNA (cccDNA) conversion. In some embodiments, the method of reducing conversion of hepatitis B virus (HBV) relaxed circular DNA (rcDNA) to covalently closed circular DNA (cccDNA) conversion comprises contacting a cell with any of the oligonucleotides disclosed herein. In some embodiments, the method of reducing conversion of hepatitis B virus (HBV) relaxed circular DNA (rcDNA) to covalently closed circular DNA (cccDNA) conversion comprises contacting a cell with any of the oligonucleotides disclosed in any of Tables 1-3. In some embodiments, the method of reducing conversion of hepatitis B virus (HBV) relaxed circular DNA (rcDNA) to covalently closed circular DNA (cccDNA) conversion comprises contacting a cell with 2, 3, 4, 5, 6, 7, 8, 9 or more of any oligonucleotides disclosed herein. In some embodiments, the method of reducing conversion of hepatitis B virus (HBV) relaxed circular DNA (rcDNA) to covalently closed circular DNA (cccDNA) conversion comprises contacting a cell with 2, 3, 4, 5, 6, 7, 8, 9 or more of the oligonucleotides disclosed in any of Tables 1-3.

Further disclosed herein are methods of targeting hepatitis B virus (HBV) covalently closed circular DNA (cccDNA) for degradation. In some embodiments, the method of targeting hepatitis B virus (HBV) covalently closed circular DNA (cccDNA) for degradation, comprises contacting a cell with any oligonucleotides disclosed herein. In some embodiments, the method of targeting hepatitis B virus (HBV) covalently closed circular DNA (cccDNA) for degradation, comprises contacting a cell with any oligonucleotides disclosed in any of Tables 1-3. In some embodiments, the method of targeting hepatitis B virus (HBV) covalently closed circular DNA (cccDNA) for degradation, comprises contacting a cell with 2, 3, 4, 5, 6, 7, 8, 9 or more of any of the oligonucleotides disclosed herein. In some embodiments, the method of targeting hepatitis B virus (HBV) covalently closed circular DNA (cccDNA) for degradation, comprises contacting a cell with 2, 3, 4, 5, 6, 7, 8, 9 or more of the oligonucleotides disclosed in any of Tables 1-3.

Further disclosed herein are methods of reducing the amount of hepatitis B virus (HBV) covalently closed circular DNA (cccDNA) in a cell. In some embodiments, the method of reducing the amount of hepatitis B virus (HBV) covalently closed circular DNA (cccDNA) in a cell, comprising contacting the cell with any oligonucleotides disclosed herein. In some embodiments, the method of reducing the amount of hepatitis B virus (HBV) covalently closed circular DNA (cccDNA) in a cell, comprising contacting the cell with any oligonucleotides disclosed in any of Tables 1-3. In some embodiments, the method of reducing the amount of hepatitis B virus (HBV) covalently closed circular DNA (cccDNA) in a cell, comprising contacting the cell with 2, 3, 4, 5, 6, 7, 8, 9 or more of any of the oligonucleotides disclosed herein. In some embodiments, the method of reducing the amount of hepatitis B virus (HBV) covalently closed circular DNA (cccDNA) in a cell, comprising contacting the cell with 2, 3, 4, 5, 6, 7, 8, 9 or more of the oligonucleotides disclosed in any of Tables 1-3.

In some embodiments, any of the methods disclosed herein further comprise detecting levels of at least one of: cccDNA or a surrogate marker of cccDNA. In some embodiments, the surrogate marker of cccDNA is selected from hepatitis B surface antigen (HBsAg), hepatitis B core antigen (HBc-Ag), hepatitis B e antigen (HBeAg), HBV polymerase, and HBV X protein (HBx).

In some embodiments, detecting comprises performing at least one of: a Southern blot, polymerase chain reaction (PCR), Invader assay, in situ hybridization, HBV DNA assay, HBV antigen assay, or HBV antibody assay. In some embodiments, the HBV antigen assay is selected from an HBs antigen assay and HBe antigen assay. In some embodiments, the HBV antibody assay is selected from anti-HBs antibody assay, anti-HBc IgM antibody assay, anti-HBc antibody assay, and anti-HBe antibody assay.

In some embodiments, the cell is from a biological sample from a subject suffering from HBV or suspected of suffering from HBV.

In some embodiments, the biological sample is a blood sample. In some embodiments, the blood sample is a serum sample.

In some embodiments, any of the methods disclosed herein further comprise contacting the cell with at least 1, 2, 3, 4, or 5 additional oligonucleotides of any one of claims 1-123, wherein the oligonucleotides of any one of claims 1-123 differ by at least 1 nucleotide.

In some embodiments, any of the methods disclosed herein further comprise contacting the cell with an anti-HBV drug.

In some embodiments, the cell is contacted with the oligonucleotide and the anti-HBV drug simultaneously.

In some embodiments, the cell is contacted with the oligonucleotide and the anti-HBV drug sequentially.

In some embodiments, the anti-HBV drug is selected from an oligonucleotide therapy, a capsid assembly modulator, a recombinant interferon, a nucleoside analog, and a nucleotide analog. In some embodiments, the anti-HBV drug is selected from the group consisting of include ALG-010133, ALG-000184, ALG-020572, ALG-125755, recombinant interferon alpha 2b, IFN-α, PEG-IFN-α-2a, lamivudine, telbivudine, adefovir dipivoxil, clevudine, entecavir, tenofovir alafenamide, tenofovir disoproxil, NVR3-778, BAY41-4109, JNJ-632, JNJ-3989 (ARO-HBV), RG6004, GSK3228836, REP-2139, REP-2165, AB-729, VIR-2218, DCR-HBVS (RG-6346), ALG-020572, ALG-125755, JNJ-6379, GLS4, ABI-H0731, JNJ-440, NZ-4, RG7907, EDP-514, AB-423, AB-506, ABI-H03733 and ABI-H2158. In some embodiments, the oligonucleotide therapy is selected from STOPS, siRNA, and ASO.

Further disclosed herein are methods of treating a hepatitis B virus infection in a subject in need thereof. In some embodiments, the method of treating a hepatitis B virus infection in a subject in need thereof, comprising administering to the subject any of the oligonucleotides disclosed herein. In some embodiments, the method of treating a hepatitis B virus infection in a subject in need thereof, comprising administering to the subject any of the oligonucleotides disclosed in any of Tables 1-3. In some embodiments, the method of treating a hepatitis B virus infection in a subject in need thereof, comprising administering to the subject 2, 3, 4, 5, 6, 7, 8, 9 or more of any of the oligonucleotides disclosed herein. In some embodiments, the method of treating a hepatitis B virus infection in a subject in need thereof, comprising administering to the subject 2, 3, 4, 5, 6, 7, 8, 9 or more of the oligonucleotides disclosed in any of Tables 1-3. In some embodiments, the method of treating a hepatitis B virus infection in a subject in need thereof, comprising administering to the subject any of the compositions disclosed herein.

In some embodiments, any of the methods disclosed herein further comprise detecting levels of at least one of: cccDNA or a surrogate marker of cccDNA in a biological sample from the subject. In some embodiments, the surrogate marker of cccDNA is selected from hepatitis B surface antigen (HBsAg), hepatitis B core antigen (HBc-Ag), hepatitis B e antigen (HBeAg), HBV polymerase, and HBV X protein (HBx).

In some embodiments, detecting comprises performing at least one of: a Southern blot, polymerase chain reaction (PCR), Invader assay, in situ hybridization, HBV DNA assay, HBV antigen assay, or HBV antibody assay. In some embodiments, the HBV antigen assay is selected from an HBs antigen assay and HBe antigen assay. In some embodiments, the HBV antibody assay is selected from anti-HBs antibody assay, anti-HBc IgM antibody assay, anti-HBc antibody assay, and anti-HBe antibody assay.

In some embodiments, the biological sample is a blood sample. In some embodiments, the blood sample is a serum sample.

In some embodiments, any of the methods disclosed herein further comprise modifying the dose or dosing regimen of the oligonucleotide administered to the subject based on the levels of the cccDNA or surrogate marker detected.

In some embodiments, the dose or dosing region of the oligonucleotide is decreased when the levels of the cccDNA or surrogate marker is decreased, wherein the levels of the cccDNA or surrogate marker is decreased as compared to (a) the levels of the cccDNA or surrogate marker in the subject from an earlier time point; or (b) levels of the cccDNA or surrogate marker in a control sample.

In some embodiments, the earlier time point is (a) prior to administering the oligonucleotide to the subject; or (b) after administering an initial dose of the oligonucleotide to the subject, but prior to administering a subsequent dose of the oligonucleotide to the subject.

In some embodiments, any of the methods disclosed herein further comprise administering to the subject one or more anti-HBV therapies.

In some embodiments, the oligonucleotide and the one or more anti-HBV therapies are administered concurrently.

In some embodiments, the oligonucleotide and the one or more anti-HBV therapies are administered sequentially.

In some embodiments, the one or more anti-HBV therapies is selected from an oligonucleotide therapy, a capsid assembly modulator, a recombinant interferon, a nucleoside analog, and a nucleotide analog. In some embodiments, the one or more anti-HBV therapies is selected from the group consisting of include ALG-010133, ALG-000184, ALG-020572, ALG-125755, recombinant interferon alpha 2b, IFN-α, PEG-IFN-α-2a, lamivudine, telbivudine, adefovir dipivoxil, clevudine, entecavir, tenofovir alafenamide, tenofovir disoproxil, NVR3-778, BAY41-4109, JNJ-632, JNJ-3989 (ARO-HBV), RG6004, GSK3228836, REP-2139, REP-2165, AB-729, VIR-2218, DCR-HBVS (RG-6346), ALG-020572, ALG-125755, JNJ-6379, GLS4, ABI-H0731, JNJ-440, NZ-4, RG7907, EDP-514, AB-423, AB-506, ABI-H03733 and ABI-H2158. In some embodiments, the oligonucleotide therapy is selected from STOPS, siRNA, and ASO.

In some embodiments, any of the methods disclosed herein further comprise administering at least 1, 2, 3, 4, or 5 additional oligonucleotides, wherein the additional oligonucleotides are any of the oligonucleotides disclosed herein, and wherein the oligonucleotides differ by at least 1 nucleotide.

In some embodiments, any of the methods disclosed herein further comprise administering at least 1, 2, 3, 4, or 5 additional oligonucleotides, wherein the additional oligonucleotides are any of the oligonucleotides disclosed in any of Tables 1-3, and wherein the oligonucleotides differ by at least 1 nucleotide.

In some embodiments, two or more of the oligonucleotides disclosed herein are administered concurrently.

In some embodiments, two or more of the oligonucleotides disclosed herein are administered sequentially.

In some embodiments, any of the oligonucleotides disclosed herein is administered by parenteral injection, intravenous (IV) infusion, or subcutaneous injection.

In some embodiments, the HBV is any one of HBV genotypes A-J. In some embodiments, the HBV is any one of HBV genotypes A-D.

Further disclosed here are uses of any of the oligonucleotides disclosed herein in the manufacture of a medicament to treat HBV infection in a subject in need thereof. Further disclosed here are uses of any of the oligonucleotides disclosed in any of Tables 1-3 in the manufacture of a medicament to treat HBV infection in a subject in need thereof. In some embodiments, the oligonucleotide is formulated for parenteral injection, intravenous (IV) infusion, or subcutaneous injection.

Further disclosed here are uses of any of the compositions disclosed herein in the manufacture of a medicament to treat HBV infection in a subject in need thereof. In some embodiments, the composition is formulated for parenteral injection, intravenous (IV) infusion, or subcutaneous injection.

### BRIEF DESCRIPTION OF THE DRAWINGS

FIG. 1 provides a schematic of strategies for targeting HBV, emphasizing on the strategies to targeting rcDNA and cccDNA.

FIG. 2 provides an exemplary schematic of an HBV genome emphasizing on the gap region as well as the promoters and regulatory elements of HBV transcripts.

FIG. 3A-3C shows the effects of the Steric Blocker SEQ ID NO: 161 of rcDNA and cccDNA in HepG2-NTCP infected with HBV. 3A illustrates a reduction of both rc and cccDNA via Southern Blot analysis. 3B illustrates the percentage of cccDNA amounts in SEQ ID NO: 161 treated cells compared with no treatment control. Each cccDNA signal is normalized with Cox3 signal. 3C. illustrates cell viability measured by CCK-8 assay.

FIG. 4A-4C shows the effects of the Steric Blocker SEQ ID NO: 93 on cccDNA in HepG2-NTCP infected with HBV. 4A illustrates a reduction of cccDNA via Southern Blot analysis. 4B illustrates the percentage of cccDNA amounts in SEQ ID NO: 93 treated cells compared with no treatment control. Each cccDNA signal is normalized with Cox3 signal. 4C. illustrates cell viability measured by CCK-8 assay.

FIG. 5A-5C shows the effects of the Steric Blocker SEQ ID NO: 95 on rc and cccDNA in HepG2-NTCP infected with HBV. 5A illustrates a reduction of cccDNA via Southern Blot analysis. 5B illustrates the percentage of cccDNA amounts in SEQ ID NO: 95 treated cells compared with no treatment control. Each cccDNA signal is normalized with Cox3 signal. 5C. illustrates cell viability measured by CCK-8 assay.

FIG. 6A-6C shows the effects of the Steric Blocker SEQ ID NO: 78 on cccDNA in HepG2-NTCP infected with HBV. 6A illustrates a reduction of cccDNA via Southern Blot analysis. 6B illustrates the percentage of cccDNA amounts in SEQ ID NO: 78 treated cells compared with no treatment control. Each cccDNA signal is normalized with Cox3 signal. 6C. illustrates cell viability measured by CCK-8 assay.

FIG. 7A-7C shows the effects of the Steric Blocker SEQ ID NO: 122 on cccDNA in HepG2-NTCP infected with HBV. 7A illustrates a reduction of cccDNA via Southern Blot analysis. 7B illustrates the percentage of cccDNA amounts in SEQ ID NO: 122 treated cells, compared with no treatment control. Each cccDNA signal is normalized with Cox3 signal. 7C. illustrates cell viability measured by CCK-8 assay.

FIG. 8A-8C shows the effects of the Steric Blocker SEQ ID NO: 75 on cccDNA in HepG2-NTCP infected with HBV. 8A illustrates a reduction of cccDNA via Southern Blot analysis. 8B illustrates the percentage of cccDNA amounts in SEQ ID NO: 75 treated cells compared with no treatment control. Each cccDNA signal is normalized with Cox3 signal. 8C. illustrates cell viability measured by CCK-8 assay.

FIG. 9A-9C shows the effects of the Steric Blocker SEQ ID NO: 77 on cccDNA in HepG2-NTCP infected with HBV. 9A illustrates a reduction of cccDNA via Southern Blot analysis. 9B illustrates the percentage of cccDNA amounts in SEQ ID NO: 77 treated cells compared with no treatment control. Each cccDNA signal is normalized with Cox3 signal. 9C. illustrates cell viability measured by CCK-8 assay.

FIG. 10A-10C shows the effects of the Steric Blocker SEQ ID NO: 171 on cccDNA in HepG2-NTCP infected with HBV. 10A illustrates a reduction of cccDNA via Southern Blot analysis. 10B illustrates the percentage of cccDNA amounts in SEQ ID NO: 171 treated cells compared with no treatment control. Each cccDNA signal is normalized with Cox3 signal. 10C. illustrates cell viability measured by CCK-8 assay.

FIG. 11A-11C shows the effects of the Steric Blocker SEQ ID NO: 161 and SEQ ID NO: 78 on cccDNA in PHH cells infected with HBV. 11A illustrates the dosing scheme of Steric Blockers SEQ ID NO: 161 and 78. 11B illustrates a reduction of cccDNA via Southern Blot analysis (left panel; treated with SEQ ID NO: 78 and right panel; treated with SEQ ID NO: 161). 11C illustrates the percentage of cccDNA amounts in Steric Blocker treated cells compared with no treatment control (left panel; treated with SEQ ID NO: 78 and right panel; treated with SEQ ID NO: 161) The cccDNA signals were normalized with mitochondrial DNA ND-1 signals.

FIG. 12A-B shows the effects of the Steric Blocker SEQ ID NO: 100 on cccDNA in PHH cells infected with HBV. 12A illustrates a reduction of cccDNA via Southern Blot analysis. 12B illustrates the percentage of cccDNA amounts in SEQ ID NO: 100 treated cells compared with no treatment control. The cccDNA signals were normalized with mitochondrial DNA ND-1 signals.

### DETAILED DESCRIPTION

Disclosed herein are oligonucleotides that are identical, complementary, hybridize or bind to hepatitis B virus (HBV) nucleic acid sequences (e.g., viral target sequence), such as the rcDNA and cccDNA forms of the HBV genome and HBV transcripts. These oligonucleotides may be referred to as HBV-targeting oligonucleotides. The oligonucleotides may be identical, complementary, hybridize or bind to the gap region of rcDNA or cccDNA. Alternatively, or additionally, the oligonucleotides may be identical, complementary, hybridize or bind to the non-gap regions of rcDNA or cccDNA. The oligonucleotides may be identical, complementary, hybridize or bind to an HBV transcript, such pgRNA, pre-Core RNA, pre-S1 RNA, pre-S2 RNA and X RNA. The oligonucleotides may be identical, complementary, hybridize or bind to a promoter or enhancer region of the HBV transcript. Alternatively, or additionally, the oligonucleotides may be identical, complementary, hybridize or bind to a region upstream of the promoter or enhancer region of the HBV transcript. The oligonucleotides may be identical, complementary, hybridize or bind to a region downstream of the promoter or enhancer region of the HBV transcript. The oligonucleotides may be identical, complementary, hybridize or bind to a region within 1000, 900, 800, 700, 600, 500, 400, 300, 200, 100, or 50 base pairs (or nucleotides) of the promoter or enhancer region of the HBV transcript. In some embodiments, the oligonucleotides are incorporated into the cccDNA form of HBV. In some embodiments, hybridization, or binding of the oligonucleotides to the viral target sequence does not activate or induce RNA silencing via the RNAse H mechanism or RNA-induced silencing complex (RISC). In some embodiments, hybridization, or binding of the oligonucleotides to a complement of the viral target sequence does not activate or induce RNA silencing via the RNAse H mechanism or RNA-induced silencing complex (RISC). Further disclosed herein are compositions and kits comprising such oligonucleotides. Further disclosed herein are uses of such oligonucleotides and compositions to reduce rcDNA to cccDNA conversion, reduce cccDNA levels, and/or treat an HBV infection. In some embodiments, binding of the oligonucleotides to the viral target results in (a) a reduction of rcDNA to cccDNA conversion; (b) a reduction in cccDNA levels in a cell that has been contacted with the oligonucleotides; (c) a reduction in the number of cells containing cccDNA; (d) a reduction in the number of virally infected cells in a subject infected with HBV; or (e) a reduction in viral titers, wherein the reduction is based on a comparison to a control cell or control sample. In some embodiments, the control cell or control sample is a cell or sample that has not been contacted with the oligonucleotides. Alternatively, the control cell or control sample is a cell or sample from a subject suffering from HBV that has not been administered the oligonucleotide. In some embodiments, the control cell or control sample is a cell or sample from a subject suffering from HBV that has been administered the oligonucleotide, wherein the control cell or control sample is obtained from the subject prior to administration of a second or subsequent dose of the oligonucleotide.

HBV is an enveloped DNA virus that belongs to the Hepadnaviridae family. It contains a small, partially double-stranded (DS), relaxed-circular DNA (rcDNA) genome that replicates by reverse transcription of an RNA intermediate, the pregenomic RNA (pgRNA). It has a genome length of between 3182 and 3248 bp, depending on its genotype. The genome encodes four overlapping open reading frames (ORFs) that are translated into viral core protein, surface proteins, polymerase/reverse transcriptase (RT), and HBx.

FIG. 2 shows an exemplary schematic of the HBV genome. An exemplary HBV genome sequence is shown in SEQ ID NO: 1, corresponding to Genbank Accession No. KC315400.1, which is incorporated by reference in its entirety. Nucleotides 2307 . . . 3215, 1 . . . 1623 of SEQ ID NO: 1 correspond to the polymerase/RT gene sequence, which encodes for the polymerase protein. Nucleotides 2848 . . . 3215, 1 . . . 835 of SEQ ID NO: 1 correspond to the PreS1/S2/S gene sequence, which encodes for the large S protein. Nucleotides 3205 . . . 3215, 1 . . . 835 of SEQ ID NO: 1 correspond to the PreS2/S gene sequence, which encodes for the middle S protein. Nucleotides 155 . . . 835 of SEQ ID NO: 1 correspond to the S gene sequence, which encodes the small S protein. Nucleotides 1374 . . . 1838 of SEQ ID NO: 1 correspond to the X gene sequence, which encodes the X protein. Nucleotides 1814 . . . 2452 of SEQ ID NO: 1 correspond to the PreC/C gene sequence, which encodes the precore/core protein. Nucleotides 1901 . . . 2452 of SEQ ID NO: 1 correspond to the C gene sequence, which encodes the core protein. The HBV genome further comprises viral regulatory elements, such as viral promoters (preS2, preS1, Core, and X) and enhancer elements (Enh1 and Enh2). Nucleotides 1624 . . . 1771 of SEQ ID NO: 1 correspond to Enh2. Nucleotides 1742 . . . 1849 of SEQ ID NO: 1 correspond to the Core promoter. Nucleotides 1818 . . . 3215, 1 . . . 1930 of SEQ ID NO: 1 correspond to the pregenomic RNA (pgRNA), which encodes the core and polymerase proteins.

Another exemplary HBV genome sequence is shown in SEQ ID NO: 2, corresponding to Genbank Accession No. AM282986.1, which is incorporated by reference in its entirety. Nucleotides 2835 . . . 3221, 2854 . . . 3221, 1 . . . 835, 1 . . . 1930, 2716 . . . 2834 of SEQ ID NO: 2 correspond to the 51 gene sequence. Nucleotides 2835 . . . 3221, 1 . . . 1930 of SEQ ID NO: 2 correspond to the large S mRNA transcript. Nucleotides 2854 . . . 3211, 1 . . . 835 of SEQ ID NO: 2 correspond to the coding sequence (CDS) for the large surface protein. Nucleotides 3185 . . . 3221, 3211 . . . 3221, 1 . . . 835, 1 . . . 1930, 2966 . . . 3154, 3185 of SEQ ID NO: 2 correspond to the S2 gene sequence. Nucleotides 3185 . . . 3221, 1 . . . 1930 of SEQ ID NO: 2 corresponds to the middle S mRNA transcript. Nucleotides 3211 . . . 3221, 1 . . . 835 of SEQ ID NO: 2 corresponds to the CDS for the middle surface protein. Nucleotides 1 . . . 3221, 1626 . . . 1817, 1901 . . . 2458 of SEQ ID NO: 2 correspond to the C gene sequence. Nucleotides 1 . . . 3221, 1814 . . . 2458 of SEQ ID NO: 2 correspond to the pre C/C gene sequence. Nucleotides 155 . . . 835 of SEQ ID NO: 2 correspond to the CDS for the small S protein/HbsAg. Nucleotides 900 . . . 1310 of SEQ ID NO: 2 correspond to Enh1. Nucleotides 950 . . . 1930 of SEQ ID NO: 2 correspond to the X gene sequence. Nucleotides 950 . . . 1310 of SEQ ID NO: 2 correspond to the X gene promoter. Nucleotides 1310 . . . 1930 of SEQ ID NO: 2 correspond to the X mRNA transcript. Nucleotides 1374 . . . 1838 of SEQ ID NO: 2 correspond to the CDS for the X protein. Nucleotides 1403 . . . 1626 of SEQ ID NO: 2 correspond to the C gene sequence. Nucleotides 1626 . . . 1817 of SEQ ID NO: 2 correspond to the core promoter. Nucleotides 1801 . . . 3221, 1 . . . 1930 of SEQ ID NO: 2 correspond to the precore mRNA transcript. Nucleotides 1814 . . . 2458 of SEQ ID NO: 2 correspond to the CDS for the precore/HBeAg. Nucleotides 1636 . . . 1744 of SEQ ID NO: 2 correspond to the Enh2.

Additional HBV genome sequences are known in the art, including the corresponding regions for the polymerase, S, X, and C genes and promoters and enhancer elements. The oligonucleotides disclosed herein are capable of targeting various HBV genotypes and are not limited to HBV having the genome of SEQ ID NO: 1 or SEQ ID NO: 2. HBV genotypes include, but are not limited to, HBV genotypes A, B, C and D. Exemplary HBV genomes include, but are not limited to, the genomic sequences disclosed as Genbank Accession Nos. JN827419.1, GQ205440.1, EU939627.1, JQ688405.1, GU815618.1, LC456127.1, GU815633.1, GU815632.1, GQ924627.1, GQ924603.1, AB073828.1, MF674449.1, MF674427.1, KJ410517.1, JQ801479.1, GU815624.1, GU815561.1, GU815559.1, EU139543.1, JQ040125.1, KJ803803.1, KJ173420.1, JX507215.1, JX429908.1, GU815672.1, GU815654.1, GU815653.1, GU815647.1, GU815628.1, GU815626.1, GU815620.1, GU815565.1, AB471855.1, GQ377547.1, AB300364.1, DQ448623.1, GU815615.1, KJ173425.1, MH061283.1, KU963956.1, KJ803802.1, KJ173414.1, KJ173409.1, KJ173407.1, KJ173365.1, GU815676.1, GU815673.1, GU815669.1, GU815668.1, GU815664.1, GU815663.1, GU815662.1, GU815659.1, GU815658.1, GU815645.1, GU815636.1, GU815623.1, GU815619.1, GU815566.1, GU815562.1, GU815555.1, GQ924610.1, GQ377558.1, DQ993697.1, AB073834.1, and JQ040171.1, each of which are incorporated by reference in their entireties.

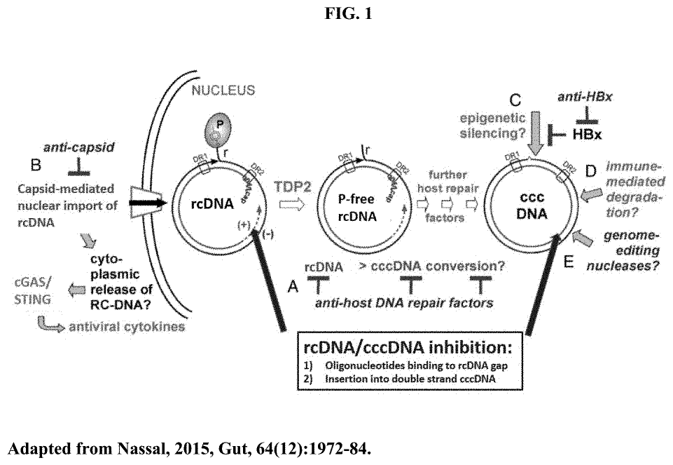

Without wishing to be bound by theory, as shown in FIG. 1, the oligonucleotides disclosed herein act as an rcDNA or cccDNA inhibitor by one or more of the following mechanisms: reducing the rcDNA to cccDNA conversion, targeting the cccDNA for degradation, or silencing cccDNA transcription. In some embodiments, the oligonucleotides reduce the rcDNA to cccDNA conversion by binding to the rcDNA gap region and acting as a steric blocker to reduce the formation of cccDNA. For fully circular cccDNA, it is known that in certain regions of the cccDNA molecule, the double strands of the cccDNA can be transiently separated during active transcription events (e.g., forming a transcription bubble) (Nur K. Mohd-Ismail et al., Int. J. Mol. Sci., 20:4276, 2019). It has also been reported that in certain DNA sequences and structures (such as quadruplex, cruciform, H-DNA, etc.), single strand DNA can exist in overall supercoiled plasmid or plasmid-like double-strand circular DNA (Kouzine et al., Cell Systems, 4:344-356, 2017). Without wishing to be bound by theory, the separation of the double stranded cccDNA or the presence of single stranded DNA in the cccDNA provide opportunities for the oligonucleotide disclosed herein to hybridize to these regions of cccDNA. In addition, the oligonucleotides disclosed herein may form triple stranded DNA (e.g., triplex-DNA) with the double stranded DNA regions of the cccDNA. Without wishing to be bound by theory, in triple-stranded DNA, the third strand (e.g., the HBV targeting oligonucleotides) binds to a B-form DNA (via Watson—Crick base-pairing) double helix by forming Hoogsteen base pairs or reversed Hoogsteen hydrogen bonds. The triplex structure could impede transcription of cccDNA or attract DNA repair mechanism, thus affecting the stability of cccDNA. The presence of the oligonucleotide in the double-stranded cccDNA may target the cccDNA for degradation or silence cccDNA transcription. In some embodiments, the oligonucleotides of the present invention differ from antisense oligonucleotides (ASOs) and siRNA such that upon binding of the oligonucleotides of the present invention to viral target sequences (e.g., HBV transcripts), the oligonucleotides do not activate or induce RNA silencing via the RNase H mechanism or RISC. Thus, in some embodiments, the binding of the oligonucleotides to the DNA or RNA forms of the HBV genome do not induce RNA silencing.

In some embodiments, the oligonucleotide comprises a nucleotide sequence comprising at least 5, 6, 7, 8, 9, or 10 nucleotides, wherein one or more of the 5, 6, 7, 8, 9, or 10 nucleotides is a modified nucleoside, wherein at least 5, 6, 7, 8, 9, or 10 nucleotides of the nucleotide sequence is identical to, complementary, hybridizes, or binds to a viral target sequence. In some embodiments, the oligonucleotide comprises a nucleotide sequence comprising at least 10 nucleotides, wherein one or more of the 10 nucleotides is a modified nucleoside, wherein at least 10 nucleotides of the nucleotide sequence are identical to, complementary, hybridizes, or binds to a viral target sequence. In some embodiments, the viral target sequence is in the rcDNA form of the HBV genome. In some embodiments, the viral target sequence is in the cccDNA form of the HBV genome. In some embodiments, the viral target sequence is in a gap region of the rcDNA or cccDNA form of the HBV genome. In some embodiments, the viral target sequence is in a non-gap region of the rcDNA or cccDNA form of the HBV genome. In some embodiments, the viral target sequence comprises a single strand of the cccDNA double strand region that is transiently opened at a transcription fork. In some embodiments, the viral target sequence comprises a single stranded region of the HBV genome. In some embodiments, the viral target sequence comprises a double stranded region of the HBV genome. In some embodiments, the oligonucleotide binds to the double stranded region of the HBV genome and forms Hoogsteen base pairs or reversed Hoogsteen hydrogen bonds. In some embodiments, the viral target sequence is in the X region of the HBV genome. In some embodiments, the viral target sequence is in the S region of the HBV genome. In some embodiments, the viral target sequence is in the HBV transcript. In some embodiments, the HBV transcript is selected from pre-genomic RNA (pgRNA), pre-Core RNA, pre-S1 RNA, pre-S2 RNA and X RNA. In some embodiments, the viral target sequence is in a promoter region of the HBV transcript. In some embodiments, the viral target sequence is in an enhancer region of the HBV transcript. In some embodiments, the viral target sequence is upstream of the promoter or enhancer region of the HBV transcript. In some embodiments, the viral target sequence is downstream of the promoter or enhancer region of the HBV transcript. In some embodiments, the viral target sequence is within about 2000, 1900, 1800, 1700, 1600, 1500, 1400, 1300, 1200, 1100, 1000, 900, 800, 700, 600, 500, 400, 300, 200, or 100 nucleotides of the promoter or enhancer region of the HBV transcript.

In some embodiments, the oligonucleotide comprises a nucleotide sequence comprising 5 to 40 nucleotides, wherein one or more of the 5 to 40 nucleotides is a modified nucleoside, wherein at least 5 consecutive nucleotides of the 5 to 40 nucleotides are identical, complementary, hybridizes, or binds to a viral target sequence. In some embodiments, the oligonucleotide comprises a nucleotide sequence comprising 5 to 40 nucleotides, wherein one or more of the 5 to 40 nucleotides is a modified nucleoside, wherein at least 10 consecutive nucleotides of the 5 to 40 nucleotides are identical, complementary, hybridizes, or binds to a viral target sequence. In some embodiments, the viral target sequence is in the rcDNA form of the HBV genome. In some embodiments, the viral target sequence is in the cccDNA form of the HBV genome. In some embodiments, the viral target sequence is in a gap region of the rcDNA or cccDNA form of the HBV genome. In some embodiments, the viral target sequence is in a non-gap region of the rcDNA or cccDNA form of the HBV genome. In some embodiments, the viral target sequence comprises a single strand of the cccDNA double strand region that is transiently opened at a transcription fork. In some embodiments, the viral target sequence comprises a single stranded region of the HBV genome. In some embodiments, the viral target sequence comprises a double stranded region of the HBV genome. In some embodiments, the oligonucleotide binds to the double stranded region of the HBV genome and forms Hoogsteen base pairs or reversed Hoogsteen hydrogen bonds. In some embodiments, the viral target sequence is in the X region of the HBV genome. In some embodiments, the viral target sequence is in the S region of the HBV genome. In some embodiments, the viral target sequence is in the HBV transcript. In some embodiments, the HBV transcript is selected from pre-genomic RNA (pgRNA), pre-Core RNA, pre-S1 RNA, pre-S2 RNA and X RNA. In some embodiments, the viral target sequence is in a promoter region of the HBV transcript. In some embodiments, the viral target sequence is in an enhancer region of the HBV transcript. In some embodiments, the viral target sequence is upstream of the promoter or enhancer region of the HBV transcript. In some embodiments, the viral target sequence is downstream of the promoter or enhancer region of the HBV transcript. In some embodiments, the viral target sequence is within about 2000, 1900, 1800, 1700, 1600, 1500, 1400, 1300, 1200, 1100, 1000, 900, 800, 700, 600, 500, 400, 300, 200, or 100 nucleotides of the promoter or enhancer region of the HBV transcript.

In some embodiments, the oligonucleotide comprises a nucleotide sequence comprising 5 to 40 nucleotides, wherein one or more of the 5 to 40 nucleotides is a locked nucleoside, wherein at least 5 consecutive nucleotides of the 5 to 40 nucleotides are identical to, complementary, hybridizes, or binds to a viral target sequence. In some embodiments, the oligonucleotide comprises a nucleotide sequence comprising 5 to 40 nucleotides, wherein one or more of the 5 to 40 nucleotides is a locked nucleoside, wherein at least 10 consecutive nucleotides of the 5 to 40 nucleotides are identical to, complementary, hybridizes, or binds to a viral target sequence. In some embodiments, the viral target sequence is in the rcDNA form of the HBV genome. In some embodiments, the viral target sequence is in the cccDNA form of the HBV genome. In some embodiments, the viral target sequence is in a gap region of the rcDNA or cccDNA form of the HBV genome. In some embodiments, the viral target sequence is in a non-gap region of the rcDNA or cccDNA form of the HBV genome. In some embodiments, the viral target sequence comprises a single strand of the cccDNA double strand region that is transiently opened at a transcription fork. In some embodiments, the viral target sequence comprises a single stranded region of the HBV genome. In some embodiments, the viral target sequence comprises a double stranded region of the HBV genome. In some embodiments, the oligonucleotide binds to the double stranded region of the HBV genome and forms Hoogsteen base pairs or reversed Hoogsteen hydrogen bonds. In some embodiments, the viral target sequence is in the X region of the HBV genome. In some embodiments, the viral target sequence is in the S region of the HBV genome. In some embodiments, the viral target sequence is in the HBV transcript. In some embodiments, the HBV transcript is selected from pre-genomic RNA (pgRNA), pre-Core RNA, pre-S1 RNA, pre-S2 RNA and X RNA. In some embodiments, the viral target sequence is in a promoter region of the HBV transcript. In some embodiments, the viral target sequence is in an enhancer region of the HBV transcript. In some embodiments, the viral target sequence is upstream of the promoter or enhancer region of the HBV transcript. In some embodiments, the viral target sequence is downstream of the promoter or enhancer region of the HBV transcript. In some embodiments, the viral target sequence is within about 2000, 1900, 1800, 1700, 1600, 1500, 1400, 1300, 1200, 1100, 1000, 900, 800, 700, 600, 500, 400, 300, 200, or 100 nucleotides of the promoter or enhancer region of the HBV transcript.

In some embodiments, the oligonucleotide comprises a nucleotide sequence comprising 5 to 40 nucleotides, wherein one or more of the 5 to 40 nucleotides is a 2′-substituted nucleoside, wherein at least 5 consecutive nucleotides of the 5 to 40 nucleotides are identical to, complementary, hybridizes, or binds to a viral target sequence. In some embodiments, the oligonucleotide comprises a nucleotide sequence comprising 5 to 40 nucleotides, wherein one or more of the 5 to 40 nucleotides is a 2′-substituted nucleoside, wherein at least 10 consecutive nucleotides of the 5 to 40 nucleotides are identical to, complementary, hybridizes, or binds to a viral target sequence. In some embodiments, the viral target sequence is in the rcDNA form of the HBV genome. In some embodiments, the viral target sequence is in the cccDNA form of the HBV genome. In some embodiments, the viral target sequence is in a gap region of the rcDNA or cccDNA form of the HBV genome. In some embodiments, the viral target sequence is in a non-gap region of the rcDNA or cccDNA form of the HBV genome. In some embodiments, the viral target sequence comprises a single strand of the cccDNA double strand region that is transiently opened at a transcription fork. In some embodiments, the viral target sequence comprises a single stranded region of the HBV genome. In some embodiments, the viral target sequence comprises a double stranded region of the HBV genome. In some embodiments, the oligonucleotide binds to the double stranded region of the HBV genome and forms Hoogsteen base pairs or reversed Hoogsteen hydrogen bonds. In some embodiments, the viral target sequence is in the X region of the HBV genome. In some embodiments, the viral target sequence is in the S region of the HBV genome. In some embodiments, the viral target sequence is in the HBV transcript. In some embodiments, the HBV transcript is selected from pre-genomic RNA (pgRNA), pre-Core RNA, pre-S1 RNA, pre-S2 RNA and X RNA. In some embodiments, the viral target sequence is in a promoter region of the HBV transcript. In some embodiments, the viral target sequence is in an enhancer region of the HBV transcript. In some embodiments, the viral target sequence is upstream of the promoter or enhancer region of the HBV transcript. In some embodiments, the viral target sequence is downstream of the promoter or enhancer region of the HBV transcript. In some embodiments, the viral target sequence is within about 2000, 1900, 1800, 1700, 1600, 1500, 1400, 1300, 1200, 1100, 1000, 900, 800, 700, 600, 500, 400, 300, 200, or 100 nucleotides of the promoter or enhancer region of the HBV transcript.

In some embodiments, the oligonucleotide comprises a nucleotide sequence comprising 5 to 40 nucleotides, wherein one or more of the 5 to 40 nucleotides is a 2′-O-methyl nucleoside, wherein at least 5 consecutive nucleotides of the 5 to 40 nucleotides are identical to, complementary, hybridizes, or binds to a viral target sequence. In some embodiments, the oligonucleotide comprises a nucleotide sequence comprising 5 to 40 nucleotides, wherein one or more of the 5 to 40 nucleotides is a 2′-O-methyl nucleoside, wherein at least 10 consecutive nucleotides of the 5 to 40 nucleotides are identical to, complementary, hybridizes, or binds to a viral target sequence. In some embodiments, the viral target sequence is in the rcDNA form of the HBV genome. In some embodiments, the viral target sequence is in the cccDNA form of the HBV genome. In some embodiments, the viral target sequence is in a gap region of the rcDNA or cccDNA form of the HBV genome. In some embodiments, the viral target sequence is in a non-gap region of the rcDNA or cccDNA form of the HBV genome. In some embodiments, the viral target sequence comprises a single strand of the cccDNA double strand region that is transiently opened at a transcription fork. In some embodiments, the viral target sequence comprises a single stranded region of the HBV genome. In some embodiments, the viral target sequence comprises a double stranded region of the HBV genome. In some embodiments, the oligonucleotide binds to the double stranded region of the HBV genome and forms Hoogsteen base pairs or reversed Hoogsteen hydrogen bonds. In some embodiments, the viral target sequence is in the X region of the HBV genome. In some embodiments, the viral target sequence is in the S region of the HBV genome. In some embodiments, the viral target sequence is in the HBV transcript. In some embodiments, the HBV transcript is selected from pre-genomic RNA (pgRNA), pre-Core RNA, pre-S1 RNA, pre-S2 RNA and X RNA. In some embodiments, the viral target sequence is in a promoter region of the HBV transcript. In some embodiments, the viral target sequence is in an enhancer region of the HBV transcript. In some embodiments, the viral target sequence is upstream of the promoter or enhancer region of the HBV transcript. In some embodiments, the viral target sequence is downstream of the promoter or enhancer region of the HBV transcript. In some embodiments, the viral target sequence is within about 2000, 1900, 1800, 1700, 1600, 1500, 1400, 1300, 1200, 1100, 1000, 900, 800, 700, 600, 500, 400, 300, 200, or 100 nucleotides of the promoter or enhancer region of the HBV transcript.

In some embodiments, a composition comprises: (a) any of the oligonucleotides disclosed herein; and (b) a pharmaceutically acceptable carrier, excipient, diluent, or adjuvant.

In some embodiments, a composition comprises (a) an oligonucleotide comprises a nucleotide sequence comprising 5 to 40 nucleotides, wherein one or more of the 5 to 40 nucleotides is a modified nucleoside, wherein at least 10 consecutive nucleotides of the 5 to 40 nucleotides are identical to, complementary, hybridizes, or binds to a viral target sequence: and (b) a pharmaceutically acceptable carrier, excipient, diluent, or adjuvant. In some embodiments, the viral target sequence is in the rcDNA form of the HBV genome. In some embodiments, the viral target sequence is in the cccDNA form of the HBV genome. In some embodiments, the viral target sequence is in a gap region of the rcDNA or cccDNA form of the HBV genome. In some embodiments, the viral target sequence is in a non-gap region of the rcDNA or cccDNA form of the HBV genome. In some embodiments, the viral target sequence comprises a single strand of the cccDNA double strand region that is transiently opened at a transcription fork. In some embodiments, the viral target sequence comprises a single stranded region of the HBV genome. In some embodiments, the viral target sequence comprises a double stranded region of the HBV genome. In some embodiments, the oligonucleotide binds to the double stranded region of the HBV genome and forms Hoogsteen base pairs or reversed Hoogsteen hydrogen bonds. In some embodiments, the viral target sequence is in the X region of the HBV genome. In some embodiments, the viral target sequence is in the S region of the HBV genome. In some embodiments, the viral target sequence is in the HBV transcript. In some embodiments, the HBV transcript is selected from pre-genomic RNA (pgRNA), pre-Core RNA, pre-S1 RNA, pre-S2 RNA and X RNA. In some embodiments, the viral target sequence is in a promoter region of the HBV transcript. In some embodiments, the viral target sequence is in an enhancer region of the HBV transcript. In some embodiments, the viral target sequence is upstream of the promoter or enhancer region of the HBV transcript. In some embodiments, the viral target sequence is downstream of the promoter or enhancer region of the HBV transcript. In some embodiments, the viral target sequence is within about 2000, 1900, 1800, 1700, 1600, 1500, 1400, 1300, 1200, 1100, 1000, 900, 800, 700, 600, 500, 400, 300, 200, or 100 nucleotides of the promoter or enhancer region of the HBV transcript.

In some embodiments, a composition comprises: (a) any of the oligonucleotides disclosed herein; and (b) an anti-HBV therapy.

In some embodiments, a composition comprises (a) an oligonucleotide comprises a nucleotide sequence comprising 5 to 40 nucleotides, wherein one or more of the 5 to 40 nucleotides is a modified nucleoside, wherein at least 10 consecutive nucleotides of the 5 to 40 nucleotides are identical to, complementary, hybridizes, or binds to a viral target sequence: and (b) an anti-HBV therapy. In some embodiments, the viral target sequence is in the rcDNA form of the HBV genome. In some embodiments, the viral target sequence is in the cccDNA form of the HBV genome. In some embodiments, the viral target sequence is in a gap region of the rcDNA or cccDNA form of the HBV genome. In some embodiments, the viral target sequence is in a non-gap region of the rcDNA or cccDNA form of the HBV genome. In some embodiments, the viral target sequence comprises a single strand of the cccDNA double strand region that is transiently opened at a transcription fork. In some embodiments, the viral target sequence comprises a single stranded region of the HBV genome. In some embodiments, the viral target sequence comprises a double stranded region of the HBV genome. In some embodiments, the oligonucleotide binds to the double stranded region of the HBV genome and forms Hoogsteen base pairs or reversed Hoogsteen hydrogen bonds. In some embodiments, the viral target sequence is in the X region of the HBV genome. In some embodiments, the viral target sequence is in the S region of the HBV genome. In some embodiments, the viral target sequence is in the HBV transcript. In some embodiments, the HBV transcript is selected from pre-genomic RNA (pgRNA), pre-Core RNA, pre-S1 RNA, pre-S2 RNA and X RNA. In some embodiments, the viral target sequence is in a promoter region of the HBV transcript. In some embodiments, the viral target sequence is in an enhancer region of the HBV transcript. In some embodiments, the viral target sequence is upstream of the promoter or enhancer region of the HBV transcript. In some embodiments, the viral target sequence is downstream of the promoter or enhancer region of the HBV transcript. In some embodiments, the viral target sequence is within about 2000, 1900, 1800, 1700, 1600, 1500, 1400, 1300, 1200, 1100, 1000, 900, 800, 700, 600, 500, 400, 300, 200, or 100 nucleotides of the promoter or enhancer region of the HBV transcript.

In some embodiments, a method of reducing conversion of hepatitis B virus (HBV) relaxed circular DNA (rcDNA) to covalently closed circular DNA (cccDNA) conversion, comprises contacting a cell with any of the oligonucleotides disclosed herein.

In some embodiments, a method of reducing conversion of hepatitis B virus (HBV) relaxed circular DNA (rcDNA) to covalently closed circular DNA (cccDNA) conversion, comprises contacting a cell with a oligonucleotide, wherein the oligonucleotide comprises a nucleotide sequence comprising 5 to 40 nucleotides, wherein one or more of the 5 to 40 nucleotides is a modified nucleoside, wherein at least 10 consecutive nucleotides of the 5 to 40 nucleotides is identical to, complementary, hybridizes, or binds to a viral target sequence. In some embodiments, the viral target sequence is in the rcDNA form of the HBV genome. In some embodiments, the viral target sequence is in the cccDNA form of the HBV genome. In some embodiments, the viral target sequence is in a gap region of the rcDNA or cccDNA form of the HBV genome. In some embodiments, the viral target sequence is in a non-gap region of the rcDNA or cccDNA form of the HBV genome. In some embodiments, the viral target sequence comprises a single strand of the cccDNA double strand region that is transiently opened at a transcription fork. In some embodiments, the viral target sequence comprises a single stranded region of the HBV genome. In some embodiments, the viral target sequence comprises a double stranded region of the HBV genome. In some embodiments, the oligonucleotide binds to the double stranded region of the HBV genome and forms Hoogsteen base pairs or reversed Hoogsteen hydrogen bonds. In some embodiments, the viral target sequence is in the X region of the HBV genome. In some embodiments, the viral target sequence is in the S region of the HBV genome. In some embodiments, the viral target sequence is in the HBV transcript. In some embodiments, the HBV transcript is selected from pre-genomic RNA (pgRNA), pre-Core RNA, pre-S1 RNA, pre-S2 RNA and X RNA. In some embodiments, the viral target sequence is in a promoter region of the HBV transcript. In some embodiments, the viral target sequence is in an enhancer region of the HBV transcript. In some embodiments, the viral target sequence is upstream of the promoter or enhancer region of the HBV transcript. In some embodiments, the viral target sequence is downstream of the promoter or enhancer region of the HBV transcript. In some embodiments, the viral target sequence is within about 2000, 1900, 1800, 1700, 1600, 1500, 1400, 1300, 1200, 1100, 1000, 900, 800, 700, 600, 500, 400, 300, 200, or 100 nucleotides of the promoter or enhancer region of the HBV transcript.

In some embodiments, a method of targeting hepatitis B virus (HBV) covalently closed circular DNA (cccDNA) for degradation, comprises contacting a cell with any of the oligonucleotides disclosed herein.

In some embodiments, a method of targeting hepatitis B virus (HBV) covalently closed circular DNA (cccDNA) for degradation, comprises contacting a cell with an oligonucleotide, wherein the oligonucleotide comprises a nucleotide sequence comprising 5 to 40 nucleotides, wherein one or more of the 5 to 40 nucleotides is a modified nucleoside, wherein at least 10 consecutive nucleotides of the 5 to 40 nucleotides are identical to, complementary, hybridizes, or binds to a viral target sequence. In some embodiments, the viral target sequence is in the rcDNA form of the HBV genome. In some embodiments, the viral target sequence is in the cccDNA form of the HBV genome. In some embodiments, the viral target sequence is in a gap region of the rcDNA or cccDNA form of the HBV genome. In some embodiments, the viral target sequence is in a non-gap region of the rcDNA or cccDNA form of the HBV genome. In some embodiments, the viral target sequence comprises a single strand of the cccDNA double strand region that is transiently opened at a transcription fork. In some embodiments, the viral target sequence comprises a single stranded region of the HBV genome. In some embodiments, the viral target sequence comprises a double stranded region of the HBV genome. In some embodiments, the oligonucleotide binds to the double stranded region of the HBV genome and forms Hoogsteen base pairs or reversed Hoogsteen hydrogen bonds. In some embodiments, the viral target sequence is in the X region of the HBV genome. In some embodiments, the viral target sequence is in the S region of the HBV genome. In some embodiments, the viral target sequence is in the HBV transcript. In some embodiments, the HBV transcript is selected from pre-genomic RNA (pgRNA), pre-Core RNA, pre-S1 RNA, pre-S2 RNA and X RNA. In some embodiments, the viral target sequence is in a promoter region of the HBV transcript. In some embodiments, the viral target sequence is in an enhancer region of the HBV transcript. In some embodiments, the viral target sequence is upstream of the promoter or enhancer region of the HBV transcript. In some embodiments, the viral target sequence is downstream of the promoter or enhancer region of the HBV transcript. In some embodiments, the viral target sequence is within about 2000, 1900, 1800, 1700, 1600, 1500, 1400, 1300, 1200, 1100, 1000, 900, 800, 700, 600, 500, 400, 300, 200, or 100 nucleotides of the promoter or enhancer region of the HBV transcript.

In some embodiments, a method of reducing the amount of hepatitis B virus (HBV) covalently closed circular DNA (cccDNA) in a cell, comprises contacting the cell with any of the oligonucleotides disclosed herein.

In some embodiments, a method of reducing the amount of hepatitis B virus (HBV) covalently closed circular DNA (cccDNA) in a cell, comprises contacting the cell with an oligonucleotide, wherein the oligonucleotide comprises a nucleotide sequence comprising 5 to 40 nucleotides, wherein one or more of the 5 to 40 nucleotides is a modified nucleoside, wherein at least 10 consecutive nucleotides of the 5 to 40 nucleotides is identical to, complementary, hybridizes, or binds to a viral target sequence. In some embodiments, the viral target sequence is in the rcDNA form of the HBV genome. In some embodiments, the viral target sequence is in the cccDNA form of the HBV genome. In some embodiments, the viral target sequence is in a gap region of the rcDNA or cccDNA form of the HBV genome. In some embodiments, the viral target sequence is in a non-gap region of the rcDNA or cccDNA form of the HBV genome. In some embodiments, the viral target sequence comprises a single strand of the cccDNA double strand region that is transiently opened at a transcription fork. In some embodiments, the viral target sequence comprises a single stranded region of the HBV genome. In some embodiments, the viral target sequence comprises a double stranded region of the HBV genome. In some embodiments, the oligonucleotide binds to the double stranded region of the HBV genome and forms Hoogsteen base pairs or reversed Hoogsteen hydrogen bonds. In some embodiments, the viral target sequence is in the X region of the HBV genome. In some embodiments, the viral target sequence is in the S region of the HBV genome. In some embodiments, the viral target sequence is in the HBV transcript. In some embodiments, the HBV transcript is selected from pre-genomic RNA (pgRNA), pre-Core RNA, pre-S1 RNA, pre-S2 RNA and X RNA. In some embodiments, the viral target sequence is in a promoter region of the HBV transcript. In some embodiments, the viral target sequence is in an enhancer region of the HBV transcript. In some embodiments, the viral target sequence is upstream of the promoter or enhancer region of the HBV transcript. In some embodiments, the viral target sequence is downstream of the promoter or enhancer region of the HBV transcript. In some embodiments, the viral target sequence is within about 2000, 1900, 1800, 1700, 1600, 1500, 1400, 1300, 1200, 1100, 1000, 900, 800, 700, 600, 500, 400, 300, 200, or 100 nucleotides of the promoter or enhancer region of the HBV transcript.

In some embodiments, a method of treating a hepatitis B virus (HBV) infection in a subject in need thereof, comprises administering to the subject any of the oligonucleotides disclosed herein. In some embodiments, the HBV is any one of genotypes A-J. In some embodiments, the HBV is any one of genotypes A-D. In some embodiments, the HBV is genotype A. In some embodiments, the HBV is genotype B. In some embodiments, the HBV is genotype C. In some embodiments, the HBV is genotype D.

In some embodiments, a method of treating a hepatitis B virus infection in a subject in need thereof, comprises administering to the subject (a) any of the oligonucleotides disclosed herein; and (b) an anti-HBV therapy. In some embodiments, the HBV is any one of genotypes A-J. In some embodiments, the HBV is any one of genotypes A-D. In some embodiments, the HBV is genotype A. In some embodiments, the HBV is genotype B. In some embodiments, the HBV is genotype C. In some embodiments, the HBV is genotype D.

In some embodiments, a method of treating a hepatitis B virus infection in a subject in need thereof, comprises administering to the subject an oligonucleotide, wherein the oligonucleotide comprises a nucleotide sequence comprising 5 to 40 nucleotides, wherein one or more of the 5 to 40 nucleotides is a modified nucleoside, wherein at least 10 consecutive nucleotides of the 5 to 40 nucleotides are identical to, complementary, hybridizes, or binds to a viral target sequence. In some embodiments, the viral target sequence is in the rcDNA form of the HBV genome. In some embodiments, the viral target sequence is in the cccDNA form of the HBV genome. In some embodiments, the viral target sequence is in a gap region of the rcDNA or cccDNA form of the HBV genome. In some embodiments, the viral target sequence is in a non-gap region of the rcDNA or cccDNA form of the HBV genome. In some embodiments, the viral target sequence comprises a single strand of the cccDNA double strand region that is transiently opened at a transcription fork. In some embodiments, the viral target sequence comprises a single stranded region of the HBV genome. In some embodiments, the viral target sequence comprises a double stranded region of the HBV genome. In some embodiments, the oligonucleotide binds to the double stranded region of the HBV genome and forms Hoogsteen base pairs or reversed Hoogsteen hydrogen bonds. In some embodiments, the viral target sequence is in the X region of the HBV genome. In some embodiments, the viral target sequence is in the S region of the HBV genome. In some embodiments, the viral target sequence is in the HBV transcript. In some embodiments, the HBV transcript is selected from pre-genomic RNA (pgRNA), pre-Core RNA, pre-S1 RNA, pre-S2 RNA and X RNA. In some embodiments, the viral target sequence is in a promoter region of the HBV transcript. In some embodiments, the viral target sequence is in an enhancer region of the HBV transcript. In some embodiments, the viral target sequence is upstream of the promoter or enhancer region of the HBV transcript. In some embodiments, the viral target sequence is downstream of the promoter or enhancer region of the HBV transcript. In some embodiments, the viral target sequence is within about 2000, 1900, 1800, 1700, 1600, 1500, 1400, 1300, 1200, 1100, 1000, 900, 800, 700, 600, 500, 400, 300, 200, or 100 nucleotides of the promoter or enhancer region of the HBV transcript. In some embodiments, the HBV is any one of genotypes A-J. In some embodiments, the HBV is any one of genotypes A-D. In some embodiments, the HBV is genotype A. In some embodiments, the HBV is genotype B. In some embodiments, the HBV is genotype C. In some embodiments, the HBV is genotype D.

In some embodiments, a method of treating a hepatitis B virus infection in a subject in need thereof, comprises administering to the subject (a) an oligonucleotide, wherein the oligonucleotide comprises a nucleotide sequence comprising 5 to 40 nucleotides, wherein one or more of the 5 to 40 nucleotides is a modified nucleoside, wherein at least 10 consecutive nucleotides of the 5 to 40 nucleotides is identical to, complementary, hybridizes, or binds to a viral target sequence; and (b) an anti-HBV therapy. In some embodiments, the viral target sequence is in the rcDNA form of the HBV genome. In some embodiments, the viral target sequence is in the cccDNA form of the HBV genome. In some embodiments, the viral target sequence is in a gap region of the rcDNA or cccDNA form of the HBV genome. In some embodiments, the viral target sequence is in a non-gap region of the rcDNA or cccDNA form of the HBV genome. In some embodiments, the viral target sequence comprises a single strand of the cccDNA double strand region that is transiently opened at a transcription fork. In some embodiments, the viral target sequence comprises a single stranded region of the HBV genome. In some embodiments, the viral target sequence comprises a double stranded region of the HBV genome. In some embodiments, the oligonucleotide binds to the double stranded region of the HBV genome and forms Hoogsteen base pairs or reversed Hoogsteen hydrogen bonds. In some embodiments, the viral target sequence is in the X region of the HBV genome. In some embodiments, the viral target sequence is in the S region of the HBV genome. In some embodiments, the viral target sequence is in the HBV transcript. In some embodiments, the HBV transcript is selected from pre-genomic RNA (pgRNA), pre-Core RNA, pre-S1 RNA, pre-S2 RNA and X RNA. In some embodiments, the viral target sequence is in a promoter region of the HBV transcript. In some embodiments, the viral target sequence is in an enhancer region of the HBV transcript. In some embodiments, the viral target sequence is upstream of the promoter or enhancer region of the HBV transcript. In some embodiments, the viral target sequence is downstream of the promoter or enhancer region of the HBV transcript. In some embodiments, the viral target sequence is within about 2000, 1900, 1800, 1700, 1600, 1500, 1400, 1300, 1200, 1100, 1000, 900, 800, 700, 600, 500, 400, 300, 200, or 100 nucleotides of the promoter or enhancer region of the HBV transcript. In some embodiments, the HBV is any one of genotypes A-J. In some embodiments, the HBV is any one of genotypes A-D. In some embodiments, the HBV is genotype A. In some embodiments, the HBV is genotype B. In some embodiments, the HBV is genotype C. In some embodiments, the HBV is genotype D.

HBV Targeting Oligonucleotides

Disclosed herein are oligonucleotides that interact with a viral target sequence of HBV. As used herein, an oligonucleotide that interacts with a viral target sequence of HBV is identical, complementary, binds, or hybridizes to the viral target sequence. As used herein, an oligonucleotide that is complementary to the viral target sequence refers to a sequence that is a complement or reverse complement of the viral target sequence. In some embodiments, the HBV is any one of genotypes A-J. In some embodiments, the HBV is any one of genotypes A-D. In some embodiments, the HBV is genotype A. In some embodiments, the HBV is genotype B. In some embodiments, the HBV is genotype C. In some embodiments, the HBV is genotype D. In some embodiments, the viral target sequence is in the rcDNA form of the HBV genome. In some embodiments, the viral target sequence is in the cccDNA form of the HBV genome. In some embodiments, the viral target sequence is in a gap region of the rcDNA or cccDNA form of the HBV genome. In some embodiments, the viral target sequence is in a non-gap region of the rcDNA or cccDNA form of the HBV genome. In some embodiments, the viral target sequence comprises a single strand of the cccDNA double strand region that is transiently opened at a transcription fork. In some embodiments, the viral target sequence comprises a single stranded region of the HBV genome. In some embodiments, the viral target sequence comprises a double stranded region of the HBV genome. In some embodiments, the oligonucleotide binds to the double stranded region of the HBV genome and forms Hoogsteen base pairs or reversed Hoogsteen hydrogen bonds. In some embodiments, the viral target sequence is in the X region of the HBV genome. In some embodiments, the viral target sequence is in the S region of the HBV genome. In some embodiments, the viral target sequence is in the HBV transcript. In some embodiments, the HBV transcript is selected from pre-genomic RNA (pgRNA), pre-Core RNA, pre-S1 RNA, pre-S2 RNA and X RNA. In some embodiments, the viral target sequence is in a promoter region of the HBV transcript. In some embodiments, the viral target sequence is in an enhancer region of the HBV transcript. In some embodiments, the viral target sequence is upstream of the promoter or enhancer region of the HBV transcript. In some embodiments, the viral target sequence is downstream of the promoter or enhancer region of the HBV transcript. In some embodiments, the viral target sequence is within about 2000, 1900, 1800, 1700, 1600, 1500, 1400, 1300, 1200, 1100, 1000, 900, 800, 700, 600, 500, 400, 300, 200, or 100 nucleotides of the promoter or enhancer region of the HBV transcript. In some embodiments, the oligonucleotides are incorporated into a cccDNA of HBV. In some embodiments, the oligonucleotide comprises a nucleotide sequence comprising at least 10 nucleotides, wherein one or more of the 10 nucleotides is a modified nucleoside, wherein at least 10 nucleotides of the nucleotide sequence are identical, complementary, hybridizes, or binds to the viral target sequence. In some embodiments, the modified nucleoside is selected from a locked nucleoside, 2′-substituted nucleoside, and 2′-O-methyl nucleoside. In some embodiments, the HBV is any one of genotypes A-J. In some embodiments, the HBV is any one of genotypes A-D. In some embodiments, the HBV is genotype A. In some embodiments, the HBV is genotype B. In some embodiments, the HBV is genotype C. In some embodiments, the HBV is genotype D.

In some embodiments, the oligonucleotide comprises a nucleotide sequence comprising 5 to 40 nucleotides, wherein one or more of the 5 to 40 nucleotides is a modified nucleoside, wherein at least 10 consecutive nucleotides of the 5 to 40 nucleotides are identical to, complementary, hybridizes, or binds to a viral target sequence. In some embodiments, the viral target sequence is in the rcDNA form of the HBV genome. In some embodiments, the viral target sequence is in the cccDNA form of the HBV genome. In some embodiments, the viral target sequence is in a gap region of the rcDNA or cccDNA form of the HBV genome. In some embodiments, the viral target sequence is in a non-gap region of the rcDNA or cccDNA form of the HBV genome. In some embodiments, the viral target sequence comprises a single strand of the cccDNA double strand region that is transiently opened at a transcription fork. In some embodiments, the viral target sequence comprises a single stranded region of the HBV genome. In some embodiments, the viral target sequence comprises a double stranded region of the HBV genome. In some embodiments, the oligonucleotide binds to the double stranded region of the HBV genome and forms Hoogsteen base pairs or reversed Hoogsteen hydrogen bonds. In some embodiments, the viral target sequence is in the X region of the HBV genome. In some embodiments, the viral target sequence is in the S region of the HBV genome. In some embodiments, the viral target sequence is in the HBV transcript. In some embodiments, the HBV transcript is selected from pre-genomic RNA (pgRNA), pre-Core RNA, pre-S1 RNA, pre-S2 RNA and X RNA. In some embodiments, the viral target sequence is in a promoter region of the HBV transcript. In some embodiments, the viral target sequence is in an enhancer region of the HBV transcript. In some embodiments, the viral target sequence is upstream of the promoter or enhancer region of the HBV transcript. In some embodiments, the viral target sequence is downstream of the promoter or enhancer region of the HBV transcript. In some embodiments, the viral target sequence is within about 2000, 1900, 1800, 1700, 1600, 1500, 1400, 1300, 1200, 1100, 1000, 900, 800, 700, 600, 500, 400, 300, 200, or 100 nucleotides of the promoter or enhancer region of the HBV transcript. In some embodiments, the HBV is any one of genotypes A-J. In some embodiments, the HBV is any one of genotypes A-D. In some embodiments, the HBV is genotype A. In some embodiments, the HBV is genotype B. In some embodiments, the HBV is genotype C. In some embodiments, the HBV is genotype D.

In some embodiments, the oligonucleotide comprises a nucleotide sequence comprising 5 to 40 nucleotides, wherein one or more of the 5 to 40 nucleotides is a locked nucleoside, wherein at least 10 consecutive nucleotides of the 5 to 40 nucleotides are identical to, complementary, hybridizes, or binds to a viral target sequence. In some embodiments, the viral target sequence is in the rcDNA form of the HBV genome. In some embodiments, the viral target sequence is in the cccDNA form of the HBV genome. In some embodiments, the viral target sequence is in a gap region of the rcDNA or cccDNA form of the HBV genome. In some embodiments, the viral target sequence is in a non-gap region of the rcDNA or cccDNA form of the HBV genome. In some embodiments, the viral target sequence comprises a single strand of the cccDNA double strand region that is transiently opened at a transcription fork. In some embodiments, the viral target sequence comprises a single stranded region of the HBV genome. In some embodiments, the viral target sequence comprises a double stranded region of the HBV genome. In some embodiments, the oligonucleotide binds to the double stranded region of the HBV genome and forms Hoogsteen base pairs or reversed Hoogsteen hydrogen bonds. In some embodiments, the viral target sequence is in the X region of the HBV genome. In some embodiments, the viral target sequence is in the S region of the HBV genome. In some embodiments, the viral target sequence is in the HBV transcript. In some embodiments, the HBV transcript is selected from pre-genomic RNA (pgRNA), pre-Core RNA, pre-S1 RNA, pre-S2 RNA and X RNA. In some embodiments, the viral target sequence is in a promoter region of the HBV transcript. In some embodiments, the viral target sequence is in an enhancer region of the HBV transcript. In some embodiments, the viral target sequence is upstream of the promoter or enhancer region of the HBV transcript. In some embodiments, the viral target sequence is downstream of the promoter or enhancer region of the HBV transcript. In some embodiments, the viral target sequence is within about 2000, 1900, 1800, 1700, 1600, 1500, 1400, 1300, 1200, 1100, 1000, 900, 800, 700, 600, 500, 400, 300, 200, or 100 nucleotides of the promoter or enhancer region of the HBV transcript. In some embodiments, the HBV is any one of genotypes A-J. In some embodiments, the HBV is any one of genotypes A-D. In some embodiments, the HBV is genotype A. In some embodiments, the HBV is genotype B. In some embodiments, the HBV is genotype C. In some embodiments, the HBV is genotype D.

In some embodiments, the oligonucleotide comprises a nucleotide sequence comprising 5 to 40 nucleotides, wherein one or more of the 5 to 40 nucleotides is a 2′-substituted nucleoside, wherein at least 10 consecutive nucleotides of the 5 to 40 nucleotides is identical to, complementary, hybridizes, or binds to a viral target sequence. In some embodiments, the viral target sequence is in the rcDNA form of the HBV genome. In some embodiments, the viral target sequence is in the cccDNA form of the HBV genome. In some embodiments, the viral target sequence is in a gap region of the rcDNA or cccDNA form of the HBV genome. In some embodiments, the viral target sequence is in a non-gap region of the rcDNA or cccDNA form of the HBV genome. In some embodiments, the viral target sequence comprises a single strand of the cccDNA double strand region that is transiently opened at a transcription fork. In some embodiments, the viral target sequence comprises a single stranded region of the HBV genome. In some embodiments, the viral target sequence comprises a double stranded region of the HBV genome. In some embodiments, the oligonucleotide binds to the double stranded region of the HBV genome and forms Hoogsteen base pairs or reversed Hoogsteen hydrogen bonds. In some embodiments, the viral target sequence is in the X region of the HBV genome. In some embodiments, the viral target sequence is in the S region of the HBV genome. In some embodiments, the viral target sequence is in the HBV transcript. In some embodiments, the HBV transcript is selected from pre-genomic RNA (pgRNA), pre-Core RNA, pre-S1 RNA, pre-S2 RNA and X RNA. In some embodiments, the viral target sequence is in a promoter region of the HBV transcript. In some embodiments, the viral target sequence is in an enhancer region of the HBV transcript. In some embodiments, the viral target sequence is upstream of the promoter or enhancer region of the HBV transcript. In some embodiments, the viral target sequence is downstream of the promoter or enhancer region of the HBV transcript. In some embodiments, the viral target sequence is within about 2000, 1900, 1800, 1700, 1600, 1500, 1400, 1300, 1200, 1100, 1000, 900, 800, 700, 600, 500, 400, 300, 200, or 100 nucleotides of the promoter or enhancer region of the HBV transcript. In some embodiments, the HBV is any one of genotypes A-J. In some embodiments, the HBV is any one of genotypes A-D. In some embodiments, the HBV is genotype A. In some embodiments, the HBV is genotype B. In some embodiments, the HBV is genotype C. In some embodiments, the HBV is genotype D.

In some embodiments, the oligonucleotide comprises a nucleotide sequence comprising 5 to 40 nucleotides, wherein one or more of the 5 to 40 nucleotides is a 2′-O-methyl nucleoside, wherein at least 10 consecutive nucleotides of the 5 to 40 nucleotides are identical to, complementary, hybridizes, or binds to a viral target sequence. In some embodiments, the viral target sequence is in the rcDNA form of the HBV genome. In some embodiments, the viral target sequence is in the cccDNA form of the HBV genome. In some embodiments, the viral target sequence is in a gap region of the rcDNA or cccDNA form of the HBV genome. In some embodiments, the viral target sequence is in a non-gap region of the rcDNA or cccDNA form of the HBV genome. In some embodiments, the viral target sequence comprises a single strand of the cccDNA double strand region that is transiently opened at a transcription fork. In some embodiments, the viral target sequence comprises a single stranded region of the HBV genome. In some embodiments, the viral target sequence comprises a double stranded region of the HBV genome. In some embodiments, the oligonucleotide binds to the double stranded region of the HBV genome and forms Hoogsteen base pairs or reversed Hoogsteen hydrogen bonds. In some embodiments, the viral target sequence is in the X region of the HBV genome. In some embodiments, the viral target sequence is in the S region of the HBV genome. In some embodiments, the viral target sequence is in the HBV transcript. In some embodiments, the HBV transcript is selected from pre-genomic RNA (pgRNA), pre-Core RNA, pre-S1 RNA, pre-S2 RNA and X RNA. In some embodiments, the viral target sequence is in a promoter region of the HBV transcript. In some embodiments, the viral target sequence is in an enhancer region of the HBV transcript. In some embodiments, the viral target sequence is upstream of the promoter or enhancer region of the HBV transcript. In some embodiments, the viral target sequence is downstream of the promoter or enhancer region of the HBV transcript. In some embodiments, the viral target sequence is within about 2000, 1900, 1800, 1700, 1600, 1500, 1400, 1300, 1200, 1100, 1000, 900, 800, 700, 600, 500, 400, 300, 200, or 100 nucleotides of the promoter or enhancer region of the HBV transcript. In some embodiments, the HBV is any one of genotypes A-J. In some embodiments, the HBV is any one of genotypes A-D. In some embodiments, the HBV is genotype A. In some embodiments, the HBV is genotype B. In some embodiments, the HBV is genotype C. In some embodiments, the HBV is genotype D.

In some embodiments, the oligonucleotide comprises a nucleotide sequence comprising at least one methylated nucleoside, wherein at least 10 consecutive nucleotides of the 5 to 40 nucleotides are identical to, complementary, hybridizes, or binds to a viral target sequence. In some embodiments, the viral target sequence is in the rcDNA form of the HBV genome. In some embodiments, the viral target sequence is in the cccDNA form of the HBV genome. In some embodiments, the viral target sequence is in a gap region of the rcDNA or cccDNA form of the HBV genome. In some embodiments, the viral target sequence is in a non-gap region of the rcDNA or cccDNA form of the HBV genome. In some embodiments, the viral target sequence comprises a single strand of the cccDNA double strand region that is transiently opened at a transcription fork. In some embodiments, the viral target sequence comprises a single stranded region of the HBV genome. In some embodiments, the viral target sequence comprises a double stranded region of the HBV genome. In some embodiments, the oligonucleotide binds to the double stranded region of the HBV genome and forms Hoogsteen base pairs or reversed Hoogsteen hydrogen bonds. In some embodiments, the viral target sequence is in the X region of the HBV genome. In some embodiments, the viral target sequence is in the S region of the HBV genome. In some embodiments, the viral target sequence is in the HBV transcript. In some embodiments, the HBV transcript is selected from pre-genomic RNA (pgRNA), pre-Core RNA, pre-S1 RNA, pre-S2 RNA and X RNA. In some embodiments, the viral target sequence is in a promoter region of the HBV transcript. In some embodiments, the viral target sequence is in an enhancer region of the HBV transcript. In some embodiments, the viral target sequence is upstream of the promoter or enhancer region of the HBV transcript. In some embodiments, the viral target sequence is downstream of the promoter or enhancer region of the HBV transcript. In some embodiments, the viral target sequence is within about 2000, 1900, 1800, 1700, 1600, 1500, 1400, 1300, 1200, 1100, 1000, 900, 800, 700, 600, 500, 400, 300, 200, or 100 nucleotides of the promoter or enhancer region of the HBV transcript. In some embodiments, the HBV is any one of genotypes A-J. In some embodiments, the HBV is any one of genotypes A-D. In some embodiments, the HBV is genotype A. In some embodiments, the HBV is genotype B. In some embodiments, the HBV is genotype C. In some embodiments, the HBV is genotype D.

In some embodiments, the viral target sequence comprises at least a portion of an HBV sequence of any one of HBV genotypes A-J. In some embodiments, the viral target sequence comprises at least a portion of an HBV sequence of HBV genotype A. In some embodiments the viral target sequence comprises at least a portion of an HBV sequence of HBV genotype B. In some embodiments, the viral target sequence comprises at least a portion of an HBV sequence of HBV genotype C. In some embodiments, the viral target sequence comprises at least a portion of an HBV sequence of HBV genotype D.

In some embodiments, the viral target sequence is in the gap region of rcDNA. In some embodiments, the gap region of the rcDNA comprises positions 1 to 1600, 200 to 1600, 300 to 1600, 400 to 1600, 500 to 1600, 600 to 1600, 650 to 1600, 700 to 1600, 750 to 1600, 800 to 1600, 850 to 1600, 900 to 1600, 950 to 1600, 1000 to 1600, 1050 to 1600, 1100 to 1600, 1150 to 1600, 1200 to 1600, 1250 to 1600, 1300 to 1600, 1350 to 1600, 1400 to 1600, 1450 to 1600, 1500 to 1600, 1550 to 1600, or 1580 to 1600 of SEQ ID NO: 1 or a comparable region in SEQ ID NO: 2 or a sequence of any of HBV genotypes A-J. In some embodiments, the viral target sequence comprises at least 5, 6, 7, 8, 9, 10, 11, 12, 13, 14, 15, 16, 17, 18, 19, 20, 21, 22, 23, 24, 25, 26, 27, 28, 29, or 30 consecutive nucleotides within the gap region. In some embodiments, the viral target sequence comprises less than 50, 45, 40, 35, 34, 33, 32, 31, 30, 29, 28, 27, 26, 25, 24, 23, 22, 21, or 20 consecutive nucleotides within the gap region. In some embodiments, the viral target sequence comprises 5 to 40 nucleotides within the gap region. In some embodiments, the viral target sequence comprises at least 5, 6, 7, 8, 9, 10, 11, 12, 13, 14, 15, 16, 17, 18, 19, 20, 21, 22, 23, 24, 25, 26, 27, 28, 29, or 30 consecutive nucleotides within positions 950-1050, 975-1025, 975-1010, 980-1010, 1150-1275, 1175-1275, 1180-1270, 1180-1250, 1180-1225, 1180-1210, 1225-1350, 1225-1325, 1225-1300, 1225-1290, 1250-1350, 1250-1325, 1250-1300, 1250-1290, 1375-1475, 1375-1450, 1375-1440, 1375-1430, 1390-1475, 1390-1450, 1390-1440, 1390-1430, 1500-1700, 1500-1650, 1500-1620, 1500-1595, 1510-1700, 1510-1650, 1510-1620, 1510-1595, 1515-1700, 1515-1650, 1515-1620, 1515-1590, or 2817-3050 of SEQ ID NO: 1 or a comparable region in SEQ ID NO: 2 or a sequence of any of HBV genotypes A-J. In some embodiments, the viral target sequence comprises 5 to 40 nucleotides within the gap region. In some embodiments, the viral target sequence comprises 5 to 40 nucleotides within positions 950-1050, 975-1025, 975-1010, 980-1010, 1150-1275, 1175-1275, 1180-1270, 1180-1250, 1180-1225, 1180-1210, 1225-1350, 1225-1325, 1225-1300, 1225-1290, 1250-1350, 1250-1325, 1250-1300, 1250-1290, 1375-1475, 1375-1450, 1375-1440, 1375-1430, 1390-1475, 1390-1450, 1390-1440, 1390-1430, 1500-1700, 1500-1650, 1500-1620, 1500-1595, 1510-1700, 1510-1650, 1510-1620, 1510-1595, 1515-1700, 1515-1650, 1515-1620, 1515-1590, or 2817-3050 of SEQ ID NO: 1 or a comparable region in SEQ ID NO: 2 or a sequence of any of HBV genotypes A-J. In some embodiments, the comparable region in SEQ ID NO: 2 or the sequence of any of HBV genotypes A-J is at least about 80%, 86%, 87%, 88%, 89%, 90%, 91%, 92%, 93%, 94%, 95%, 96%, 97%, 98%, or 99% identical to the region in SEQ ID NO: 1. In some embodiments, the comparable region in SEQ ID NO: 2 or the sequence of any of HBV genotypes A-J is based on an alignment of the sequence of SEQ ID NO: 2 or HBV genotypes A-J to the sequence of SEQ ID NO: 1.

In some embodiments, the viral target sequence is in a non-gap region of the rcDNA. In some embodiments, the non-gap region of the rcDNA comprises positions 1601 to 3215, 1601 to 3100, 1601 to 2900, 1601 to 2800, 1601 to 2700, 1601 to 2600, 1601 to 2500, 1601 to 2400, 1601 to 2300, 1601 to 2250, 1601 to 2200, 1601 to 2150, 1601 to 2100, 1601 to 2050, 1601 to 2000, 1601 to 1950, 1601 to 1900, 1601 to 1850, 1601 to 1800, 1601 to 1750, or 1601 to 1700 of SEQ ID NO: 1 or a comparable region in SEQ ID NO: 2 or a sequence of any of HBV genotypes A-J. In some embodiments, the viral target sequence comprises at least 5, 6, 7, 8, 9, 10, 11, 12, 13, 14, 15, 16, 17, 18, 19, 20, 21, 22, 23, 24, 25, 26, 27, 28, 29, or 30 consecutive nucleotides within the non-gap region. In some embodiments, the viral target sequence comprises less than 50, 45, 40, 35, 34, 33, 32, 31, 30, 29, 28, 27, 26, 25, 24, 23, 22, 21, or 20 consecutive nucleotides within the non-gap region. In some embodiments, the viral target sequence comprises 5 to 40 nucleotides within the non-gap region. In some embodiments, the viral target sequence comprises at least 5, 6, 7, 8, 9, 10, 11, 12, 13, 14, 15, 16, 17, 18, 19, 20, 21, 22, 23, 24, 25, 26, 27, 28, 29, or 30 consecutive nucleotides within positions 1601 to 3215, 1601 to 3100, 1601 to 2900, 1601 to 2800, 1601 to 2700, 1601 to 2600, 1601 to 2500, 1601 to 2400, 1601 to 2300, 1601 to 2250, 1601 to 2200, 1601 to 2150, 1601 to 2100, 1601 to 2050, 1601 to 2000, 1601 to 1950, 1601 to 1900, 1601 to 1850, 1601 to 1800, 1601 to 1750, or 1601 to 1700 of SEQ ID NO: 1 or a comparable region in SEQ ID NO: 2 or a sequence of any of HBV genotypes A-J. In some embodiments, the viral target sequence comprises 5 to 40 nucleotides within positions 1601 to 3215, 1601 to 3100, 1601 to 2900, 1601 to 2800, 1601 to 2700, 1601 to 2600, 1601 to 2500, 1601 to 2400, 1601 to 2300, 1601 to 2250, 1601 to 2200, 1601 to 2150, 1601 to 2100, 1601 to 2050, 1601 to 2000, 1601 to 1950, 1601 to 1900, 1601 to 1850, 1601 to 1800, 1601 to 1750, or 1601 to 1700 of SEQ ID NO: 1 or a comparable region in SEQ ID NO: 2 or a sequence of any of HBV genotypes A-J. In some embodiments, the comparable region in SEQ ID NO: 2 or the sequence of any of HBV genotypes A-J is at least about 80%, 86%, 87%, 88%, 89%, 90%, 91%, 92%, 93%, 94%, 95%, 96%, 97%, 98%, or 99% identical to the region in SEQ ID NO: 1. In some embodiments, the comparable region in SEQ ID NO: 2 or the sequence of any of HBV genotypes A-J is based on an alignment of the sequence of SEQ ID NO: 2 or HBV genotypes A-J to the sequence of SEQ ID NO: 1.

In some embodiments, the viral target sequence is in an X region of the rcDNA or cccDNA. In some embodiments, the viral target sequence comprises 5 to 40 nucleotides within the X region. In some embodiments, the viral target sequence comprises at least 5, 6, 7, 8, 9, 10, 11, 12, 13, 14, 15, 16, 17, 18, 19, 20, 21, 22, 23, 24, 25, 26, 27, 28, 29, or 30 consecutive nucleotides within the X region. In some embodiments, the viral target sequence comprises less than 50, 45, 40, 35, 34, 33, 32, 31, 30, 29, 28, 27, 26, 25, 24, 23, 22, 21, or 20 consecutive nucleotides within the X region. In some embodiments, the viral target sequence comprises 5 to 40 nucleotides within the X region. In some embodiments, the viral target sequence comprises at least 5, 6, 7, 8, 9, 10, 11, 12, 13, 14, 15, 16, 17, 18, 19, 20, 21, 22, 23, 24, 25, 26, 27, 28, 29, or 30 consecutive nucleotides within positions 1374 to 1603, 1400 to 1603, 1450 to 1603, 1500 to 1603, or 1550 to 1603 of SEQ ID NO: 1 or a comparable region in SEQ ID NO: 2 or a sequence of any of HBV genotypes A-J. In some embodiments, the viral target sequence comprises 5 to 40 nucleotides within positions 1374 to 1603, 1400 to 1603, 1450 to 1603, 1500 to 1603, or 1550 to 1603 of SEQ ID NO: 1 or a comparable region in SEQ ID NO: 2 or a sequence of any of HBV genotypes A-J. In some embodiments, the comparable region in SEQ ID NO: 2 or the sequence of any of HBV genotypes A-J is at least about 80%, 86%, 87%, 88%, 89%, 90%, 91%, 92%, 93%, 94%, 95%, 96%, 97%, 98%, or 99% identical to the region in SEQ ID NO: 1. In some embodiments, the comparable region in SEQ ID NO: 2 or the sequence of any of HBV genotypes A-J is based on an alignment of the sequence of SEQ ID NO: 2 or HBV genotypes A-J to the sequence of SEQ ID NO: 1.

In some embodiments, the viral target sequence is in an S region of the rcDNA or cccDNA. In some embodiments, the viral target sequence comprises at least 5, 6, 7, 8, 9, 10, 11, 12, 13, 14, 15, 16, 17, 18, 19, 20, 21, 22, 23, 24, 25, 26, 27, 28, 29, or 30 consecutive nucleotides within the S region. In some embodiments, the viral target sequence comprises less than 50, 45, 40, 35, 34, 33, 32, 31, 30, 29, 28, 27, 26, 25, 24, 23, 22, 21, or 20 consecutive nucleotides within the S region. In some embodiments, the viral target sequence comprises 5 to 40 nucleotides within the S region. In some embodiments, the viral target sequence comprises at least 5, 6, 7, 8, 9, 10, 11, 12, 13, 14, 15, 16, 17, 18, 19, 20, 21, 22, 23, 24, 25, 26, 27, 28, 29, or 30 consecutive nucleotides within positions 155 to 1373, 200 to 1373, 300 to 1373, 400 to 1373, 500 to 1373, 600 to 1373, 650 to 1373, 700 to 1373, 750 to 1373, or 800 to 1373 of SEQ ID NO: 1 or a comparable region in SEQ ID NO: 2 or a sequence of any of HBV genotypes A-J. In some embodiments, the viral target sequence comprises 5 to 40 nucleotides within positions 155 to 1373, 200 to 1373, 300 to 1373, 400 to 1373, 500 to 1373, 600 to 1373, 650 to 1373, 700 to 1373, 750 to 1373, or 800 to 1373 of SEQ ID NO: 1 or a comparable region in SEQ ID NO: 2 or a sequence of any of HBV genotypes A-J. In some embodiments, the comparable region in SEQ ID NO: 2 or the sequence of any of HBV genotypes A-J is at least about 80%, 86%, 87%, 88%, 89%, 90%, 91%, 92%, 93%, 94%, 95%, 96%, 97%, 98%, or 99% identical to the region in SEQ ID NO: 1. In some embodiments, the comparable region in SEQ ID NO: 2 or the sequence of any of HBV genotypes A-J is based on an alignment of the sequence of SEQ ID NO: 2 or HBV genotypes A-J to the sequence of SEQ ID NO: 1.

In some embodiments, the viral target sequence is in the HBV transcript. In some embodiments, the HBV transcript is selected from pre-genomic RNA (pgRNA), pre-Core RNA, pre-S1 RNA, pre-S2 RNA and X RNA. In some embodiments, the viral target sequence is in the pgRNA. In some embodiments, the pgRNA comprises the nucleotide sequence corresponding to nucleotides 1818 . . . 3215, 1 . . . 1930 of SEQ ID NO: 1 or a comparable region in SEQ ID NO: 2 or a sequence of any of HBV genotypes A-J. In some embodiments, the viral target sequence comprises at least 5, 6, 7, 8, 9, 10, 11, 12, 13, 14, 15, 16, 17, 18, 19, 20, 21, 22, 23, 24, 25, 26, 27, 28, 29, or 30 consecutive nucleotides within the pgRNA. In some embodiments, the viral target sequence comprises less than 50, 45, 40, 35, 34, 33, 32, 31, 30, 29, 28, 27, 26, 25, 24, 23, 22, 21, or 20 consecutive nucleotides within the pgRNA. In some embodiments, the viral target sequence comprises 5 to 40 nucleotides within the pgRNA. In some embodiments, the viral target sequence comprises at least 5, 6, 7, 8, 9, 10, 11, 12, 13, 14, 15, 16, 17, 18, 19, 20, 21, 22, 23, 24, 25, 26, 27, 28, 29, or 30 consecutive nucleotides within positions 1818-3215 or 1-1930 of SEQ ID NO: 1 or a comparable region in SEQ ID NO: 2 or a sequence of any of HBV genotypes A-J. In some embodiments, the viral target sequence comprises 5 to 40 nucleotides within positions 1818-3215 or 1-1930 of SEQ ID NO: 1 or a comparable region in SEQ ID NO: 2 or a sequence of any of HBV genotypes A-J. In some embodiments, the comparable region in SEQ ID NO: 2 or the sequence of any of HBV genotypes A-J is at least about 80%, 86%, 87%, 88%, 89%, 90%, 91%, 92%, 93%, 94%, 95%, 96%, 97%, 98%, or 99% identical to the region in SEQ ID NO: 1. In some embodiments, the comparable region in SEQ ID NO: 2 or the sequence of any of HBV genotypes A-J is based on an alignment of the sequence of SEQ ID NO: 2 or HBV genotypes A-J to the sequence of SEQ ID NO: 1.

In some embodiments, the viral target sequence is in the pre-Core RNA. In some embodiments, the pre-Core RNA comprises the nucleotide sequence corresponding to nucleotides 1814-2452 of SEQ ID NO: 1 or a comparable region in SEQ ID NO: 2 or a sequence of any of HBV genotypes A-J. In some embodiments, the viral target sequence comprises at least 5, 6, 7, 8, 9, 10, 11, 12, 13, 14, 15, 16, 17, 18, 19, 20, 21, 22, 23, 24, 25, 26, 27, 28, 29, or 30 consecutive nucleotides within the pre-Core RNA. In some embodiments, the viral target sequence comprises less than 50, 45, 40, 35, 34, 33, 32, 31, 30, 29, 28, 27, 26, 25, 24, 23, 22, 21, or 20 consecutive nucleotides within the pre-Core RNA. In some embodiments, the viral target sequence comprises 5 to 40 nucleotides within the pre-Core RNA. In some embodiments, the viral target sequence comprises at least 5, 6, 7, 8, 9, 10, 11, 12, 13, 14, 15, 16, 17, 18, 19, 20, 21, 22, 23, 24, 25, 26, 27, 28, 29, or 30 consecutive nucleotides within positions 1814-2452 of SEQ ID NO: 1 or a comparable region in SEQ ID NO: 2 or a sequence of any of HBV genotypes A-J. In some embodiments, the viral target sequence comprises 5 to 40 nucleotides within positions 1814-2452 of SEQ ID NO: 1 or a comparable region in SEQ ID NO: 2 or a sequence of any of HBV genotypes A-J. In some embodiments, the comparable region in SEQ ID NO: 2 or the sequence of any of HBV genotypes A-J is at least about 80%, 86%, 87%, 88%, 89%, 90%, 91%, 92%, 93%, 94%, 95%, 96%, 97%, 98%, or 99% identical to the region in SEQ ID NO: 1. In some embodiments, the comparable region in SEQ ID NO: 2 or the sequence of any of HBV genotypes A-J is based on an alignment of the sequence of SEQ ID NO: 2 or HBV genotypes A-J to the sequence of SEQ ID NO: 1.

In some embodiments, the viral target sequence is in the pre-S1 RNA. In some embodiments, the pre-S1 RNA comprises the nucleotide sequence corresponding to nucleotides 2848 . . . 3215, 1 . . . 835 of SEQ ID NO: 1 or a comparable region in SEQ ID NO: 2 or a sequence of any of HBV genotypes A-J. In some embodiments, the viral target sequence comprises at least 5, 6, 7, 8, 9, 10, 11, 12, 13, 14, 15, 16, 17, 18, 19, 20, 21, 22, 23, 24, 25, 26, 27, 28, 29, or 30 consecutive nucleotides within the pre-S1 RNA. In some embodiments, the viral target sequence comprises less than 50, 45, 40, 35, 34, 33, 32, 31, 30, 29, 28, 27, 26, 25, 24, 23, 22, 21, or 20 consecutive nucleotides within the pre-S1 RNA. In some embodiments, the viral target sequence comprises 5 to 40 nucleotides within the pre-S1 RNA. In some embodiments, the viral target sequence comprises at least 5, 6, 7, 8, 9, 10, 11, 12, 13, 14, 15, 16, 17, 18, 19, 20, 21, 22, 23, 24, 25, 26, 27, 28, 29, or 30 consecutive nucleotides within positions 1848-3215 or 1-835 of SEQ ID NO: 1 or a comparable region in SEQ ID NO: 2 or a sequence of any of HBV genotypes A-J. In some embodiments, the viral target sequence comprises 5 to 40 nucleotides within positions 1848-3215 or 1-835 of SEQ ID NO: 1 or a comparable region in SEQ ID NO: 2 or an HBV of any of genotypes A-J. In some embodiments, the viral target sequence comprises at least 5, 6, 7, 8, 9, 10, 11, 12, 13, 14, 15, 16, 17, 18, 19, 20, 21, 22, 23, 24, 25, 26, 27, 28, 29, or 30 consecutive nucleotides within positions 2817-3050 of SEQ ID NO: 1 or a comparable region in SEQ ID NO: 2 or a sequence of any of HBV genotypes A-J. In some embodiments, the viral target sequence comprises 5 to 40 nucleotides within positions 2817-3050 of SEQ ID NO: 1 or a comparable region in SEQ ID NO: 2 or a sequence of any of HBV genotypes A-J. In some embodiments, the comparable region in SEQ ID NO: 2 or the sequence of any of HBV genotypes A-J is at least about 80%, 86%, 87%, 88%, 89%, 90%, 91%, 92%, 93%, 94%, 95%, 96%, 97%, 98%, or 99% identical to the region in SEQ ID NO: 1. In some embodiments, the comparable region in SEQ ID NO: 2 or the sequence of any of HBV genotypes A-J is based on an alignment of the sequence of SEQ ID NO: 2 or HBV genotypes A-J to the sequence of SEQ ID NO: 1.

In some embodiments, the viral target sequence is in the pre-S2 RNA. In some embodiments, the pre-S2 RNA comprises the nucleotide sequence corresponding to nucleotides 3205 . . . 3215, 1 . . . 835 of SEQ ID NO: 1 or a comparable region in SEQ ID NO: 2 or a sequence of any of HBV genotypes A-J. In some embodiments, the viral target sequence comprises at least 5, 6, 7, 8, 9, 10, 11, 12, 13, 14, 15, 16, 17, 18, 19, 20, 21, 22, 23, 24, 25, 26, 27, 28, 29, or 30 consecutive nucleotides within the pre-S2 RNA. In some embodiments, the viral target sequence comprises less than 50, 45, 40, 35, 34, 33, 32, 31, 30, 29, 28, 27, 26, 25, 24, 23, 22, 21, or 20 consecutive nucleotides within the pre-S2 RNA. In some embodiments, the viral target sequence comprises 5 to 40 nucleotides within the pre-S2 RNA. In some embodiments, the viral target sequence comprises at least 5, 6, 7, 8, 9, 10, 11, 12, 13, 14, 15, 16, 17, 18, 19, 20, 21, 22, 23, 24, 25, 26, 27, 28, 29, or 30 consecutive nucleotides within positions 3205-3215 or 1-835 of SEQ ID NO: 1 or a comparable region in SEQ ID NO: 2 or a sequence of any of HBV genotypes A-J. In some embodiments, the viral target sequence comprises 5 to 40 nucleotides within positions 3205-3215 or 1-835 of SEQ ID NO: 1 or a comparable region in SEQ ID NO: 2 or a sequence of any of HBV genotypes A-J. In some embodiments, the comparable region in SEQ ID NO: 2 or the sequence of any of HBV genotypes A-J is at least about 80%, 86%, 87%, 88%, 89%, 90%, 91%, 92%, 93%, 94%, 95%, 96%, 97%, 98%, or 99% identical to the region in SEQ ID NO: 1. In some embodiments, the comparable region in SEQ ID NO: 2 or the sequence of any of HBV genotypes A-J is based on an alignment of the sequence of SEQ ID NO: 2 or HBV genotypes A-J to the sequence of SEQ ID NO: 1.

In some embodiments, the viral target sequence is in the X RNA. In some embodiments, the X RNA comprises the nucleotide sequence corresponding to nucleotides 1374 . . . 1838 of SEQ ID NO: 1 or a comparable region in SEQ ID NO: 2 or a sequence of any of HBV genotypes A-J. In some embodiments, the viral target sequence comprises at least 5, 6, 7, 8, 9, 10, 11, 12, 13, 14, 15, 16, 17, 18, 19, 20, 21, 22, 23, 24, 25, 26, 27, 28, 29, or 30 consecutive nucleotides within the X RNA. In some embodiments, the viral target sequence comprises less than 50, 45, 40, 35, 34, 33, 32, 31, 30, 29, 28, 27, 26, 25, 24, 23, 22, 21, or 20 consecutive nucleotides within the X RNA. In some embodiments, the viral target sequence comprises 5 to 40 nucleotides within the X RNA. In some embodiments, the viral target sequence comprises at least 5, 6, 7, 8, 9, 10, 11, 12, 13, 14, 15, 16, 17, 18, 19, 20, 21, 22, 23, 24, 25, 26, 27, 28, 29, or 30 consecutive nucleotides within positions 1374-1838 of SEQ ID NO: 1 or a comparable region in SEQ ID NO: 2 or a sequence of any of HBV genotypes A-J. In some embodiments, the viral target sequence comprises 5 to 40 nucleotides within positions 1374-1838 of SEQ ID NO: 1 or a comparable region in SEQ ID NO: 2 or a sequence of any of HBV genotypes A-J. In some embodiments, the comparable region in SEQ ID NO: 2 or the sequence of any of HBV genotypes A-J is at least about 80%, 86%, 87%, 88%, 89%, 90%, 91%, 92%, 93%, 94%, 95%, 96%, 97%, 98%, or 99% identical to the region in SEQ ID NO: 1. In some embodiments, the comparable region in SEQ ID NO: 2 or the sequence of any of HBV genotypes A-J is based on an alignment of the sequence of SEQ ID NO: 2 or HBV genotypes A-J to the sequence of SEQ ID NO: 1.

In some embodiments, the viral target sequence is in a promoter region of the HBV transcript. In some embodiments, the viral target sequence is in an enhancer region of the HBV transcript. In some embodiments, the viral target sequence is upstream of the promoter or enhancer region of the HBV transcript. In some embodiments, the viral target sequence is downstream of the promoter or enhancer region of the HBV transcript. In some embodiments, the viral target sequence is within about 2000, 1900, 1800, 1700, 1600, 1500, 1400, 1300, 1200, 1100, 1000, 900, 800, 700, 600, 500, 400, 300, 200, or 100 nucleotides of the promoter or enhancer region of the HBV transcript.

In some embodiments, the viral target sequence is in the promoter region of the pre-Core RNA (pre-Core RNA promoter). In some embodiments, the pre-Core RNA promoter comprises the nucleotide sequence corresponding to nucleotides 1742 . . . 1849 of SEQ ID NO: 1 or a comparable region in SEQ ID NO: 2 or a sequence of any of HBV genotypes A-J. In some embodiments, the viral target sequence comprises at least 5, 6, 7, 8, 9, 10, 11, 12, 13, 14, 15, 16, 17, 18, 19, 20, 21, 22, 23, 24, 25, 26, 27, 28, 29, or 30 consecutive nucleotides within the pre-Core RNA promoter. In some embodiments, the viral target sequence comprises less than 50, 45, 40, 35, 34, 33, 32, 31, 30, 29, 28, 27, 26, 25, 24, 23, 22, 21, or 20 consecutive nucleotides within the pre-Core RNA promoter. In some embodiments, the viral target sequence comprises 5 to 40 nucleotides within the pre-Core RNA promoter. In some embodiments, the viral target sequence comprises at least 5, 6, 7, 8, 9, 10, 11, 12, 13, 14, 15, 16, 17, 18, 19, 20, 21, 22, 23, 24, 25, 26, 27, 28, 29, or 30 consecutive nucleotides within positions 1742-1849 of SEQ ID NO: 1 or a comparable region in SEQ ID NO: 2 or a sequence of any of HBV genotypes A-J. In some embodiments, the viral target sequence comprises 5 to 40 nucleotides within positions 1742-1849 of SEQ ID NO: 1 or a comparable region in SEQ ID NO: 2 or a sequence of any of HBV genotypes A-J. In some embodiments, the comparable region in SEQ ID NO: 2 or the sequence of any of HBV genotypes A-J is at least about 80%, 86%, 87%, 88%, 89%, 90%, 91%, 92%, 93%, 94%, 95%, 96%, 97%, 98%, or 99% identical to the region in SEQ ID NO: 1. In some embodiments, the comparable region in SEQ ID NO: 2 or the sequence of any of HBV genotypes A-J is based on an alignment of the sequence of SEQ ID NO: 2 or HBV genotypes A-J to the sequence of SEQ ID NO: 1.

In some embodiments, the viral target sequence is in the promoter region of the pre-S1 RNA (pre-S1 promoter). In some embodiments, the pre-S1 RNA promoter comprises the nucleotide sequence corresponding to nucleotides 2800-2900 of SEQ ID NO: 1 or a comparable region in SEQ ID NO: 2 or a sequence of any of HBV genotypes A-J. In some embodiments, the viral target sequence comprises at least 5, 6, 7, 8, 9, 10, 11, 12, 13, 14, 15, 16, 17, 18, 19, 20, 21, 22, 23, 24, 25, 26, 27, 28, 29, or 30 consecutive nucleotides within the pre-S1 RNA promoter. In some embodiments, the viral target sequence comprises less than 50, 45, 40, 35, 34, 33, 32, 31, 30, 29, 28, 27, 26, 25, 24, 23, 22, 21, or 20 consecutive nucleotides within the pre-S1 RNA promoter. In some embodiments, the viral target sequence comprises 5 to 40 nucleotides within the pre-S1 RNA promoter. In some embodiments, the viral target sequence comprises at least 5, 6, 7, 8, 9, 10, 11, 12, 13, 14, 15, 16, 17, 18, 19, 20, 21, 22, 23, 24, 25, 26, 27, 28, 29, or 30 consecutive nucleotides within positions 2800-2900 of SEQ ID NO: 1 or a comparable region in SEQ ID NO: 2 or a sequence of any of HBV genotypes A-J. In some embodiments, the viral target sequence comprises 5 to 40 nucleotides within positions 2800-2900 of SEQ ID NO: 1 or a comparable region in SEQ ID NO: 2 or a sequence of any of HBV genotypes A-J. In some embodiments, the comparable region in SEQ ID NO: 2 or the sequence of any of HBV genotypes A-J is at least about 80%, 86%, 87%, 88%, 89%, 90%, 91%, 92%, 93%, 94%, 95%, 96%, 97%, 98%, or 99% identical to the region in SEQ ID NO: 1. In some embodiments, the comparable region in SEQ ID NO: 2 or the sequence of any of HBV genotypes A-J is based on an alignment of the sequence of SEQ ID NO: 2 or HBV genotypes A-J to the sequence of SEQ ID NO: 1.

In some embodiments, the viral target sequence is in the promoter of the pre-S2 RNA (pre-S2 promoter). In some embodiments, the pre-S2 RNA promoter comprises the nucleotide sequence corresponding to nucleotides 3100-3215 of SEQ ID NO: 1 or a comparable region in SEQ ID NO: 2 or a sequence of any of HBV genotypes A-J. In some embodiments, the viral target sequence comprises at least 5, 6, 7, 8, 9, 10, 11, 12, 13, 14, 15, 16, 17, 18, 19, 20, 21, 22, 23, 24, 25, 26, 27, 28, 29, or 30 consecutive nucleotides within the pre-S2 RNA promoter. In some embodiments, the viral target sequence comprises less than 50, 45, 40, 35, 34, 33, 32, 31, 30, 29, 28, 27, 26, 25, 24, 23, 22, 21, or 20 consecutive nucleotides within the pre-S2 RNA promoter. In some embodiments, the viral target sequence comprises 5 to 40 nucleotides within the pre-S2 RNA promoter. In some embodiments, the viral target sequence comprises at least 5, 6, 7, 8, 9, 10, 11, 12, 13, 14, 15, 16, 17, 18, 19, 20, 21, 22, 23, 24, 25, 26, 27, 28, 29, or 30 consecutive nucleotides within positions 3100-3215 of SEQ ID NO: 1 or a comparable region in SEQ ID NO: 2 or a sequence of any of HBV genotypes A-J. In some embodiments, the viral target sequence comprises 5 to 40 nucleotides within positions 3100-3215 of SEQ ID NO: 1 or a comparable region in SEQ ID NO: 2 or a sequence of any of HBV genotypes A-J. In some embodiments, the comparable region in SEQ ID NO: 2 or the sequence of any of HBV genotypes A-J is at least about 80%, 86%, 87%, 88%, 89%, 90%, 91%, 92%, 93%, 94%, 95%, 96%, 97%, 98%, or 99% identical to the region in SEQ ID NO: 1. In some embodiments, the comparable region in SEQ ID NO: 2 or the sequence of any of HBV genotypes A-J is based on an alignment of the sequence of SEQ ID NO: 2 or HBV genotypes A-J to the sequence of SEQ ID NO: 1.

In some embodiments, the viral target sequence is in the promoter of X RNA (X RNA promoter). In some embodiments, the X RNA promoter comprises the nucleotide sequence corresponding to nucleotides 1200-1400 of SEQ ID NO: 1 or a comparable region in SEQ ID NO: 2 or a sequence of any of HBV genotypes A-J. In some embodiments, the viral target sequence comprises at least 5, 6, 7, 8, 9, 10, 11, 12, 13, 14, 15, 16, 17, 18, 19, 20, 21, 22, 23, 24, 25, 26, 27, 28, 29, or 30 consecutive nucleotides within the X RNA promoter. In some embodiments, the viral target sequence comprises less than 50, 45, 40, 35, 34, 33, 32, 31, 30, 29, 28, 27, 26, 25, 24, 23, 22, 21, or 20 consecutive nucleotides within the X RNA promoter. In some embodiments, the viral target sequence comprises 5 to 40 nucleotides within the X RNA promoter. In some embodiments, the viral target sequence comprises at least 5, 6, 7, 8, 9, 10, 11, 12, 13, 14, 15, 16, 17, 18, 19, 20, 21, 22, 23, 24, 25, 26, 27, 28, 29, or 30 consecutive nucleotides within positions 1200-1400 of SEQ ID NO: 1 or a comparable region in SEQ ID NO: 2 or a sequence of any of HBV genotypes A-J. In some embodiments, the viral target sequence comprises 5 to 40 nucleotides within positions 1200-1400 of SEQ ID NO: 1 or a comparable region in SEQ ID NO: 2 or a sequence of any of HBV genotypes A-J. In some embodiments, the comparable region in SEQ ID NO: 2 or the sequence of any of HBV genotypes A-J is at least about 80%, 86%, 87%, 88%, 89%, 90%, 91%, 92%, 93%, 94%, 95%, 96%, 97%, 98%, or 99% identical to the region in SEQ ID NO: 1. In some embodiments, the comparable region in SEQ ID NO: 2 or the sequence of any of HBV genotypes A-J is based on an alignment of the sequence of SEQ ID NO: 2 or HBV genotypes A-J to the sequence of SEQ ID NO: 1.

In some embodiments, the viral target sequence is in the promoter of core RNA (core RNA promoter). In some embodiments, the core RNA promoter comprises the nucleotide sequence corresponding to nucleotides 1750-1900 of SEQ ID NO: 1 or a comparable region in SEQ ID NO: 2 or a sequence of any of HBV genotypes A-J. In some embodiments, the viral target sequence comprises at least 5, 6, 7, 8, 9, 10, 11, 12, 13, 14, 15, 16, 17, 18, 19, 20, 21, 22, 23, 24, 25, 26, 27, 28, 29, or 30 consecutive nucleotides within the core RNA promoter. In some embodiments, the viral target sequence comprises less than 50, 45, 40, 35, 34, 33, 32, 31, 30, 29, 28, 27, 26, 25, 24, 23, 22, 21, or 20 consecutive nucleotides within the core RNA promoter. In some embodiments, the viral target sequence comprises 5 to 40 nucleotides within the core RNA promoter. In some embodiments, the viral target sequence comprises at least 5, 6, 7, 8, 9, 10, 11, 12, 13, 14, 15, 16, 17, 18, 19, 20, 21, 22, 23, 24, 25, 26, 27, 28, 29, or 30 consecutive nucleotides within positions 1750-1900 of SEQ ID NO: 1 or a comparable region in SEQ ID NO: 2 or a sequence of any of HBV genotypes A-J. In some embodiments, the viral target sequence comprises 5 to 40 nucleotides within positions 1750-1900 of SEQ ID NO: 1 or a comparable region in SEQ ID NO: 2 or a sequence of any of HBV genotypes A-J. In some embodiments, the comparable region in SEQ ID NO: 2 or the sequence of any of HBV genotypes A-J is at least about 80%, 86%, 87%, 88%, 89%, 90%, 91%, 92%, 93%, 94%, 95%, 96%, 97%, 98%, or 99% identical to the region in SEQ ID NO: 1. In some embodiments, the comparable region in SEQ ID NO: 2 or the sequence of any of HBV genotypes A-J is based on an alignment of the sequence of SEQ ID NO: 2 or HBV genotypes A-J to the sequence of SEQ ID NO: 1.

In some embodiments, the viral target sequence is in an enhancer region of the HBV genome. In some embodiments, the enhancer is enhancer 1 (Enh1). In some embodiments, Enh1 comprises the nucleotide sequence corresponding to nucleotides 900-1400 of SEQ ID NO: 1 or a comparable region in SEQ ID NO: 2 or a sequence of any of HBV genotypes A-J. In some embodiments, the enhancer is enhancer 2 (Enh2). In some embodiments, Enh2 comprises the nucleotide sequence corresponding to nucleotides 1550-1900 of SEQ ID NO: 1 or a comparable region in SEQ ID NO: 2 or a sequence of any of HBV genotypes A-J. In some embodiments, the viral target sequence comprises at least 5, 6, 7, 8, 9, 10, 11, 12, 13, 14, 15, 16, 17, 18, 19, 20, 21, 22, 23, 24, 25, 26, 27, 28, 29, or 30 consecutive nucleotides within the enhancer region. In some embodiments, the viral target sequence comprises less than 50, 45, 40, 35, 34, 33, 32, 31, 30, 29, 28, 27, 26, 25, 24, 23, 22, 21, or 20 consecutive nucleotides within the enhancer region. In some embodiments, the viral target sequence comprises 5 to 40 nucleotides within the enhancer region. In some embodiments, the viral target sequence comprises at least 5, 6, 7, 8, 9, 10, 11, 12, 13, 14, 15, 16, 17, 18, 19, 20, 21, 22, 23, 24, 25, 26, 27, 28, 29, or 30 consecutive nucleotides within positions 900-1400 or 1550-1900 of SEQ ID NO: 1 or a comparable region in SEQ ID NO: 2 or a sequence of any of HBV genotypes A-J. In some embodiments, the viral target sequence comprises 5 to 40 nucleotides within positions 900-1400 or 1550-1900 of SEQ ID NO: 1 or a comparable region in SEQ ID NO: 2 or a sequence of any of HBV genotypes A-J. In some embodiments, the comparable region in SEQ ID NO: 2 or the sequence of any of HBV genotypes A-J is at least about 80%, 86%, 87%, 88%, 89%, 90%, 91%, 92%, 93%, 94%, 95%, 96%, 97%, 98%, or 99% identical to the region in SEQ ID NO: 1. In some embodiments, the comparable region in SEQ ID NO: 2 or the sequence of any of HBV genotypes A-J is based on an alignment of the sequence of SEQ ID NO: 2 or HBV genotypes A-J to the sequence of SEQ ID NO: 1.

In some embodiments, the nucleotide sequence is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical to 5 to 40 consecutive nucleotides starting at position 1181, 1255, 1256, 1257, 1258, 1259, 1260, 1261, 1263, 1264, 1265, 1266, 1267, 1268, 1393, 1394, 1410, 1411, 1412, 1515, 1516, 1517, 1523, 1527, 1528, 1529, 1530, 1531, 1577, or 2817 of SEQ ID NO: 1, or a comparable position in SEQ ID NO: 2 or the sequence of any of HBV genotypes A-J, along the entire length of the nucleotide sequence. In some embodiments, the nucleotide sequence is at least 70% identical to 5 to 40 consecutive nucleotides starting at position 1181, 1255, 1256, 1257, 1258, 1259, 1260, 1261, 1263, 1264, 1265, 1266, 1267, 1268, 1393, 1394, 1410, 1411, 1412, 1515, 1516, 1517, 1523, 1527, 1528, 1529, 1530, 1531, 1577, or 2817 of SEQ ID NO: 1, or a comparable position in SEQ ID NO: 2 or the sequence of any of HBV genotypes A-J, along the entire length of the nucleotide sequence. In some embodiments, the nucleotide sequence is at least 80% identical to 5 to 40 consecutive nucleotides starting at position 1181, 1255, 1256, 1257, 1258, 1259, 1260, 1261, 1263, 1264, 1265, 1266, 1267, 1268, 1393, 1394, 1410, 1411, 1412, 1515, 1516, 1517, 1523, 1527, 1528, 1529, 1530, 1531, 1577, or 2817 of SEQ ID NO: 1, or a comparable position in SEQ ID NO: 2 or the sequence of any of HBV genotypes A-J, along the entire length of the nucleotide sequence. In some embodiments, the nucleotide sequence is at least 85% identical to 5 to 40 consecutive nucleotides starting at position 1181, 1255, 1256, 1257, 1258, 1259, 1260, 1261, 1263, 1264, 1265, 1266, 1267, 1268, 1393, 1394, 1410, 1411, 1412, 1515, 1516, 1517, 1523, 1527, 1528, 1529, 1530, 1531, 1577, or 2817 of SEQ ID NO: 1, or a comparable position in SEQ ID NO: 2 or the sequence of any of HBV genotypes A-J, along the entire length of the nucleotide sequence. In some embodiments, the nucleotide sequence is at least 90% identical to 5 to 40 consecutive nucleotides starting at position 1181, 1255, 1256, 1257, 1258, 1259, 1260, 1261, 1263, 1264, 1265, 1266, 1267, 1268, 1393, 1394, 1410, 1411, 1412, 1515, 1516, 1517, 1523, 1527, 1528, 1529, 1530, 1531, 1577, or 2817 of SEQ ID NO: 1, or a comparable position in SEQ ID NO: 2 or the sequence of any of HBV genotypes A-J, along the entire length of the nucleotide sequence.

In some embodiments, the nucleotide sequence is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical to 5 to 40 consecutive nucleotides within positions 950-1050, 975-1025, 975-1010, 980-1010, 1150-1275, 1175-1275, 1180-1270, 1180-1250, 1180-1225, 1180-1210, 1225-1350, 1225-1325, 1225-1300, 1225-1290, 1250-1350, 1250-1325, 1250-1300, 1250-1290, 1375-1475, 1375-1450, 1375-1440, 1375-1430, 1390-1475, 1390-1450, 1390-1440, 1390-1430, 1500-1700, 1500-1650, 1500-1620, 1500-1595, 1510-1700, 1510-1650, 1510-1620, 1510-1595, 1515-1700, 1515-1650, 1515-1620, 1515-1590, or 2817-3050 of SEQ ID NO: 1, or a comparable position in SEQ ID NO: 2 or the sequence of any of HBV genotypes A-J, along the entire length of the nucleotide sequence. In some embodiments, the nucleotide sequence is at least 70% identical to 5 to 40 consecutive nucleotides within positions 950-1050, 975-1025, 975-1010, 980-1010, 1150-1275, 1175-1275, 1180-1270, 1180-1250, 1180-1225, 1180-1210, 1225-1350, 1225-1325, 1225-1300, 1225-1290, 1250-1350, 1250-1325, 1250-1300, 1250-1290, 1375-1475, 1375-1450, 1375-1440, 1375-1430, 1390-1475, 1390-1450, 1390-1440, 1390-1430, 1500-1700, 1500-1650, 1500-1620, 1500-1595, 1510-1700, 1510-1650, 1510-1620, 1510-1595, 1515-1700, 1515-1650, 1515-1620, 1515-1590, or 2817-3050 of SEQ ID NO: 1, or a comparable position in SEQ ID NO: 2 or the sequence of any of HBV genotypes A-J, along the entire length of the nucleotide sequence. In some embodiments, the nucleotide sequence is at least 80% identical to 5 to 40 consecutive nucleotides within positions 950-1050, 975-1025, 975-1010, 980-1010, 1150-1275, 1175-1275, 1180-1270, 1180-1250, 1180-1225, 1180-1210, 1225-1350, 1225-1325, 1225-1300, 1225-1290, 1250-1350, 1250-1325, 1250-1300, 1250-1290, 1375-1475, 1375-1450, 1375-1440, 1375-1430, 1390-1475, 1390-1450, 1390-1440, 1390-1430, 1500-1700, 1500-1650, 1500-1620, 1500-1595, 1510-1700, 1510-1650, 1510-1620, 1510-1595, 1515-1700, 1515-1650, 1515-1620, 1515-1590, or 2817-3050 of SEQ ID NO: 1, or a comparable position in SEQ ID NO: 2 or the sequence of any of HBV genotypes A-J, along the entire length of the nucleotide sequence. In some embodiments, the nucleotide sequence is at least 85% identical to 5 to 40 consecutive nucleotides within positions 950-1050, 975-1025, 975-1010, 980-1010, 1150-1275, 1175-1275, 1180-1270, 1180-1250, 1180-1225, 1180-1210, 1225-1350, 1225-1325, 1225-1300, 1225-1290, 1250-1350, 1250-1325, 1250-1300, 1250-1290, 1375-1475, 1375-1450, 1375-1440, 1375-1430, 1390-1475, 1390-1450, 1390-1440, 1390-1430, 1500-1700, 1500-1650, 1500-1620, 1500-1595, 1510-1700, 1510-1650, 1510-1620, 1510-1595, 1515-1700, 1515-1650, 1515-1620, 1515-1590, or 2817-3050 of SEQ ID NO: 1, or a comparable position in SEQ ID NO: 2 or the sequence of any of HBV genotypes A-J, along the entire length of the nucleotide sequence. In some embodiments, the nucleotide sequence is at least 90% identical to 5 to 40 consecutive nucleotides within positions 950-1050, 975-1025, 975-1010, 980-1010, 1150-1275, 1175-1275, 1180-1270, 1180-1250, 1180-1225, 1180-1210, 1225-1350, 1225-1325, 1225-1300, 1225-1290, 1250-1350, 1250-1325, 1250-1300, 1250-1290, 1375-1475, 1375-1450, 1375-1440, 1375-1430, 1390-1475, 1390-1450, 1390-1440, 1390-1430, 1500-1700, 1500-1650, 1500-1620, 1500-1595, 1510-1700, 1510-1650, 1510-1620, 1510-1595, 1515-1700, 1515-1650, 1515-1620, 1515-1590, or 2817-3050 of SEQ ID NO: 1, or a comparable position in SEQ ID NO: 2 or the sequence of any of HBV genotypes A-J, along the entire length of the nucleotide sequence.

In some embodiments, the nucleotide sequence is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% complementary to 5 to 40 consecutive nucleotides starting at position 1181, 1255, 1256, 1257, 1258, 1259, 1260, 1261, 1263, 1264, 1265, 1266, 1267, 1268, 1393, 1394, 1410, 1411, 1412, 1515, 1516, 1517, 1523, 1527, 1528, 1529, 1530, 1531, 1577, or 2817 of SEQ ID NO: 1, or a comparable position in SEQ ID NO: 2 or the sequence of any of HBV genotypes A-J, along the entire length of the nucleotide sequence. In some embodiments, the nucleotide sequence is at least 70% complementary to 5 to 40 consecutive nucleotides starting at position 1181, 1255, 1256, 1257, 1258, 1259, 1260, 1261, 1263, 1264, 1265, 1266, 1267, 1268, 1393, 1394, 1410, 1411, 1412, 1515, 1516, 1517, 1523, 1527, 1528, 1529, 1530, 1531, 1577, or 2817 of SEQ ID NO: 1, or a comparable position in SEQ ID NO: 2 or the sequence of any of HBV genotypes A-J, along the entire length of the nucleotide sequence. In some embodiments, the nucleotide sequence is at least 80% complementary to 5 to 40 consecutive nucleotides starting at position 1181, 1255, 1256, 1257, 1258, 1259, 1260, 1261, 1263, 1264, 1265, 1266, 1267, 1268, 1393, 1394, 1410, 1411, 1412, 1515, 1516, 1517, 1523, 1527, 1528, 1529, 1530, 1531, 1577, or 2817 of SEQ ID NO: 1, or a comparable position in SEQ ID NO: 2 or the sequence of any of HBV genotypes A-J, along the entire length of the nucleotide sequence. In some embodiments, the nucleotide sequence is at least 85% complementary to 5 to 40 consecutive nucleotides starting at position 1181, 1255, 1256, 1257, 1258, 1259, 1260, 1261, 1263, 1264, 1265, 1266, 1267, 1268, 1393, 1394, 1410, 1411, 1412, 1515, 1516, 1517, 1523, 1527, 1528, 1529, 1530, 1531, 1577, or 2817 of SEQ ID NO: 1, or a comparable position in SEQ ID NO: 2 or the sequence of any of HBV genotypes A-J, along the entire length of the nucleotide sequence. In some embodiments, the nucleotide sequence is at least 90% complementary to 5 to 40 consecutive nucleotides starting at position 1181, 1255, 1256, 1257, 1258, 1259, 1260, 1261, 1263, 1264, 1265, 1266, 1267, 1268, 1393, 1394, 1410, 1411, 1412, 1515, 1516, 1517, 1523, 1527, 1528, 1529, 1530, 1531, 1577, or 2817 of SEQ ID NO: 1, or a comparable position in SEQ ID NO: 2 or the sequence of any of HBV genotypes A-J, along the entire length of the nucleotide sequence.

In some embodiments, the nucleotide sequence is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% complementary to 5 to 40 consecutive nucleotides within positions 950-1050, 975-1025, 975-1010, 980-1010, 1150-1275, 1175-1275, 1180-1270, 1180-1250, 1180-1225, 1180-1210, 1225-1350, 1225-1325, 1225-1300, 1225-1290, 1250-1350, 1250-1325, 1250-1300, 1250-1290, 1375-1475, 1375-1450, 1375-1440, 1375-1430, 1390-1475, 1390-1450, 1390-1440, 1390-1430, 1500-1700, 1500-1650, 1500-1620, 1500-1595, 1510-1700, 1510-1650, 1510-1620, 1510-1595, 1515-1700, 1515-1650, 1515-1620, 1515-1590, or 2817-3050 of SEQ ID NO: 1, or a comparable position in SEQ ID NO: 2 or the sequence of any of HBV genotypes A-J, along the entire length of the nucleotide sequence. In some embodiments, the nucleotide sequence is at least 70% complementary to 5 to 40 consecutive nucleotides within positions 950-1050, 975-1025, 975-1010, 980-1010, 1150-1275, 1175-1275, 1180-1270, 1180-1250, 1180-1225, 1180-1210, 1225-1350, 1225-1325, 1225-1300, 1225-1290, 1250-1350, 1250-1325, 1250-1300, 1250-1290, 1375-1475, 1375-1450, 1375-1440, 1375-1430, 1390-1475, 1390-1450, 1390-1440, 1390-1430, 1500-1700, 1500-1650, 1500-1620, 1500-1595, 1510-1700, 1510-1650, 1510-1620, 1510-1595, 1515-1700, 1515-1650, 1515-1620, 1515-1590, or 2817-3050 of SEQ ID NO: 1, or a comparable position in SEQ ID NO: 2 or the sequence of any of HBV genotypes A-J, along the entire length of the nucleotide sequence. In some embodiments, the nucleotide sequence is at least 80% complementary to 5 to 40 consecutive nucleotides within positions 950-1050, 975-1025, 975-1010, 980-1010, 1150-1275, 1175-1275, 1180-1270, 1180-1250, 1180-1225, 1180-1210, 1225-1350, 1225-1325, 1225-1300, 1225-1290, 1250-1350, 1250-1325, 1250-1300, 1250-1290, 1375-1475, 1375-1450, 1375-1440, 1375-1430, 1390-1475, 1390-1450, 1390-1440, 1390-1430, 1500-1700, 1500-1650, 1500-1620, 1500-1595, 1510-1700, 1510-1650, 1510-1620, 1510-1595, 1515-1700, 1515-1650, 1515-1620, 1515-1590, or 2817-3050 of SEQ ID NO: 1, or a comparable position in SEQ ID NO: 2 or the sequence of any of HBV genotypes A-J, along the entire length of the nucleotide sequence. In some embodiments, the nucleotide sequence is at least 85% complementary to 5 to 40 consecutive nucleotides within positions 950-1050, 975-1025, 975-1010, 980-1010, 1150-1275, 1175-1275, 1180-1270, 1180-1250, 1180-1225, 1180-1210, 1225-1350, 1225-1325, 1225-1300, 1225-1290, 1250-1350, 1250-1325, 1250-1300, 1250-1290, 1375-1475, 1375-1450, 1375-1440, 1375-1430, 1390-1475, 1390-1450, 1390-1440, 1390-1430, 1500-1700, 1500-1650, 1500-1620, 1500-1595, 1510-1700, 1510-1650, 1510-1620, 1510-1595, 1515-1700, 1515-1650, 1515-1620, 1515-1590, or 2817-3050 of SEQ ID NO: 1, or a comparable position in SEQ ID NO: 2 or the sequence of any of HBV genotypes A-J, along the entire length of the nucleotide sequence. In some embodiments, the nucleotide sequence is at least 90% complementary to 5 to 40 consecutive nucleotides within positions 950-1050, 975-1025, 975-1010, 980-1010, 1150-1275, 1175-1275, 1180-1270, 1180-1250, 1180-1225, 1180-1210, 1225-1350, 1225-1325, 1225-1300, 1225-1290, 1250-1350, 1250-1325, 1250-1300, 1250-1290, 1375-1475, 1375-1450, 1375-1440, 1375-1430, 1390-1475, 1390-1450, 1390-1440, 1390-1430, 1500-1700, 1500-1650, 1500-1620, 1500-1595, 1510-1700, 1510-1650, 1510-1620, 1510-1595, 1515-1700, 1515-1650, 1515-1620, 1515-1590, or 2817-3050 of SEQ ID NO: 1, or a comparable position in SEQ ID NO: 2 or the sequence of any of HBV genotypes A-J, along the entire length of the nucleotide sequence.

In some embodiments, the nucleotide sequence hybridizes under high stringency conditions to 5 to 40 consecutive nucleotides starting at position 1181, 1255, 1256, 1257, 1258, 1259, 1260, 1261, 1263, 1264, 1265, 1266, 1267, 1268, 1393, 1394, 1410, 1411, 1412, 1515, 1516, 1517, 1523, 1527, 1528, 1529, 1530, 1531, 1577, or 2817 of SEQ ID NO: 1 or a comparable region in SEQ ID NO: 2 or a sequence of any of HBV genotypes A-J. In some embodiments, the comparable region in SEQ ID NO: 2 or the sequence of any of HBV genotypes A-J is at least about 80%, 86%, 87%, 88%, 89%, 90%, 91%, 92%, 93%, 94%, 95%, 96%, 97%, 98%, or 99% identical to the region in SEQ ID NO: 1. In some embodiments, the comparable region in SEQ ID NO: 2 or the sequence of any of HBV genotypes A-J is based on an alignment of the sequence of SEQ ID NO: 2 or HBV genotypes A-J to the sequence of SEQ ID NO: 1.

In some embodiments, the nucleotide sequence hybridizes under high stringency conditions to 5 to 40 consecutive nucleotides within positions 950-1050, 975-1025, 975-1010, 980-1010, 1150-1275, 1175-1275, 1180-1270, 1180-1250, 1180-1225, 1180-1210, 1225-1350, 1225-1325, 1225-1300, 1225-1290, 1250-1350, 1250-1325, 1250-1300, 1250-1290, 1375-1475, 1375-1450, 1375-1440, 1375-1430, 1390-1475, 1390-1450, 1390-1440, 1390-1430, 1500-1700, 1500-1650, 1500-1620, 1500-1595, 1510-1700, 1510-1650, 1510-1620, 1510-1595, 1515-1700, 1515-1650, 1515-1620, 1515-1590, or 2817-3050 of SEQ ID NO: 1 or a comparable region in SEQ ID NO: 2 or a sequence of any of HBV genotypes A-J. In some embodiments, the comparable region in SEQ ID NO: 2 or the sequence of any of HBV genotypes A-J is at least about 80%, 86%, 87%, 88%, 89%, 90%, 91%, 92%, 93%, 94%, 95%, 96%, 97%, 98%, or 99% identical to the region in SEQ ID NO: 1. In some embodiments, the comparable region in SEQ ID NO: 2 or the sequence of any of HBV genotypes A-J is based on an alignment of the sequence of SEQ ID NO: 2 or HBV genotypes A-J to the sequence of SEQ ID NO: 1.

In some embodiments, the nucleotide sequence preferentially hybridizes to 5 to 40 consecutive nucleotides starting at position 1181, 1255, 1256, 1257, 1258, 1259, 1260, 1261, 1263, 1264, 1265, 1266, 1267, 1268, 1393, 1394, 1410, 1411, 1412, 1515, 1516, 1517, 1523, 1527, 1528, 1529, 1530, 1531, 1577, or 2817 of SEQ ID NO: 1, or a comparable position in SEQ ID NO: 2 or a sequence of any of HBV genotypes A-J, as compared to other positions within SEQ ID NO: 1, or SEQ ID NO: 2 or the sequence of any of HBV genotypes A-J. In some embodiments, the comparable region in SEQ ID NO: 2 or the sequence of any of HBV genotypes A-J is at least about 80%, 86%, 87%, 88%, 89%, 90%, 91%, 92%, 93%, 94%, 95%, 96%, 97%, 98%, or 99% identical to the region in SEQ ID NO: 1. In some embodiments, the comparable region in SEQ ID NO: 2 or the sequence of any of HBV genotypes A-J is based on an alignment of the sequence of SEQ ID NO: 2 or HBV genotypes A-J to the sequence of SEQ ID NO: 1.

In some embodiments, the nucleotide sequence preferentially hybridizes to 5 to 40 consecutive nucleotides within positions 950-1050, 975-1025, 975-1010, 980-1010, 1150-1275, 1175-1275, 1180-1270, 1180-1250, 1180-1225, 1180-1210, 1225-1350, 1225-1325, 1225-1300, 1225-1290, 1250-1350, 1250-1325, 1250-1300, 1250-1290, 1375-1475, 1375-1450, 1375-1440, 1375-1430, 1390-1475, 1390-1450, 1390-1440, 1390-1430, 1500-1700, 1500-1650, 1500-1620, 1500-1595, 1510-1700, 1510-1650, 1510-1620, 1510-1595, 1515-1700, 1515-1650, 1515-1620, 1515-1590, or 2817-3050 of SEQ ID NO: 1 as compared to other positions within SEQ ID NO: 1. In some embodiments, the comparable region in SEQ ID NO: 2 or the sequence of any of HBV genotypes A-J is at least about 80%, 86%, 87%, 88%, 89%, 90%, 91%, 92%, 93%, 94%, 95%, 96%, 97%, 98%, or 99% identical to the region in SEQ ID NO: 1. In some embodiments, the comparable region in SEQ ID NO: 2 or the sequence of any of HBV genotypes A-J is based on an alignment of the sequence of SEQ ID NO: 2 or HBV genotypes A-J to the sequence of SEQ ID NO: 1.

In some embodiments, the nucleotide sequence comprises 10 to 100, 10 to 90, 10 to 80, 10 to 70, 10 to 60, 10 to 50, 10 to 40, 10 to 35, 5 to 40, 10 to 25, 10 to 23, 10 to 22, 10 to 20, 14 to 60, 14 to 50, 14 to 40, 14 to 35, 14 to 30, 14 to 25, 14 to 24, 14 to 23, 14 to 22, 14 to 20, 14 to 19, 14 to 18, 14 to 17, 15 to 100, 15 to 90, 15 to 80, 15 to 70, 15 to 60, 15 to 50, 15 to 40, 15 to 35, 15 to 30, 15 to 25, 15 to 22, 15 to 21, 15 to 20, 15 to 19, 15 to 18, or 15 to 17 nucleotides. In some embodiments, the nucleotide sequence comprises 14 to 22 nucleotides. In some embodiments, the nucleotide sequence comprises 15 to 22 nucleotides. In some embodiments, the nucleotide sequence comprises 15 to 17 nucleotides. In some embodiments, the nucleotide sequence comprises at least 10, 11, 12, 13, 14, 15, 16, 17, 18, 19, 20, 21, or 22 nucleotides. In some embodiments, the nucleotide sequence comprises at least 15 nucleotides. In some embodiments, the nucleotide sequence comprises at least 16 nucleotides. In some embodiments, the nucleotide sequence comprises at least 17 nucleotides. In some embodiments, the nucleotide sequence comprises less than or equal to 35, 34, 33, 32, 31, 30, 29, 28, 27, 26, 25, 24, 23, 22, 21, 20, 19, 18, or 17 nucleotides. In some embodiments, the nucleotide sequence comprises less than or equal to 22 nucleotides. In some embodiments, the nucleotide sequence comprises less than or equal to 21 nucleotides. In some embodiments, the nucleotide sequence comprises less than or equal to 20 nucleotides. In some embodiments, the nucleotide sequence comprises less than or equal to 19 nucleotides. In some embodiments, the nucleotide sequence comprises less than or equal to 18 nucleotides.

In some embodiments, at least 2, 3, 4, 5, 6, 7, 8, 9, 10, 11, 12, 13, 14, 15, 16, 17, 18, 19, 20, 21, 22, 23, 24 or more of the nucleotides in the nucleotide sequence are modified nucleosides. In some embodiments, fewer than or equal to 25, 24, 23, 22, 21, 20, 19, 18, 17, 16, 15, 14, 13, 12, 10, 11, 10, 9, 8, 7, 6, 5, 4, 3, or 2 of the nucleotides in the nucleotide sequence are modified nucleosides. In some embodiments, at least 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, 97%, 99%, or 100% of the nucleotides in the nucleotide sequence are modified nucleosides. In some embodiments, less than or equal to 100%, 99%, 97%, 95%, 90%, 85%, 80%, 75%, 70%, 65%, 60%, 55%, 50%, 45%, 40%, 35%, 30%, 25%, or 20% of the nucleotides in the nucleotide sequence are modified nucleosides.

In some embodiments, any of the oligonucleotides disclosed herein comprise a nucleotide modification pattern of (XY)ₙ (“alternating nucleosides”), wherein X represents a first class of modified nucleosides, and Y represents a second class of modified nucleosides, wherein X and Y are different, and n is a number between 1 to 15. In some embodiments, the modified nucleosides are selected locked nucleosides, 2′-substituted nucleosides, and 2′-O-methyl nucleosides. In some embodiments, the modified nucleosides are selected locked nucleosides and 2′-substituted nucleosides. In some embodiments, the modified nucleosides are selected locked nucleosides and 2′-O-methyl nucleosides. In some embodiments, the first class of modified nucleosides is locked nucleosides, and the second class of modified nucleosides is 2′-O-methyl nucleosides. In some embodiments, the first class of modified nucleosides is 2′-O-methyl nucleosides, and the second class of modified nucleosides is locked nucleosides. In some embodiments, n is between 1 to 10, 2 to 10, 3 to 10, 4 to 10, 5 to 10, 6 to 10, 7 to 10, 4 to 11, 4 to 12, 4 to 13, 4 to 14, 4 to 15, 5 to 11, 5 to 12, 5 to 13, 5 to 14, 5 to 15, 6 to 11, 6 to 12, 6 to 13, 6 to 14, 6 to 15, 7 to 11, 7 to 12, 7 to 13, 7 to 14, or 7 to 15. In some embodiments, n is at least 4, 5, 6, 7, 8, 9, 10, or 11. In some embodiments, n is at least 7. In some embodiments, the nucleotide sequence comprises at least 15, 16, or 17 nucleotides. In some embodiments, the alternating nucleosides comprise different nucleobases. In some embodiments, the alternating nucleosides comprise at least 2 different nucleobases. In some embodiments, the alternating nucleosides comprise at least 3 different nucleobases. In some embodiments, the alternating nucleosides comprise at least 4 different nucleobases. In some embodiments, two consecutive nucleosides of the alternating nucleosides comprise two different nucleotide modifications but contain the same nucleobase. For instance, the two consecutive nucleosides of the alternating nucleosides comprise a 2′-O-methyl nucleoside and a locked nucleoside, wherein the nucleobase for the 2′-O-methyl nucleoside and locked nucleoside are the same (e.g., both adenine). In some embodiments, two consecutive nucleosides of the alternating nucleosides comprise two different nucleotide modifications and two different nucleobases. For instance, the two consecutive nucleosides of the alternating nucleosides comprise a 2′-O-methyl nucleoside and a locked nucleoside, wherein the nucleobase for the 2′-O-methyl nucleoside and locked nucleoside are different (e.g., a 2′-O-methyl adenosine and an LNA comprising 5-methyl cytosine). In some embodiments, at least two consecutive nucleosides of the alternating nucleosides comprise two different nucleotide modifications but contain the same nucleobase. In some embodiments, the alternating nucleosides comprise an alternating pattern of LNA and 2′-substituted nucleosides (or vice versa). In some embodiments, a pair of alternating LNA and 2′-substituted nucleosides contain the same nucleobase. In some embodiments, a pair of alternating LNA and 2′-substituted nucleosides contain different nucleobases. In some embodiments, the alternating nucleosides comprise an alternating pattern of LNA and 2′-O-methyl nucleosides (or vise versa). In some embodiments, a pair of alternating LNA and 2′-O-methyl nucleosides contain the same nucleobase. In some embodiments, a pair of alternating LNA and 2′-O-methyl nucleosides contain different nucleobases. In some embodiments, a pair of alternating 2′-O-methyl nucleosides and LNA contain the same nucleobase. In some embodiments, a pair of alternating 2′-O-methyl nucleosides and LNA contain different nucleobases.

In some embodiments, the modified nucleoside is a locked nucleoside, a 2′-substituted nucleoside, or a 2′-O-methyl nucleoside. In some embodiments, the nucleotide sequence of any of the oligonucleotides disclosed herein comprise two or more different modified nucleosides selected from a locked nucleoside, 2′-substituted nucleosides, and 2′-O-methyl nucleosides.

In some embodiments, at least 1, 2, 3, 4, 5, 6, 7, 8, 9, 10, 11, 12, 13, 14, 15, 16, 17, 18, 19, 20, 21, 22, 23, 24 or more of the nucleotides in the nucleotide sequence are locked nucleosides. In some embodiments, fewer than or equal to 25, 24, 23, 22, 21, 20, 19, 18, 17, 16, 15, 14, 13, 12, 10, 11, 10, 9, 8, 7, 6, 5, 4, 3, or 2 of the nucleotides in the nucleotide sequence are locked nucleosides. In some embodiments, at least 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, 97%, 99%, or 100% of the nucleotides in the nucleotide sequence are locked nucleosides. In some embodiments, less than or equal to 100%, 99%, 97%, 95%, 90%, 85%, 80%, 75%, 70%, 65%, 60%, 55%, 50%, 45%, 40%, 35%, 30%, 25%, or 20% of the nucleotides in the nucleotide sequence are locked nucleosides.

In some embodiments, the locked nucleoside is selected from any of the locked nucleosides shown in Table 4. In some embodiments, the locked nucleoside is selected from

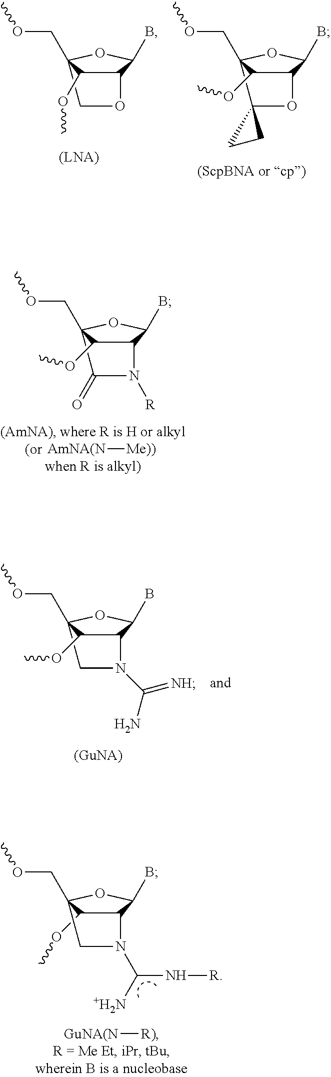

Other suitable locked nucleotides are included in PCT/JP2010/068409, PCT/JP2013/075370, PCT/JP2015/054308, PCT/JP2018/006061, and/or PCT/JP2018/006062, which are incorporated by reference in their entirety.

In some embodiments, at least 1, 2, 3, 4, 5, 6, 7, 8, 9, 10, 11, 12, 13, 14, 15, 16, 17, 18, 19, 20, 21, 22, 23, 24 or more of the nucleotides in the nucleotide sequence are 2′-substituted nucleosides. In some embodiments, fewer than or equal to 25, 24, 23, 22, 21, 20, 19, 18, 17, 16, 15, 14, 13, 12, 10, 11, 10, 9, 8, 7, 6, 5, 4, 3, or 2 of the nucleotides in the nucleotide sequence are 2′-substituted nucleosides. In some embodiments, at least 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, 97%, 99%, or 100% of the nucleotides in the nucleotide sequence are 2′-substituted nucleosides. In some embodiments, less than or equal to 100%, 99%, 97%, 95%, 90%, 85%, 80%, 75%, 70%, 65%, 60%, 55%, 50%, 45%, 40%, 35%, 30%, 25%, or 20% of the nucleotides in the nucleotide sequence are 2′-substituted nucleosides. In some embodiments, the 2′-substituted nucleoside is selected from any of the 2′-substituted nucleosides shown in Table 4. Suitable 2′-substituted nucleosides include, but are not limited to, 2′-O-methoxy nucleotides (e.g., mA, mU, mG, mC, etc.), 2′-O-methoxyethylribose nucleosides (e.g., moeA, moeT, moeG, etc.), and 5-methyl (5m) nucleotides (e.g., (5m)C, moe(5m)C, etc.).

In some embodiments, at least 1, 2, 3, 4, 5, 6, 7, 8, 9, 10, 11, 12, 13, 14, 15, 16, 17, 18, 19, 20, 21, 22, 23, 24 or more of the nucleotides in the nucleotide sequence are 2′-O-methoxy-ethyl (2′-MOE) nucleotides. In some embodiments, fewer than or equal to 25, 24, 23, 22, 21, 20, 19, 18, 17, 16, 15, 14, 13, 12, 10, 11, 10, 9, 8, 7, 6, 5, 4, 3, or 2 of the nucleotides in the nucleotide sequence are 2′-MOE nucleosides. In some embodiments, at least 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, 97%, 99%, or 100% of the nucleotides in the nucleotide sequence are 2′-MOE nucleosides. In some embodiments, less than or equal to 100%, 99%, 97%, 95%, 90%, 85%, 80%, 75%, 70%, 65%, 60%, 55%, 50%, 45%, 40%, 35%, 30%, 25%, or 20% of the nucleotides in the nucleotide sequence are 2′-MOE nucleosides. In some embodiments, the 2′-MOE nucleoside is selected from any of the 2′-MOE nucleosides shown in Table 4.

In some embodiments, at least 1, 2, 3, 4, 5, 6, 7, 8, 9, 10, 11, 12, 13, 14, 15, 16, 17, 18, 19, 20, 21, 22, 23, 24 or more of the nucleotides in the nucleotide sequence are 2′-O-methyl nucleosides. In some embodiments, fewer than or equal to 25, 24, 23, 22, 21, 20, 19, 18, 17, 16, 15, 14, 13, 12, 10, 11, 10, 9, 8, 7, 6, 5, 4, 3, or 2 of the nucleotides in the nucleotide sequence are 2′-O-methyl nucleosides. In some embodiments, at least 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, 97%, 99%, or 100% of the nucleotides in the nucleotide sequence are 2′-O-methyl nucleosides. In some embodiments, less than or equal to 100%, 99%, 97%, 95%, 90%, 85%, 80%, 75%, 70%, 65%, 60%, 55%, 50%, 45%, 40%, 35%, 30%, 25%, or 20% of the nucleotides in the nucleotide sequence are 2′-O-methyl nucleosides. In some embodiments, the nucleotide comprises at least 20, 21, or 22 nucleotides. In some embodiments, the 2′-O-methyl nucleoside is selected from any of the 2′-O-methyl nucleosides shown in Table 4.

In some embodiments, at least 1, 2, 3, 4, 5, 6, 7, 8, 9, 10, 11, 12, 13, 14, 15, 16, 17, 18, 19, 20, 21, 22, 23, 24 or more of the nucleotides in the nucleotide sequence are 2′-O-methyl nucleosides. In some embodiments, fewer than or equal to 25, 24, 23, 22, 21, 20, 19, 18, 17, 16, 15, 14, 13, 12, 10, 11, 10, 9, 8, 7, 6, 5, 4, 3, or 2 of the nucleotides in the nucleotide sequence are 2′-O-methyl nucleosides. In some embodiments, at least 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, 97%, 99%, or 100% of the nucleotides in the nucleotide sequence are 2′-O-methyl nucleosides. In some embodiments, less than or equal to 100%, 99%, 97%, 95%, 90%, 85%, 80%, 75%, 70%, 65%, 60%, 55%, 50%, 45%, 40%, 35%, 30%, 25%, or 20% of the nucleotides in the nucleotide sequence are 2′-O-methyl nucleosides.

In some embodiments, at least 1, 2, 3, 4, 5, 6, 7, 8, 9, 10, 11, 12, 13, 14, 15, or more of the nucleotides in the nucleotide sequence are 5-methylcytosines ((5m)C). In some embodiments, fewer than or equal to 20, 19, 18, 17, 16, 15, 14, 13, 12, 10, 11, 10, 9, 8, 7, 6, 5, 4, 3, or 2 of the nucleotides in the nucleotide sequence are (5m)C. In some embodiments, at least 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50% of the nucleotides in the nucleotide sequence are (5m)C. In some embodiments, less than or equal to 75%, 70%, 65%, 60%, 55%, 50%, 45%, 40%, 35%, 30%, 25%, 20%, 15%, or 10% of the nucleotides in the nucleotide sequence are (5m)C.

In some embodiments, at least 1, 2, 3, 4, 5, 6, 7, 8, 9, 10, 11, 12, 13, 14, 15, 16, 17, 18, 19, 20, 21, 22, 23, 24 or more of the nucleotides in the nucleotide sequence are deoxyribonucleotides. In some embodiments, fewer than or equal to 25, 24, 23, 22, 21, 20, 19, 18, 17, 16, 15, 14, 13, 12, 10, 11, 10, 9, 8, 7, 6, 5, 4, 3, or 2 of the nucleotides in the nucleotide sequence are deoxyribonucleotides. In some embodiments, at least 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, 97%, 99%, or 100% of the nucleotides in the nucleotide sequence are deoxyribonucleotides. In some embodiments, less than or equal to 100%, 99%, 97%, 95%, 90%, 85%, 80%, 75%, 70%, 65%, 60%, 55%, 50%, 45%, 40%, 35%, 30%, 25%, or 20% of the nucleotides in the nucleotide sequence are deoxyribonucleotides.

In some embodiments, at least 1, 2, 3, 4, 5, 6, 7, 8, 9, 10, 11, 12, 13, 14, 15, 16, 17, 18, 19, 20, 21, 22, 23, 24 or more of the nucleotides in the nucleotide sequence are ribonucleotides. In some embodiments, fewer than or equal to 25, 24, 23, 22, 21, 20, 19, 18, 17, 16, 15, 14, 13, 12, 10, 11, 10, 9, 8, 7, 6, 5, 4, 3, or 2 of the nucleotides in the nucleotide sequence are ribonucleotides. In some embodiments, at least 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, 97%, 99%, or 100% of the nucleotides in the nucleotide sequence are ribonucleotides. In some embodiments, less than or equal to 100%, 99%, 97%, 95%, 90%, 85%, 80%, 75%, 70%, 65%, 60%, 55%, 50%, 45%, 40%, 35%, 30%, 25%, or 20% of the nucleotides in the nucleotide sequence are ribonucleotides.

In some embodiments, at least 1, 2, 3, 4, 5, 6, 7, 8, 9, 10, 11, 12, 13, 14, 15, 16, 17, 18, 19, 20, 21, 22, 23, 24 or more of the nucleotides in the nucleotide sequence are purines. In some embodiments, fewer than or equal to 25, 24, 23, 22, 21, 20, 19, 18, 17, 16, 15, 14, 13, 12, 10, 11, 10, 9, 8, 7, 6, 5, 4, 3, or 2 of the nucleotides in the nucleotide sequence are purines. In some embodiments, at least 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, 97%, 99%, or 100% of the nucleotides in the nucleotide sequence are purines. In some embodiments, less than or equal to 100%, 99%, 97%, 95%, 90%, 85%, 80%, 75%, 70%, 65%, 60%, 55%, 50%, 45%, 40%, 35%, 30%, 25%, or 20% of the nucleotides in the nucleotide sequence are purines.

In some embodiments, at least 1, 2, 3, 4, 5, 6, 7, 8, 9, 10, 11, 12, 13, 14, 15, 16, 17, 18, 19, 20, 21, 22, 23, 24 or more of the nucleotides in the nucleotide sequence are pyrimidines. In some embodiments, fewer than or equal to 25, 24, 23, 22, 21, 20, 19, 18, 17, 16, 15, 14, 13, 12, 10, 11, 10, 9, 8, 7, 6, 5, 4, 3, or 2 of the nucleotides in the nucleotide sequence are pyrimidines. In some embodiments, at least 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, 97%, 99%, or 100% of the nucleotides in the nucleotide sequence are pyrimidines. In some embodiments, less than or equal to 100%, 99%, 97%, 95%, 90%, 85%, 80%, 75%, 70%, 65%, 60%, 55%, 50%, 45%, 40%, 35%, 30%, 25%, or 20% of the nucleotides in the nucleotide sequence are pyrimidines.

In some embodiments, at least 1, 2, 3, 4, 5, 6, 7, 8, 9, 10, 11, 12, 13, 14, 15, 16, 17, 18, 19, 20, 21, 22, 23, 24 or more of the nucleotides in the nucleotide sequence independently comprise any of the modified nucleosides shown in Table 4. In some embodiments, at least 5 or more of the nucleotides in the nucleotide sequence independently comprise any of the modified nucleosides shown in Table 4. In some embodiments, at least 10 or more of the nucleotides in the nucleotide sequence independently comprise any of the modified nucleosides shown in Table 4. In some embodiments, at least 15 or more of the nucleotides in the nucleotide sequence independently comprise any of the modified nucleosides shown in Table 4. Although the exemplary modified nucleosides shown in Table 4 depict nucleosides with specific nitrogen-containing bases (e.g., adenine (A), cytosine (C), guanine (G), and thymine (T) bases), the nitrogen-containing bases are interchangeable and, in some embodiments, include the uracil (U) base. For instance, any of the A, C, T, or G bases depicted in the modified nucleosides shown in Table 4 may be replaced with an A, C, T, G, or U base. In some embodiments, the uracil base replaces the thymine base in the modified nucleoside shown in Table 4.

TABLE 4
Exemplary Modified Nucleosides

| Abbreviation | Structure |
|---|---|
| 2′-OMe-A | 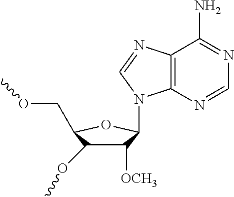 |
| 2′-O-MOE-A | 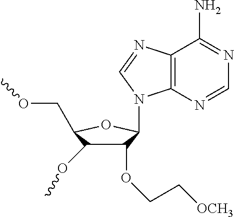 |
| LNA-A | 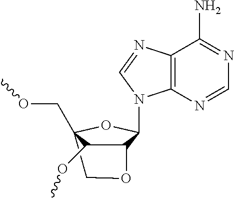 |
| | LNA-A |
| 2′-O-Propargyl-A | 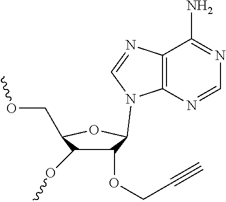 |
| 2′-F-A | 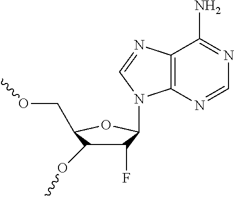 |
| 2′-araF-A | 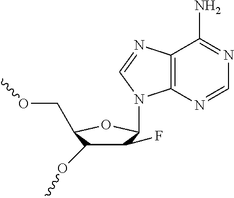 |
| 3′-OMe-A | 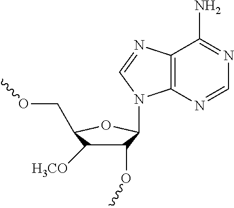 |
| UNA-A | 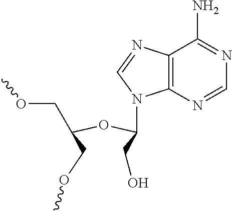 |
| 2′-NH₂-A | 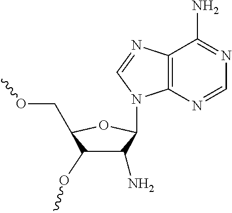 |
| GNA-A | 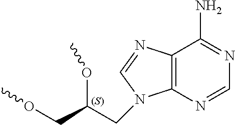 |
| ENA-A | 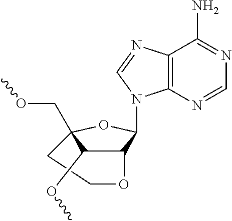 |
| 2′-O-Butynyl-A | 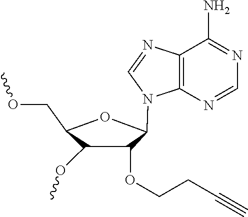 |
| scp-BNA-A | 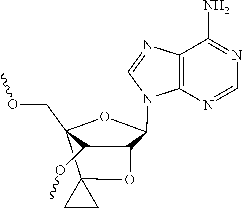 |
| AmNA(NMe)-A | 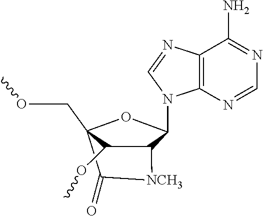 |
| nmLNA-A | 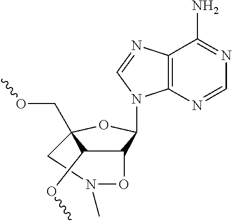 |
| 4etl-A | 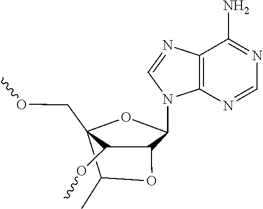 |
| Ribo-A | 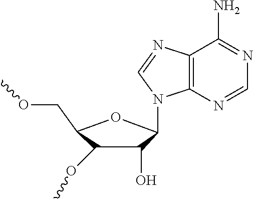 |
| 2′-OMe-(5m)C | 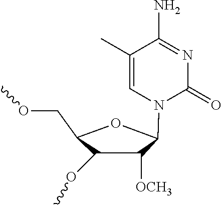 |
| 2′-O-MOE-(5m)C | 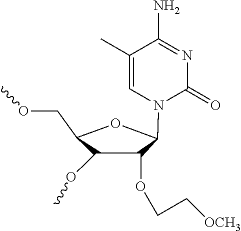 |
| LNA-(5m)C | 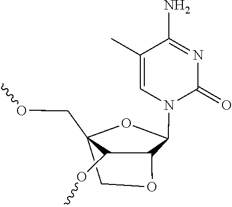 |
| | LNA-5mC |
| 2′-O-Propargyl-(5m)C | 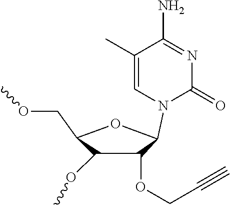 |
| 2′-F-(5m)C | 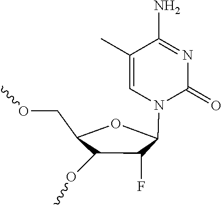 |
| 2′-F-C | 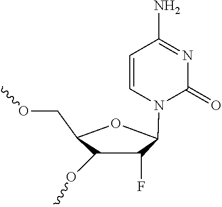 |
| 2′-araF-(5m)C | 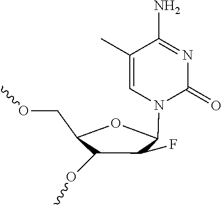 |
| 3′-OMe-(5m)C | 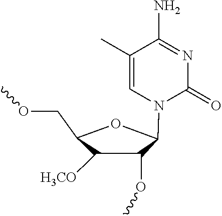 |
| UNA-(5m)C | 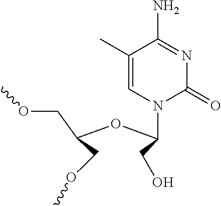 |
| 2′-NH₂-(5m)C | 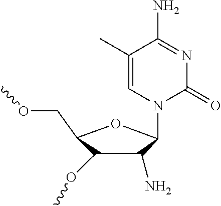 |
| GNA-(5m)C | 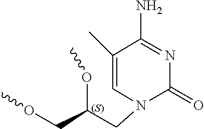 |
| ENA-(5m)C | 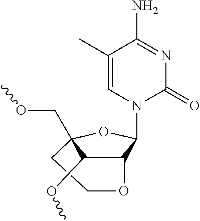 |
| 2′-O-Butynyl-(5m)C | 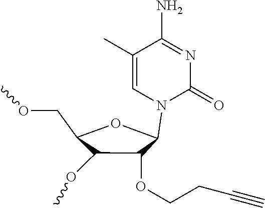 |
| scp-BNA-(5m)C | 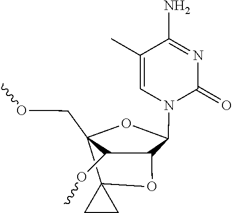 |
| AmNA-(NMe)-(5m)C | 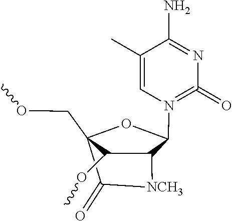 |
| 4etl-(5m)C | 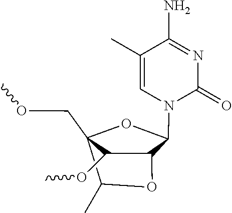 |
| nmLNA-(5m)C | 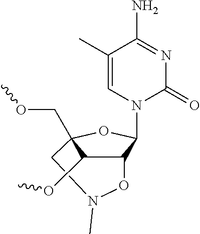 |
| Ribo-C | 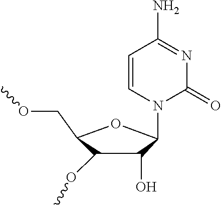 |
| Ribo-(5m)C | 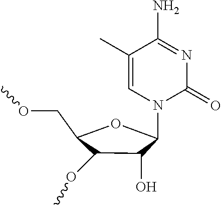 |
| 4etl-G | 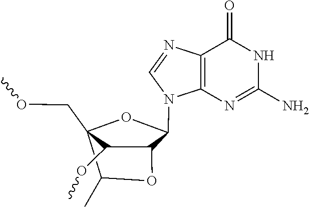 |
| Ribo-G | 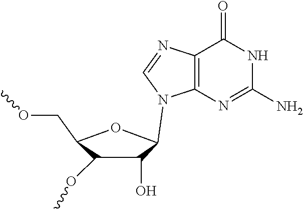 |
| 2′-OMe-G | 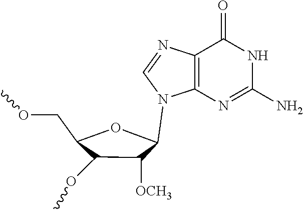 |
| 2′-O-MOE-G | 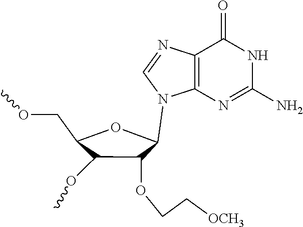 |
| LNA-G | 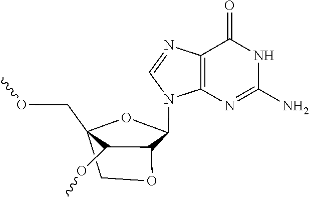 |
| 2′-O-Propargyl-G | 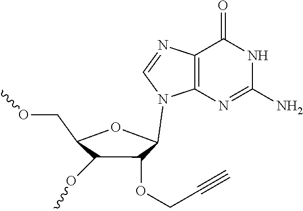 |
| 2′-F-G | 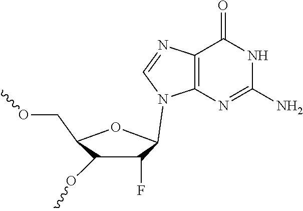 |
| 2′-araF-G | 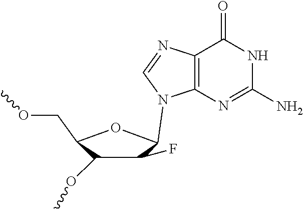 |
| 3′-OMe-G | 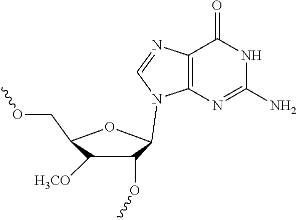 |
| UNA-G | 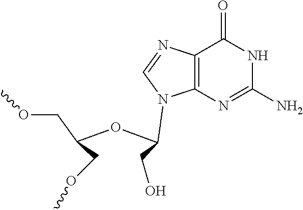 |
| 2′-NH2-G | 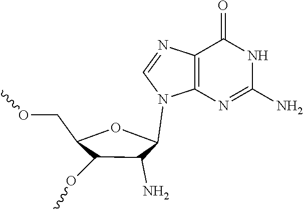 |
| GNA-G | 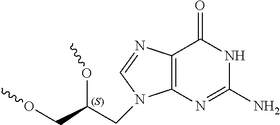 |
| ENA-G | 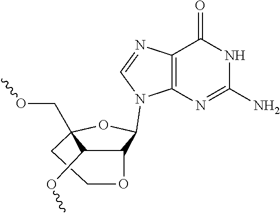 |
| 2′-O-Butynyl-G | 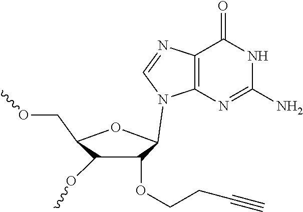 |
| scp-BNA-G | 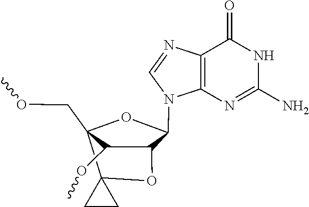 |
| AmNA-(NMe)-G | 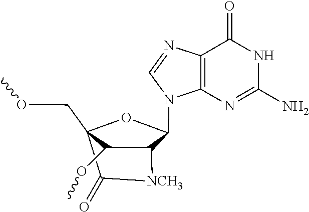 |
| GuNA-(N-R)-G | 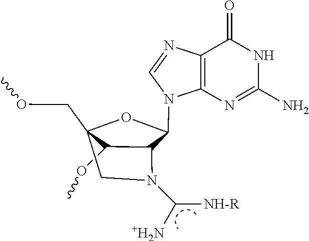 |
| | R = Me, Et, iPr, tBu |
| nmLNA-G | 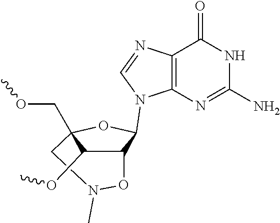 |
| 4etl- T | 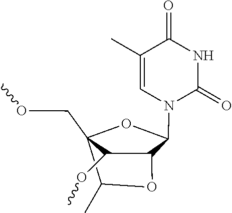 |
| Ribo-T | 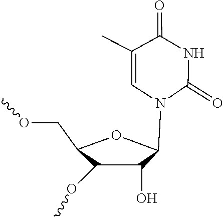 |
| 2′-OMe-T | 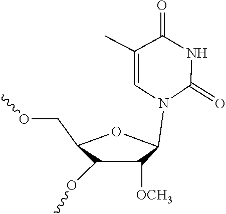 |
| 2′-O-MOE-T | 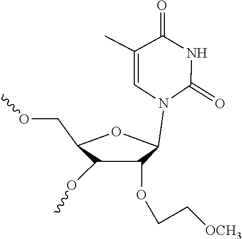 |
| LNA-T | 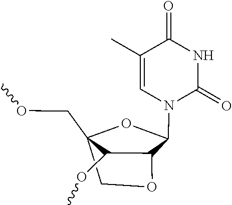 |
| 2′-O-Propargyl-T | 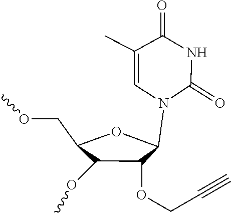 |
| 2′-F- T | 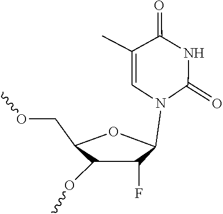 |
| 2′-araF-T | 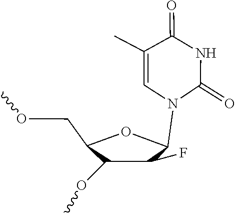 |
| 3′-OMe-T | 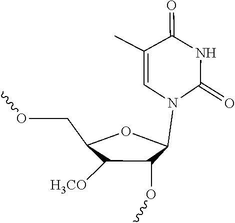 |
| UNA-T | 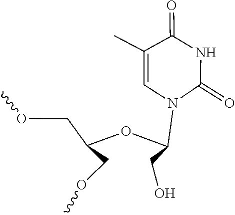 |
| 2′-NH2-T | 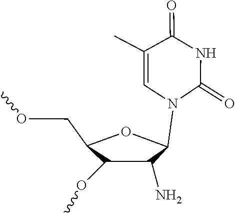 |
| GNA-T | 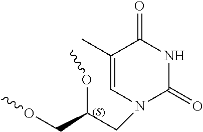 |
| ENA-T | 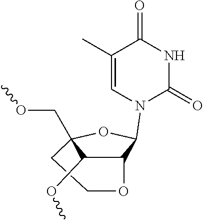 |
| 2′-O-Butynyl-T | 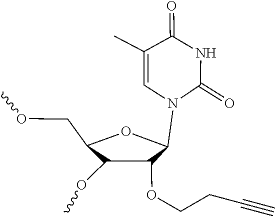 |
| scp-BNA-T | 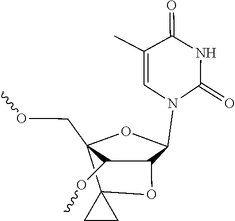 |
| AmNA-(NMe)-T | 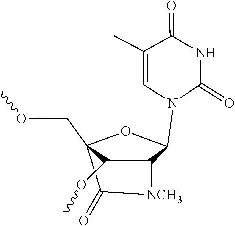 |
| GuNA-(N-R)-T | 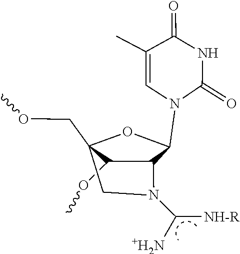 |
| | R = Me, Et, iPr, tBu |
| nmLNA-T | 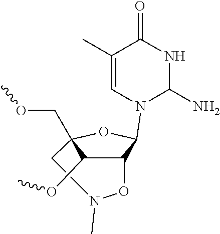 |
| DAP | 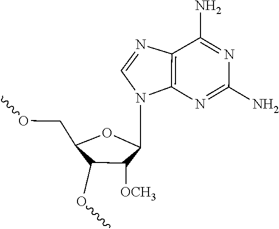 |
| G-clamp | 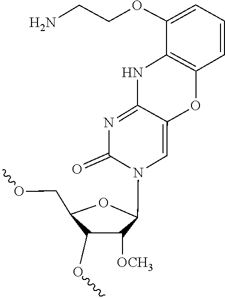 |

In addition to or alternatively, the the disclosed one or more (e.g., 1, 2, 3, 4, 5, 6, 7, 8, 9, 10, 11, 12, 13, 14, 15, etc.) modified nucleotide(s) having a modified nucleobase. For example, the oligonucleotide (i.e., steric blocker) can include one or more (e.g., 1, 2, 3, 4, 5, 6, 7, 8, 9, 10, 11, 12, 13, 14, 15, etc.) modified nucleotide(s) having a protected or unprotected version of the following:

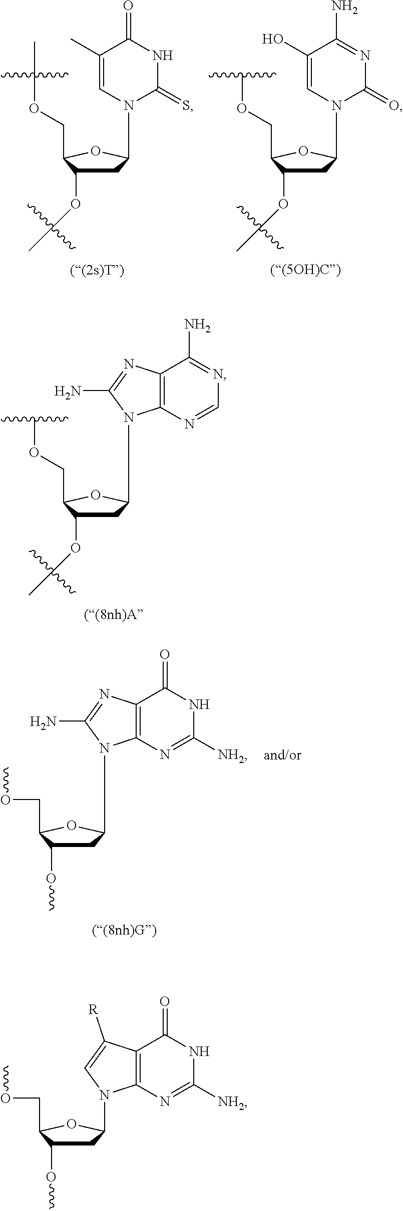

where R is a halogen or R′—C≡C—; and R′ is C₆₋₁₂ aryl, 5- to 12-membered heteroaryl, hydroxy-C₁₋₆ alkyl, or C₁₋₇ alkanoyloxy. In some embodiments, the central region includes one modified nucleotide (e.g., (2s)T or (50H)C) at the 1^{st}, 2^{nd}, 3^{rd} or 4^{th} gap nucleoside position (from the 5′ end). In some embodiments, the modified nucleotide is at the 3^{rd} gap nucleoside position (from the 5′ end). In some embodiments, the modified nucleotide is a nucleotide having the structure of:

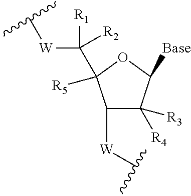

wherein:
W is independently O, N, or S;
R₁, R₂, and R₅ are independently H or D;

### R₃ is H or F;

R₄ is F or OCH₃; and

### Base is

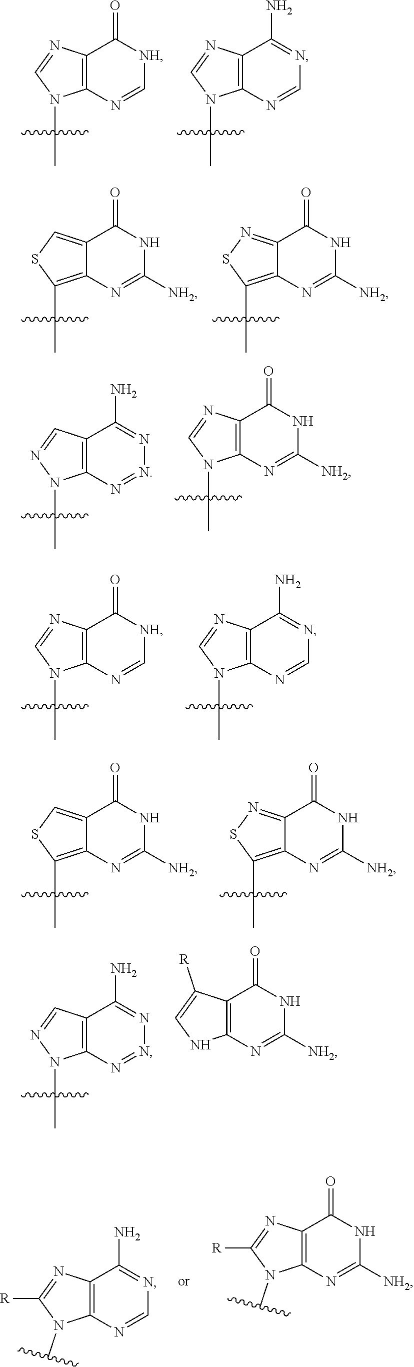

wherein:
R is a halogen or R′—C≡C—; and
R′ represents C₆₋₁₂ aryl, 5- to 12-membered heteroaryl, hydroxy-C₁₋₆ alkyl, or C₁₋₇ alkanoyloxy.
In some embodiments, C₁₋₇ alkanoyl includes, but is not limited to, formyl, acetyl, ethyl carbonyl, n-propyl carbonyl, isopropyl carbonyl, n-butyl carbonyl, isobutyl carbonyl, t-butyl carbonyl, n-pentyl carbonyl, and n-hexyl carbonyl. Other modified nucleotides include those in PCT/JP2018/006061, which is incorporated by reference in its entirety.

As used herein, unless otherwise indicated, “aryl” refers to a carbocyclic (all carbon) ring that has a fully delocalized pi-electron system. The “aryl” group can be made up of two or more fused rings (rings that share two adjacent carbon atoms). When the aryl is a fused ring system, then the ring that is connected to the rest of the molecule has a fully delocalized pi-electron system. The other ring(s) in the fused ring system may or may not have a fully delocalized pi-electron system. Examples of aryl groups include, without limitation, the radicals of benzene, naphthalene and azulene.

As used herein, unless otherwise indicated, “heteroaryl” refers to a ring that has a fully delocalized pi-electron system and contains one or more heteroatoms (e.g., one to three heteroatoms, or one to four heteroatoms, or one to five heteroatoms) independently selected from the group consisting of nitrogen, oxygen, and sulfur in the ring. The “heteroaryl” group can be made up of two or more fused rings (rings that share two adjacent carbon atoms). When the heteroaryl is a fused ring system, then the ring that is connected to the rest of the molecule has a fully delocalized pi-electron system. The other ring(s) in the fused ring system may or may not have a fully delocalized pi-electron system. Examples of heteroaryl rings include, without limitation, furan, thiophene, pyrrole, oxazole, thiazole, imidazole, pyrazole, isoxazole, isothiazole, triazole, thiadiazole, pyridine, pyridazine, pyrimidine, pyrazine and triazine.

In some embodiments, any of the modified nucleosides further comprise a phosphorothioate group. In some embodiments, at least 2, 3, 4, 5, 6, 7, 8, 9, 10, 11, 12, 13, 14, 15, 16, 17, 18, 19, 20, 21, 22, 23, 24 or more of the modified nucleosides further comprise a phosphorothioate group.

In some embodiments, any of nucleotides in the nucleotide sequence further comprise a phosphorothioate group. In some embodiments, at least 2, 3, 4, 5, 6, 7, 8, 9, 10, 11, 12, 13, 14, 15, 16, 17, 18, 19, 20, 21, 22, 23, 24 or more of the nucleotides in the nucleotide sequence further comprise a phosphorothioate group.

In some embodiments, at least 2, 3, 4, 5, 6, 7, 8, 9, 10, 11, 12, 13, 14, 15, 16, 17, 18, 19, 20, 21, 22, 23, 24 or more of the nucleotides in the nucleotide sequence are linked by phosphorothioate linkages. In some embodiments, fewer than or equal to 25, 24, 23, 22, 21, 20, 19, 18, 17, 16, 15, 14, 13, 12, 10, 11, 10, 9, 8, 7, 6, 5, 4, 3, or 2 of the nucleotides in the nucleotide sequence are linked by phosphorothioate linkages. In some embodiments, at least 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, 97%, 99%, or 100% of the nucleotides in the nucleotide sequence are linked by phosphorothioate linkages. In some embodiments, less than or equal to 100%, 99%, 97%, 95%, 90%, 85%, 80%, 75%, 70%, 65%, 60%, 55%, 50%, 45%, 40%, 35%, 30%, 25%, or 20% of the nucleotides in the nucleotide sequence are linked by phosphorothioate linkages.

In some embodiments, at least 2, 3, 4, 5, 6, 7, 8, 9, 10, 11, 12, 13, 14, 15, 16, 17, 18, 19, 20, 21, 22, 23, 24 or more of the nucleotides in the nucleotide sequence are linked by phosphodiester linkages. In some embodiments, fewer than or equal to 25, 24, 23, 22, 21, 20, 19, 18, 17, 16, 15, 14, 13, 12, 10, 11, 10, 9, 8, 7, 6, 5, 4, 3, or 2 of the nucleotides in the nucleotide sequence are linked by phosphodiester linkages. In some embodiments, at least 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, 97%, 99%, or 100% of the nucleotides in the nucleotide sequence are linked by phosphodiester linkages. In some embodiments, less than or equal to 100%, 99%, 97%, 95%, 90%, 85%, 80%, 75%, 70%, 65%, 60%, 55%, 50%, 45%, 40%, 35%, 30%, 25%, or 20% of the nucleotides in the nucleotide sequence are linked by phosphodiester linkages.

In some embodiments, any of the oligonucleotide disclosed herein further comprise a tissue targeting conjugate. In some embodiments, the tissue targeting conjugate is attached to the oligonucleotide and targets (e.g., directs) the oligonucleotide to a cell, tissue, or organ. In some embodiments, the cell is from a tissue or organ. In some embodiments, the tissue is selected from muscle, epithelial, connective, and nervous tissue. In some embodiments, the organ is selected from integumentary, skeletal, muscular, nervous, endocrine, cardiovascular, lymphatic, respiratory, digestive, urinary, and reproductive systems. In some embodiments, the organ is selected from the brain, lungs, heart, kidney, liver, bladder, stomach, intestines, and appendix. In some embodiments, the organ is the liver. In some embodiments, the tissue targeting conjugate targets the oligonucleotide to the liver. As used herein, targeting an oligonucleotide to a cell, tissue, or organ comprises facilitating the uptake (e.g., internalization) or localization of the oligonucleotide to the cell, tissue, or organ.

In some embodiments, the tissue targeting conjugate comprises a galactosamine. In some embodiments, the galactosamine is N-acetylgalactosamine (GalNAc) of Formula (I):

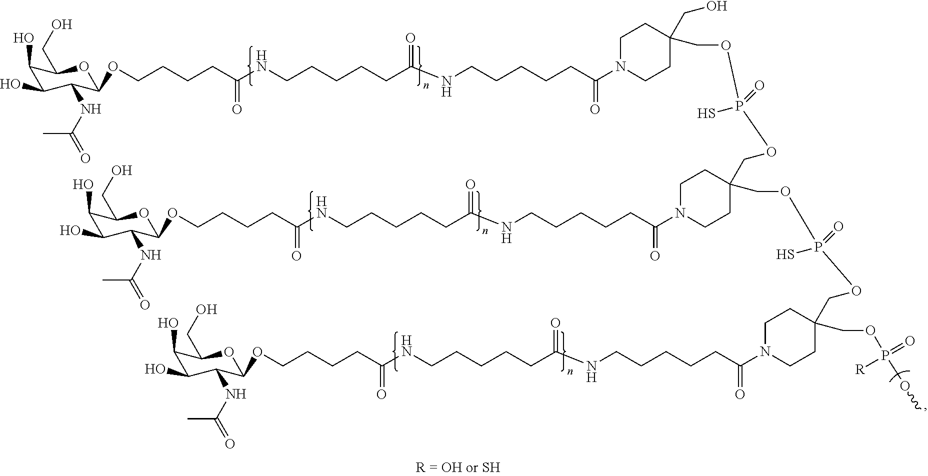

wherein each n is independently 1 or 2.

In some embodiments, the galactosamine is N-acetylgalactosamine (GalNAc) of Formula (II):

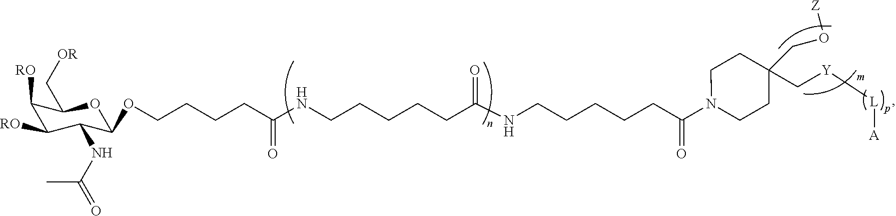

wherein
m is 1, 2, 3, 4, or 5;
each n is independently 1 or 2;
p is 0 or 1;
each R is independently H;
each Y is independently selected from —O—P(═O)(SH)—, —O—P(═O)(O)—, —O—P(═O)(OH)—, and —O—P(S)S—;
Z is H or a second protecting group;
either L is a linker or L and Y in combination are a linker; and
A is H, OH, a third protecting group, an activated group, or an oligonucleotide.

In some embodiments, the tissue targeting conjugate (e.g., galactosamine) is attached to the 3′ end of the nucleotide sequence. In some embodiments, the tissue targeting conjugate (e.g., galactosamine) is attached to the 5′ end of the nucleotide sequence. In some embodiments, the tissue targeting conjugate (e.g., galactosamine) is attached to the nucleotide sequence via one or more linkages independently selected from a phosphodiester linkage, phosphorothioate linkage, or phosphorodithioate linkage.

In some embodiments, the tissue targeting conjugate (e.g., galactosamine) is attached to the nucleotide sequence via a linker sequence. In some embodiments, the linker sequence comprises at least 1, 2, 3, 4, 5, 6, 7, 8, 9, 10, 11, 12, 13, 14, or 15 nucleotides. In some embodiments, the linker sequence comprises 1 to 15, 2 to 15, 3 to 15, 1 to 12, 1 to 10, 1 to 8, 1 to 7, 1 to 6, 1 to 5, 2 to 12, 2 to 10, 2 to 8, 2 to 7, 2 to 6, 2 to 5, 3 to 12, 3 to 10, 3 to 8, 3 to 7, 3 to 6 or 3 to 5 nucleotides. In some embodiments, the linker sequence comprises 2 nucleosides. In some embodiments, the linker sequence comprises 3 nucleosides. In some embodiments, the linker sequence comprises 4 nucleosides. In some embodiments, the linker sequence is located between the tissue targeting conjugate (e.g., galactosamine) and the nucleotide sequence. In some embodiments, the tissue targeting conjugate (e.g., galactosamine) is attached to the linker sequence via one or more linkages independently selected from a phosphodiester linkage, phosphorothioate linkage, or phosphorodithioate linkage.

In some embodiments, the nucleotide sequence is selected from a sequence as shown in Tables 1-3. In some embodiments, the nucleotide sequence comprises a sequence selected from the group consisting of SEQ ID NO: 78, 100, 161, and 171. In some embodiments, the nucleotide sequence comprises at least 10, 11, 12, 13, 14, 15, 16, 17, 18, 19, 20, 21, or 22 consecutive nucleotides of a sequence shown in any one of Tables 1-3. In some embodiments, the nucleotide sequence is at least 80%, 81%, 82%, 83%, 84%, 85%, 86%, 87%, 88%, 89%, 90%, 91%, 92%, 93%, 94%, 95%, 96%, 97%, 98%, 99%, or 100% identical to a sequence shown in any one of Tables 1-3. In some embodiments, the nucleotide sequence comprises fewer than or equal to 5, 4, 3, 2, or 1 mismatches to a sequence shown in any one of Tables 1-3.

In some embodiments, the nucleotide sequence of the oligonucleotide differs from the viral target sequence by less than or equal to 5, 4, 3, 2, or 1 nucleotide. In some embodiments, the nucleotide sequence of the oligonucleotide differs from the viral target sequence by less than or equal to 5 nucleotides. In some embodiments, the nucleotide sequence of the oligonucleotide differs from the viral target sequence by less than or equal to 4 nucleotides. In some embodiments, the nucleotide sequence of the oligonucleotide differs from the viral target sequence by less than or equal to 3 nucleotides. In some embodiments, the nucleotide sequence of the oligonucleotide differs from the viral target sequence by less than or equal to 2 nucleotides. In some embodiments, the nucleotide sequence of the oligonucleotide differs from the viral target sequence by less than or equal to 1 nucleotide.

In some embodiments, the nucleotide sequence of the oligonucleotide is complementary to the viral target sequence, wherein the complementarity of the nucleotide sequence to the viral target sequence has a mismatch of less than or equal to 5, 4, 3, 2, or 1 nucleotide. In some embodiments, the complementarity of the nucleotide sequence to the viral target sequence has a mismatch of less than or equal to 5 nucleotides. In some embodiments, the complementarity of the nucleotide sequence to the viral target sequence has a mismatch of less than or equal to 4 nucleotides. In some embodiments, the complementarity of the nucleotide sequence to the viral target sequence has a mismatch of less than or equal to 3 nucleotides. In some embodiments, the complementarity of the nucleotide sequence to the viral target sequence has a mismatch of less than or equal to 2 nucleotides. In some embodiments, the complementarity of the nucleotide sequence to the viral target sequence has a mismatch of less than or equal to 1 nucleotide.

In some embodiments, any of the oligonucleotides disclosed herein has a melting temperature (Tm) for the complementary viral target sequence of between 50 to 90° C., 55 to 90° C., 60 to 90° C., 70 to 90° C., 75 to 90° C., 80 to 90° C., or 80 to 85° C. In some embodiments, any of the oligonucleotides disclosed herein has a Tm for the complementary viral target sequence of at least 50° C., 51° C., 52° C., 53° C., 54° C., 55° C., 56° C., 57° C., 58° C., 59° C., 60° C., 61° C., 62° C., 63° C., 64° C., 65° C., 66° C., 67° C., 68° C., 69° C., 70° C., 71° C., 72° C., 73° C., 74° C., 75° C., 76° C., 77° C., 78° C., 79° C., 80° C., 81° C., 82° C., 83° C., 84° C., 85° C., 86° C., 87° C., 88° C., 89° C., or 90° C.

In some embodiments, any of the oligonucleotides disclosed herein has a Tm for the complementary viral target sequence of less than or equal to 90° C., 89° C., 88° C., 87° C., 86° C., or 85° C.

Plasmids, Viral Vectors, and Particles

In some embodiments, any of the oligonucleotides disclosed herein may be delivered or administered to a subject via any suitable method, such as liposomes, plasmid, viral vector, or particle. Techniques for oligonucleotide delivery or administration via liposomes, plasmid, viral vector, or particle are known in the art, for instance, Dias and Stein, Mol Cancer Ther, 1(5):347-355, 2002; Batista-Duharte et al., 10(2):pii:E316, 2020; Imbert et al., Genes (Basel), 8(2): pii:E51, 2017, Garanto et al, Sensory (Ophthalmic and Auditory Diseases), 22:S46-S47, 2014, Bisset et al., Hum Mol Genet, 24:4971-4983, 2015, Yang et al., Mol Ther Nucleic Acids, 19:1357-1367, 2020; Cheng et al., Mol Pharm, 15(10):4722-4732, 2018; Cheng et al., Pharm Res, 34(2):310-320, 2017; and Ramelli et al., Mol Ther Nucleic Acids, 19:1000-1014, 2020, which are incorporated by reference in their entirety. Further disclosed herein are plasmids, viral vectors, and particles comprising any of the oligonucleotides disclosed herein.

In some embodiments, any of the oligonucleotides disclosed herein may be delivered or administered to a subject via a liposome. In some embodiments, the liposome comprises an oligonucleotide comprising a nucleotide sequence comprising 5 to 40 nucleotides, wherein one or more of the 5 to 40 nucleotides is a modified nucleoside, wherein at least 10 consecutive nucleotides of the 5 to 40 nucleotides are identical to, complementary, hybridizes, or binds to a viral target sequence in an rcDNA or cccDNA form of a hepatitis B virus (HBV) genome. In some embodiments, the viral target sequence is in the rcDNA. In some embodiments, the viral target sequence is in the cccDNA. In some embodiments, the viral target sequence is in the gap region of the rcDNA. In some embodiments, the viral target sequence is in the non-gap region of the rcDNA. In some embodiments, the viral target sequence is in an X region of the rcDNA or cccDNA. In some embodiments, the viral target sequence is in an S region of the rcDNA or cccDNA.

In some embodiments, the liposome comprises an oligonucleotide comprising a nucleotide sequence, wherein the nucleotide sequence comprises at least 1, 2, 3, 4, 5, 6, 7, 8, 9, 10, 11, 12, 13, 14, 15, 16, 17, 18, 19, 20, 21, or 22 or more modified nucleosides, wherein the modified nucleosides are independently selected from a locked nucleoside, 2′-substituted nucleotide, and methylated nucleoside, and wherein at least 10 nucleotides of the nucleotide sequence is identical to, complementary, hybridizes, or binds to a viral target sequence in an rcDNA or cccDNA form of a hepatitis B virus (HBV) genome. In some embodiments, the viral target sequence is in the rcDNA. In some embodiments, the viral target sequence is in the cccDNA. In some embodiments, the viral target sequence is in the gap region of the rcDNA. In some embodiments, the viral target sequence is in the non-gap region of the rcDNA. In some embodiments, the viral target sequence is in an X region of the rcDNA or cccDNA. In some embodiments, the viral target sequence is in an S region of the rcDNA or cccDNA.

In some embodiments, the liposome comprises an oligonucleotide comprising a nucleotide sequence, wherein at least 10%, 15% 20%, 25%, 30%, 35%, 40%, 45%, 50%, 55%, 60%, 65%, 70%, 75%, 80%, 85%, 90%, 95%, 97%, 99%, or 100% of the nucleotides of the nucleotide sequence are modified nucleosides, wherein the modified nucleosides are selected from a locked nucleoside, 2′-substituted nucleoside, and methylated nucleoside, and wherein at least 10 nucleotides of the nucleotide sequence is identical to, complementary, hybridizes, or binds to a viral target sequence in an rcDNA or cccDNA form of a hepatitis B virus (HBV) genome. In some embodiments, the viral target sequence is in the rcDNA. In some embodiments, the viral target sequence is in the cccDNA. In some embodiments, the viral target sequence is in the gap region of the rcDNA. In some embodiments, the viral target sequence is in the non-gap region of the rcDNA. In some embodiments, the viral target sequence is in an X region of the rcDNA or cccDNA. In some embodiments, the viral target sequence is in an S region of the rcDNA or cccDNA.

In some embodiments, any of the oligonucleotides disclosed herein may be delivered or administered to a subject via a plasmid. In some embodiments, the plasmid comprises an oligonucleotide comprising a nucleotide sequence comprising 5 to 40 nucleotides, wherein one or more of the 5 to 40 nucleotides is a modified nucleoside, wherein at least 10 consecutive nucleotides of the 5 to 40 nucleotides are identical to, complementary, hybridizes, or binds to a viral target sequence in an rcDNA or cccDNA form of a hepatitis B virus (HBV) genome. In some embodiments, the viral target sequence is in the rcDNA. In some embodiments, the viral target sequence is in the cccDNA. In some embodiments, the viral target sequence is in the gap region of the rcDNA. In some embodiments, the viral target sequence is in the non-gap region of the rcDNA. In some embodiments, the viral target sequence is in an X region of the rcDNA or cccDNA. In some embodiments, the viral target sequence is in an S region of the rcDNA or cccDNA. In some embodiments, the plasmid further comprises a selectable marker. In some embodiments, the selectable marker is an antibiotic resistance gene. In some embodiments, the selectable marker is an affinity tag.

In some embodiments, the plasmid comprises an oligonucleotide comprising a nucleotide sequence, wherein the nucleotide sequence comprises at least 1, 2, 3, 4, 5, 6, 7, 8, 9, 10, 11, 12, 13, 14, 15, 16, 17, 18, 19, 20, 21, or 22 or more modified nucleosides, wherein the modified nucleosides are independently selected from a locked nucleoside, 2′-substituted nucleoside, and 2′-O-methyl nucleoside, and wherein at least 10 nucleotides of the nucleotide sequence is identical to, complementary, hybridizes, or binds to a viral target sequence in an rcDNA or cccDNA form of a hepatitis B virus (HBV) genome. In some embodiments, the viral target sequence is in the rcDNA. In some embodiments, the viral target sequence is in the cccDNA. In some embodiments, the viral target sequence is in the gap region of the rcDNA. In some embodiments, the viral target sequence is in the non-gap region of the rcDNA. In some embodiments, the viral target sequence is in an X region of the rcDNA or cccDNA. In some embodiments, the viral target sequence is in an S region of the rcDNA or cccDNA. In some embodiments, the plasmid further comprises a selectable marker. In some embodiments, the selectable marker is an antibiotic resistance gene. In some embodiments, the selectable marker is an affinity tag.

In some embodiments, the plasmid comprises an oligonucleotide comprising a nucleotide sequence, wherein at least 10%, 15% 20%, 25%, 30%, 35%, 40%, 45%, 50%, 55%, 60%, 65%, 70%, 75%, 80%, 85%, 90%, 95%, 97%, 99%, or 100% of the nucleotides of the nucleotide sequence are modified nucleosides, wherein the modified nucleosides are selected from a locked nucleoside, 2′-substituted nucleoside, and 2′-O-methyl nucleoside, and wherein at least 10 nucleotides of the nucleotide sequence is identical to, complementary, hybridizes, or binds to a viral target sequence in an rcDNA or cccDNA form of a hepatitis B virus (HBV) genome. In some embodiments, the viral target sequence is in the rcDNA. In some embodiments, the viral target sequence is in the cccDNA. In some embodiments, the viral target sequence is in the gap region of the rcDNA. In some embodiments, the viral target sequence is in the non-gap region of the rcDNA. In some embodiments, the viral target sequence is in an X region of the rcDNA or cccDNA. In some embodiments, the viral target sequence is in an S region of the rcDNA or cccDNA. In some embodiments, the plasmid further comprises a selectable marker. In some embodiments, the selectable marker is an antibiotic resistance gene. In some embodiments, the selectable marker is an affinity tag.

In some embodiments, any of the oligonucleotides disclosed herein may be delivered or administered to a subject via a viral vector. In some embodiments, the viral vector comprises an oligonucleotide comprising a nucleotide sequence comprising 5 to 40 nucleotides, wherein one or more of the 5 to 40 nucleotides is a modified nucleoside, wherein at least 10 consecutive nucleotides of the 5 to 40 nucleotides are identical to, complementary, hybridizes, or binds to a viral target sequence in an rcDNA or cccDNA form of a hepatitis B virus (HBV) genome. In some embodiments, the viral target sequence is in the rcDNA. In some embodiments, the viral target sequence is in the cccDNA. In some embodiments, the viral target sequence is in the gap region of the rcDNA. In some embodiments, the viral target sequence is in the non-gap region of the rcDNA. In some embodiments, the viral target sequence is in an X region of the rcDNA or cccDNA. In some embodiments, the viral target sequence is in an S region of the rcDNA or cccDNA. In some embodiments, the viral vector further comprises a selectable marker. In some embodiments, the selectable marker is an antibiotic resistance gene. In some embodiments, the selectable marker is an affinity tag. In some embodiments, the viral vector is an adeno-associated viral (AAV) vector. In some embodiments, the viral vector further comprises one or more inverted terminal repeats (ITRs).

In some embodiments, the viral vector comprises an oligonucleotide comprising a nucleotide sequence, wherein the nucleotide sequence comprises at least 1, 2, 3, 4, 5, 6, 7, 8, 9, 10, 11, 12, 13, 14, 15, 16, 17, 18, 19, 20, 21, or 22 or more modified nucleosides, wherein the modified nucleosides are independently selected from a locked nucleoside, 2′-substituted nucleoside, and 2′-O-methyl nucleoside, and wherein at least 10 nucleotides of the nucleotide sequence is identical to, complementary, hybridizes, or binds to a viral target sequence an rcDNA or cccDNA form of a hepatitis B virus (HBV) genome. In some embodiments, the viral target sequence is in the rcDNA. In some embodiments, the viral target sequence is in the cccDNA. In some embodiments, the viral target sequence is in the gap region of the rcDNA. In some embodiments, the viral target sequence is in the non-gap region of the rcDNA. In some embodiments, the viral target sequence is in an X region of the rcDNA or cccDNA. In some embodiments, the viral target sequence is in an S region of the rcDNA or cccDNA. In some embodiments, the viral vector further comprises a selectable marker. In some embodiments, the selectable marker is an antibiotic resistance gene. In some embodiments, the selectable marker is an affinity tag. In some embodiments, the viral vector is an adeno-associated viral (AAV) vector. In some embodiments, the viral vector further comprises one or more inverted terminal repeats (ITRs).

In some embodiments, the viral vector comprises an oligonucleotide comprising a nucleotide sequence, wherein at least 10%, 15% 20%, 25%, 30%, 35%, 40%, 45%, 50%, 55%, 60%, 65%, 70%, 75%, 80%, 85%, 90%, 95%, 97%, 99%, or 100% of the nucleotides of the nucleotide sequence are modified nucleosides, wherein the modified nucleosides are selected from a locked nucleoside, 2′-substituted nucleoside, and 2′-O-methyl nucleoside, and wherein at least 10 nucleotides of the nucleotide sequence is identical to, complementary, hybridizes, or binds to a viral target sequence in an rcDNA or cccDNA form of a hepatitis B virus (HBV) genome. In some embodiments, the viral target sequence is in the rcDNA. In some embodiments, the viral target sequence is in the cccDNA. In some embodiments, the viral target sequence is in the gap region of the rcDNA. In some embodiments, the viral target sequence is in the non-gap region of the rcDNA. In some embodiments, the viral target sequence is in an X region of the rcDNA or cccDNA. In some embodiments, the viral target sequence is in an S region of the rcDNA or cccDNA. In some embodiments, the viral vector further comprises a selectable marker. In some embodiments, the selectable marker is an antibiotic resistance gene. In some embodiments, the selectable marker is an affinity tag. In some embodiments, the viral vector is an adeno-associated viral (AAV) vector. In some embodiments, the viral vector further comprises one or more inverted terminal repeats (ITRs).

In some embodiments, any of the oligonucleotides disclosed herein may be delivered or administered to a subject via particles. In some embodiments, the particle comprises an oligonucleotide comprising a nucleotide sequence comprising 5 to 40 nucleotides, wherein one or more of the 5 to 40 nucleotides is a modified nucleoside, wherein at least 10 consecutive nucleotides of the 5 to 40 nucleotides are identical to, complementary, hybridizes, or binds to a viral target sequence in an rcDNA or cccDNA form of a hepatitis B virus (HBV) genome. In some embodiments, the viral target sequence is in the rcDNA. In some embodiments, the viral target sequence is in the cccDNA. In some embodiments, the viral target sequence is in the gap region of the rcDNA. In some embodiments, the viral target sequence is in the non-gap region of the rcDNA. In some embodiments, the viral target sequence is in an X region of the rcDNA or cccDNA. In some embodiments, the viral target sequence is in an S region of the rcDNA or cccDNA. In some embodiments, the particle is a biodegradable particle. In some embodiments, the particle is a lipid nanoparticle (LNP).

In some embodiments, the particle comprises an oligonucleotide comprising a nucleotide sequence, wherein the nucleotide sequence comprises at least 1, 2, 3, 4, 5, 6, 7, 8, 9, 10, 11, 12, 13, 14, 15, 16, 17, 18, 19, 20, 21, or 22 or more modified nucleosides, wherein the modified nucleosides are independently selected from a locked nucleoside, 2′-substituted nucleoside, and 2′-O-methyl nucleoside, and wherein at least 10 nucleotides of the nucleotide sequence is identical to, complementary, hybridizes, or binds to a viral target sequence in an rcDNA or cccDNA form of a hepatitis B virus (HBV) genome. In some embodiments, the viral target sequence is in the rcDNA. In some embodiments, the viral target sequence is in the cccDNA. In some embodiments, the viral target sequence is in the gap region of the rcDNA. In some embodiments, the viral target sequence is in the non-gap region of the rcDNA. In some embodiments, the viral target sequence is in an X region of the rcDNA or cccDNA. In some embodiments, the viral target sequence is in an S region of the rcDNA or cccDNA.

In some embodiments, the particle comprises an oligonucleotide comprising a nucleotide sequence, wherein at least 10%, 15% 20%, 25%, 30%, 35%, 40%, 45%, 50%, 55%, 60%, 65%, 70%, 75%, 80%, 85%, 90%, 95%, 97%, 99%, or 100% of the nucleotides of the nucleotide sequence are modified nucleosides, wherein the modified nucleosides are selected from a locked nucleoside, 2′-substituted nucleoside, and 2′-O-methyl nucleoside, and wherein at least 10 nucleotides of the nucleotide sequence is identical to, complementary, hybridizes, or binds to a viral target sequence in an rcDNA or cccDNA form of a hepatitis B virus (HBV) genome. In some embodiments, the viral target sequence is in the rcDNA. In some embodiments, the viral target sequence is in the cccDNA. In some embodiments, the viral target sequence is in the gap region of the rcDNA. In some embodiments, the viral target sequence is in the non-gap region of the rcDNA. In some embodiments, the viral target sequence is in an X region of the rcDNA or cccDNA. In some embodiments, the viral target sequence is in an S region of the rcDNA or cccDNA.

In some embodiments, any of the oligonucleotides disclosed herein do not result in cleavage of the viral target sequence. In some embodiments, upon hybridization of the oligonucleotide to the viral target sequence, any of the oligonucleotides disclosed herein do not activate or induce an RNA interference mechanism. In some embodiments, upon hybridization of the oligonucleotide to the viral target sequence, any of the oligonucleotides disclosed herein do not activate or induce the RNAse H mechanism. In some embodiments, upon hybridization of the oligonucleotide to the viral target sequence, any of the oligonucleotides disclosed herein do not activate or induce the RISC.

In some embodiments, any of the oligonucleotides disclosed herein reduce conversion of the rcDNA to cccDNA. In some embodiments, the conversion of rcDNA to cccDNA is reduced by at least about 10%, 20%, 30%, 35%, 40%, 50%, 55%, 60%, 65%, 70%, 75%, 80%, 85%, 90%, 95%, 100%, 110%, 120%, 125%, 130%, 140%, 150%, 175%, 200%, 225%, 250%, 275%, 300% as compared to the level of conversion of rcDNA to cccDNA in (a) a cell that has not been contacted with the oligonucleotide; (b) a sample from a subject that has not been treated with the oligonucleotide; (c) a sample from a subject prior to treatment with the oligonucleotide; or (d) a sample from a subject prior to a second or subsequent treatment with the oligonucleotide. In some embodiments, the conversion of rcDNA to cccDNA is reduced by at least about 1.5, 2, 2.5, 3, 3.5, 3, 3.5, 4, 4.5, 5, 5.5, 6, 6.5, 7, 7.5, 8, 8.5, 9, 9.5, 10, 11, 12, 13, 14, 15, 16, 17, 18, 19, or 20-fold as compared to the level of conversion of rcDNA to cccDNA in (a) a cell that has not been contacted with the oligonucleotide; (b) a sample from a subject that has not been treated with the oligonucleotide; (c) a sample from a subject prior to treatment with the oligonucleotide; or (d) a sample from a subject prior to a second or subsequent treatment with the oligonucleotide. Methods for detecting levels of rcDNA to cccDNA conversion are known in the art and include, but are not limited to, cell-based cccDNA assay or any other methods of detecting cccDNA-dependent surrogates. Exemplary methods for detecting cccDNA-dependent surrogates are described herein and include, for example, in Cai et al., Identification of disubstituted sulfonamide compounds as specific inhibitors of hepatitis B virus covalently closed circular DNA formation, Antiviral Agents, doi:10.1128/AAC.00473-12.

In some embodiments, any of the oligonucleotides disclosed herein reduce the amount of cccDNA. In some embodiments, the amount of cccDNA is reduced by at least about 10%, 20%, 30%, 35%, 40%, 50%, 55%, 60%, 65%, 70%, 75%, 80%, 85%, 90%, 95%, 100%, 110%, 120%, 125%, 130%, 140%, 150%, 175%, 200%, 225%, 250%, 275%, 300% as compared to the amount of cccDNA in (a) a cell that has not been contacted with the oligonucleotide; (b) a sample from a subject that has not been treated with the oligonucleotide; (c) a sample from a subject prior to treatment with the oligonucleotide; or (d) a sample from a subject prior to a second or subsequent treatment with the oligonucleotide. In some embodiments, the amount of cccDNA is reduced by at least about 1.5, 2, 2.5, 3, 3.5, 3, 3.5, 4, 4.5, 5, 5.5, 6, 6.5, 7, 7.5, 8, 8.5, 9, 9.5, 10, 11, 12, 13, 14, 15, 16, 17, 18, 19, or 20-fold as compared to the amount of cccDNA in (a) a cell that has not been contacted with the oligonucleotide; (b) a sample from a subject that has not been treated with the oligonucleotide; (c) a sample from a subject prior to treatment with the oligonucleotide; or (d) a sample from a subject prior to a second or subsequent treatment with the oligonucleotide. Methods for detecting amounts of cccDNA are known in the art and include, but are not limited to, cell-based cccDNA assay or any other methods of detecting cccDNA-dependent surrogates. Exemplary methods for detecting cccDNA-dependent surrogates are described herein and include, for example, in Cai et al., Identification of disubstituted sulfonamide compounds as specific inhibitors of hepatitis B virus covalently closed circular DNA formation, Antiviral Agents, doi:10.1128/AAC.00473-12.

In some embodiments, any of the oligonucleotides disclosed herein result in degradation of cccDNA. In some embodiments, the level of cccDNA degradation is increased by at least about 10%, 20%, 30%, 35%, 40%, 50%, 55%, 60%, 65%, 70%, 75%, 80%, 85%, 90%, 95%, 100%, 110%, 120%, 125%, 130%, 140%, 150%, 175%, 200%, 225%, 250%, 275%, 300% as compared to the level of cccDNA degradation in (a) a cell that has not been contacted with the oligonucleotide; (b) a sample from a subject that has not been treated with the oligonucleotide; (c) a sample from a subject prior to treatment with the oligonucleotide; or (d) a sample from a subject prior to a second or subsequent treatment with the oligonucleotide. In some embodiments, the level of cccDNA degradation is increased by at least about 1.5, 2, 2.5, 3, 3.5, 3, 3.5, 4, 4.5, 5, 5.5, 6, 6.5, 7, 7.5, 8, 8.5, 9, 9.5, 10, 11, 12, 13, 14, 15, 16, 17, 18, 19, or 20-fold as compared to the level of cccDNA degradation in (a) a cell that has not been contacted with the oligonucleotide; (b) a sample from a subject that has not been treated with the oligonucleotide; (c) a sample from a subject prior to treatment with the oligonucleotide; or (d) a sample from a subject prior to a second or subsequent treatment with the oligonucleotide. Methods for detecting levels of cccDNA degradation are known in the art and include, but are not limited to, cell-based cccDNA assay or any other methods of detecting cccDNA-dependent surrogates. Exemplary methods for detecting cccDNA-dependent surrogates are described herein and include, for example, in Cai et al., Identification of disubstituted sulfonamide compounds as specific inhibitors of hepatitis B virus covalently closed circular DNA formation, Antiviral Agents, doi:10.1128/AAC.00473-12.

In some embodiments, any of the oligonucleotides disclosed herein reduce the amount of one or more HBV transcripts (e.g., pgRNA, pre-Core RNA, pre-S1 RNA, pre-S2 RNA, or X RNA). In some embodiments, the amount of the one or more HBV transcripts is decreased by at least about 10%, 20%, 30%, 35%, 40%, 50%, 55%, 60%, 65%, 70%, 75%, 80%, 85%, 90%, 95%, 100%, 110%, 120%, 125%, 130%, 140%, 150%, 175%, 200%, 225%, 250%, 275%, 300% as compared to the amount of the HBV transcript in (a) a cell that has not been contacted with the oligonucleotide; (b) a sample from a subject that has not been treated with the oligonucleotide; (c) a sample from a subject prior to treatment with the oligonucleotide; or (d) a sample from a subject prior to a second or subsequent treatment with the oligonucleotide. In some embodiments, the amount of the one or more HBV transcripts is decreased by at least about 1.5, 2, 2.5, 3, 3.5, 3, 3.5, 4, 4.5, 5, 5.5, 6, 6.5, 7, 7.5, 8, 8.5, 9, 9.5, 10, 11, 12, 13, 14, 15, 16, 17, 18, 19, or 20-fold as compared to the amount of the HBV transcript in (a) a cell that has not been contacted with the oligonucleotide; (b) a sample from a subject that has not been treated with the oligonucleotide; (c) a sample from a subject prior to treatment with the oligonucleotide; or (d) a sample from a subject prior to a second or subsequent treatment with the oligonucleotide. Methods for detecting levels of HBV transcripts are known in the art and include, but are not limited to, antibody-based or nucleotide-based detection assays or any other methods of detecting HBV transcripts that are described herein.

Anti HBV Therapy

The compositions, kits, methods, and uses disclosed herein may comprise one or more anti-HBV therapies. In some embodiments, an anti-HBV therapy is an antiviral medication, interferon injection, or liver transplant. In some embodiments, the antiviral medication is selected from entecavir (Baraclude), tenofovir (Viread), lamivudine (Epivir), adefovir (Hepsera) and telbivudine (Tyzeka). In some embodiments, the interferon injection is an interferon alfa-2b (intron A) injection.

Compositions

Further disclosed herein are compositions comprising any of the oligonucleotides disclosed herein. In some embodiments, the composition comprises: (a) any of the oligonucleotides disclosed herein; and (b) a pharmaceutically acceptable carrier, excipient, diluent, or adjuvant. In some embodiments, the composition further comprises an anti-HBV therapy.

In some embodiments, the composition comprises: (a) an oligonucleotide comprising a nucleotide sequence comprising 5 to 40 nucleotides, wherein one or more of the 5 to 40 nucleotides is a modified nucleoside, wherein at least 10 consecutive nucleotides of the 5 to 40 nucleotides is identical to, complementary, hybridizes, or binds to a viral target sequence in an rcDNA or cccDNA form of a hepatitis B virus (HBV) genome; and (b) a pharmaceutically acceptable carrier, excipient, diluent, or adjuvant. In some embodiments, the composition further comprises an anti-HBV therapy. In some embodiments, the viral target sequence is in the rcDNA. In some embodiments, the viral target sequence is in the cccDNA. In some embodiments, the viral target sequence is in the gap region of the rcDNA. In some embodiments, the viral target sequence is in the non-gap region of the rcDNA. In some embodiments, the viral target sequence is in an X region of the rcDNA or cccDNA. In some embodiments, the viral target sequence is in an S region of the rcDNA or cccDNA.

In some embodiments, the composition comprises: (a) an oligonucleotide comprising a nucleotide sequence, wherein the nucleotide sequence comprises at least 1, 2, 3, 4, 5, 6, 7, 8, 9, 10, 11, 12, 13, 14, 15, 16, 17, 18, 19, 20, 21, or 22 or more modified nucleosides, wherein the modified nucleosides are independently selected from a locked nucleoside, 2′-substituted nucleoside, and 2′-O-methyl nucleoside, and wherein at least 10 nucleotides of the nucleotide sequence is identical to, complementary, hybridizes, or binds to a viral target sequence in an rcDNA or cccDNA form of a hepatitis B virus (HBV) genome; and (b) a pharmaceutically acceptable carrier, excipient, diluent, or adjuvant. In some embodiments, the nucleotide sequence comprises at least 5 modified nucleosides. In some embodiments, the nucleotide sequence comprises at least 10 modified nucleosides. In some embodiments, the nucleotide sequence comprises at least 15 modified nucleosides. In some embodiments, the nucleotide comprises at least 17 modified nucleosides. In some embodiments, the nucleotide comprises at least 22 modified nucleosides. In some embodiments, the composition further comprises an anti-HBV therapy. In some embodiments, the viral target sequence is in the rcDNA. In some embodiments, the viral target sequence is in the cccDNA. In some embodiments, the viral target sequence is in the gap region of the rcDNA. In some embodiments, the viral target sequence is in the non-gap region of the rcDNA. In some embodiments, the viral target sequence is in an X region of the rcDNA or cccDNA. In some embodiments, the viral target sequence is in an S region of the rcDNA or cccDNA.

In some embodiments, the composition comprises: (a) an oligonucleotide comprising a nucleotide sequence, wherein at least 10%, 15% 20%, 25%, 30%, 35%, 40%, 45%, 50%, 55%, 60%, 65%, 70%, 75%, 80%, 85%, 90%, 95%, 97%, 99%, or 100% of the nucleotides of the nucleotide sequence are modified nucleosides, wherein the modified nucleosides are selected from a locked nucleoside, 2′-substituted nucleoside, and 2′-O-methyl nucleoside, and wherein at least 10 nucleotides of the nucleotide sequence is identical to, complementary, hybridizes, or binds to a viral target sequence in an rcDNA or cccDNA form of a hepatitis B virus (HBV) genome; and (b) a pharmaceutically acceptable carrier, excipient, diluent, or adjuvant. In some embodiments, the composition further comprises an anti-HBV therapy. In some embodiments, the viral target sequence is in the rcDNA. In some embodiments, the viral target sequence is in the cccDNA. In some embodiments, the viral target sequence is in the gap region of the rcDNA. In some embodiments, the viral target sequence is in the non-gap region of the rcDNA. In some embodiments, the viral target sequence is in an X region of the rcDNA or cccDNA. In some embodiments, the viral target sequence is in an S region of the rcDNA or cccDNA.

In some embodiments, the composition comprises: (a) any of the oligonucleotides disclosed herein; and (b) an anti-HBV therapy. In some embodiments, the oligonucleotide and the anti-HBV therapy are in separate containers. In some embodiments, the oligonucleotide and the anti-HBV therapy are in the same container. In some embodiments, the composition further comprises any of the pharmaceutically acceptable carriers, diluents, or adjuvants disclosed herein.

In some embodiments, the composition comprises: (a) an oligonucleotide comprising a nucleotide sequence comprising 5 to 40 nucleotides, wherein one or more of the 5 to 40 nucleotides is a modified nucleoside, wherein at least 10 consecutive nucleotides of the 5 to 40 nucleotides are identical to, complementary, hybridizes, or binds to a viral target sequence in an rcDNA or cccDNA form of a hepatitis B virus (HBV) genome; and (b) an anti-HBV therapy. In some embodiments, the oligonucleotide and the anti-HBV therapy are in separate containers. In some embodiments, the oligonucleotide and the anti-HBV therapy are in the same container. In some embodiments, the composition further comprises any of the pharmaceutically acceptable carriers, diluents, or adjuvants disclosed herein. In some embodiments, the viral target sequence is in the rcDNA. In some embodiments, the viral target sequence is in the cccDNA. In some embodiments, the viral target sequence is in the gap region of the rcDNA. In some embodiments, the viral target sequence is in the non-gap region of the rcDNA. In some embodiments, the viral target sequence is in an X region of the rcDNA or cccDNA. In some embodiments, the viral target sequence is in an S region of the rcDNA or cccDNA.

In some embodiments, the composition comprises: (a) an oligonucleotide comprising a nucleotide sequence, wherein the nucleotide sequence comprises at least 1, 2, 3, 4, 5, 6, 7, 8, 9, 10, 11, 12, 13, 14, 15, 16, 17, 18, 19, 20, 21, or 22 or more modified nucleosides, wherein the modified nucleosides are independently selected from a locked nucleoside, 2′-substituted nucleoside, and 2′-O-methyl nucleoside, and wherein at least 10 nucleotides of the nucleotide sequence is identical to, complementary, hybridizes, or binds to a viral target sequence in an rcDNA or cccDNA form of a hepatitis B virus (HBV) genome; and (b) an anti-HBV therapy. In some embodiments, the oligonucleotide and the anti-HBV therapy are in separate containers. In some embodiments, the oligonucleotide and the anti-HBV therapy are in the same container. In some embodiments, the composition further comprises any of the pharmaceutically acceptable carriers, diluents, or adjuvants disclosed herein. In some embodiments, the viral target sequence is in the rcDNA. In some embodiments, the viral target sequence is in the cccDNA. In some embodiments, the viral target sequence is in the gap region of the rcDNA. In some embodiments, the viral target sequence is in the non-gap region of the rcDNA. In some embodiments, the viral target sequence is in an X region of the rcDNA or cccDNA. In some embodiments, the viral target sequence is in an S region of the rcDNA or cccDNA.

In some embodiments, the composition comprises: (a) an oligonucleotide comprising a nucleotide sequence, wherein at least 10%, 15% 20%, 25%, 30%, 35%, 40%, 45%, 50%, 55%, 60%, 65%, 70%, 75%, 80%, 85%, 90%, 95%, 97%, 99%, or 100% of the nucleotides of the nucleotide sequence are modified nucleosides, wherein the modified nucleosides are selected from a locked nucleoside, 2′-substituted nucleoside, and 2′-O-methyl nucleoside, and wherein at least 10 nucleotides of the nucleotide sequence is identical to, complementary, hybridizes, or binds to a viral target sequence in an rcDNA or cccDNA form of a hepatitis B virus (HBV) genome; and (b) an anti-HBV therapy. In some embodiments, the oligonucleotide and the anti-HBV therapy are in separate containers. In some embodiments, the oligonucleotide and the anti-HBV therapy are in the same container. In some embodiments, the composition further comprises any of the pharmaceutically acceptable carriers, diluents, or adjuvants disclosed herein. In some embodiments, the viral target sequence is in the rcDNA. In some embodiments, the viral target sequence is in the cccDNA. In some embodiments, the viral target sequence is in the gap region of the rcDNA. In some embodiments, the viral target sequence is in the non-gap region of the rcDNA. In some embodiments, the viral target sequence is in an X region of the rcDNA or cccDNA. In some embodiments, the viral target sequence is in an S region of the rcDNA or cccDNA.

Kits

Further disclosed herein are kits comprising (a) any of the oligonucleotides, compositions, liposomes, plasmids, viral vectors, and/or particles disclosed herein; and (b) instructions for use. In some embodiments, the kit comprises an oligonucleotide comprising a nucleotide sequence comprising 5 to 40 nucleotides, wherein one or more of the 5 to 40 nucleotides is a modified nucleoside, wherein at least 10 consecutive nucleotides of the 5 to 40 nucleotides are identical to, complementary, hybridizes, or binds to a viral target sequence in an rcDNA or cccDNA form of a hepatitis B virus (HBV) genome. In some embodiments, the viral target sequence is in the rcDNA. In some embodiments, the viral target sequence is in the cccDNA. In some embodiments, the viral target sequence is in the gap region of the rcDNA. In some embodiments, the viral target sequence is in the non-gap region of the rcDNA. In some embodiments, the viral target sequence is in an X region of the rcDNA or cccDNA. In some embodiments, the viral target sequence is in an S region of the rcDNA or cccDNA.

In some embodiments, the kit comprises an oligonucleotide comprising a nucleotide sequence, wherein the nucleotide sequence comprises at least 1, 2, 3, 4, 5, 6, 7, 8, 9, 10, 11, 12, 13, 14, 15, 16, 17, 18, 19, 20, 21, or 22 or more modified nucleosides, wherein the modified nucleosides are independently selected from a locked nucleoside, 2′-substituted nucleoside, and 2′-O-methyl nucleoside, and wherein at least 10 nucleotides of the nucleotide sequence is identical to, complementary, hybridizes, or binds to a viral target sequence in an rcDNA or cccDNA form of a hepatitis B virus (HBV) genome. In some embodiments, the viral target sequence is in the rcDNA. In some embodiments, the viral target sequence is in the cccDNA. In some embodiments, the viral target sequence is in the gap region of the rcDNA. In some embodiments, the viral target sequence is in the non-gap region of the rcDNA. In some embodiments, the viral target sequence is in an X region of the rcDNA or cccDNA. In some embodiments, the viral target sequence is in an S region of the rcDNA or cccDNA.

In some embodiments, the kit comprises an oligonucleotide comprising a nucleotide sequence, wherein at least 10%, 15% 20%, 25%, 30%, 35%, 40%, 45%, 50%, 55%, 60%, 65%, 70%, 75%, 80%, 85%, 90%, 95%, 97%, 99%, or 100% of the nucleotides of the nucleotide sequence are modified nucleosides, wherein the modified nucleosides are selected from a locked nucleoside, 2′-substituted nucleoside and 2′-O-methyl nucleoside, and wherein at least 10 nucleotides of the nucleotide sequence is identical to, complementary, hybridizes, or binds to a viral target sequence in an rcDNA or cccDNA form of a hepatitis B virus (HBV) genome. In some embodiments, the viral target sequence is in the rcDNA. In some embodiments, the viral target sequence is in the cccDNA. In some embodiments, the viral target sequence is in the gap region of the rcDNA. In some embodiments, the viral target sequence is in the non-gap region of the rcDNA. In some embodiments, the viral target sequence is in an X region of the rcDNA or cccDNA. In some embodiments, the viral target sequence is in an S region of the rcDNA or cccDNA.

Methods

Further disclosed herein are methods of using any of the oligonucleotides disclosed herein to reduce conversion of HBC rcDNA to cccDNA. In some embodiments, a method of reducing conversion of hepatitis B virus (HBV) relaxed circular DNA (rcDNA) to covalently closed circular DNA (cccDNA) conversion, comprises contacting a cell with any of the oligonucleotides disclosed herein. In some embodiments, at least about 5%, 10%, 15%, 20%, 25%, 30%, 35%, 40%, 45%, 50%, 55%, 60%, 65%, 70%, 75%, 80%, 85%, 90%, 95%, 97%, 99%, or 100% of the rcDNA to cccDNA conversion is reduced as compared to the amount of rcDNA to cccDNA conversion in a cell that has not been contacted with any of the oligonucleotides disclosed herein. In some embodiments, at least about 25% of the rcDNA to cccDNA conversion is reduced as compared to the amount of the rcDNA to cccDNA conversion is reduced in a cell that has not been contacted with any of the oligonucleotides disclosed herein. In some embodiments, at least about 50% of the rcDNA to cccDNA conversion is reduced as compared to the amount of the rcDNA to cccDNA conversion is reduced in a cell that has not been contacted with any of the oligonucleotides disclosed herein. In some embodiments, the amount of rcDNA to cccDNA conversion is determined by directly detecting levels of cccDNA. In some embodiments, the amount of rcDNA to cccDNA conversion is determined by detecting levels of one or more surrogate markers of cccDNA.

In some embodiments, a method of reducing conversion of hepatitis B virus (HBV) relaxed circular DNA (rcDNA) to covalently closed circular DNA (cccDNA) conversion, comprises contacting a cell with an oligonucleotide comprising a nucleotide sequence comprising 5 to 40 nucleotides, wherein one or more of the 5 to 40 nucleotides is a modified nucleoside, wherein at least 10 consecutive nucleotides of the 5 to 40 nucleotides is identical to, complementary, hybridizes, or binds to a viral target sequence in an rcDNA or cccDNA form of a hepatitis B virus (HBV) genome. In some embodiments, the viral target sequence is in the rcDNA. In some embodiments, the viral target sequence is in the cccDNA. In some embodiments, the viral target sequence is in the gap region of the rcDNA. In some embodiments, the viral target sequence is in the non-gap region of the rcDNA. In some embodiments, the viral target sequence is in an X region of the rcDNA or cccDNA. In some embodiments, the viral target sequence is in an S region of the rcDNA or cccDNA. In some embodiments, at least about 5%, 10%, 15%, 20%, 25%, 30%, 35%, 40%, 45%, 50%, 55%, 60%, 65%, 70%, 75%, 80%, 85%, 90%, 95%, 97%, 99%, or 100% of the rcDNA to cccDNA conversion is reduced as compared to the amount of rcDNA to cccDNA conversion in a cell that has not been contacted with any of the oligonucleotides disclosed herein. In some embodiments, at least about 25% of the rcDNA to cccDNA conversion is reduced as compared to the amount of the rcDNA to cccDNA conversion is reduced in a cell that has not been contacted with any of the oligonucleotides disclosed herein. In some embodiments, at least about 50% of the rcDNA to cccDNA conversion is reduced as compared to the amount of the rcDNA to cccDNA conversion is reduced in a cell that has not been contacted with any of the oligonucleotides disclosed herein. In some embodiments, the amount of rcDNA to cccDNA conversion is determined by directly detecting levels of cccDNA. In some embodiments, the amount of rcDNA to cccDNA conversion is determined by detecting levels of one or more surrogate markers of cccDNA.

In some embodiments, a method of reducing conversion of hepatitis B virus (HBV) relaxed circular DNA (rcDNA) to covalently closed circular DNA (cccDNA) conversion, comprises contacting a cell with an oligonucleotide comprising a nucleotide sequence, wherein the nucleotide sequence comprises at least 1, 2, 3, 4, 5, 6, 7, 8, 9, 10, 11, 12, 13, 14, 15, 16, 17, 18, 19, 20, 21, or 22 or more modified nucleosides, wherein the modified nucleosides are independently selected from a locked nucleoside, 2′-substituted nucleoside, and 2′-O-methyl nucleoside, and wherein at least 10 nucleotides of the nucleotide sequence is identical to, complementary, hybridizes, or binds to a viral target sequence in an rcDNA or cccDNA form of a hepatitis B virus (HBV) genome. In some embodiments, the viral target sequence is in the rcDNA. In some embodiments, the viral target sequence is in the cccDNA. In some embodiments, the viral target sequence is in the gap region of the rcDNA. In some embodiments, the viral target sequence is in the non-gap region of the rcDNA. In some embodiments, the viral target sequence is in an X region of the rcDNA or cccDNA. In some embodiments, the viral target sequence is in an S region of the rcDNA or cccDNA. In some embodiments, at least about 5%, 10%, 15%, 20%, 25%, 30%, 35%, 40%, 45%, 50%, 55%, 60%, 65%, 70%, 75%, 80%, 85%, 90%, 95%, 97%, 99%, or 100% of the rcDNA to cccDNA conversion is reduced as compared to the amount of rcDNA to cccDNA conversion in a cell that has not been contacted with any of the oligonucleotides disclosed herein. In some embodiments, at least about 25% of the rcDNA to cccDNA conversion is reduced as compared to the amount of the rcDNA to cccDNA conversion is reduced in a cell that has not been contacted with any of the oligonucleotides disclosed herein. In some embodiments, at least about 50% of the rcDNA to cccDNA conversion is reduced as compared to the amount of the rcDNA to cccDNA conversion is reduced in a cell that has not been contacted with any of the oligonucleotides disclosed herein. In some embodiments, the amount of rcDNA to cccDNA conversion is determined by directly detecting levels of cccDNA.

In some embodiments, the amount of rcDNA to cccDNA conversion is determined by detecting levels of one or more surrogate markers of cccDNA.

In some embodiments, a method of reducing conversion of hepatitis B virus (HBV) relaxed circular DNA (rcDNA) to covalently closed circular DNA (cccDNA) conversion, comprises contacting a cell with an oligonucleotide comprising a nucleotide sequence, wherein at least 10%, 15% 20%, 25%, 30%, 35%, 40%, 45%, 50%, 55%, 60%, 65%, 70%, 75%, 80%, 85%, 90%, 95%, 97%, 99%, or 100% of the nucleotides of the nucleotide sequence are modified nucleosides, wherein the modified nucleosides are selected from a locked nucleoside, 2′-substituted nucleoside, and 2′-O-methyl nucleoside, and wherein at least 10 nucleotides of the nucleotide sequence is identical to, complementary, hybridizes, or binds to a viral target sequence in an rcDNA or cccDNA form of a hepatitis B virus (HBV) genome. In some embodiments, the viral target sequence is in the rcDNA. In some embodiments, the viral target sequence is in the cccDNA. In some embodiments, the viral target sequence is in the gap region of the rcDNA. In some embodiments, the viral target sequence is in the non-gap region of the rcDNA. In some embodiments, the viral target sequence is in an X region of the rcDNA or cccDNA. In some embodiments, the viral target sequence is in an S region of the rcDNA or cccDNA. In some embodiments, at least about 5%, 10%, 15%, 20%, 25%, 30%, 35%, 40%, 45%, 50%, 55%, 60%, 65%, 70%, 75%, 80%, 85%, 90%, 95%, 97%, 99%, or 100% of the rcDNA to cccDNA conversion is reduced as compared to the amount of rcDNA to cccDNA conversion in a cell that has not been contacted with any of the oligonucleotides disclosed herein. In some embodiments, at least about 25% of the rcDNA to cccDNA conversion is reduced as compared to the amount of the rcDNA to cccDNA conversion is reduced in a cell that has not been contacted with any of the oligonucleotides disclosed herein. In some embodiments, at least about 50% of the rcDNA to cccDNA conversion is reduced as compared to the amount of the rcDNA to cccDNA conversion is reduced in a cell that has not been contacted with any of the oligonucleotides disclosed herein. In some embodiments, the amount of rcDNA to cccDNA conversion is determined by directly detecting levels of cccDNA. In some embodiments, the amount of rcDNA to cccDNA conversion is determined by detecting levels of one or more surrogate markers of cccDNA.

Further disclosed herein are methods of using any of the oligonucleotides disclosed herein to target cccDNA for degradation. In some embodiments, a method of targeting hepatitis B virus (HBV) covalently closed circular DNA (cccDNA) for degradation, comprises contacting a cell with any of the oligonucleotides disclosed herein. In some embodiments, at least about 5%, 10%, 15%, 20%, 25%, 30%, 35%, 40%, 45%, 50%, 55%, 60%, 65%, 70%, 75%, 80%, 85%, 90%, 95%, 97%, 99%, or 100% of the cccDNA is degraded as compared to the amount of cccDNA in a cell that has not been contacted with any of the oligonucleotides disclosed herein. In some embodiments, at least about 25% of the cccDNA is degraded as compared to the amount of cccDNA in a cell that has not been contacted with any of the oligonucleotides disclosed herein. In some embodiments, at least about 50% of the cccDNA is degraded as compared to the amount of cccDNA in a cell that has not been contacted with any of the oligonucleotides disclosed herein. In some embodiments, the amount of cccDNA that is degraded is determined by directly detecting levels of cccDNA. In some embodiments, the amount of cccDNA that is degraded is determined by detecting levels of one or more surrogate markers of cccDNA.

In some embodiments, a method of targeting hepatitis B virus (HBV) covalently closed circular DNA (cccDNA) for degradation, comprises contacting a cell with an oligonucleotide comprising a nucleotide sequence comprising 5 to 40 nucleotides, wherein one or more of the 5 to 40 nucleotides is a modified nucleoside, wherein at least 10 consecutive nucleotides of the 5 to 40 nucleotides is identical to, complementary, hybridizes, or binds to a viral target sequence in an rcDNA or cccDNA form of a hepatitis B virus (HBV) genome. In some embodiments, the viral target sequence is in the rcDNA. In some embodiments, the viral target sequence is in the cccDNA. In some embodiments, the viral target sequence is in the gap region of the rcDNA. In some embodiments, the viral target sequence is in the non-gap region of the rcDNA. In some embodiments, the viral target sequence is in an X region of the rcDNA or cccDNA. In some embodiments, the viral target sequence is in an S region of the rcDNA or cccDNA. In some embodiments, at least about 5%, 10%, 15%, 20%, 25%, 30%, 35%, 40%, 45%, 50%, 55%, 60%, 65%, 70%, 75%, 80%, 85%, 90%, 95%, 97%, 99%, or 100% of the cccDNA is degraded as compared to the amount of cccDNA in a cell that has not been contacted with any of the oligonucleotides disclosed herein. In some embodiments, at least about 25% of the cccDNA is degraded as compared to the amount of cccDNA in a cell that has not been contacted with any of the oligonucleotides disclosed herein. In some embodiments, at least about 50% of the cccDNA is degraded as compared to the amount of cccDNA in a cell that has not been contacted with any of the oligonucleotides disclosed herein. In some embodiments, the amount of cccDNA that is degraded is determined by directly detecting levels of cccDNA. In some embodiments, the amount of cccDNA that is degraded is determined by detecting levels of one or more surrogate markers of cccDNA.

In some embodiments, a method of targeting hepatitis B virus (HBV) covalently closed circular DNA (cccDNA) for degradation, comprises contacting a cell with an oligonucleotide comprising a nucleotide sequence, wherein the nucleotide sequence comprises at least 1, 2, 3, 4, 5, 6, 7, 8, 9, 10, 11, 12, 13, 14, 15, 16, 17, 18, 19, 20, 21, or 22 or more modified nucleosides, wherein the modified nucleosides are independently selected from a locked nucleoside, 2′-substituted nucleoside, and 2′-O-methyl nucleoside, and wherein at least 10 nucleotides of the nucleotide sequence is identical to, complementary, hybridizes, or binds to a viral target sequence in an rcDNA or cccDNA form of a hepatitis B virus (HBV) genome. In some embodiments, the viral target sequence is in the rcDNA. In some embodiments, the viral target sequence is in the cccDNA. In some embodiments, the viral target sequence is in the gap region of the rcDNA. In some embodiments, the viral target sequence is in the non-gap region of the rcDNA. In some embodiments, the viral target sequence is in an X region of the rcDNA or cccDNA. In some embodiments, the viral target sequence is in an S region of the rcDNA or cccDNA. In some embodiments, at least about 5%, 10%, 15%, 20%, 25%, 30%, 35%, 40%, 45%, 50%, 55%, 60%, 65%, 70%, 75%, 80%, 85%, 90%, 95%, 97%, 99%, or 100% of the cccDNA is degraded as compared to the amount of cccDNA in a cell that has not been contacted with any of the oligonucleotides disclosed herein. In some embodiments, at least about 25% of the cccDNA is degraded as compared to the amount of cccDNA in a cell that has not been contacted with any of the oligonucleotides disclosed herein. In some embodiments, at least about 50% of the cccDNA is degraded as compared to the amount of cccDNA in a cell that has not been contacted with any of the oligonucleotides disclosed herein. In some embodiments, the amount of cccDNA that is degraded is determined by directly detecting levels of cccDNA. In some embodiments, the amount of cccDNA that is degraded is determined by detecting levels of one or more surrogate markers of cccDNA.

In some embodiments, a method of targeting hepatitis B virus (HBV) covalently closed circular DNA (cccDNA) for degradation, comprises contacting a cell with an oligonucleotide comprising a nucleotide sequence, wherein at least 10%, 15% 20%, 25%, 30%, 35%, 40%, 45%, 50%, 55%, 60%, 65%, 70%, 75%, 80%, 85%, 90%, 95%, 97%, 99%, or 100% of the nucleotides of the nucleotide sequence are modified nucleosides, wherein the modified nucleosides are selected from a locked nucleoside, 2′-substituted nucleoside, and 2′-O-methyl nucleoside, and wherein at least 10 nucleotides of the nucleotide sequence is identical to, complementary, hybridizes, or binds to a viral target sequence in a rcDNA gap region of a hepatitis B virus (HBV). In some embodiments, at least about 5%, 10%, 15%, 20%, 25%, 30%, 35%, 40%, 45%, 50%, 55%, 60%, 65%, 70%, 75%, 80%, 85%, 90%, 95%, 97%, 99%, or 100% of the cccDNA is degraded as compared to the amount of cccDNA in a cell that has not been contacted with any of the oligonucleotides disclosed herein. In some embodiments, at least about 25% of the cccDNA is degraded as compared to the amount of cccDNA in a cell that has not been contacted with any of the oligonucleotides disclosed herein. In some embodiments, at least about 50% of the cccDNA is degraded as compared to the amount of cccDNA in a cell that has not been contacted with any of the oligonucleotides disclosed herein. In some embodiments, the amount of cccDNA that is degraded is determined by directly detecting levels of cccDNA. In some embodiments, the amount of cccDNA that is degraded is determined by detecting levels of one or more surrogate markers of cccDNA.

Further disclosed herein are methods of using any of the oligonucleotides disclosed herein to reduce the amount of cccDNA in a cell. In some embodiments, a method of reducing the amount of hepatitis B virus (HBV) covalently closed circular DNA (cccDNA) in a cell, comprises contacting a cell with any of the oligonucleotides disclosed herein. In some embodiments, at least about 5%, 10%, 15%, 20%, 25%, 30%, 35%, 40%, 45%, 50%, 55%, 60%, 65%, 70%, 75%, 80%, 85%, 90%, 95%, 97%, 99%, or 100% of the cccDNA is reduced as compared to the amount of cccDNA in a cell that has not been contacted with any of the oligonucleotides disclosed herein. In some embodiments, at least about 25% of the cccDNA is reduced as compared to the amount of cccDNA in a cell that has not been contacted with any of the oligonucleotides disclosed herein. In some embodiments, at least about 50% of the cccDNA is reduced as compared to the amount of cccDNA in a cell that has not been contacted with any of the oligonucleotides disclosed herein. In some embodiments, the amount of cccDNA that is reduced is determined by directly detecting levels of cccDNA. In some embodiments, the amount of cccDNA that is reduced is determined by detecting levels of one or more surrogate markers of cccDNA.

In some embodiments, a method of reducing the amount of hepatitis B virus (HBV) covalently closed circular DNA (cccDNA) in a cell, comprises contacting a cell with an oligonucleotide comprising a nucleotide sequence comprising 5 to 40 nucleotides, wherein one or more of the 5 to 40 nucleotides is a modified nucleoside, wherein at least 10 consecutive nucleotides of the 5 to 40 nucleotides is identical to, complementary, hybridizes, or binds to a viral target sequence in an rcDNA or cccDNA form of a hepatitis B virus (HBV) genome. In some embodiments, the viral target sequence is in the rcDNA. In some embodiments, the viral target sequence is in the cccDNA. In some embodiments, the viral target sequence is in the gap region of the rcDNA. In some embodiments, the viral target sequence is in the non-gap region of the rcDNA. In some embodiments, the viral target sequence is in an X region of the rcDNA or cccDNA. In some embodiments, the viral target sequence is in an S region of the rcDNA or cccDNA. In some embodiments, at least about 5%, 10%, 15%, 20%, 25%, 30%, 35%, 40%, 45%, 50%, 55%, 60%, 65%, 70%, 75%, 80%, 85%, 90%, 95%, 97%, 99%, or 100% of the cccDNA is reduced as compared to the amount of cccDNA in a cell that has not been contacted with any of the oligonucleotides disclosed herein. In some embodiments, at least about 25% of the cccDNA is reduced as compared to the amount of cccDNA in a cell that has not been contacted with any of the oligonucleotides disclosed herein. In some embodiments, at least about 50% of the cccDNA is reduced as compared to the amount of cccDNA in a cell that has not been contacted with any of the oligonucleotides disclosed herein. In some embodiments, the amount of cccDNA that is reduced is determined by directly detecting levels of cccDNA. In some embodiments, the amount of cccDNA that is reduced is determined by detecting levels of one or more surrogate markers of cccDNA.

In some embodiments, a method of reducing the amount of hepatitis B virus (HBV) covalently closed circular DNA (cccDNA) in a cell, comprises contacting a cell with an oligonucleotide comprising a nucleotide sequence, wherein the nucleotide sequence comprises at least 1, 2, 3, 4, 5, 6, 7, 8, 9, 10, 11, 12, 13, 14, 15, 16, 17, 18, 19, 20, 21, or 22 or more modified nucleosides, wherein the modified nucleosides are independently selected from a locked nucleoside, 2′-substituted nucleoside, and 2′-O-methyl nucleoside, and wherein at least 10 nucleotides of the nucleotide sequence is identical to, complementary, hybridizes, or binds to a viral target sequence in an rcDNA or cccDNA form of a hepatitis B virus (HBV) genome. In some embodiments, the viral target sequence is in the rcDNA. In some embodiments, the viral target sequence is in the cccDNA. In some embodiments, the viral target sequence is in the gap region of the rcDNA. In some embodiments, the viral target sequence is in the non-gap region of the rcDNA. In some embodiments, the viral target sequence is in an X region of the rcDNA or cccDNA. In some embodiments, the viral target sequence is in an S region of the rcDNA or cccDNA. In some embodiments, at least about 5%, 10%, 15%, 20%, 25%, 30%, 35%, 40%, 45%, 50%, 55%, 60%, 65%, 70%, 75%, 80%, 85%, 90%, 95%, 97%, 99%, or 100% of the cccDNA is reduced as compared to the amount of cccDNA in a cell that has not been contacted with any of the oligonucleotides disclosed herein. In some embodiments, at least about 25% of the cccDNA is reduced as compared to the amount of cccDNA in a cell that has not been contacted with any of the oligonucleotides disclosed herein. In some embodiments, at least about 50% of the cccDNA is reduced as compared to the amount of cccDNA in a cell that has not been contacted with any of the oligonucleotides disclosed herein. In some embodiments, the amount of cccDNA that is reduced is determined by directly detecting levels of cccDNA. In some embodiments, the amount of cccDNA that is reduced is determined by detecting levels of one or more surrogate markers of cccDNA.

In some embodiments, a method of reducing the amount of hepatitis B virus (HBV) covalently closed circular DNA (cccDNA) in a cell, comprises contacting a cell with an oligonucleotide comprising a nucleotide sequence, wherein at least 10%, 15% 20%, 25%, 30%, 35%, 40%, 45%, 50%, 55%, 60%, 65%, 70%, 75%, 80%, 85%, 90%, 95%, 97%, 99%, or 100% of the nucleotides of the nucleotide sequence are modified nucleosides, wherein the modified nucleosides are selected from a locked nucleoside, 2′-substituted nucleoside, and 2′-O-methyl nucleoside, and wherein at least 10 nucleotides of the nucleotide sequence is identical to, complementary, hybridizes, or binds to a viral target sequence in an rcDNA or cccDNA form of a hepatitis B virus (HBV) genome. In some embodiments, the viral target sequence is in the rcDNA. In some embodiments, the viral target sequence is in the cccDNA. In some embodiments, the viral target sequence is in the gap region of the rcDNA. In some embodiments, the viral target sequence is in the non-gap region of the rcDNA. In some embodiments, the viral target sequence is in an X region of the rcDNA or cccDNA. In some embodiments, the viral target sequence is in an S region of the rcDNA or cccDNA. In some embodiments, at least about 5%, 10%, 15%, 20%, 25%, 30%, 35%, 40%, 45%, 50%, 55%, 60%, 65%, 70%, 75%, 80%, 85%, 90%, 95%, 97%, 99%, or 100% of the cccDNA is reduced as compared to the amount of cccDNA in a cell that has not been contacted with any of the oligonucleotides disclosed herein. In some embodiments, at least about 25% of the cccDNA is reduced as compared to the amount of cccDNA in a cell that has not been contacted with any of the oligonucleotides disclosed herein. In some embodiments, at least about 50% of the cccDNA is reduced as compared to the amount of cccDNA in a cell that has not been contacted with any of the oligonucleotides disclosed herein. In some embodiments, the amount of cccDNA that is reduced is determined by directly detecting levels of cccDNA. In some embodiments, the amount of cccDNA that is reduced is determined by detecting levels of one or more surrogate markers of cccDNA.

In some embodiments, any of the methods disclosed herein further comprise detecting levels of at least one of: cccDNA or a surrogate marker of cccDNA. In some embodiments, the surrogate marker of cccDNA is selected from hepatitis B surface antigen (HBsAg), hepatitis B core antigen (HBc-Ag), hepatitis B e antigen (HBeAg), HBV polymerase, and HBV X protein (HBx). In some embodiments, the detecting comprises performing at least one of: a Southern blot, polymerase chain reaction (PCR), Invader assay, in situ hybridization, HBV DNA assay, HBV antigen assay, or HBV antibody assay. In some embodiments, PCR is selected from quantitative PCR (qPCR), competitive qPCR, semi-nested and nested qPCR, droplet-digital PCR, rolling circle amplification qPCR, rolling circle amplification in-situ qPCR, and magnetic capture hybridization qPCR. In some embodiments, the HBV antigen assay is selected from an HBs antigen assay and HBe antigen assay. In some embodiments, the HBV antibody assay is selected from anti-HBs antibody assay, anti-HBc IgM antibody assay, anti-HBc antibody assay, and anti-HBe antibody assay. Methods for detecting HBV cccDNA are known in the art, for instance, as described in Li et al., Viruses, 9(6):pii:E139, 2017; and Singh et al., J Virol. Methods, 118(2):159-67, 2004, which are incorporated by reference in their entireties. Methods of performing an HBV DNA assay, HBV antigen assay or HBV antibody assay are known in the art, for instance, as described in Pawlotsky, J Hepatol, 39 Suppl 1:S31-5, 2003; Avellon et. al., J Med Virol, doi: 10.1002/jmv.25862, 2020, Scheiblauer et al., Vox Sang, 98(3p2):403-414, 2010; Mizuochi et al., J Virol Methods, 136(1-2):254-6, 2006; El-Sherif et al., J Gastroenterol, 44(4):359-64, 2009, which are incorporated by reference in their entireties.

In some embodiments, the cell is from a biological sample from a subject suffering from HBV or suspected of suffering from HBV. In some embodiments, the biological sample is a blood sample. In some embodiments, the blood sample is a serum sample.

Method of Treatment

Further disclosed herein are methods of using any of the oligonucleotides disclosed herein to treat a hepatitis B virus infection in a subject in need thereof. In some embodiments, a method of treating a hepatitis B virus infection in a subject in need thereof, comprising administering to the subject any of the oligonucleotides disclosed herein or a composition comprising any of the oligonucleotides disclosed herein.

In some embodiments, a method of treating a hepatitis B virus infection in a subject in need thereof, comprising administering to the subject an oligonucleotide comprising a nucleotide sequence comprising 5 to 40 nucleotides, wherein one or more of the 5 to 40 nucleotides is a modified nucleoside, wherein at least 10 consecutive nucleotides of the 5 to 40 nucleotides is identical to, complementary, hybridizes, or binds to a viral target sequence in an rcDNA or cccDNA form of a hepatitis B virus (HBV) genome. In some embodiments, the viral target sequence is in the rcDNA. In some embodiments, the viral target sequence is in the cccDNA. In some embodiments, the viral target sequence is in the gap region of the rcDNA. In some embodiments, the viral target sequence is in the non-gap region of the rcDNA. In some embodiments, the viral target sequence is in an X region of the rcDNA or cccDNA. In some embodiments, the viral target sequence is in an S region of the rcDNA or cccDNA.

In some embodiments, a method of treating a hepatitis B virus infection in a subject in need thereof, comprising administering to the subject an oligonucleotide comprising a nucleotide sequence, wherein the nucleotide sequence comprises at least 1, 2, 3, 4, 5, 6, 7, 8, 9, 10, 11, 12, 13, 14, 15, 16, 17, 18, 19, 20, 21, or 22 or more modified nucleosides, wherein the modified nucleosides are independently selected from a locked nucleoside, 2′-substituted nucleoside, and 2′-O-methyl nucleoside, and wherein at least 10 nucleotides of the nucleotide sequence is identical to, complementary, hybridizes, or binds to a viral target sequence in an rcDNA or cccDNA form of a hepatitis B virus (HBV) genome. In some embodiments, the viral target sequence is in the rcDNA. In some embodiments, the viral target sequence is in the cccDNA. In some embodiments, the viral target sequence is in the gap region of the rcDNA. In some embodiments, the viral target sequence is in the non-gap region of the rcDNA. In some embodiments, the viral target sequence is in an X region of the rcDNA or cccDNA. In some embodiments, the viral target sequence is in an S region of the rcDNA or cccDNA.

In some embodiments, a method of treating a hepatitis B virus infection in a subject in need thereof, comprising administering to the subject an oligonucleotide comprising a nucleotide sequence, wherein at least 10%, 15% 20%, 25%, 30%, 35%, 40%, 45%, 50%, 55%, 60%, 65%, 70%, 75%, 80%, 85%, 90%, 95%, 97%, 99%, or 100% of the nucleotides of the nucleotide sequence are modified nucleosides, wherein the modified nucleosides are selected from a locked nucleoside, 2′-substituted nucleoside, and 2′-O-methyl nucleoside, and wherein at least 10 nucleotides of the nucleotide sequence is identical to, complementary, hybridizes, or binds to a viral target sequence in an rcDNA or cccDNA form of a hepatitis B virus (HBV) genome. In some embodiments, the viral target sequence is in the rcDNA. In some embodiments, the viral target sequence is in the cccDNA. In some embodiments, the viral target sequence is in the gap region of the rcDNA. In some embodiments, the viral target sequence is in the non-gap region of the rcDNA. In some embodiments, the viral target sequence is in an X region of the rcDNA or cccDNA. In some embodiments, the viral target sequence is in an S region of the rcDNA or cccDNA.

In some embodiments, any of the methods of treating an HBV infection disclosed herein further comprise detecting levels of at least one of: cccDNA or a surrogate marker of cccDNA in a biological sample from the subject. In some embodiments, the surrogate marker of cccDNA is selected from hepatitis B surface antigen (HBsAg), hepatitis B core antigen (HBc-Ag), hepatitis B e antigen (HBeAg), HBV polymerase, and HBV X protein (HBx). In some embodiments, the detecting comprises performing at least one of: a Southern blot, polymerase chain reaction (PCR), Invader assay, in situ hybridization, HBV DNA assay, HBV antigen assay, or HBV antibody assay. In some embodiments, PCR is selected from quantitative PCR (qPCR), competitive qPCR, semi-nested and nested qPCR, droplet-digital PCR, rolling circle amplification qPCR, rolling circle amplification in-situ qPCR, and magnetic capture hybridization qPCR. In some embodiments, the HBV antigen assay is selected from an HBs antigen assay and HBe antigen assay. In some embodiments, the HBV antibody assay is selected from anti-HBs antibody assay, anti-HBc IgM antibody assay, anti-HBc antibody assay, and anti-HBe antibody assay.

In some embodiments, the biological sample is a blood sample. In some embodiments, the blood sample is a serum sample.

In some embodiments, any of the methods of treating an HBV infection disclosed herein further comprise modifying the dose or dosing regimen of the oligonucleotide administered to the subject based on the levels of the cccDNA or surrogate marker detected. In some embodiments, the dose or dosing region of the oligonucleotide is decreased when the levels of the cccDNA or surrogate marker is decreased, wherein the levels of the cccDNA or surrogate marker is decreased as compared to (a) the levels of the cccDNA or surrogate marker in the subject from an earlier time point; or (b) levels of the cccDNA or surrogate marker in a control sample. In some embodiments, the earlier time point is (a) prior to administering the oligonucleotide to the subject; or (b) after administering an initial dose of the oligonucleotide to the subject, but prior to administering a subsequent dose of the oligonucleotide to the subject.

In some embodiments, any of the methods of treating an HBV infection disclosed herein further comprise administering to the subject one or more anti-HBV therapies. In some embodiments, any of the methods of treating an HBV infection disclosed herein further comprise modifying the dose or dosing regimen of the anti-HBV therapy administered to the subject based on the levels of the cccDNA or surrogate marker detected. In some embodiments, the oligonucleotide and the one or more anti-HBV therapies are administered concurrently. In some embodiments, the oligonucleotide and the one or more anti-HBV therapies are administered sequentially.

In some embodiments, the oligonucleotide is administered by parenteral injection, intravenous (IV) infusion, or subcutaneous injection.

In some embodiments, the oligonucleotide is administered at least 1, 2, 3, 4, or 5 times a day. In some embodiments, the oligonucleotide is administered at least 1, 2, 3, 4, 6, 7, 8, 9, 10, 11, 12, 13, or 14 times a week. In some embodiments, the oligonucleotide is administered at least 1, 2, 3, 4, 6, 7, 8, 9, 10, 11, 12, 13, 14, 15, 16, 17, 18, 19, 20, 21, 22, 23, 24, 25, 26, 27, 28, 29, 30, or 31 times a month. In some embodiments, the oligonucleotide is administered at least every 1, 2, 3, 4, 6, 7, 8, 9, 10, 11, 12, 13, 14 15, 16, 17, 18, 19, 20, 21, 22, 23, or 24 hours. In some embodiments, the oligonucleotide is administered at least every 1, 2, 3, 4, 6, 7, 8, 9, 10, 11, 12, 13, or 14 days. In some embodiments, the oligonucleotide is administered at least every 1, 2, 3, 4, 6, 7, 8, 9, 10, 11, 12, 13, or 14 weeks. In some embodiments, the oligonucleotide is administered for at least 1, 2, 3, 4, 6, 7, 8, 9, 10, 11, 12, 13, or 14 days. In some embodiments, the oligonucleotide is administered for at least 1, 2, 3, 4, 6, 7, 8, 9, 10, 11, 12, 13, or 14 weeks. In some embodiments, the oligonucleotide is administered for at least 1, 2, 3, 4, 6, 7, 8, 9, 10, 11, 12, 13, or 14 months. In some embodiments, the oligonucleotide is administered for at least 1, 2, 3, 4, 6, 7, 8, 9, 10, 11, 12, 13, or 14 years.

In some embodiments, the oligonucleotide is administered at a dose of at least 1, 5, 10, 15, 20, 25, 30, 35, 40, 45, 50, 55, 60, 65, 70, 75, 80, 85, 90, 95, 100, 110, 120, 130, 140, 150, 160, 170, 180, 190, 200, 225, 250, 275, 300, 325, 350, 375, 400, 425, 450, 475, or 500 mg. In some embodiments, the oligonucleotide is administered at a total daily dose of at least 1, 5, 10, 15, 20, 25, 30, 35, 40, 45, 50, 55, 60, 65, 70, 75, 80, 85, 90, 95, 100, 110, 120, 130, 140, 150, 160, 170, 180, 190, 200, 225, 250, 275, 300, 325, 350, 375, 400, 425, 450, 475, or 500 mg. In some embodiments, the oligonucleotide is administered at a dose of less than or equal to 1000, 950, 900, 850, 800, 750, 700, 650, 600, 550, 500, 450, 400, 350, 300, 250, 200, 150, 100, 90, 80, 70, 60, 50, 40, 30, or 20 mg. In some embodiments, the oligonucleotide is administered at a total daily dose of less than or equal to 1000, 950, 900, 850, 800, 750, 700, 650, 600, 550, 500, 450, 400, 350, 300, 250, 200, 150, 100, 90, 80, 70, 60, 50, 40, 30, or 20 mg.

Further disclosed herein are uses of any of the oligonucleotides disclosed herein in the manufacture of a medicament to treat HBV infection in a subject in need thereof. In some embodiments, the oligonucleotide comprises a nucleotide sequence comprising 5 to 40 nucleotides, wherein one or more of the 5 to 40 nucleotides is a modified nucleoside, wherein at least 10 consecutive nucleotides of the 5 to 40 nucleotides are identical to, complementary, hybridizes, or binds to a viral target sequence in an rcDNA or cccDNA form of a hepatitis B virus (HBV) genome. In some embodiments, the viral target sequence is in the rcDNA. In some embodiments, the viral target sequence is in the cccDNA. In some embodiments, the viral target sequence is in the gap region of the rcDNA. In some embodiments, the viral target sequence is in the non-gap region of the rcDNA. In some embodiments, the viral target sequence is in an X region of the rcDNA or cccDNA. In some embodiments, the viral target sequence is in an S region of the rcDNA or cccDNA. In some embodiments, the oligonucleotide comprises a nucleotide sequence, wherein the nucleotide sequence comprises at least 1, 2, 3, 4, 5, 6, 7, 8, 9, 10, 11, 12, 13, 14, 15, 16, 17, 18, 19, 20, 21, or 22 or more modified nucleosides, wherein the modified nucleosides are independently selected from a locked nucleoside, 2′-substituted nucleoside, and 2′-O-methyl nucleoside, and wherein at least 10 nucleotides of the nucleotide sequence is identical to, complementary, hybridizes, or binds to a viral target sequence in a rcDNA gap region of a hepatitis B virus (HBV). In some embodiments, the oligonucleotide comprises nucleotide sequence, wherein at least 10%, 15% 20%, 25%, 30%, 35%, 40%, 45%, 50%, 55%, 60%, 65%, 70%, 75%, 80%, 85%, 90%, 95%, 97%, 99%, or 100% of the nucleotides of the nucleotide sequence are modified nucleosides, wherein the modified nucleosides are selected from a locked nucleoside, 2′-substituted nucleoside, and 2′-O-methyl nucleoside, and wherein at least 10 nucleotides of the nucleotide sequence is identical to, complementary, hybridizes, or binds to a viral target sequence in an rcDNA or cccDNA form of a hepatitis B virus (HBV) genome. In some embodiments, the oligonucleotide is formulated for parenteral injection, intravenous (IV) infusion, or subcutaneous injection.

Further disclosed herein are uses of any of the compositions disclosed herein in the manufacture of a medicament to treat HBV infection in a subject in need thereof. In some embodiments, the composition comprises an oligonucleotide comprising a nucleotide sequence, wherein the nucleotide sequence comprises 5 to 40 nucleotides, wherein one or more of the 5 to 40 nucleotides is a modified nucleoside, wherein at least 10 consecutive nucleotides of the 5 to 40 nucleotides are identical to, complementary, hybridizes, or binds to a viral target sequence in an rcDNA or cccDNA form of a hepatitis B virus (HBV) genome. In some embodiments, the viral target sequence is in the rcDNA. In some embodiments, the viral target sequence is in the cccDNA. In some embodiments, the viral target sequence is in the gap region of the rcDNA. In some embodiments, the viral target sequence is in the non-gap region of the rcDNA. In some embodiments, the viral target sequence is in an X region of the rcDNA or cccDNA. In some embodiments, the viral target sequence is in an S region of the rcDNA or cccDNA. In some embodiments, the composition comprises an oligonucleotide comprising a nucleotide sequence, wherein the nucleotide sequence comprises at least 1, 2, 3, 4, 5, 6, 7, 8, 9, 10, 11, 12, 13, 14, 15, 16, 17, 18, 19, 20, 21, or 22 or more modified nucleosides, wherein the modified nucleosides are independently selected from a locked nucleoside, 2′-substituted nucleoside, and 2′-O-methyl nucleoside, and wherein at least 10 nucleotides of the nucleotide sequence is identical to, complementary, hybridizes, or binds to a viral target sequence in a rcDNA gap region of a hepatitis B virus (HBV). In some embodiments, the composition comprises an oligonucleotide comprising nucleotide sequence, wherein at least 10%, 15% 20%, 25%, 30%, 35%, 40%, 45%, 50%, 55%, 60%, 65%, 70%, 75%, 80%, 85%, 90%, 95%, 97%, 99%, or 100% of the nucleotides of the nucleotide sequence are modified nucleosides, wherein the modified nucleosides are selected from a locked nucleoside, 2′-substituted nucleoside, and 2′-O-methyl nucleoside, and wherein at least 10 nucleotides of the nucleotide sequence is identical to, complementary, hybridizes, or binds to a viral target sequence in an rcDNA or cccDNA form of a hepatitis B virus (HBV) genome. In some embodiments, the oligonucleotide is formulated for parenteral injection, intravenous (IV) infusion, or subcutaneous injection.

### EXAMPLES

### Example 1: Synthesis of Oligonucleotides

The DNA, 2′-OMe, 2′-MOE and LNA phosphoramidite monomers were procured from Thermo Fischer Milwaukee, Chemgenes and Hongene Biotech USA Inc. All the monomers were dried in vacuum desiccator with desiccants (P₂O₅, RT, 24 h). Universal solid supports (CPG) were obtained from ChemGenes. The chemicals and solvents for synthesis workflow were purchased from commercially available sources (VWR/Sigma Aldrich) and used without any purification or treatment. Solvent (Acetonitrile) and solutions (amidite and activator) were stored over molecular sieves during synthesis.

The control and target oligonucleotide sequences were synthesized on an Expedite 8909 and ABI-394 synthesizers using modified coupling steps. The solid support was controlled pore glass and the monomers contained standard protecting groups. Each chimeric oligonucleotide was individually synthesized using commercially available 5′-O-(4,4′-dimethoxytrityl)-3′-O-(2-cyanoethyl-N, N-diisopropyl) DNA, 2′-OMe, 2′-MOE and or LNA phosphoramidite monomers of 6-N-benzoyladenosine (A^{Bz}), 4-N-acetylcytidine (C^{Ac}), 2-N-isobutyrylguanosine (G^{iBu}), and Uridine (U) or Thymidine (T), according to standard solid phase Phosphoramidite synthesis protocols. The phosphoramidites were prepared as 0.1 M solutions in anhydrous acetonitrile. 5-Ethylthiotetrazole was used as activator, 3% dichloroacetic acid in dichloromethane was used to detritylate, acetic anhydride in THF and 16% N-methylimidazole in THF were used to cap, 0.02 mM I₂/H₂O/Pyridine as oxidizing agent and DDTT (dimethylamino-methylidene) amino)-3H-1,2,4-dithiazaoline-3-thione was used as the sulfur-transfer agent for the synthesis of oligoribonucleotide phosphorothioates. An extended coupling of 0.1M solution of phosphoramidite in CH₃CN in the presence of 5-(ethylthio)-1H-tetrazole activator to a solid bound oligonucleotide followed by extended capping, oxidation and deprotection afforded modified oligonucleotides. The stepwise coupling efficiency of all modified phosphoramidites was more than 98.5%.

Deprotection and cleavage from the solid support was achieved with mixture of ammonia:methylamine (1:1, AMA) for 15 min at 65° C. When the universal linker was used, the deprotection was left for 90 min at 65° C. or solid supports were heated with aqueous ammonia (28%) solution at 55° C. for 8-16 h to deprotect the base labile protecting groups.

After filtering to remove the solid support, the deprotection solution was removed under vacuum in a GeneVac centrifugal evaporator or used as such for next step.

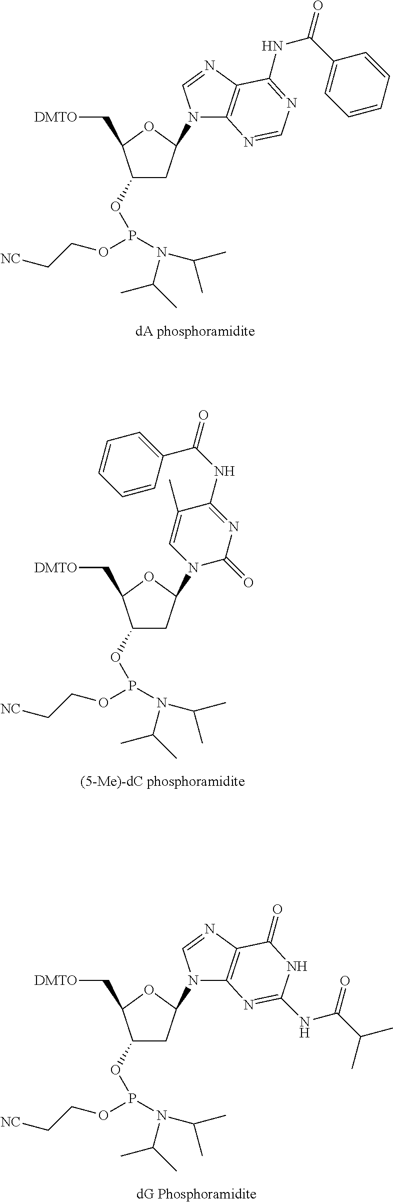

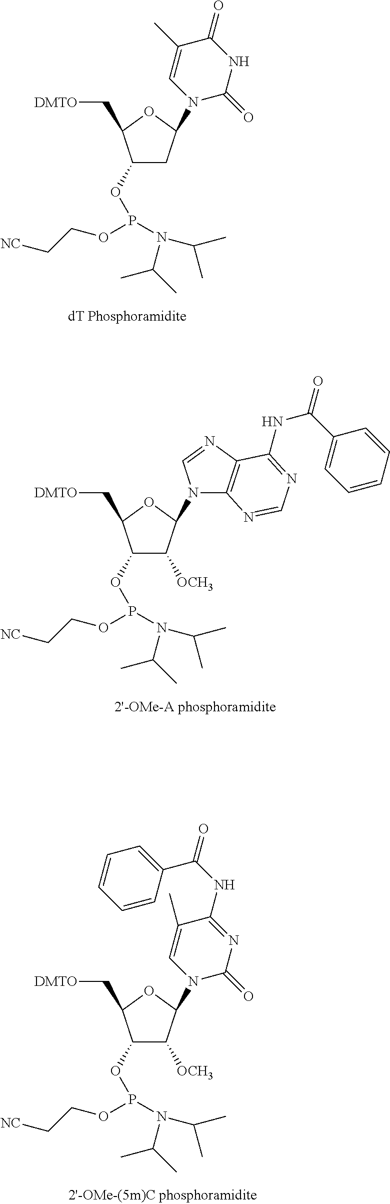

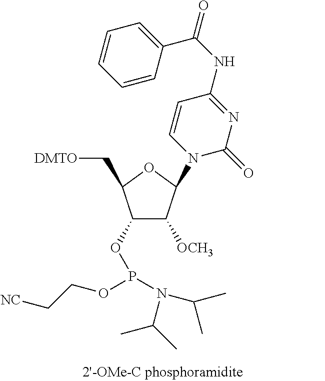

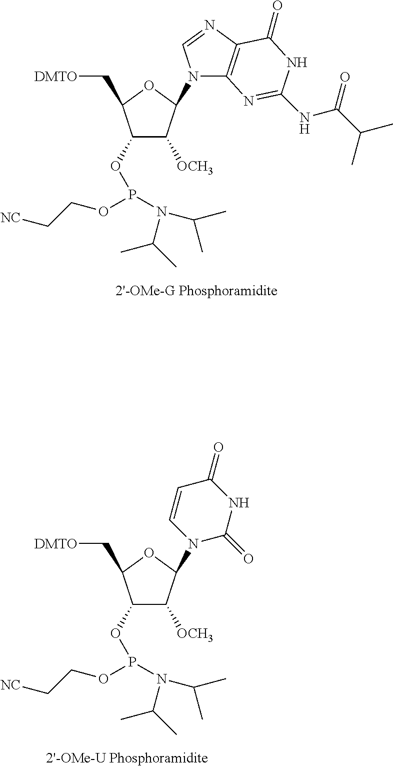

The 2′-MOE Phosphoramidites

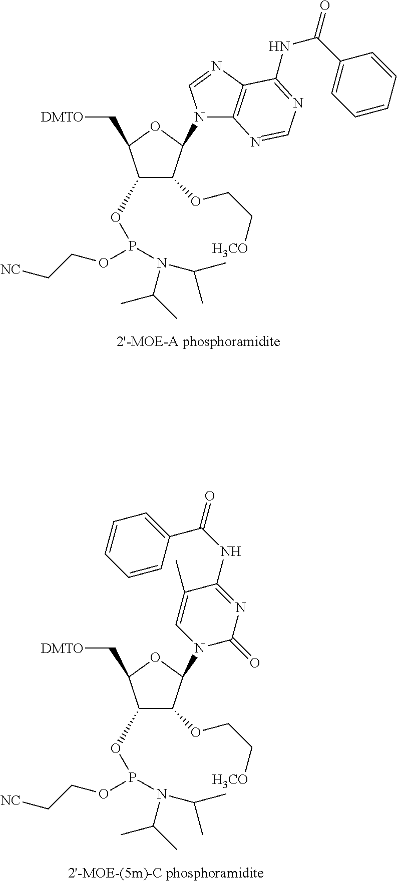

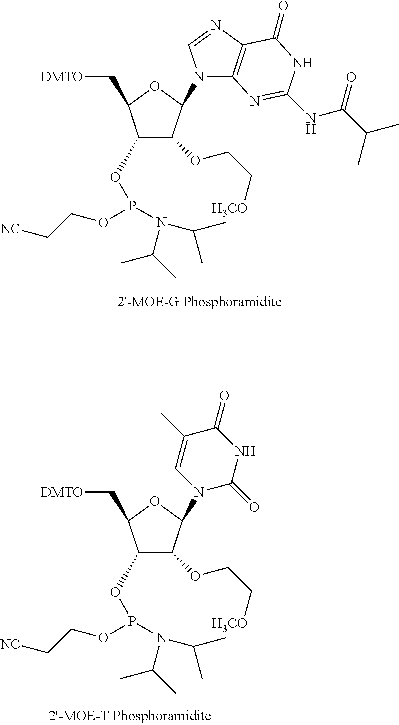

The Locked Nucleic Acid (LNA) Phosphoramidite

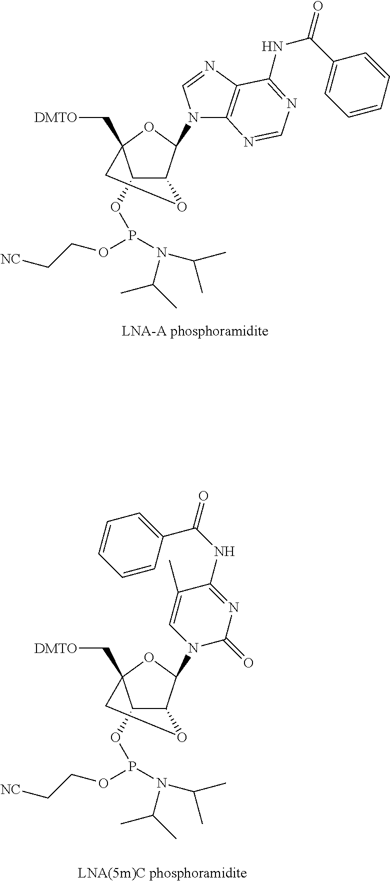

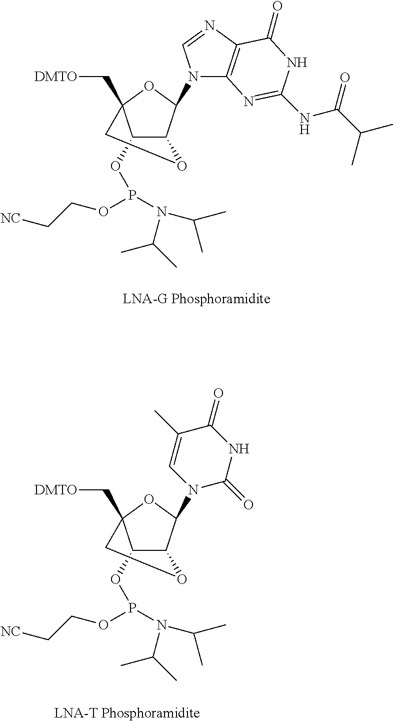

The 2,6-Diaminopurine and G-Clamp Phosphoramidite

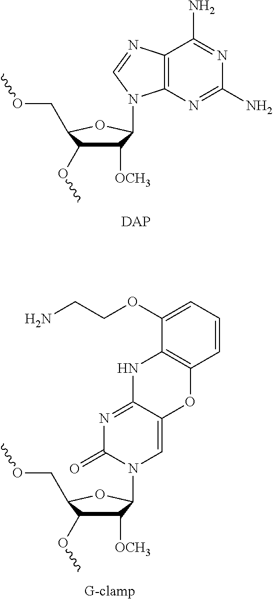

Modified Sequences:

The AmNA (N-Me)T, AmNA (N-Me)-4-N-benzoyl (5m) cytidine ((5m)C^{Bz}), AmNA (N-Me)-4-N-benzoylcytidine (A^{Bz}), and AmNA (N-Me)-2-N-pac (G^{pac}), were purchased from Luxna Biotech, whereas scp-BNA-T, scp-BNA-6-N-benzoyladenosine (A^{Bz}), scp-BNA-4-N-benzoyl-5 methyl cytidine ((5m)C^{Bz}), scp-BNA-2-N-iguanosine (G^{iBu}) phosphoramidite monomers were synthesized by following the procedure described in references (Takao Yamaguchi, Masahiko Horiba and Satoshi Obika; Chem. Commun., 2015, 51, 9737-9740; Masahiko Horiba, Takao Yamaguchi, and Satoshi Obika; Journal of Organic Chemistry, 2016, 81, 11000-11008). All the monomers were dried in a vacuum desiccator with desiccants (KOH and P205, at room temperature for 24 hours). In case of AmNA- and scp-BNA-modifications, the synthesis was carried out on a 1 μmol scale in a 3′ to 5′ direction with the phosphoramidite monomers diluted to a concentration of 0.12 M in anhydrous CH₃CN in the presence of 0.3 M 5-(benzylthio)-1H-tetrazole (BTT) activator (coupling time 16 min) to a solid bound oligonucleotide, followed by modified capping, oxidation and deprotection afforded modified oligonucleotides. The stepwise coupling efficiency of all modified phosphoramidites was more than 97%. The DDTT (dimethylamino-methylidene) amino)-3H-1,2,4-dithiazaoline-3-thione was used as the sulfur-transfer agent for the synthesis of oligoribonucleotide phosphorothioates. Oligonucleotide-bearing solid supports were washed with 20% DEA solution in acetonitrile for 15 min then column was washed thoroughly with CH₃CN. The support was heated at 65° C. with diisopropylamine:water:methanol (1:1:2) for 8 h in heat block to cleave from support and deprotect the base labile protecting groups.

AmNA (N-Me) Monomers

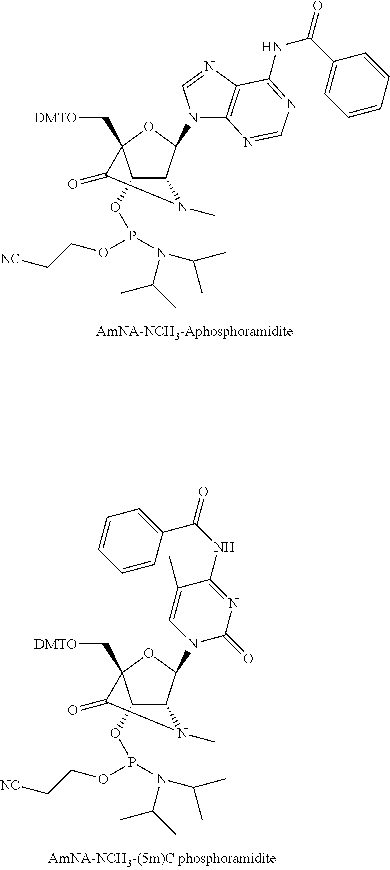

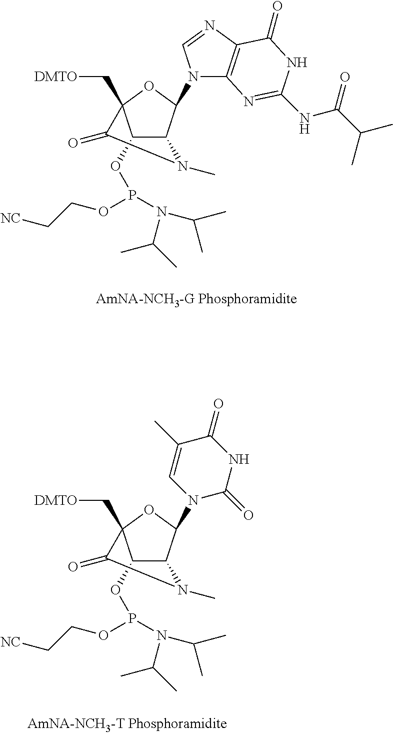

Scp-BNA Monomers

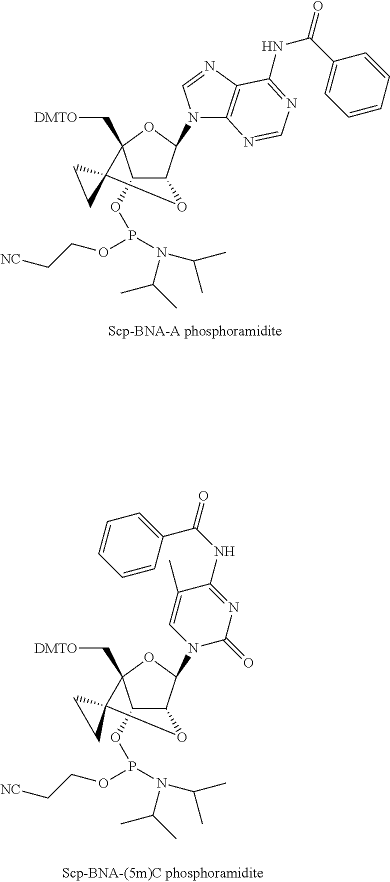

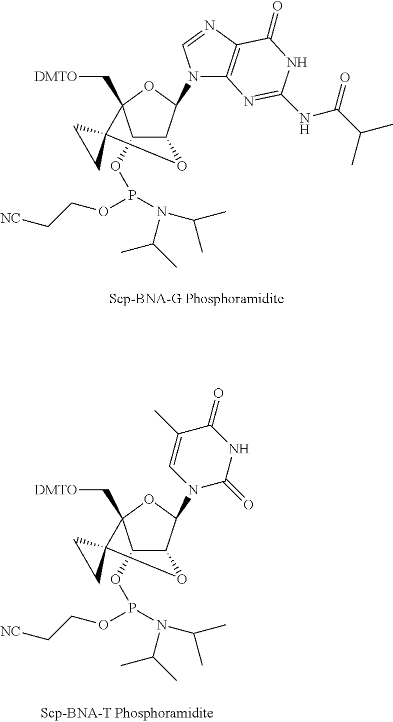

5′ and 3′-GalNac conjugated oligonucleotides were synthesized with various lengths of GalNAc moieties, e.g., as described below.

GalNAc Phosphoramidites

| GalNAc building blocks | After attachment to Oligos |
|---|---|
| 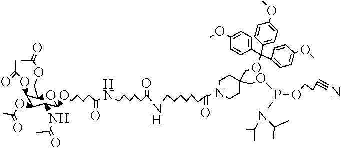 GalNAc-4 phosphoramidite | 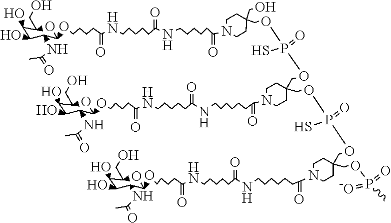 |
| 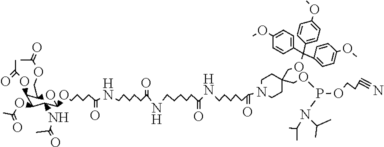 GalNAc-6 phosphoramidite | 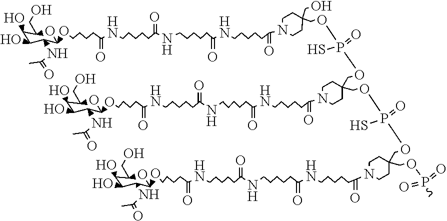 |

Quantitation of Crude Oligomer or Raw Analysis

Samples were dissolved in deionized water and quantified as follows: first, Nanodrop UV spectrophotometer was blanked with water (2 ul). NanoDrop instruments can measure a wide concentration ranges of nucleic acids. The most accurate quantification results can be achieved by measuring diluted oligonucleotides with an absorbance <50.

Determine the approximate absorbance of an oligonucleotide stock solution using the Beer-Lambert equation:

A=εbc

where:

### A===Absorbance

Molar attenuation coefficient (L/(mole·cm))
b=Path length (cm)
c Concentration (M, mole/L)

Crude HPLC/LC-MS analysis

The 0.1 OD of the crude samples were used for crude MS analysis. After confirming the crude LC-MS data, then the purification step was performed.

HPLC Purification

The modified oligonucleotides were purified by anion-exchange HPLC. The buffers were 20 mM sodium phosphate in 10% CH₃CN, pH 8.5 (buffer A) and 20 mM sodium phosphate in 10% CH₃CN, 1.8 M NaBr, pH 8.5 (buffer B). Fractions containing full-length oligonucleotides were pooled, desalted, and lyophilized.

Desalting of Purified Oligomer

The purified dry oligomer was then desalted using Sephadex G-25 M (Amersham Biosciences). The cartridge was conditioned with 10 mL of deionized water thrice. The purified oligonucleotide was dissolved thoroughly in 2.5 mL deionized water and the mixture was applied to the cartridge with very slow drop wise elution. The salt free oligomer was eluted with 3.5 ml deionized water directly into a screw cap vial.

Final HPLC and Electrospray LC/MS Analysis

Approximately 0.10 OD of oligomer was pipetted into HPLC autosampler vials for IEX-HPLC and LC/MS analysis. Analytical HPLC and ES-LC-MS established the integrity of the chimeric oligonucleotides.

Post-Synthetic Conjugation of GalNAc esters to Oligonucleotides

5′-C6-Amino Precursor Synthesis

The sequences were synthesized at 10 μmol scale using universal support (Loading 65 μmol/g). At the 5′-terminal C6-NH₂ linker was introduced using 6-(4-monomethoxytritylamino)hexyl-(2-cyanoethyl)-(N, N-diisopropyl)-phosphoramidite in 0.1 M acetonitrile with coupling time of 10 minutes. The oligonucleotide-bearing solid support was heated with aqueous ammonia (28%) solution at 55° C. for 8 h to deprotect the base labile protecting groups. After IEX purification and desalting, the C6-NH₂ modified oligonucleotide was used to perform post-synthetic conjugation.

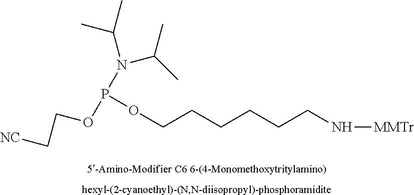

### Amino Modifier

Oligonucleotide after C6-Amino Coupling:

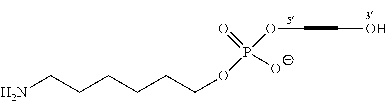

GalNAc Ester for Conjugation

| | GalNAc ester | After attachment to Oligonucleotide |
|---|---|---|
| GalNAc- 1 ester | 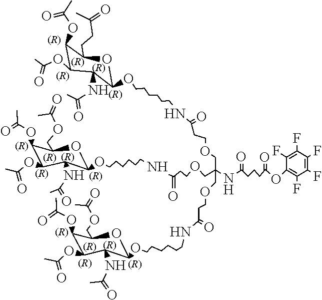 | 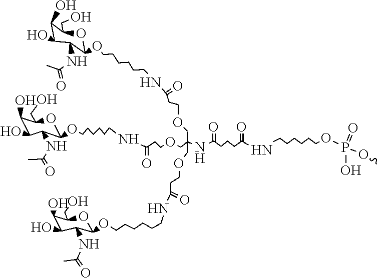 |
| GalNAc2 ester | 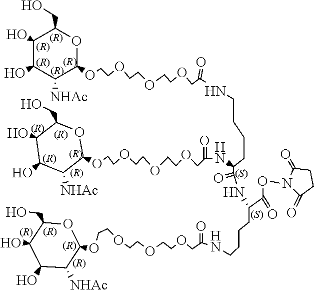 | 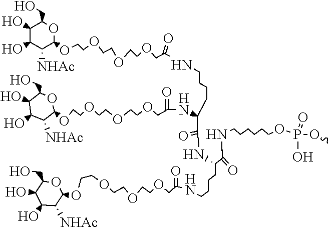 |

Post-Synthetic Conjugation of 5′-GalNAc Synthesis

The 5′-C6-NH₂ modified sequences were dissolved in 0.2 M sodium bicarbonate buffer, pH 8.5 to give 0.015 mM solution and 5-7 mol equivalent of GalNAc ester in DMSO was added. The reaction mixture was stirred at room temperature for 4 h. The sample was analyzed to confirm if any unreacted amino modified oligonucleotide is present. To this, aqueous ammonia (28 wt. %) was added (5× reaction volume) and stirred at room temperature for 2-3 h. Reaction mixture was concentrated under reduced pressure and residue was dissolved in water and purified by HPLC on a strong anion exchange column.

Lipid Conjugated Oligonucleotides

The Cholesterol, Tocopherol and Palmitoyl building blocks (phosphoramidite and lipid loaded on resin) were procured from commercial sources to make 5′ and 3′conjugated oligonucleotides. The synthesis was performed on ABI-394 or Expedite 8909 using the same procedure as described above.

Crude HPLC/LC-MS Analysis

The conjugated oligonucleotides were analyzed on Agilent 1200 system by using a Luna C₈ column 100 mM HFIP, 7 mM TEA as buffer A and acetonitrile as buffer B with a 15-55% gradient 20 min. Flow rate and column temperature were 1.0 mL/min and 60° C., respectively.

HPLC Purification

The Cholesterol, Tocopherol, or Palmitoyl oligonucleotides were purified by a reverse-phase HPLC column (Sepax GP-C8 column) using 50 mM sodium acetate in 10% acetonitrile (buffer A) and 100% acetonitrile (buffer B). Fractions containing full-length oligonucleotide product were pooled, desalted, and lyophilized.

### 5′-Tocopherol (Vitamin E) Attached Via TEG Linker

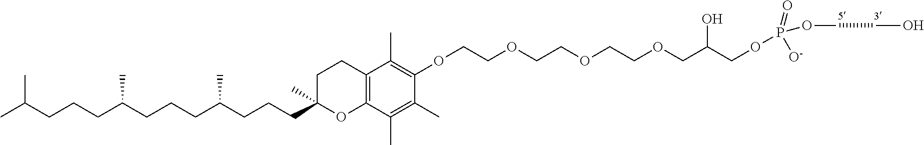

### 3′-Tocopherol (Vitamin E) Attached Via TEG Linker

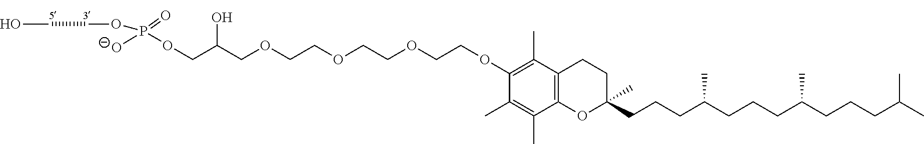

### 5′-Cholesterol Attached Via TEG Linker

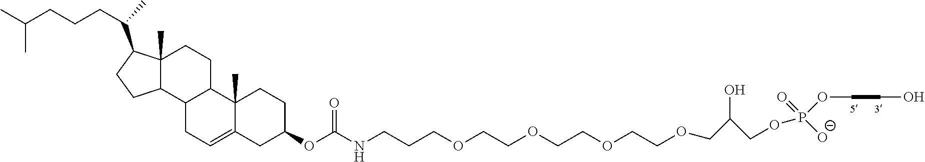

### 3′-Cholesterol Attached Via TEG Linker

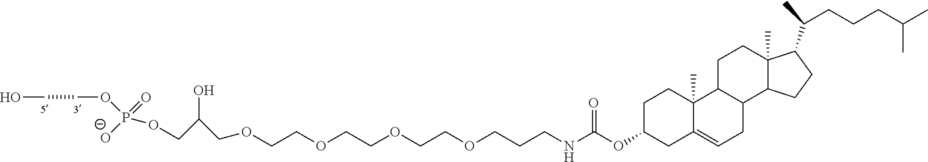

### 5′-Palmitoyl Conjugated Oligonucleotides

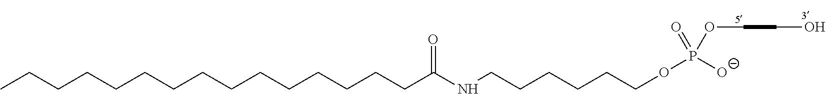

### 3′-Palmitoyl Conjugated Oligonucleotides

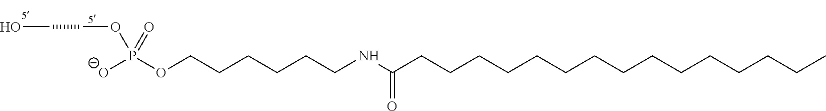

### Example 2: Determination of EC50 and CC50 Values of Steric Blockers

This protocol was designed to examine whether rc-cccDNA steric blockers can inhibit cccDNA surrogate HBsAg release in vitro. Steric Blockers corresponding to SEQ ID NOs: 65-127 were assessed using this protocol.

Cryo-preserved PHH was thawed and quickly mixed with thawing and plating medium (William's E medium, Thermo Fisher Scientific, A1217601) supplemented with primary hepatocyte thawing and plating supplements (Thermo Fisher Scientific, CM3000). Seed cells at 80 k/well for 96-well plate and incubate in 37° C. and 5% CO₂ incubator overnight.

After overnight incubation, cells were infected with HBV MOI 200 with infection medium supplemented with 2% DMSO and 4% PEG.

After infection for overnight, the viral inoculum was removed, and the cells are washed three times with prewarmed wash medium. Then refill with fresh PHH culturing medium (Dulbecco's Modification of Eagle's Medium (DMEM) with 2 mM glutamine and 1% sodium pyruvate (Corning;10-092-CM), further supplemented with 10% fetal bovine serum (Sigma, 16L571), 1% penicillin and streptomycin (Catalog No. 30-002-CI, Corning), 20 mM HEPES (Corning, 25-060-CI), 15 μg/mL L-proline (Sigma, P5607), 0.25 μg/mL insulin (ThermoFisher Scientific, 12585014), 5 ng/mL Human epidermal growth factor (ThermoFisher Scientific, PHG6045), 50 nM dexamethasone, 0.1 mM ascorbic acid (Sigma, 49752).

Four days post washing, the cells were replenished with 90μl fresh medium and then transfected with oligos as shown below.

Dilute oligos in Opti-MEM I (Life Technology, Cat #: 31985-070) to 20× of final concentration, mix with equal volume Opti-MEM I containing Lipofectamine RNAiMAX (Invitrogen, Cat #: 13778-150), pipet 3 times and incubate for 10-20 min at room temperature. Add 10 ul oligo:RNAiMAX mixture to the cells, mix gently and put the plates back to incubator.

After 3 days, the medium was refreshed. On day 11, the supernatant was harvested for HBsAg quantitation and the cells were assayed for viability. The HBsAg was measured using the HBsAg CLIA (DiaSino, DS187701), and the cell viability was measured using the CellTiter-Glo® Luminescent Cell Viability Assay (PromegaG7572) following the manufacturers' protocols.

The EC50 and CC50 results for Steric Blockers corresponding to SEQ ID NOs: 65-127, which were assessed using this protocol, are shown in Table 3.

### Example 3: Determination of EC50 and CC50 Values of Steric Blockers

This protocol was designed to examine whether rc-cccDNA steric blockers can inhibit cccDNA surrogate HBsAg release in vitro after cccDNA level had established and stabilized (typically 4-5 days post HBV infection). A HepG2-NTCP cell line was used, which continuously expressed the HBV receptor, NTCP. The HepG2-NTCP cells were maintained in DMEM/F12 (catalog #: 10-092-CM, Corning) medium with 10% FBS, 1% penicillin and streptomycin, 1% glutamine, 1% non-essential amino acids and 1% sodium pyruvate. The cells were trypsinized at 37° C. and diluted to 0.2×10⁶/ml with the maintenance medium. In short, the cells were seeded at 20,000/well in 96-well plate and infected with HBV at 200 moi. The cells were transfected with rc-cccDNA steric blockers on day 4 as well as day 7 post infection and the HBsAg in supernatant was measured on day 10 after infection (day 6 after treatment). ASOs corresponding to SEQ ID NOs: 127-198 were assessed using this protocol.

Transfection Protocol:

Prepare Transfection Mixture:

A: A master mix was made by mixing RNAiMAX (catalog: 13778-150, Thermo fisher. 0.3 ul/well for 96-well plate) with Opti-MEM I (5.2 ul/well). At least 20% extra volume was maintained, and the mixture was vortexed and incubated for 5 min at RT.

B: Serial dilutions of rc-cccDNA steric blockers (3-fold) were prepared with Opti-MEM I at 20× of final concentration (8-point dose response).

A and B were mixed at equal volumes, incubated another 5-10 min, and then added to plates.

Specifically, 11 μl of mixed A and B was added to each well, followed by 100 μl HepG2-NTCP cells, and the plates were swirled for 10 seconds by hand.

The plates were then incubated at 37° C. for 3 days, and medium was refreshed, but there were no further transfections with rc-cccDNA steric blockers.

On day 6 after treatment, the supernatant was harvested. The HBsAg was measured with ELISA kit (catalog: DS187701, Diasino), and cell viability was measured with CellTiter-Glo (Promega). The EC50 and CC50 results for Steric Blockers corresponding to SEQ ID NOs: 128-198, which were assessed using this protocol, are shown in Table 3.

### Example 4: Inhibition of rcDNA and cccDNA by Steric Blockers in HepG2-NTCP Cells Infected with D Type HBV

Culture and Treatment of Cells

On day −2 (2 days before infection), HepG2-NTCP cells as described in Example 3 were seeded into 48-well plates at a density of 7.5×104 cells/well (0.25 ml/well). The final concentration of FBS in the seeding medium is 2%. The cells were incubated at 37° C. and 5% CO2. On day −1 (1 day before infection), the medium was changed with DMEM containing 2% FBS and 2% DMSO. On day 0, the HepG2-NTCP cells were infected with D type HBV. On day 1, medium was changed with DMEM containing 2% FBS and 1% DMSO. On day 4, the Steric Blockers were transfected into the HepG2-NTCP by RNAiMAX as described in Example 3. The Steric Blockers transfected were either SEQ ID NO: 161 at a concentration of either 500 nM, 167 nM, 55.6 nM, 18.5 nM, 6.17 nM, or 2.06 nM, SEQ ID NO: 93, SEQ ID NO: 95, SEQ ID NO: 78, SEQ ID NO: 122, SEQ ID NO: 75, SEQ ID NO: 77, or SEQ ID NO: 171 at a concentration of either 500 nM, 166.7 nM, 55.6 nM, 18.5 nM, 6.17 nM, or 2.06 nM. The cells were cultured at 37° C. and 5% CO2 for 3 days. On day 7, the cells were re-transfected with the Steric Blockers (procedure same as day 4). On day 10, the cell culture supernatants were collected for the determinations of HBsAg by ELISA and cell viability by Cell Counting Kit-8 (CCK-8, Dojindo Molecular Technologies SKU: CK04). Cells are harvested for cccDNA detection.

Measurement of cccDNA by Southern Blot

Extraction of protein-free viral DNA (cccDNA and protein-free rcDNA) was carried out by using a modified Hirt extraction procedure. Briefly, cells from 48-well plates (three wells pooled) were lysed using 10 mM Tris-HCl (pH 7.5), 10 mM EDTA, and 0.7% SDS. After 30 minutes of incubation at room temperature, 5 M NaCl was added to the cells and the cells were incubated at 4° C. overnight. The lysate was clarified by centrifugation at 12,000 g for 30 minutes at 4° C. and extracted three times with Phenol:chloroform:isoamyl alcohol (25:24:1). DNA was precipitated with 0.7 volume of isopropanol and incubate at −20° C. overnight, then dissolved in AE buffer.

The Hirt DNAs were resolved on a 1.2% agarose gel and transferred onto a positive-charged Nylon membrane. For the detection of HBV DNAs, the membrane was probed with a DIG-labeled HBV DNA probe. Hybridization was carried out in 10 ml of the hybridization buffer with a 1-hour prehybridization at 60° C. and overnight hybridization at 60° C., followed by 2×5-minute wash with 2×SSC, 0.1% SDS at room temperature and 4×15 minute wash with 0.2×SSC, 0.1% SDS at 60° C. The membrane was incubated with a blocking buffer for 60 minutes and followed by 60 minutes incubation with the antibody solution. After equilibration with the detection buffer for 10 minutes, the membranes were rinsed with CDP-star and followed by analysis with ImageQuant™ LAS 4010 (GE Healthcare) at room temperature. The relative density of the cccDNA band in the Southern blot was quantified with ImageQuant™ LAS 4010 software.

Quantitation of Mitochondrial Cox3 Gene by qPCR and cccDNA Normalization

The Cox3 gene was quantified by qPCR using the Hirt DNA samples. Plasmid containing the Cox3 gene was used as a standard whereby the standard had a 10-fold serial dilution, and the range of the standard used was between 101-1.0×108 copies/W. 2 μl of the diluted plasmid standard or samples was added to PCR plates. qPCR was run at 50° C. for 2 minutes, 95° C. for 2 min, then cycling at 95° C. for 5 seconds, 60° C. for 30 seconds for 40 cycles, 95° C. for 15 seconds, 60° C. for 15 seconds, 95° C. for 15 seconds.

The cox3 normalization was calculated using the following formula:

Relative Cox3 level=cox3 copy number from the Steric Blocker treated sample/the average cox3 copy number of control sample (no Steric Blocker treatment).

cccDNA normalized by Cox3 was calculated using the following formula:

Relative cccDNA normalized by cox3 gene=density of cccDNA band quantification/Relative cox3 level of sample.

Results

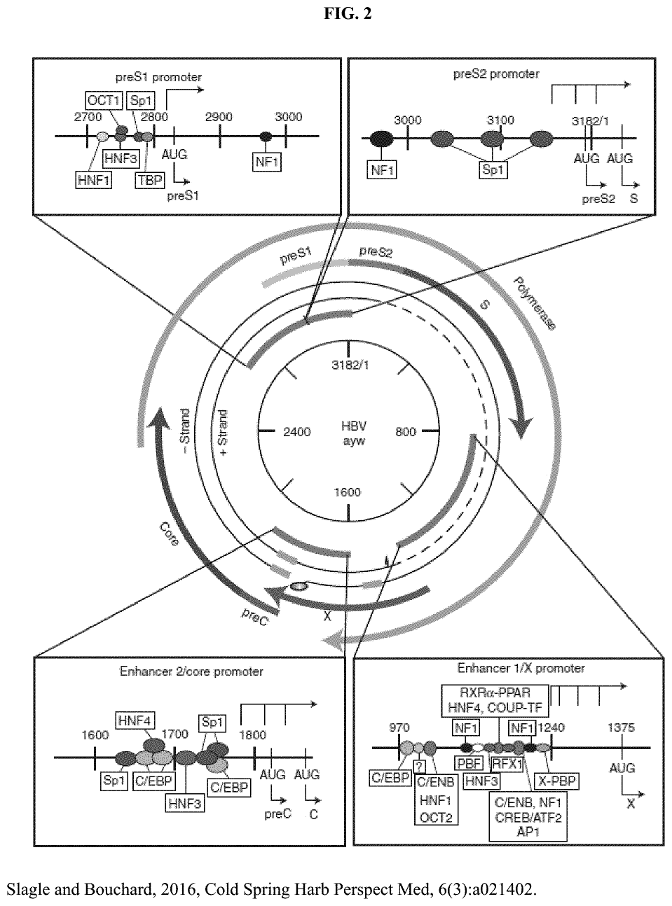

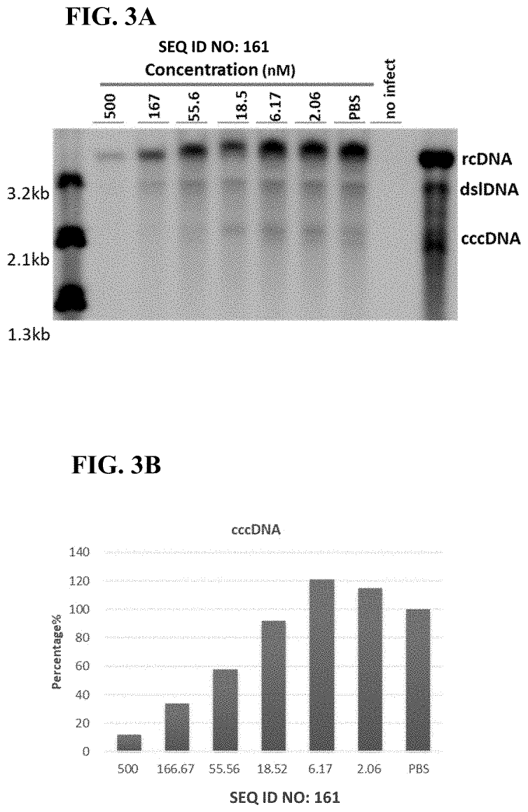

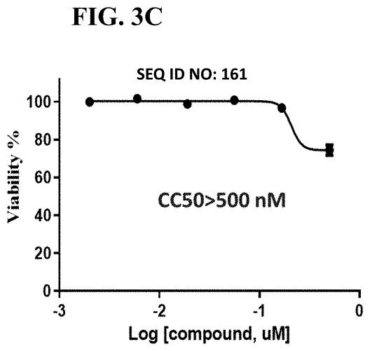

HepG2-NTCP cells transfected with the Steric Blocker corresponding to SEQ ID NO: 161 (lnTpsmGpsln(5m)Cps m(5m)CpslnApsmAps ln(5m)CpsmUpslnGps mGpslnApsmUps ln(5m)Cpsm(5m)CpslnT) inhibits rcDNA and cccDNA 4 days post infection at differing concentrations compared with PBS control determined by Southern Blot (FIG. 3A) and by calculating the percentage of cccDNA (FIG. 3B) when compared to PBS control. Cells were viable at all treatment concentrations determined by cell count analysis (FIG. 3C).

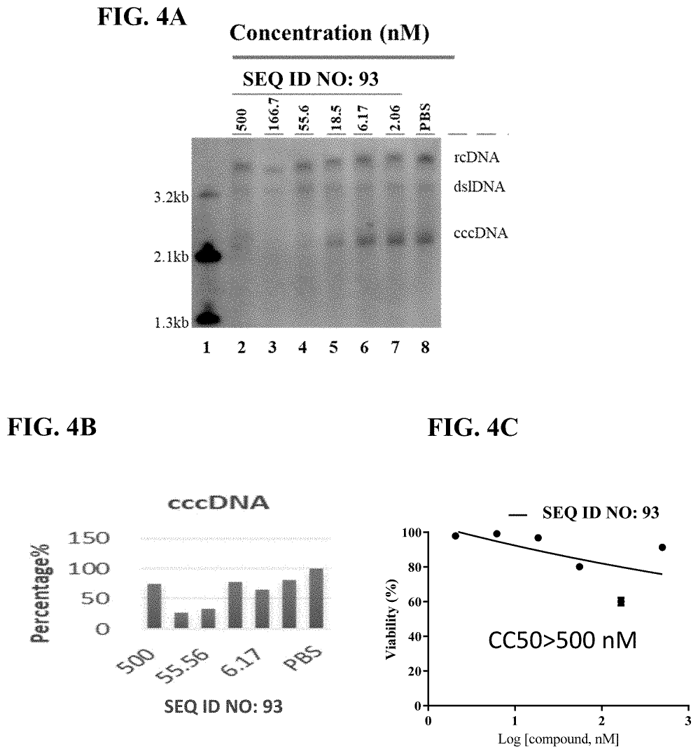

HepG2-NTCP cells transfected with the Steric Blocker corresponding to SEQ ID NO: 93 (lnTpsmGpsln(5m)CpsmCpslnApsmApsln(5m)CpsmUpslnGpsmGpslnApsmUpsln(5m)CpsmCp slnT) inhibits cccDNA 4 days post infection at differing concentrations compared with PBS control determined by Southern Blot (FIG. 4A) and by calculating the percentage of cccDNA (FIG. 4B) when compared to PBS control. Cells were viable at all treatment concentrations determined by cell count analysis (FIG. 4C).

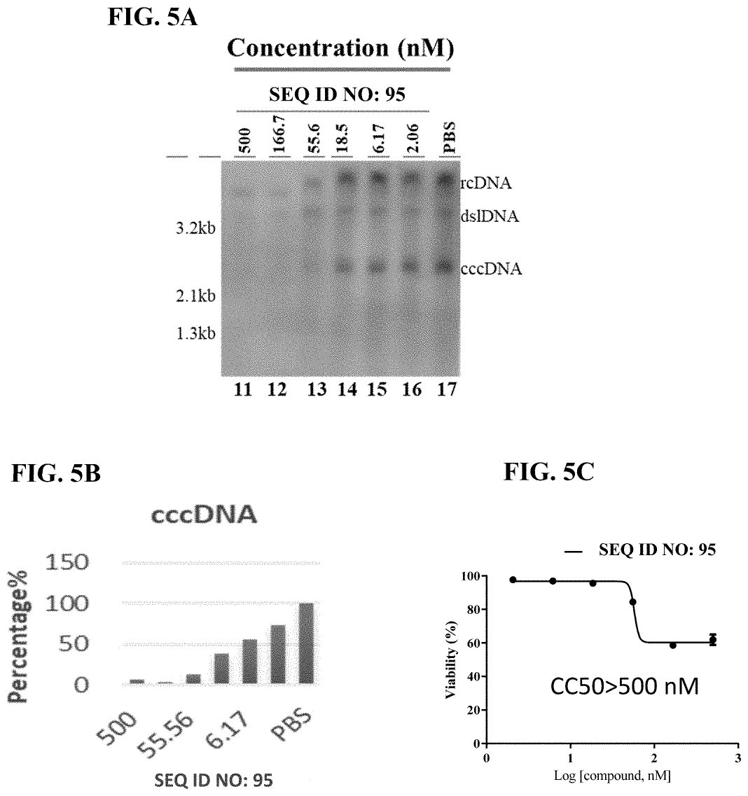

HepG2-NTCP cells transfected with the Steric Blocker corresponding to SEQ ID NO: 95 (ln(5m)CpsmUpslnGpsmCpsln(5m)CpsmApslnApsmCpslnTpsmGpslnGpsmApslnTpsmCpsln(5m)CpsmU) inhibits rcDNA and cccDNA 4 days post infection at differing concentrations compared with PBS control determined by Southern Blot (FIG. 5A) and by calculating the percentage of cccDNA (FIG. 5B) when compared to PBS control. Cells were viable at allow to mid range treatment concentrations determined by cell count analysis. For two highest concentrations, viability dropped down to 50% (FIG. 5C).

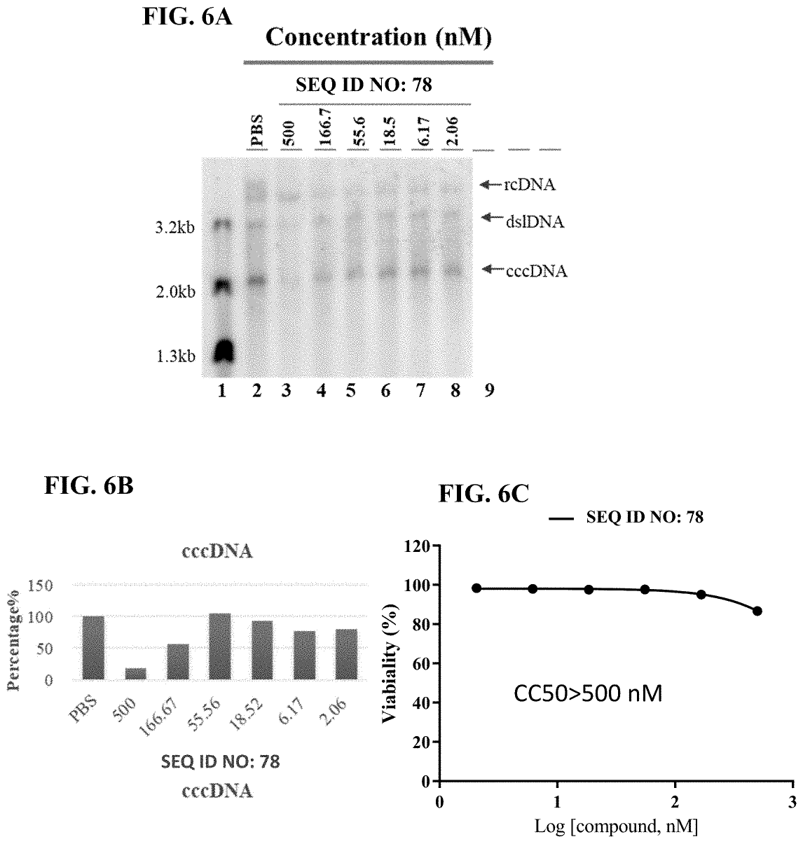

HepG2-NTCP cells transfected with the Steric Blocker corresponding to SEQ ID NO: 78 (ln(5m)CpslnGpsln(5m)CpslnApsln(5m)Cpsln(5m)CpslnTpsln(5m)CpslnTpsln(5m)CpslnTpsln TpslnTpslnApsln(5m)C) inhibits cccDNA 4 days post infection at differing concentrations compared with PBS control determined by Southern Blot (FIG. 6A) and by calculating the percentage of cccDNA (FIG. 6B) when compared to PBS control. Cells were viable at all treatment concentrations determined by cell count analysis (FIG. 6C).

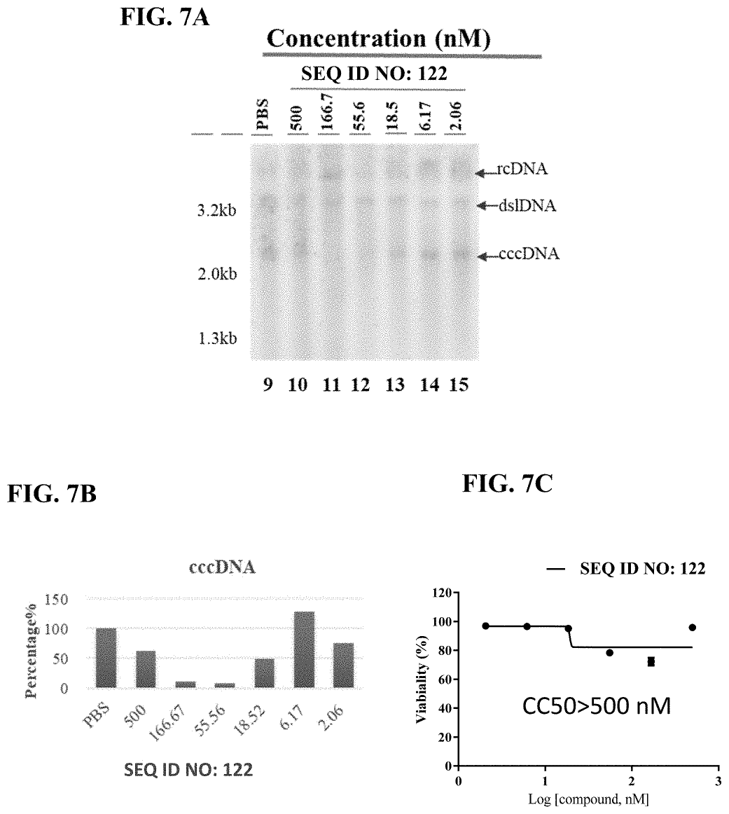

HepG2-NTCP cells transfected with the Steric Blocker corresponding to SEQ ID NO: 122 (lnTpsmGpsln(5m)CpsmCpslnGpsmApslnTpsmCpsln(5m)CpsmApslnTpsmApsln(5m)CpsmUp slnG) inhibits cccDNA 4 days post infection at differing concentrations compared with PBS control determined by Southern Blot (FIG. 7A) and by calculating the percentage of cccDNA (FIG. 7B) when compared to PBS control. Cells were viable at all treatment concentrations determined by cell count analysis (FIG. 7C).

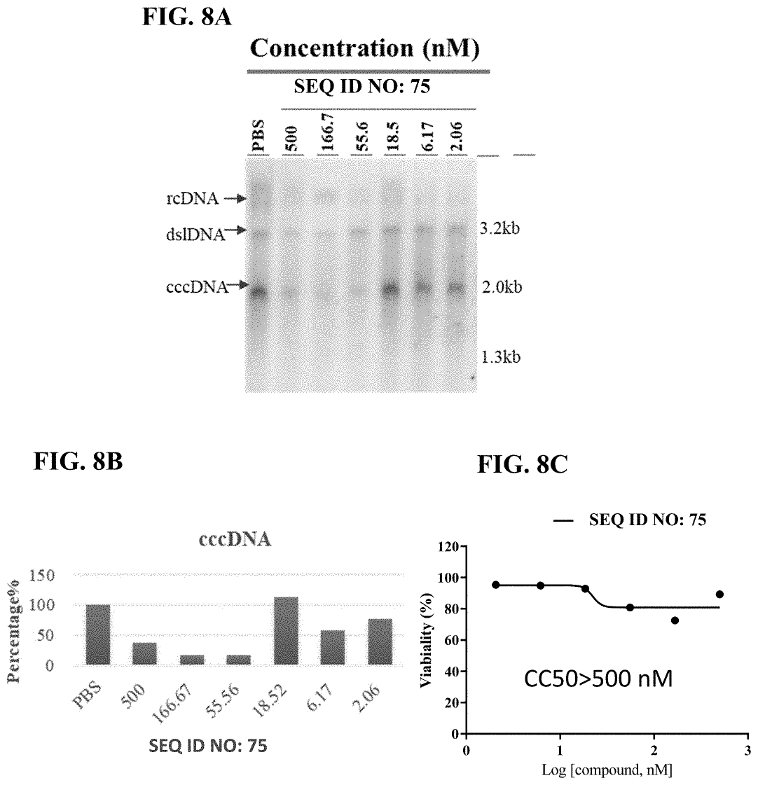

HepG2-NTCP cells transfected with the Steric Blocker corresponding to SEQ ID NO: 75 (ln(5m)CpsmGpsln(5m)CpsmApsln(5m)CpsmCpslnTpsmCpslnTpsmCpslnTpsmUpslnTpsmAps ln(5m)CpsmG) inhibits cccDNA 4 days post infection at differing concentrations compared with PBS control determined by Southern Blot (FIG. 8A) and by calculating the percentage of cccDNA (FIG. 8B) when compared to PBS control. Cells were viable at all treatment concentrations determined by cell count analysis (FIG. 8C).

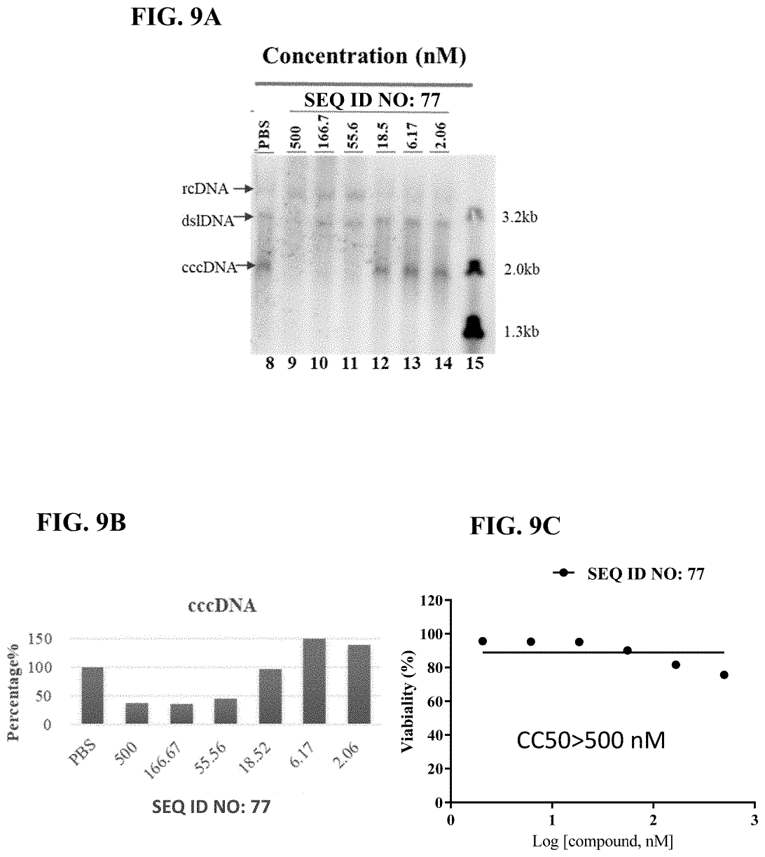

HepG2-NTCP cells transfected with the Steric Blocker corresponding to SEQ ID NO: 77 (lnGpsln(5m)CpslnApsln(5m)Cpsln(5m)CpslnTpsln(5m)CpslnTpsln(5m)CpslnTpslnTpslnTpsln Apsln(5m)CpslnGpsln(5m)C) inhibits cccDNA 4 days post infection at differing concentrations compared with PBS control determined by Southern Blot (FIG. 9A) and by calculating the percentage of cccDNA (FIG. 9B) when compared to PBS control. Cells were viable at all treatment concentrations determined by cell count analysis (FIG. 9C).

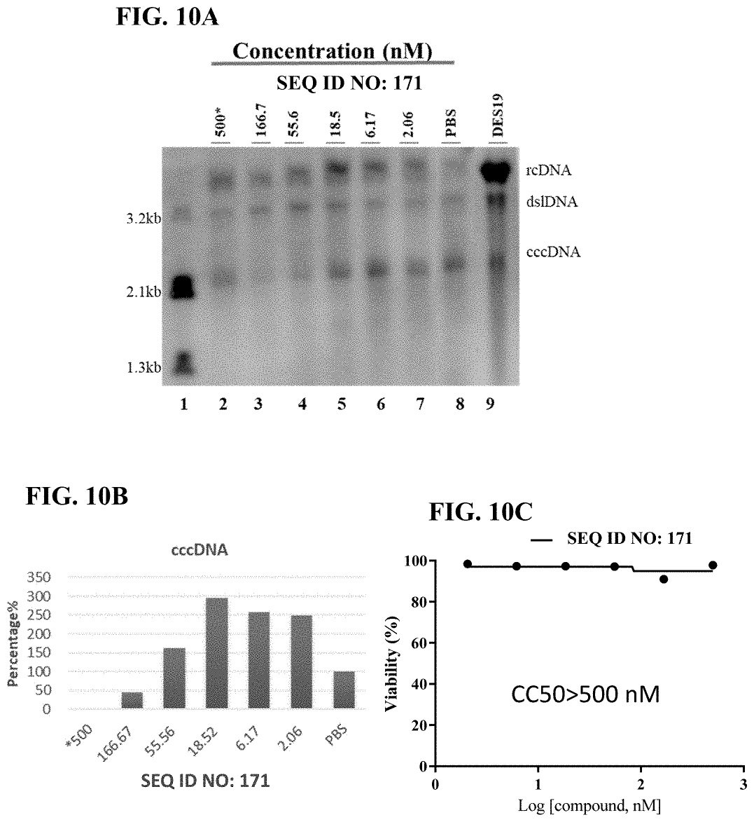

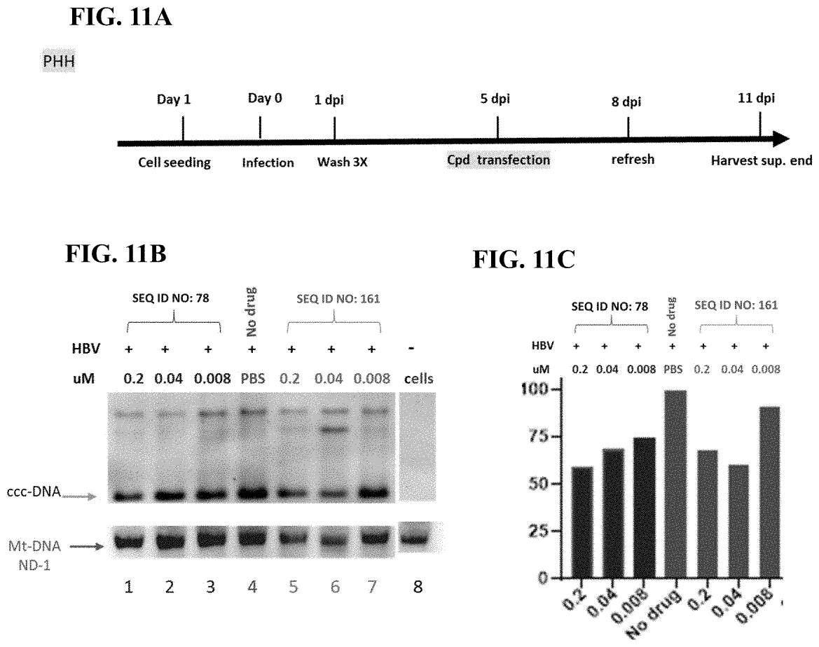

HepG2-NTCP cells transfected with the Steric Blocker corresponding to SEQ ID NO: 171 (ln(5m)CpsmApslnApsmGpslnApsmApslnTpsmApslnTpsmGpslnGpsmUpslnGpsmApsln(5m)C psm(5m)C) inhibits cccDNA 4 days post infection at differing concentrations compared with PBS control determined by Southern Blot (FIG. 10A) and by calculating the percentage of cccDNA (FIG. 10B) when compared to PBS control. Cells were viable at all treatment concentrations determined by cell count analysis (FIG. 10C).

### Example 5: Inhibition of cccDNA by Steric Blockers in HBV Infected PHH Cells

Culture and Treatment of Cells

The infection of PHH cells and treatment with the Steric Blockers was performed as described in Example 2. The cells were treated with the Steric Blocker SEQ ID NO: 161 or SEQ ID NO: 78 at a concentration of either 0.2 uM, 0.04 uM, or 0.008 uM, or SEQ ID NO: 100 at a concentration of 1 uM, 0.5 uM, 0.25 uM, 0.125 uM, or 0.06 uM.

Measurement of cccDNA by Southern Blott

Added to the cells was 1500 ul TE (10:10) and 100 ul of 10% SDS to each well of the 6 well plates (˜one millions of cells). The plates were gently mix and incubated for 30 minutes at room temperature. The cell lysate was transferred to a 2 mL centrifuge tube, 400 ul of 5 M NaCl was added to the cell lysate and the tube was and gently inverted at 4° C. for at least 16 h. The cell lysate was centrifuge at 14,500×g for 30 minutes at 4° C. The supernatant was transferred to a fresh 2 ml centrifuge tube. An equal volume of phenol (˜2 mL) was added to the supernatant and mixed thoroughly by hand shaking for 10 seconds. The tubes were centrifuge at 3,500×g for 10 minutes at 4° C. and the aqueous phase was transferred to a fresh 2 ml tube. The step (phenol extraction step) was repeated once. Equal volume of phenol/chloroform was added to the tube and the tub was mixed by hand shaking, centrifuge at 3,500×g for 10 min at 4° C., and transfer the aqueous phase to a fresh 15 mL tube. This step (phenol extraction step was repeated once). Two volumes of 100% ethanol were added to the 15 mL tube and the tube was inverting 10 times. The sample was incubated at room temperature overnight to precipitate the DNA. The next day, the tube was spun in a centrifuge at 3,500×g for 30 minutes at 4° C., and the supernatant was discarded. 2 ml of 75% ethanol was added to wash the DNA pellet. The tub was spun at 3,500×g for 15 minutes at 4° C. The supernatant was discarded. The pellet was air dry for 10 minutes at room temperature. The DNA pellet was dissolved in TE buffer (10:1).

The extracted DNA was loaded on to a 1.2% agarose gel and the gel was run at 30V overnight and transferred onto a positive-charged Nylon membrane. For the detection of HBV DNAs, the membrane was probed with a DIG-labeled HBV DNA probe. Hybridization was carried out in 10 ml of the hybridization buffer with a 1-hour pre hybridization at 45° C. and the overnight hybridization at 45° C., followed by a 2×5-minute wash with 2×SSC, 0.1% SDS at room temperature and a 4×15-minute wash with 0.2×SSC, 0.1% SDS at 55° C. The membrane was incubated with blocking buffer for 60 minutes followed by a 60-minute incubation with the antibody solution. After equilibration with the detection buffer for 3 minutes, the membranes were rinsed with CDP-Star™ (ThermoFisher) and followed by analysis with FluorChem (Protein Simple, San Jose, Calif.) gel image system at room temperature. As for internal control, the membrane was washed and probed with mitochondrial-ND1 probe overnight at 55° C., the wash was repeat wash and analysis with FluorShemn, a gel image system was performed. The relative density of the cccDNA band and ND1 band in the Southern blot was quantified with Image J software (imagej.nih.gov/ij/).

Quantification of cccDNA

The ND1 normalization was calculated using the following formula:

Relative ND1 level=density of ND1 of Steric Blocker treated sample/average density of ND1 control (no Steric Blocker treatment).

cccDNA normalization was calculated using the following formula:

Relative cccDNA normalized by ND1 gene=density of cccDNA band quantification/Relative ND1 level of Steric Blocker treated sample.

Results

PHH HBV infected cells transfected with the Steric Blocker corresponding to SEQ ID NO: 161 and SEQ ID NO: 78 (FIG. 11A) reduces cccDNA at differing concentrations compared with PBS control determined by Southern Blott (FIG. 11B; left panel SEQ ID NO: 78; right panel SEQ ID NO: 161) and by calculating the percentage of cccDNA (FIG. 11C; left panel SEQ ID NO: 78; right panel SEQ ID NO: 161).

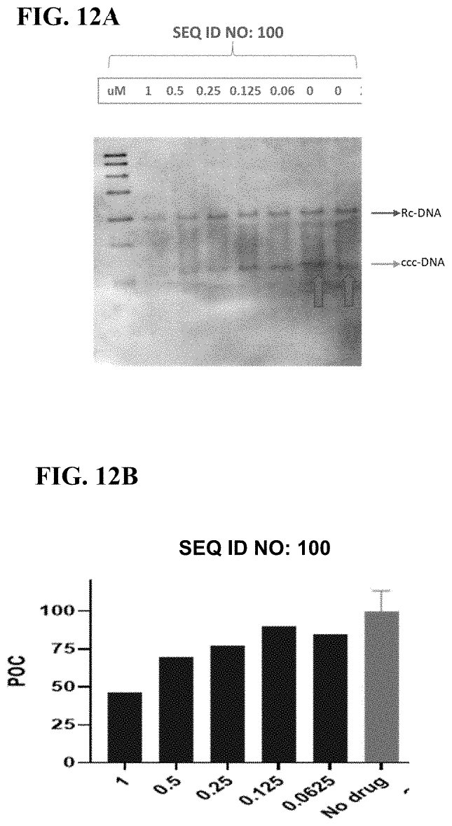

PHH HBV infected cells transfected with the Steric Blocker corresponding to SEQ ID NO: 100 (lnGpsmApslnTpsmCpsln(5m)CpsmApslnTpsmApsln(5m)CpsmUpslnGpsmCpslnGpsmGpslnA psmA) reduces cccDNA at differing concentrations compared with control determined by Southern Blot (FIG. 12A) and by calculating the percentage of cccDNA (FIG. 12B).

### Definitions

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the present application and relevant art and should not be interpreted in an idealized or overly formal sense unless expressly so defined herein. While not explicitly defined below, such terms should be interpreted according to their common meaning.

The terminology used in the description herein is for the purpose of describing particular embodiments only and is not intended to be limiting of the invention. All publications, patent applications, patents and other references mentioned herein are incorporated by reference in their entirety.

The practice of the present technology will employ, unless otherwise indicated, conventional techniques of tissue culture, immunology, molecular biology, microbiology, cell biology, and recombinant DNA, which are within the skill of the art.

Unless the context indicates otherwise, it is specifically intended that the various features of the invention described herein can be used in any combination. Moreover, the disclosure also contemplates that in some embodiments, any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combination.

Unless explicitly indicated otherwise, all specified embodiments, features, and terms intend to include both the recited embodiment, feature, or term and biological equivalents thereof.

All numerical designations, e.g., pH, temperature, time, concentration, and molecular weight, including ranges, are approximations which are varied (+) or (−) by increments of 1.0 or 0.1, as appropriate, or alternatively by a variation of +/−15%, or alternatively 10%, or alternatively 5%, or alternatively 2% and such ranges are included. It is to be understood, although not always explicitly stated, that all numerical designations are preceded by the term “about”. It also is to be understood, although not always explicitly stated, that the reagents described herein are merely exemplary and that equivalents of such are known in the art.

As used herein, the terms “increased”, “decreased”, “reduced”, “high”, “low” or any grammatical variation thereof refer to a variation of about 99%, 98%, 97%, 96%, 95%, 94%, 93%, 92%, 91%, 90%, 89%, 88%, 87%, 86%, 85%, 84%, 83%, 82%, 81%, 80%, 75%, 70%, 65%, 60%, 55%, 50%, 45%, 40%, 35%, 30%, 25%, 20%, 15%, 10%, 5%, 1%, 0.5%, or even 0.1% of the reference composition, virus, viral titers, polypeptide, protein, etc.

The terms or “acceptable,” “effective,” or “sufficient” when used to describe the selection of any components, ranges, dose forms, etc. disclosed herein intend that said component, range, dose form, etc. is suitable for the disclosed purpose.

Also as used herein, “and/or” refers to and encompasses any and all possible combinations of one or more of the associated listed items, as well as the lack of combinations when interpreted in the alternative (“or”).

It is to be inferred without explicit recitation and unless otherwise intended, that when the present disclosure relates to a polypeptide, protein, polynucleotide, or antibody, an equivalent or a biologically equivalent of such is intended within the scope of this disclosure. As used herein, the term “biological equivalent thereof” is intended to be synonymous with “equivalent thereof” when referring to a reference protein, antibody, polypeptide, or nucleic acid, intends those having minimal sequence identity while still maintaining desired structure or functionality. Unless specifically recited herein, it is contemplated that any polynucleotide, polypeptide, or protein mentioned herein also includes equivalents thereof. For example, an equivalent intends at least about 70% homology or identity, or at least 80% homology or identity and alternatively, or at least about 85%, or alternatively at least about 90%, or alternatively at least about 95%, or alternatively 98% percent homology or identity across the length of the reference sequence and exhibits substantially equivalent biological activity to the reference protein, polypeptide, or nucleic acid. Alternatively, when referring to polynucleotides, an equivalent thereof is a polynucleotide that hybridizes under stringent conditions to the reference polynucleotide or its complement.

An equivalent of a protein or a polypeptide (referred to herein as the reference) shares at least 50% (or at least 60%, or at least 70%, or at least 80%, or at least 90%) identity to the reference and retains the reference's function and manufacturability.

An equivalent of a polynucleotide (referred to herein as the reference) shares at least 50% (or at least 60%, or at least 70%, or at least 80%, or at least 90%) identity to the reference, and encodes the same polypeptide as the one encoded by the reference or encodes an equivalent of the polypeptide encoded by the reference.

To arrive at a position or a consecutive segment of a test sequence equivalent to (or corresponding to) an/a amino acid/nucleotide residue or a consecutive segment of a reference sequence, a sequence alignment is performed between the test and reference sequences. The positions or segments aligned to each other are determined as equivalents.

The term “affinity tag” refers to a polypeptide that may be included within a fusion protein to allow detection of the fusion protein and/or purification of the fusion protein from the cellular milieu using a ligand that is able to bind to, i.e., has affinity for, the affinity tag. The ligand may be, but is not limited to, an antibody, a resin, or a complementary polypeptide. An affinity tag may comprise a small peptide, commonly a peptide of approximately 4 to 16 amino acids in length, or it may comprise a larger polypeptide. Commonly used affinity tags include polyarginine, FLAG, V5, polyhistidine, c-Myc, Strep II, maltose binding protein (MBP), N-utilization substance protein A (NusA), thioredoxin (Trx), and glutathione S-transferase (GST), among others (for examples, see GST Gene Fusion System Handbook—Sigma-Aldrich). In an embodiment the affinity tag is a polyhistidine tag, for example a His6 tag. The inclusion of an affinity tag in a fusion protein allows the fusion protein to be purified from the cellular milieu by affinity purification, using an affinity medium that can tightly and specifically bind the affinity tag. The affinity medium may comprise, for example, a metal-charged resin or a ligand covalently linked to a stationary phase (matrix) such as agarose or metal beads. For example, polyhistidine tagged fusion proteins (also referred to as His tagged fusion proteins) can be recovered by immobilized metal ion chromatography using Ni²⁺ or Co²⁺ loaded resins, anti-FLAG affinity gels may be used to capture FLAG tagged fusion proteins, and glutathione cross-linked to a solid support such as agarose may be used to capture GST tagged fusion proteins.

As used herein the terms “purification”, “purifying”, or “separating” refer to the process of isolating one or more biomaterials (e.g., polynucleotides, polypeptides, or viral vectors) from a complex mixture, such as a cell lysate or a mixture of polypeptides. The purification, separation, or isolation need not be complete, i.e., some components of the complex mixture may remain with the one or more biomaterials (e.g., polynucleotides, polypeptides, or viral vectors) after the purification process. However, the product of purification should be enriched for the one or more biomaterials (e.g., polynucleotides, polypeptides, or viral vectors) relative to the complex mixture before purification and a significant portion of the other components initially present within the complex mixture should be removed by the purification process.

The term “cell” as used herein may refer to either a prokaryotic or eukaryotic cell, optionally obtained from a subject or a commercially available source.

“Eukaryotic cells” comprise all the life kingdoms except monera. They can be easily distinguished through a membrane-bound nucleus. Animals, plants, fungi, and protists are eukaryotes or organisms whose cells are organized into complex structures by internal membranes and a cytoskeleton. The most characteristic membrane-bound structure is the nucleus. Unless specifically recited, the term “host” includes a eukaryotic host, including, for example, yeast, higher plant, insect, and mammalian cells. Non-limiting examples of eukaryotic cells or hosts include simian, bovine, porcine, murine, rat, avian, reptilian, and human, e.g., HEK293 cells, Chinese Hamster Ovary (CHO) cells, 293T cells, and muscle cells. Examples of muscle cells include, but are not limited to, skeletal muscle cells, cardiac muscle cells, and smooth muscle cells.

“Prokaryotic cells” that usually lack a nucleus or any other membrane-bound organelles and are divided into two domains, bacteria, and archaea. In addition to chromosomal DNA, these cells can also contain genetic information in a circular loop called an episome. Bacterial cells are very small, roughly the size of an animal mitochondrion (about 1-2 μm in diameter and 10 μm long). Prokaryotic cells feature three major shapes: rod shaped, spherical, and spiral. Instead of going through elaborate replication processes like eukaryotes, bacterial cells divide by binary fission. Examples include but are not limited to Bacillus bacteria, E. coli bacterium, and Salmonella bacterium.

The term “encode” as it is applied to nucleic acid sequences refers to a polynucleotide which is said to “encode” a polypeptide if, in its native state or when manipulated by methods well known to those skilled in the art, can be transcribed and/or translated to produce the mRNA for the polypeptide and/or a fragment thereof. The antisense strand is the complement of such a nucleic acid, and the encoding sequence can be deduced therefrom.

The terms “equivalent” or “biological equivalent” are used interchangeably when referring to a particular molecule, biological, or cellular material and intend those having minimal homology while still maintaining desired structure or functionality (for example, having a similar function or activity). It should be understood, without being explicitly stated that when referring to an equivalent or biological equivalent to a reference polypeptide, protein, or polynucleotide, that an equivalent or biological equivalent has the recited structural relationship to the reference polypeptide, protein, or polynucleotide and equivalent or substantially equivalent biological activity. For example, non-limiting examples of equivalent polypeptides, proteins, or polynucleotides include a polypeptide, protein or polynucleotide having at least 60%, or alternatively at least 65%, or alternatively at least 70%, or alternatively at least 75%, or alternatively 80%, or alternatively at least 85%, or alternatively at least 90%, or alternatively at least 95% identity thereto or for polypeptide, polynucleotide or protein sequences across the length of the reference polypeptide, polynucleotide, or protein. Alternatively, an equivalent polypeptide is one that is encoded by a polynucleotide or its complement that hybridizes under conditions of high stringency to a polynucleotide encoding such reference polypeptide sequences and that have substantially equivalent or equivalent biological activity. Conditions of high stringency are described herein and incorporated herein by reference. Alternatively, an equivalent thereof is a polypeptide encoded by a polynucleotide or a complement thereto, having at least 70%, or alternatively at least 75%, or alternatively 80%, or alternatively at least 85%, or alternatively at least 90%, or alternatively at least 95% identity, or at least 97% sequence identity across the length of the reference polynucleotide to the reference polynucleotide, e.g., the wild-type polynucleotide. Such equivalent polypeptides have the same biological activity as the polypeptide encoded by the reference polynucleotide.

Non-limiting examples of equivalent polynucleotides, include a polynucleotide having at least 60%, or alternatively at least 65%, or alternatively at least 70%, or alternatively at least 75%, or alternatively 80%, or alternatively at least 85%, or alternatively at least 90%, or alternatively at least 95%, or alternatively at least 97%, identity to a reference polynucleotide. An equivalent also intends a polynucleotide or its complement that hybridizes under conditions of high stringency to a reference polynucleotide. Such equivalent polynucleotides have the same biological activity as the reference polynucleotide.

A polynucleotide or polynucleotide region (or a polypeptide or polypeptide region) having a certain percentage (for example, 80%, 85%, 90%, or 95%) of “sequence identity” to another sequence means that, when aligned, that percentage of bases (or amino acids) are the same in comparing the two sequences across the length of the reference polynucleotide. The alignment and the percent homology or sequence identity can be determined using software programs known in the art, for example those described in Current Protocols in Molecular Biology (Ausubel et al., eds. 1987) Supplement 30, section 7.7.18, Table 7.7.1. In certain embodiments, default parameters are used for alignment. A non-limiting exemplary alignment program is BLAST, using default parameters. In particular, exemplary programs include BLASTN and BLASTP, using the following default parameters: Genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+SwissProtein+SPupdate+PIR. Details of these programs can be found at the following Internet address: ncbi.nlm.nih.gov/cgi-bin/BLAST. Sequence identity and percent identity can be determined by incorporating them into clustalW (available at the web address:genome.jp/tools/clustalw/, last accessed on Jan. 13, 2017).

“Homology” or “identity” or “similarity” refers to sequence similarity between two peptides or between two nucleic acid molecules. Homology can be determined by comparing a position in each sequence that may be aligned for purposes of comparison. When a position in the compared sequence is occupied by the same base or amino acid, then the molecules are homologous at that position. A degree of homology between sequences is a function of the number of matching or homologous positions shared by the sequences. An “unrelated” or “non-homologous” sequence shares less than 40% identity, or alternatively less than 25% identity, with one of the sequences of the present disclosure.

As used herein, the term “at least 90% identical” refers to an identity of two compared sequences (polynucleotides or polypeptides) of about 90% to about 100%. It also includes an identity of at least at least 91%, at least 92%, at least 93%, at least 94%, at least 95%, at least 96%, at least 97%, at least 98%, at least 99%, about 91% to about 100%, about 92% to about 100%, about 93% to about 100%, about 94% to about 100%, about 95% to about 100%, about 96% to about 100%, about 97% to about 100%, about 98% to about 100%, or about 99% to about 100%.

As used herein, the terms “retain” “similar” and “same” are used interchangeably while describing a function, an activity or an functional activity of a polynucleotide, a protein and/or a peptide, referring to a functional activity of at least about 20% (including but not limited to: at least about 30%, at least about 40%, at least about 50%, at least about 60%, at least about 70%, at least about 80%, at least about 90%, at least about 95%, at least about 97%, or about 100%) of the activity of the reference protein, polynucleotide and/or peptide.

“Hybridization” refers to a reaction in which one or more polynucleotides react to form a complex that is stabilized via hydrogen bonding between the bases of the nucleotide residues. The hydrogen bonding may occur by Watson-Crick base pairing, Hoogsteen binding, or in any other sequence-specific manner. The complex may comprise two strands forming a duplex structure, three or more strands forming a multi-stranded complex, a single self-hybridizing strand, or any combination of these. A hybridization reaction may constitute a step in a more extensive process, such as the initiation of a PCR reaction, or the enzymatic cleavage of a polynucleotide by a ribozyme.

Examples of stringent hybridization conditions include: incubation temperatures of about 25° C. to about 37° C.; hybridization buffer concentrations of about 6×SSC to about 10×SSC; formamide concentrations of about 0% to about 25%; and wash solutions from about 4×SSC to about 8×SSC. Examples of moderate hybridization conditions include: incubation temperatures of about 40° C. to about 50° C.; buffer concentrations of about 9×SSC to about 2×SSC; formamide concentrations of about 30% to about 50%; and wash solutions of about 5×SSC to about 2×SSC. Examples of high stringency conditions include: incubation temperatures of about 55° C. to about 68° C.; buffer concentrations of about 1×SSC to about 0.1×SSC; formamide concentrations of about 55% to about 75%; and wash solutions of about 1×SSC, 0.1×SSC, or deionized water. In general, hybridization incubation times are from 5 minutes to 24 hours, with 1, 2, or more washing steps, and wash incubation times are about 1, 2, or 15 minutes. SSC is 0.15 M NaCl and 15 mM citrate buffer. It is understood that equivalents of SSC using other buffer systems can be employed. In one aspect, an equivalent polynucleotide is one that hybridizes under stringent conditions to a reference polynucleotide or its complement. In another aspect, an equivalent polypeptide is a polypeptide that is encoded by a polynucleotide is one that hybridizes under stringent conditions to a reference polynucleotide or its complement.

As used herein, “expression” refers to the process by which polynucleotides are transcribed into mRNA and/or the process by which the transcribed mRNA is subsequently being translated into peptides, polypeptides, or proteins. If the polynucleotide is derived from genomic DNA, expression may include splicing of the mRNA in a eukaryotic cell.

As used herein, the term “functional” may be used to modify any molecule, biological, or cellular material to intend that it accomplishes a particular, specified effect.

As used herein, the terms “nucleic acid sequence” and “polynucleotide” are used interchangeably to refer to a polymeric form of nucleotides of any length, either ribonucleotides or deoxyribonucleotides. Thus, this term includes, but is not limited to, single-, double-, or multi-stranded DNA or RNA, genomic DNA, complementary DNA (cDNA), DNA-RNA hybrids, or a polymer comprising purine and pyrimidine bases or other natural, chemically, or biochemically modified, non-natural, or derivatized nucleotide bases. In certain embodiments, the polynucleotide comprises and/or encodes a messenger RNA (mRNA), a short hairpin RNA, and/or small hairpin RNA. In one embodiment, the polynucleotide is or encodes an mRNA. In certain embodiments, the polynucleotide is a double-strand (ds) DNA, such as an engineered ds DNA or ds cDNA synthesized from a single-stranded RNA.

The term “modified nucleoside” refers to any nucleoside that is not the canonical ribonucleoside or deoxyribonucleoside. A canonical ribonucleoside or deoxyribonucleoside comprises a nitrogenous base (e.g., adenine, guanine, thymine, uracil, and cytosine) and a five-carbon sugar (e.g., ribose or deoxyribose). A modified nucleoside includes any modification of a canonical ribonucleoside or deoxyribonucleoside. The modification of the canonical ribonucleoside or deoxyribonucleoside may occur in the nucleobase and/or five-carbon sugar. Examples of modified nucleosides include, but are not limited, LNAs, 2′-substituted nucleosides, and 2′-O-methyl nucleosides. A modified nucleoside also includes a canonical ribonucleoside or deoxyribonucleoside that further contains a phosphorothioate group instead of a phosphate group, which is found in canonical ribonucleotides and deoxyribonucleotides. In some embodiments, a modified nucleoside is any of the nucleosides shown in Table 4.

The terms “locked nucleic acid” or “LNA” means a nucleoside comprising a bicyclic sugar moiety comprising a 4′-CH₂—O-2′ bridge. Examples of LNAs include, but are not limited to LNA, scpBNA, AmNA (N—H), AmNA (N-Me), GuNA, GuNA (N—R) where R is selected from Me, Et, i-Pr, t-Bu.

The term “2′-substituted nucleoside” means a nucleoside comprising a substituent at the 2′-position other than H or OH. Unless otherwise indicated, a 2′-substituted nucleoside is not a bicyclic nucleoside. Examples of 2′-substituted nucleosides include, but are not limited to, 2′-0-methoxy-ethyl (2′-MOE) nucleosides and 2′-O-methyl nucleosides. Examples of 2′-O-methyl nucleosides include, but are not limited to, 2′-O-methyl nucleosides and 5-methylcytosines ((5m)C).

The term “protein”, “peptide” and “polypeptide” are used interchangeably and in their broadest sense to refer to a compound of two or more subunits of amino acids, amino acid analogs or peptidomimetics. The subunits may be linked by peptide bonds. In another aspect, the subunit may be linked by other bonds, e.g., ester, ether, etc. A protein or peptide must contain at least two amino acids and no limitation is placed on the maximum number of amino acids which may comprise a protein's or peptide's sequence. As used herein the term “amino acid” refers to either natural and/or unnatural or synthetic amino acids, including glycine and both the D and L optical isomers, amino acid analogs and peptidomimetics.

As used herein, a consecutive amino acid sequence refers to a sequence having at least two amino acids. However, it is noted that a consecutive amino acid sequence of a first part and a second part does not limit the amino acid sequence to have the first part directly conjugated to the second part. It is also possible that the first part is linked to the second part via a third part, such as a link, thus forming one consecutive amino acid sequence.

As used herein, the terms “conjugate,” “conjugated,” “conjugating,” and “conjugation” refer to the formation of a bond between molecules, and between two amino acid sequences and/or two polypeptides. Conjugation can be direct (i.e. a bond) or indirect (i.e. via a further molecule). The conjugation can be covalent or non-covalent.

As used herein a consecutive amino acid sequence may comprise two or more polypeptides conjugated with each other directly or indirectly (for example via a linker or linkage).

As used herein, the term “recombinant expression system” refers to a genetic construct or constructs for the expression of certain genetic material formed by recombination.

As used herein, the term “viral capsid” or “capsid” refers to the proteinaceous shell or coat of a viral particle. Capsids function to encapsidate, protect, transport, and release into host cell a viral genome. Capsids are generally comprised of oligomeric structural subunits of protein (“capsid proteins”). As used herein, the term “encapsidated” means enclosed within a viral capsid.

As used herein, a biological sample, or a sample, can be obtained from a subject, cell line or cultured cell or tissue. Exemplary samples include, but are not limited to, cell sample, tissue sample, liquid samples such as blood and other liquid samples of biological origin (including, but not limited to, ocular fluids (aqueous and vitreous humor), peripheral blood, sera, plasma, ascites, urine, cerebrospinal fluid (C SF), sputum, saliva, bone marrow, synovial fluid, aqueous humor, amniotic fluid, cerumen, breast milk, bronchoalveolar lavage fluid, semen, prostatic fluid, Cowper's fluid or pre-ejaculatory fluid, female ejaculate, sweat, tears, cyst fluid, pleural and peritoneal fluid, pericardial fluid, ascites, lymph, chyme, chyle, bile, interstitial fluid, menses, pus, sebum, vomit, vaginal secretions/flushing, synovial fluid, mucosal secretion, stool water, pancreatic juice, lavage fluids from sinus cavities, bronchopulmonary aspirates, blastocyle cavity fluid, or umbilical cord blood.

As used herein, the term “detectable marker” refers to at least one marker capable of directly or indirectly, producing a detectable signal. A non-exhaustive list of this marker includes enzymes which produce a detectable signal, for example by colorimetry, fluorescence, luminescence, such as horseradish peroxidase, alkaline phosphatase, β-galactosidase, Glucose-6-phosphate dehydrogenase, chromophores such as fluorescent, luminescent dyes, groups with electron density detected by electron microscopy or by their electrical property such as conductivity, amperometry, voltammetry, impedance, detectable groups, for example whose molecules are of sufficient size to induce detectable modifications in their physical and/or chemical properties, such detection may be accomplished by optical methods such as diffraction, surface plasmon resonance, surface variation, the contact angle change or physical methods such as atomic force spectroscopy, tunnel effect, or radioactive molecules such as ³²P, ³⁵S, ⁸⁹Zr or ¹²⁵I.

As used herein, the term “purification marker” refers to at least one marker useful for purification or identification. A non-exhaustive list of this marker includes His, lacZ, GST, maltose-binding protein, NusA, BCCP, c-myc, CaM, FLAG, GFP, YFP, cherry, thioredoxin, poly(NANP), V5, Snap, HA, chitin-binding protein, Softag 1, Softag 3, Strep, or S-protein. Suitable direct or indirect fluorescence marker comprise FLAG, GFP, YFP, RFP, dTomato, cherry, Cy3, Cy 5, Cy 5.5, Cy 7, DNP, AMCA, Biotin, Digoxigenin, Tamra, Texas Red, rhodamine, Alexa fluors, FITC, TRITC or any other fluorescent dye or hapten.

As used herein, an epitope tag is a biological structure or sequence, such as a protein or carbohydrate, which acts as an antigen that is recognized by an antibody. In certain embodiments, an epitope tag is used interchangeably with a purification marker and/or an affinity tag.

A “composition” is intended to mean a combination of two or more compounds, such as a combination of an antisense oligonucleotide, polypeptide, polynucleotide, viral vector, or antibody and another compound or composition. Alternatively, or additionally, a “composition” may refer to a combination of two or more compounds, such as two or more antisense oligonucleotides, polypeptides, polynucleotides, viral vectors, or antibodies.

A “pharmaceutical composition” is intended to include the combination of an antisense oligonucleotide, polypeptide, polynucleotide, or antibody with a carrier, inert or active, such as a solid support or phosphate buffered saline solution or water, making the composition suitable for diagnostic or therapeutic use in vitro, in vivo or ex vivo.

As used herein, the term “pharmaceutically acceptable carrier” encompasses any of the standard pharmaceutical carriers, such as a phosphate buffered saline solution, water, and emulsions, such as an oil/water or water/oil emulsion, and various types of wetting agents. The compositions also can include stabilizers and preservatives. For examples of carriers, stabilizers, and adjuvants, see Martin (1975) Remington's Pharm. Sci., 15th Ed. (Mack Publ. Co., Easton).

A “subject,” “individual” or “patient” is used interchangeably herein, and refers to a vertebrate, preferably a mammal, more preferably a human. Mammals include, but are not limited to, murines, rats, rabbits, simians, bovines, ovines, porcine, canines, felines, farm animals, sport animals, pets, equine, and primate, particularly human. Besides being useful for human treatment, the present invention is also useful for veterinary treatment of companion mammals, exotic animals, and domesticated animals, including mammals, rodents, and the like which is susceptible to RNA and in particular, HIV viral infection. In one embodiment, the mammals include horses, dogs, and cats. In another embodiment of the present invention, the human is an adolescent or infant under the age of eighteen years of age.

“Treating” or “treatment” of a disease includes: (1) preventing the disease, i.e., causing the clinical symptoms of the disease not to develop in a patient that may be predisposed to the disease but does not yet experience or display symptoms of the disease; (2) inhibiting the disease, i.e., arresting or reducing the development of the disease or its clinical symptoms; or (3) relieving the disease, i.e., causing regression of the disease or its clinical symptoms. In one aspect, the term “treatment” excludes prevention or prophylaxis.

The term “suffering” as it related to the term “treatment” refers to a patient or individual who has been diagnosed with or is predisposed to a disease.

An “effective amount” is an amount sufficient to effect beneficial or desired results. An effective amount can be administered in one or more administrations, applications, or dosages. Such delivery is dependent on several variables including the time period for which the individual dosage unit is to be used, the bioavailability of the therapeutic agent, the route of administration, etc. It is understood, however, that specific dose levels of the therapeutic agents of the present invention for any particular subject depends upon a variety of factors including the activity of the specific compound employed, the age, body weight, general health, sex, and diet of the subject, the time of administration, the rate of excretion, the drug combination, and the severity of the particular disorder being treated and form of administration. Treatment dosages generally may be titrated to optimize safety and efficacy. Typically, dosage-effect relationships from in vitro and/or in vivo tests initially can provide useful guidance on the proper doses for patient administration. In general, one will desire to administer an amount of the compound that is effective to achieve a serum level commensurate with the concentrations found to be effective in vitro. Determination of these parameters is well within the skill of the art. These considerations, as well as effective formulations and administration procedures are well known in the art and are described in standard textbooks. Consistent with this definition, as used herein, the term “therapeutically effective amount” is an amount sufficient to inhibit RNA virus replication ex vivo, in vitro, or in vivo.

The term administration shall include without limitation, administration by oral, parenteral (e.g., intramuscular, intraperitoneal, intravenous, ICV, intracisternal injection or infusion, subcutaneous injection, or implant), by inhalation spray nasal, vaginal, rectal, sublingual, urethral (e.g., urethral suppository) or topical routes of administration (e.g., gel, ointment, cream, aerosol, etc.) and can be formulated, alone or together, in suitable dosage unit formulations containing conventional non-toxic pharmaceutically acceptable carriers, adjuvants, excipients, and vehicles appropriate for each route of administration. The invention is not limited by the route of administration, the formulation or dosing schedule.

As used in the specification and claims, the singular form “a”, “an” and “the” include plural references unless the context clearly dictates otherwise. For example, the term “a cell” includes a plurality of cells, including mixtures thereof.

As used herein, the term “comprising” or “comprises” is intended to mean that the compositions and methods include the recited elements, but not excluding others. “Consisting essentially of” when used to define compositions and methods, shall mean excluding other elements of any essential significance to the combination for the stated purpose. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions of this invention or process steps to produce a composition or achieve an intended result. Embodiments defined by each of these transition terms are within the scope of this invention.

The term “isolated” as used herein with respect to nucleic acids, such as DNA or RNA, refers to molecules separated from other DNAs or RNAs, respectively that are present in the natural source of the macromolecule. The term “isolated nucleic acid” is meant to include nucleic acid fragments which are not naturally occurring as fragments and would not be found in the natural state.

The term “isolated” is also used herein to refer to polypeptides, proteins and/or host cells that are isolated from other cellular proteins and is meant to encompass both purified and recombinant polypeptides. In other embodiments, the term “isolated” means separated from constituents, cellular and otherwise, in which the cell, tissue, polynucleotide, peptide, polypeptide, protein, antibody or fragment(s) thereof, which are normally associated in nature. For example, an isolated cell is a cell that is separated form tissue or cells of dissimilar phenotype or genotype. As is apparent to those of skill in the art, a non-naturally occurring polynucleotide, peptide, polypeptide, protein, antibody, or fragment(s) thereof, does not require “isolation” to distinguish it from its naturally occurring counterpart.

Tables

TABLE 1
Oligonucleotide sequences without
modified nucleosides

| SEQ | |
|---|---|
| ID | |
| NO: | Sequence (5′ → 3′) |
| 3 | CCGACCACGGGGCGCACCUCUC |
| 4 | CGACCACGGGGCGCACCUCUCU |
| 5 | CGACCACGGGGCGCACCUCUC |
| 6 | GACCACGGGGCGCACCUCUCU |
| 7 | GGGGCGCACCUCUCUUUACGCG |
| 8 | CCGACCACGGGGCGCACCUCUC |
| 9 | CGACCACGGGGCGCACCUCUCU |
| 10 | CGACCACGGGGCGCACCUCUC |
| 11 | GACCACGGGGCGCACCUCUCU |
| 12 | GGGGCGCACCUCUCUUUACGCG |
| 13 | CGCACCTCTCTTTACG |
| 14 | GCACCTCTCTTTACG |
| 15 | GCACCTCTCTTTACGC |
| 16 | CGCACCTCTCTTTAC |
| 17 | CGGGACGTCCTTTGT |
| 18 | CCGTGTGCACTTCGC |
| 19 | CCTCTCTTTACGCGG |
| 20 | ACCTCTCTTTACGCG |
| 21 | CACCTCTCTTTACGC |
| 22 | CACCTCTCTTTACGCGG |
| 23 | GCACCTCTCTTTACG |
| 24 | CGCACCTCTCTTTAC |
| 25 | CGCACCTCTCTTTACGC |
| 26 | CGCACCTCTCTTTACGCG |
| 27 | CGGGACGTCCTTTGT |
| 28 | GCGGGACGTCCTTTG |
| 29 | GCGGGACGTCCTTTGT |
| 30 | CGCGGGACGTCCTTTGT |
| 31 | TGCCAACTGGATCCT |
| 32 | CTGCCAACTGGATCC |
| 33 | CTGCCAACTGGATCCT |
| 34 | CCATACTGCGGAACT |
| 35 | TCCATACTGCGGAACT |
| 36 | ATCCATACTGCGGAA |
| 37 | ATCCATACTGCGGAACT |
| 38 | GATCCATACTGCGGAA |
| 39 | CGATCCATACTGCGG |
| 40 | CCGATCCATACTGCG |
| 41 | CCGATCCATACTGCGG |
| 42 | CTGCCGATCCATACT |
| 43 | CTGCCGATCCATACTG |
| 44 | TCTGCCGATCCATAC |
| 45 | TCTGCCGATCCATACT |
| 46 | CTCTGCCGATCCATAC |
| 47 | CTCTGCCGATCCATACT |
| 48 | TCCTCTGCCGATCCA |
| 49 | TCCTCTGCCGATCCAT |
| 50 | AGTGTTTGCTGACGC |
| 51 | ACCTCTCTTTACGCG |
| 52 | CACCTCTCTTTACGC |
| 53 | AGTGTTTGCTGACGC |
| 54 | ACCTCTCTTTACGCGG |
| 55 | CACCTCTCTTTACGCG |
| 56 | GCACCTCTCTTTACGC |
| 57 | GCACCTCTCTTTACGCG |
| 58 | CGCGGGACGTCCTTTG |
| 59 | CGATCCATACTGCGGAA |
| 60 | TGCCGATCCATACTG |
| 61 | TCTGCCGATCCATACTG |
| 62 | CCTCTGCCGATCCAT |
| 63 | CTCCTCTGCCGATCC |
| 64 | CTCCTCTGCCGATCCA |
| 199 | GATCCATACTGCGGAA |
| 200 | GAUCCAUACUGCGGAA |
| 201 | GAUCCAUACUGCGGAA |
| 202 | GATCCATACTGCGGAA |
| 203 | GATCCAUACUGCGGAA |
| 204 | GATCCATACTGCGGAA |
| 205 | AGUGUUUGCUGACGC |
| 206 | AGUGUUUGCUGACGC |
| 207 | AGTGTTTGCTGACGC |
| 208 | AGTGTUTGCUGACGC |
| 209 | AGTGUUUGCUGACGC |
| 210 | AGTGTTTGCTGACGC |
| 211 | AGUGUTUGCTGACGC |
| 212 | CCGACCACGGGGCGCA |
| 213 | CGACCACGGGGCGCAC |
| 214 | ACGGGGCGCACCTCTC |
| 215 | GACCACGGGGCGCACC |
| 216 | GGGGCGCACCTCTCTU |
| 217 | GGGGCGCACCTCTCTT |
| 218 | CCGACCACGGGGCGCACC |
| 219 | CGACCACGGGGCGCACCCT |
| 220 | CCACGGGGCGCACCTCTC |
| 221 | GACCACGGGGCGCACCCUC |
| 222 | GGGGCGCACCTCTCTUTA |
| 223 | GGGGCGCACCTCTCTUT |
| 224 | CGGGACGUCCUUUGU |
| 225 | CGGGACGTCCTTTGT |
| 226 | CGGGACGTCCTTTGT |
| 227 | AGATCCATACUGCGGAA |
| 228 | CGGGACGUCCUTTGT |
| 229 | CGGGACGTCCUTUGU |
| 230 | GAUCCAUACTGCGGAA |
| 231 | CGGGACGUCCUTTGT |
| 232 | AGTGTUTGCUGACGC |
| 233 | TGCCAACUGGAUCCT |
| 234 | GCACCUCUCUTUACG |
| 235 | GATCCATACUGCGGAA |
| 236 | CUGCCAACTGGATCCU |
| 237 | GCGGGACGTCCUTUGU |
| 238 | GGTCACCATATUCUTG |
| 239 | TCACCATATUCUTGGG |
| 240 | CACCAUAUTCTUGGGA |
| 241 | CCAUAUTCTUGGGAAC |
| 242 | AUAUTCTUGGGAACAA |
| 243 | CAAGAATATGGUGACC |
| 244 | CCCAAGAATATGGUGA |
| 245 | TCCCAAGAAUAUGGTG |
| 246 | GUTCCCAAGAAUAUGG |
| 247 | TUGUTCCCAAGAAUAU |
| 248 | TUGGGGTGGAGCCCTC |
| 249 | TUGGGGTGGAGCCCTCA |
| 250 | TGGGGUGGAGCCCUCA |
| 251 | GGAGCCCUCAGGCUCA |
| 252 | CCCUCAGGCUCAGGGC |
| 253 | GAGGGCTCCACCCCAA |
| 254 | TGAGGGCUCCACCCCAA |
| 255 | TGAGGGCUCCACCCCA |
| 256 | TGAGCCTGAGGGCUCC |
| 257 | GCCCTGAGCCTGAGGG |
| 258 | UGCCAACTGGATCCU |
| 259 | UGCCAACTGGATCCT |
| 260 | TGCCAACTGGATCCT |
| 261 | TGCCAACTGGATCCT |
| 262 | TGCCAACUGGAUCCT |
| 263 | TGCCAACUGGAUCCT |
| 264 | TGCCAACUGGAUCCT |
| 265 | TGCCAACUGGAUCCT |
| 266 | TGCCAACUGGAUCCT |
| 267 | TGCCAACUGGAUCCT |
| 268 | CTGCCAACUGGAUCCT |
| 233; | 5′-TGCCAACUGGAUCCT-3′ |
| 269 | (SEQ ID NO: 233) |
| | 3′-ACGGTTGACCTAGGA-5′ |
| | (SEQ ID NO: 269) |
| 243; | 5′-CAAGAATATGGUGACC-3′ |
| 270 | (SEQ ID NO: 243) |
| | 3′-GTTCTTATACCACTGG-5′ |
| | (SEQ ID NO: 270) |

TABLE 2
Oligonucleotide sequences with modified nucleosides

| SEQ | |
|---|---|
| ID | |
| NO: | Sequence (5′ → 3′) |
| 65 | mCpsmCpsmGpsmApsmCpsmCpsmApsmCpsmGpsmGpsmGpsmGpsmCpsmG |
| | psmCpsmApsmCpsmCpsmUpsmCpsmUpsmC |
| 66 | mCpsmGpsmApsmCpsmCpsmApsmCpsmGpsmGpsmGpsmGpsmCpsmGpsmC |
| | psmApsmCpsmCpsmUpsmCpsmUpsmCpsmU |
| 67 | mCpsmGpsmApsmCpsmCpsmApsmCpsmGpsmGpsmGpsmGpsmCpsmGpsmC |
| | psmApsmCpsmCpsmUpsmCpsmUpsmC |
| 68 | mGpsmApsmCpsmCpsmApsmCpsmGpsmGpsmGpsmGpsmCpsmGpsmCpsmA |
| | psmCpsmCpsmUpsmCpsmUpsmCpsmU |
| 69 | mGpsmGpsmGpsmGpsmCpsmGpsmCpsmApsmCpsmCpsmUpsmCpsmUpsmC |
| | psmUpsmUpsmUpsmApsmCpsmGpsmCpsmG |
| 70 | mCpsln(5m)CpsmGpslnApsmCpsln(5m)CpsmApsln(5m)CpsmGpslnGpsmGpsl |
| | nGpsmCpslnGpsmCpslnApsmCpsln(5m)CpsmUpsln(5m)CpsmUpsln(5m)C |
| 71 | mCpslnGpsmApsln(5m)CpsmCpslnApsmCpslnGpsmGpslnGpsmGpsln(5m)Cps |
| | mGpsln(5m)CpsmApsln(5m)CpsmCpslnTpsmCpslnTpsmCpslnT |
| 72 | mGpslnApsmCpsln(5m)CpsmApsln(5m)CpsmGpslnGpsmGpslnGpsmCpslnGps |
| | mCpslnApsmCpsln(5m)CpsmUpsln(5m)CpsmUpsln(5m)CpslnT |
| 73 | mGpslnApsmCpsln(5m)CpsmApsln(5m)CpsmGpslnGpsmGpslnGpsmCpslnGps |
| | mCpslnApsmCpsln(5m)CpsmUpsln(5m)CpsmUpsln(5m)CpslnT |
| 74 | mGpslnGpsmGpslnGpsmCpslnGpsmCpslnApsmCpsln(5m)CpsmUpsln(5m)Cps |
| | mUpsln(5m)CpsmUpslnTpsmUpslnApsmCpslnGpsmCpslnG |
| 75 | ln(5m)CpsmGpsln(5m)CpsmApsln(5m)CpsmCpslnTpsmCpslnTpsmCpslnTpsm |
| | UpslnTpsmApsln(5m)CpsmG |
| 76 | lnGpsln(5m)CpslnApsln(5m)Cpsln(5m)CpslnTpsln(5m)CpslnTpsln(5m)CpslnT |
| | pslnTpslnTpslnApsln(5m)CpslnG |
| 77 | lnGpsln(5m)CpslnApsln(5m)Cpsln(5m)CpslnTpsln(5m)CpslnTpsln(5m)CpslnT |
| | pslnTpslnTpslnApsln(5m)CpslnGpsln(5m)C |
| 78 | ln(5m)CpslnGpsln(5m)CpslnApsln(5m)Cpsln(5m)CpslnTpsln(5m)CpslnTpsln(5 |
| | m)CpslnTpslnTpslnTpslnApsln(5m)C |
| 79 | ln(5m)CpslnGpslnGpslnGpslnApsln(5m)CpslnGpslnTpsln(5m)Cpsln(5m)Cpsln |
| | TpslnTpslnTpslnGpslnT |
| 80 | ln(5m)CpsmCpslnGpsmUpslnGpsmUpslnGpsmCpslnApsmCpslnTpsmUpsln(5 |
| | m)CpsmGpsln(5m)C |
| 81 | ln(5m)CpsmCpslnTpsmCpslnTpsmCpslnTpsmUpslnTpsmApsln(5m)CpsmGpsl |
| | n(5m)CpsmGpslnG |
| 82 | lnApsmCpsln(5m)CpsmUpsln(5m)CpsmUpsln(5m)CpsmUpslnTpsmUpslnAps |
| | mCpslnGpsmCpslnG |
| 83 | ln(5m)CpsmApsln(5m)CpsmCpslnTpsmCpslnTpsmCpslnTpsmUpslnTpsmApsl |
| | n(5m)CpsmGpsln(5m)C |
| 84 | ln(5m)CpsmApsln(5m)CpsmCpslnTpsmCpslnTpsmCpslnTpsmUpslnTpsmApsl |
| | n(5m)CpsmGpsln(5m)CpsmGpslnG |
| 85 | lnGpsmCpslnApsmCpsln(5m)CpsmUpsln(5m)CpsmUpsln(5m)CpsmUpslnTps |
| | mUpslnApsmCpslnG |
| 86 | ln(5m)CpsmGpsln(5m)CpsmApsln(5m)CpsmCpslnTpsmCpslnTpsmCpslnTpsm |
| | UpslnTpsmApsln(5m)C |
| 87 | ln(5m)CpsmGpsln(5m)CpsmApsln(5m)CpsmCpslnTpsmCpslnTpsmCpslnTpsm |
| | UpslnTpsmApsln(5m)CpsmGpsln(5m)C |
| 88 | ln(5m)CpsmGpsln(5m)CpsmApsln(5m)CpsmCpslnTpsmCpslnTpsmCpslnTpsm |
| | UpslnTpsmApsln(5m)CpsmGpsln(5m)CpsmG |
| 89 | ln(5m)CpsmGpslnGpsmGpslnApsmCpslnGpsmUpsln(5m)CpsmCpslnTpsmUps |
| | lnTpsmGpslnT |
| 90 | lnGpsmCpslnGpsmGpslnGpsmApsln(5m)CpsmGpslnTpsmCpsln(5m)CpsmUps |
| | lnTpsmUpslnG |
| 91 | lnGpsmCpslnGpsmGpslnGpsmApsln(5m)CpsmGpslnTpsmCpsln(5m)CpsmUps |
| | lnTpsmUpslnGpsmU |
| 92 | ln(5m)CpsmGpsln(5m)CpsmGpslnGpsmGpslnApsmCpslnGpsmUpsln(5m)Cps |
| | mCpslnTpsmUpslnTpsmGpslnT |
| 93 | lnTpsmGpsln(5m)CpsmCpslnApsmApsln(5m)CpsmUpslnGpsmGpslnApsmUps |
| | ln(5m)CpsmCpslnT |
| 94 | ln(5m)CpsmUpslnGpsmCpsln(5m)CpsmApslnApsmCpslnTpsmGpslnGpsmAps |
| | lnTpsmCpsln(5m)C |
| 95 | ln(5m)CpsmUpslnGpsmCpsln(5m)CpsmApslnApsmCpslnTpsmGpslnGpsmAps |
| | lnTpsmCpsln(5m)CpsmU |
| 96 | ln(5m)CpsmCpslnApsmUpslnApsmCpslnTpsmGpsln(5m)CpsmGpslnGpsmAps |
| | lnApsmCpslnT |
| 97 | lnTpsmCpsln(5m)CpsmApslnTpsmApsln(5m)CpsmUpslnGpsmCpslnGpsmGpsl |
| | nApsmApsln(5m)CpsmU |
| 98 | lnApsmUpsln(5m)CpsmCpslnApsmUpslnApsmCpslnTpsmGpsln(5m)CpsmGps |
| | lnGpsmApslnA |
| 99 | lnApsmUpsln(5m)CpsmCpslnApsmUpslnApsmCpslnTpsmGpsln(5m)CpsmGps |
| | lnGpsmApslnApsmCpslnT |
| 100 | lnGpsmApslnTpsmCpsln(5m)CpsmApslnTpsmApsln(5m)CpsmUpslnGpsmCpsl |
| | nGpsmGpslnApsmA |
| 101 | ln(5m)CpsmGpslnApsmUpsln(5m)CpsmCpslnApsmUpslnApsmCpslnTpsmGps |
| | ln(5m)CpsmGpslnG |
| 102 | ln(5m)CpsmCpslnGpsmApslnTpsmCpsln(5m)CpsmApslnTpsmApsln(5m)Cpsm |
| | UpslnGpsmCpslnG |
| 103 | ln(5m)CpsmCpslnGpsmApslnTpsmCpsln(5m)CpsmApslnTpsmApsln(5m)Cpsm |
| | UpslnGpsmCpslnGpsmG |
| 104 | ln(5m)CpsmUpslnGpsmCpsln(5m)CpsmGpslnApsmUpsln(5m)CpsmCpslnAps |
| | mUpslnApsmCpslnT |
| 105 | ln(5m)CpsmUpslnGpsmCpsln(5m)CpsmGpslnApsmUpsln(5m)CpsmCpslnAps |
| | mUpslnApsmCpslnTpsmG |
| 106 | lnTpsmCpslnTpsmGpsln(5m)CpsmCpslnGpsmApslnTpsmCpsln(5m)CpsmApsl |
| | nTpsmApsln(5m)C |
| 107 | lnTpsmCpslnTpsmGpsln(5m)CpsmCpslnGpsmApslnTpsmCpsln(5m)CpsmApsl |
| | nTpsmApsln(5m)CpsmU |
| 108 | ln(5m)CpsmUpsln(5m)CpsmUpslnGpsmCpsln(5m)CpsmGpslnApsmUpsln(5m) |
| | CpsmCpslnApsmUpslnApsmC |
| 109 | ln(5m)CpsmUpsln(5m)CpsmUpslnGpsmCpsln(5m)CpsmGpslnApsmUpsln(5m) |
| | CpsmCpslnApsmUpslnApsmCpslnT |
| 110 | lnTpsmCpsln(5m)CpsmUpsln(5m)CpsmUpslnGpsmCpsln(5m)CpsmGpslnAps |
| | mUpsln(5m)CpsmCpslnA |
| 111 | lnTpsmCpsln(5m)CpsmUpsln(5m)CpsmUpslnGpsmCpsln(5m)CpsmGpslnAps |
| | mUpsln(5m)CpsmCpslnApsmU |
| 112 | lnApsmGpslnTpsmGpslnTpsmUpslnTpsmGpsln(5m)CpsmUpslnGpsmApsln(5 |
| | m)CpsmGpsln(5m)C |
| 113 | lnApsln(5m)Cpsln(5m)CpslnTpsln(5m)CpslnTpsln(5m)CpslnTpslnTpslnTpslnA |
| | psln(5m)CpslnGpsln(5m)CpslnG |
| 114 | ln(5m)CpslnApsln(5m)Cpsln(5m)CpslnTpsln(5m)CpslnTpsln(5m)CpslnTpslnT |
| | pslnTpslnApsln(5m)CpslnGpsln(5m)C |
| 115 | lnApslnGpslnTpslnGpslnTpslnTpslnTpslnGpsln(5m)CpslnTpslnGpslnApsln(5m) |
| | CpslnGpsln(5m)C |
| 116 | lnApsmCpsln(5m)CpsmUpsln(5m)CpsmUpsln(5m)CpsmUpslnTpsmUpslnAps |
| | mCpslnGpsmCpslnGpsmG |
| 117 | ln(5m)CpsmApsln(5m)CpsmCpslnTpsmCpslnTpsmCpslnTpsmUpslnTpsmApsl |
| | n(5m)CpsmGpsln(5m)CpsmG |
| 118 | lnGpsmCpslnApsmCpsln(5m)CpsmUpsln(5m)CpsmUpsln(5m)CpsmUpslnTps |
| | mUpslnApsmCpslnGpsmC |
| 119 | lnGpsmCpslnApsmCpsln(5m)CpsmUpsln(5m)CpsmUpsln(5m)CpsmUpslnTps |
| | mUpslnApsmCpslnGpsmCpslnG |
| 120 | ln(5m)CpsmGpsln(5m)CpsmGpslnGpsmGpslnApsmCpslnGpsmUpsln(5m)Cps |
| | mCpslnTpsmUpslnTpsmG |
| 121 | ln(5m)CpsmGpslnApsmUpsln(5m)CpsmCpslnApsmUpslnApsmCpslnTpsmGps |
| | ln(5m)CpsmGpslnGpsmApslnA |
| 122 | lnTpsmGpsln(5m)CpsmCpslnGpsmApslnTpsmCpsln(5m)CpsmApslnTpsmApsl |
| | n(5m)CpsmUpslnG |
| 123 | lnTpsmCpslnTpsmGpsln(5m)CpsmCpslnGpsmApslnTpsmCpsln(5m)CpsmApsl |
| | nTpsmApsln(5m)CpsmUpslnG |
| 124 | ln(5m)CpsmCpslnTpsmCpslnTpsmGpsln(5m)CpsmCpslnGpsmApslnTpsmCpsl |
| | n(5m)CpsmApslnT |
| 125 | ln(5m)CpsmUpsln(5m)CpsmCpslnTpsmCpslnTpsmGpsln(5m)CpsmCpslnGpsm |
| | ApslnTpsmCpsln(5m)C |
| 126 | ln(5m)CpsmUpsln(5m)CpsmCpslnTpsmCpslnTpsmGpsln(5m)CpsmCpslnGpsm |
| | ApslnTpsmCpsln(5m)CpsmA |
| 127 | lnGpslnApslnTpsln(5m)Cpsln(5m)CpslnApslnTpslnApsln(5m)CpslnTpslnGpsln(5m)C |
| | pslnGpslnGpslnApslnA |
| 128 | mGpsmApsmUps mCpsmCpsmAps mUpsmApsmCps mUpsmGpsmCps |
| | mGpsmGpsmApsmA |
| 129 | mGpsmApsmUpsm(5m)Cpsm(5m)CpsmApsmUpsmApsm(5m)CpsmUpsmGpsm(5m)C |
| | psmGpsmGpsmApsmA |
| 130 | moeGpsmoeApsmoeTpsmoe(5m)Cpsmoe(5m)CpsmoeApsmoeTpsmoeApsmoe(5m)Cp |
| | smoeTpsmoeGpsmoe(5m)CpsmoeGpsmoeGpsmoeApsmoeA |
| 131 | lnGpslnApslnTpsln(5m)Cpsm(5m)CpsmApsmUpsmApsm(5m)CpsmUpsmGpsm(5m)C |
| | pslnGpslnGpslnApslnA |
| 132 | lnGpsApsTpsln(5m)Cps(5m)CpsApslnTpsAps(5m)CpslnTpsGps(5m)CpslnGpsGpsAps |
| | lnA |
| 133 | mApsmGpsmUpsmGpsmUpsmUpsmUpsmGpsmCpsmUpsmGpsmApsmCpsmGpsmC |
| 134 | mApsmGpsmUpsmGpsmUpsmUpsmUpsmGpsm(5m)CpsmUpsmGpsmApsm(5m)Cps |
| | mGpsm(5m)C |
| 135 | moeApsmoeGpsmoeTpsmoeGpsmoeTpsmoeTpsmoeTpsmoeGpsmoe(5m)CpsmoeTps |
| | moeGpsmoeApsmoe(5m)CpsmoeGpsmoe(5m)C |
| 136 | moeApsmGpsmoeTpsmGpsmoeTpsmUpsmoeTpsmGpsmoe(5m)CpsmUpsmoeGpsmA |
| | psmoe(5m)CpsmGpsmoe(5m)C |
| 137 | lnApslnGpslnTpslnGpsmUpsmUpsmUpsmGpsm(5m)CpsmUpsmGpslnApsln(5m)Cpsl |
| | nGpsln(5m)C |
| 138 | lnApsGpsTpslnGpsTpsTpslnTpsGps(5m)CpslnTpsGpsApsln(5m)CpsGpsln(5m)C |
| 139 | mApslnGpsmUps lnGpsmUpslnTps mUpslnGpsm(5m)CpslnTpsmGpslnAps |
| | m(5m)CpslnGpsm(5m)C |
| 140 | ln(5m)Cpsm(5m)CpslnGpsmApsln(5m)Cpsm(5m)CpslnApsm(5m)CpslnGpsmGpslnGp |
| | smGpsln(5m)CpsmGpsln(5m)CpsmA |
| 141 | ln(5m)CpsmGpslnApsm(5m)Cpsln(5m)CpsmApsln(5m)CpsmGpslnGpsmGpslnGpsm( |
| | 5m)CpslnGpsm(5m)CpslnApsm(5m)C |
| 142 | lnApsm(5m)CpslnGpsmGpslnGpsmGpsln(5m)CpsmGpsln(5m)CpsmApsln(5m)Cpsm( |
| | 5m)CpslnTpsm(5m)CpslnTpsm(5m)C |
| 143 | lnGpsmApsln(5m)Cpsm(5m)CpslnApsm(5m)CpslnGpsmGpslnGpsmGpsln(5m)CpsmG |
| | psln(5m)CpsmApsln(5m)Cpsm(5m)C |
| 144 | lnGpsmGpslnGpsmGpsln(5m)CpsmGpsln(5m)CpsmApsln(5m)Cpsm(5m)CpslnTpsm(5 |
| | m)CpslnTpsm(5m)CpslnTpsmU |
| 145 | lnGpsmGpslnGpsmGpsln(5m)CpsmGpsln(5m)CpsmApsln(5m)Cpsm(5m)CpslnTpsm(5 |
| | m)CpslnTpsm(5m)CpslnTpslnT |
| 146 | ln(5m)Cpsm(5m)CpslnGpsmApsln(5m)Cpsm(5m)CpslnApsm(5m)CpslnGpsmGpslnGp |
| | smGpsln(5m)CpsmGpsln(5m)CpsmApsln(5m)Cpsm(5m)C |
| 147 | ln(5m)CpsmGpslnApsm(5m)Cpsln(5m)CpsmApsln(5m)CpsmGpslnGpsmGpslnGpsm( |
| | 5m)CpslnGpsm(5m)CpslnApsm(5m)Cpsln(5m)Cpsm(5m)CpslnT |
| 148 | ln(5m)Cpsm(5m)CpslnApsm(5m)CpslnGpsmGpslnGpsmGpsln(5m)CpsmGpsln(5m)Cp |
| | smApsln(5m)Cpsm(5m)CpslnTpsm(5m)CpslnTpsm(5m)C |
| 149 | lnGpsmApsln(5m)Cpsm(5m)CpslnApsm(5m)CpslnGpsmGpslnGpsmGpsln(5m)CpsmG |
| | psln(5m)CpsmApsln(5m)Cpsm(5m)Cpsln(5m)CpsmUpsln(5m)C |
| 150 | lnGpsmGpslnGpsmGpsln(5m)CpsmGpsln(5m)CpsmApsln(5m)Cpsm(5m)CpslnTpsm(5 |
| | m)CpslnTpsm(5m)CpslnTpsmUpslnTpsmA |
| 151 | lnGpsmGpslnGpsmGpsln(5m)CpsmGpsln(5m)CpsmApsln(5m)Cpsm(5m)CpslnTpsm(5 |
| | m)CpslnTpsm(5m)CpslnTpsmUpslnT |
| 152 | m(5m)CpsmGpsmGpsmGpsmApsm(5m)CpsmGpsmUpsm(5m)Cpsm(5m)CpsmUpsmU |
| | psmUpsmGpsmU |
| 153 | moe(5m)CpsmoeGpsmoeGpsmoeGpsmoeApsmoe(5m)CpsmoeGpsmoeTpsmoe(5m)Cp |
| | smoe(5m)CpsmoeTpsmoeTpsmoeTpsmoeGpsmoeT |
| 154 | ln(5m)CpsGpsGpslnGpsAps(5m)CpslnGpsTps(5m)Cpsln(5m)CpsTpsTpslnTpsGpslnT |
| 155 | GalNac4-ps2-p-mA-- |
| | lnGpsmApslnTpsmCpsln(5m)CpsmApslnTpsmApsln(5m)CpsmUpslnGpsmCpslnGpsm |
| | GpslnApsmA |
| 156 | ln(5m)CpslnGpslnGpslnGpsmApsm(5m)CpsmGpsmUpsm(5m)Cpsm(5m)CpsmUpslnT |
| | pslnTpslnGpslnT |
| 157 | m(5m)CpslnGpsmGpslnGpsmApsln(5m)Cps |
| | mGpslnTpsm(5m)Cpsln(5m)CpsmUpslnTpsmUpslnGpsmU |
| 158 | mGpslnApsmUps ln(5m)Cpsm(5m)CpslnAps mUpslnApsm(5m)Cps |
| | lnTpsmGpsln(5m)Cps mGpslnGpsmAps lnA |
| 159 | moe(5m)CpsmoeGpsmoeGpsmoeGpsmApsm(5m)CpsmGpsmUpsm(5m)Cpsm(5m)Cps |
| | mUpslnTpslnTpslnGpslnT |
| 160 | lnApsmGpslnTps mGpslnTpsmUps lnTpsmGpsln(5m)Cps mUpslnGpsmAps |
| | ln(5m)CpsmGpsln(5m)C |
| 161 | lnTpsmGpsln(5m)Cps m(5m)CpslnApsmAps ln(5m)CpsmUpslnGps mGpslnApsmUps |
| | ln(5m)Cpsm(5m)CpslnT |
| 162 | lnGpsm(5m)CpslnAps m(5m)Cpsln(5m)CpsmUps ln(5m)CpsmUpsln(5m)Cps |
| | mUpslnTpsmUps lnApsm(5m)CpslnG |
| 163 | lnGpsmApslnTps m(5m)Cpsln(5m)CpsmAps lnTpsmApsln(5m)Cps |
| | mUpslnGpsm(5m)Cps lnGpsmGpslnAps mA |
| 164 | ln(5m)CpsmUpslnGps m(5m)Cpsln(5m)CpsmAps lnApsm(5m)CpslnTps |
| | mGpslnGpsmApslnTpsm(5m)Cpsln (5m)Cps mU |
| 165 | lnGpsm(5m)CpslnGps mGpslnGpsmAps ln(5m)CpsmGpslnTps |
| | m(5m)Cpsln(5m)CpsmUps lnTps mUpslnGps mU |
| 166 | lnGpsmGpslnTps m(5m)CpslnApsm(5m)Cps ln(5m)CpsmApslnTps mApslnTpsmUps |
| | ln(5m)CpsmUpslnTps mG |
| 167 | lnTpsm(5m)CpslnAps m(5m)Cpsln(5m)CpsmApslnTpsmApslnTps |
| | mUpsln(5m)CpsmUps lnTpsmGpslnGps mG |
| 168 | ln(5m)CpsmApsln(5m)Cpsm(5m)CpslnApsmUpslnApsmUpslnTpsm(5m)CpslnTpsmU |
| | pslnGpsmGpslnGpsmA |
| 169 | ln(5m)Cpsm(5m)CpslnApsmUpslnApsmUpslnTpsm(5m)CpslnTpsmUpslnGpsmGpsln |
| | GpsmApslnApsm(5m)C |
| 170 | lnApsmUpslnApsmUpslnTpsm(5m)CpslnTpsmUpslnGpsmGpslnGpsmApslnApsm(5m) |
| | CpslnApsmA |
| 171 | ln(5m)CpsmApslnApsmGpslnApsmApslnTpsmApslnTpsmGpslnGpsmUpslnGpsmApsl |
| | n(5m)Cpsm(5m)C |
| 172 | ln(5m)Cpsm(5m)Cpsln(5m)CpsmApslnApsmGpslnApsmApslnTpsmApslnTpsmGpsln |
| | GpsmUpslnGpsmA |
| 173 | lnTpsm(5m)Cpsln(5m)Cpsm(5m)CpslnApsmApslnGpsmApslnApsmUpslnApsmUpsln |
| | GpsmGpslnTpsmG |
| 174 | lnGpsmUpslnTpsm(5m)Cpsln(5m)Cpsm(5m)CpslnApsmApslnGpsmApslnApsmUpsln |
| | ApsmUpslnGpsmG |
| 175 | lnTpsmUpslnGpsmUpslnTpsm(5m)Cpsln(5m)Cpsm(5m)CpslnApsmApslnGpsmApsln |
| | ApsmUpslnApsmU |
| 176 | lnTpsmUpslnGpsmGpslnGpsmGpslnTpsmGpslnGpsmApslnGpsm(5m)Cpsln(5m)Cpsm |
| | (5m)CpslnTpsm(5m)C |
| 177 | lnTpsmUpslnGpsmGpslnGpsmGpslnTpsmGpslnGpsmApslnGpsm(5m)Cpsln(5m)Cpsm |
| | (5m)CpslnTpsm(5m)CpslnA |
| 178 | lnTpsmGpslnGpsmGpslnGpsmUpslnGpsmGpslnApsmGpsln(5m)Cpsm(5m)Cpsln(5m) |
| | CpsmUpsln(5m)CpsmA |
| 179 | lnGpsmGpslnApsmGpsln(5m)Cpsm(5m)Cpsln(5m)CpsmUpsln(5m)CpsmApslnGpsmG |
| | psln(5m)CpsmUpsln(5m)CpsmA |
| 180 | ln(5m)Cpsm(5m)Cpsln(5m)CpsmUpsln(5m)CpsmApslnGpsmGpsln(5m)CpsmUpsln(5 |
| | m)CpsmApslnGpsmGpslnGpsm(5m)C |
| 181 | lnGpsmApslnGpsmGpslnGpsm(5m)CpslnTpsm(5m)Cpsln(5m)CpsmApsln(5m)Cpsm(5 |
| | m)Cpsln(5m)Cpsm(5m)CpslnApsmA |
| 182 | lnTpsmGpslnApsmGpslnGpsmGpsln(5m)CpsmUpsln(5m)Cpsm(5m)CpslnApsm(5m)C |
| | psln(5m)Cpsm(5m)Cpsln(5m)CpsmApslnA |
| 183 | lnTpsmGpslnApsmGpslnGpsmGpsln(5m)CpsmUpsln(5m)Cpsm(5m)CpslnApsm(5m)C |
| | psln(5m)Cpsm(5m)Cpsln(5m)CpsmA |
| 184 | lnTpsmGpslnApsmGpsln(5m)Cpsm(5m)CpslnTpsmGpslnApsmGpslnGpsmGpsln(5m) |
| | CpsmUpsln(5m)Cpsm(5m)C |
| 185 | lnGpsm(5m)Cpsln(5m)Cpsm(5m)CpslnTpsmGpslnApsmGpsln(5m)Cpsm(5m)CpslnTp |
| | smGpslnApsmGpslnGpsmG |
| 186 | mUpslnGpsmCps ln(5m)CpsmApslnAps mCpslnTpsmGps lnGpsmApslnTps |
| | mCpsln(5m)CpsmU |
| 187 | mUpsGpsln(5m)Cps m(5m)CpsApslnAps mCpsTpslnGps mGpsApslnTps |
| | mCps(5m)CpslnT |
| 188 | lnTpsGpsln(5m)Cps (5m)CpslnApsAps ln(5m)CpsTpslnGps GpslnApsTps |
| | ln(5m)Cps(5m)CpslnT |
| 189 | lnTpsGps(5m)Cps ln(5m)CpsApsAps ln(5m)CpsTpsGps lnGpsApsTps |
| | ln(5m)Cps(5m)CpslnT |
| 190 | lnTpsmGpsln(5m)Cps mCpslnApsmAps cp(5m)CpsmUpslnGps mGpslnApsmUps |
| | ln(5m)CpsmCpslnT |
| 191 | lnTpsmGpsln(5m)Cps m(5m)CpslnApsmAps cp(5m)CpsmUpscpGps mGpslnApsmUps |
| | ln(5m)Cpsm(5m)CpslnT |
| 192 | cpTpsmGpscp(5m)Cps m(5m)CpscpApsmAps cp(5m)CpsmUpscpGps |
| | mGpscpApsmUps cp(5m)Cpsm(5m)CpscpT |
| 193 | lnTpsmGpsln(5m)Cps mCpslnApsmAps am(5m)CpsmUpslnGps mGpslnApsmUps |
| | ln(5m)CpsmCpslnT |
| 194 | lnTpsmGpsln(5m)Cps mCpslnApsmAps am(5m)CpsmUpsamGps mGpslnApsmUps |
| | ln(5m)CpsmCpslnT |
| 195 | amTpsmGpsam(5m)Cps m(5m)CpsamApsmAps am(5m)CpsmUpsamGps |
| | mGpsamApsmUps am(5m)Cpsm(5m)CpsamT |
| 196 | mCpslnTpsmGps ln(5m)CpsmCpslnAps mApsln(5m)CpsmUps lnGpsmGpslnAps |
| | mUpsln(5m)CpsmCps lnT |
| 161; | 5′- |
| 197 | lnTpsmGpsln(5m)Cpsm(5m)CpslnApsmApsln(5m)CpsmUpslnGpsmGpslnApsmUpsln( |
| | 5m)Cpsm(5m)CpslnT-3′ (SEQ ID NO: 161) |
| | 3′-Aps(5m)CpsGps GpsTpsTps GpsAps(5m)Cps (5m)CpsTpsAps GpsGpsA-5′ |
| | (SEQ ID NO: 197) |
| 171; | 5′- |
| 198 | ln(5m)CpsmApslnApsmGpslnApsmApslnTpsmApslnTpsmGpslnGpsmUpslnGpsmApsln |
| | (5m)Cpsm(5m)C-3′ (SEQ ID NO: 171) |
| | 3′-GpsTpsTps (5m)CpsTpsTps ApsTpsAps (5m)Cps(5m)CpsAps(5m)CpsTpsGps G-5′ |
| | (SEQ ID NO: 198) |

A, C, (5m)C, G, T = Deoxy nucleoside
mA, mC, m(5m)C, mG, mU = 2′-O-methyl nucleoside;
lnA, lnG, ln(5m)C, lnT = locked nucleoside,;
ps = phosphorothioate linkage
lnX = locked nucleic acid (LNA) (e.g., lnG = locked nucleic acid (LNA) G);
amX = an amNA as disclosed in Table 4;
(5m)lnX = locked nucleic acid (LNA)-5 methyl nucleotide (e.g., (5m)lnC = LNA-5methyl C);
(5m)X = 5 methyl nucleotide (e.g., (5m)C = 5 methyl C);
mX = 2′-O-methoxy nucleotide (e.g., mA = 2′-O-methoxy A);
cpX = scpX = cyclopropyl nucleotide (e.g., cp (5m) C = scp(5m)C = cyclopropyl(5m)C);
moeX = 2′-O-methoxyethylribose nucleotide (e.g., moeG = 2′-O-methoxyethylribose G);
moe(5m)X = 2′-O-methoxyethylribose 5 methyl nucleotide (e.g., moe(5m)C = 2′-O-
methoxyethylribose 5 methyl C).

TABLE 3
EC50 and CC50 of Oligonucleotides

| SEQ | | | |
|---|---|---|---|
| ID | | | |
| NO: | Sequence (5′ → 3′) | EC50* | CC50** |
| 65 | mCpsmCpsmGpsmApsmCpsmCpsmApsmCpsmGpsm | B | B |
| | GpsmGpsmGpsmCpsmGpsmCpsmApsmCpsmCpsmUp | | |
| | smCpsmUpsmC | | |
| 66 | mCpsmGpsmApsmCpsmCpsmApsmCpsmGpsmGpsm | B | B |
| | GpsmGpsmCpsmGpsmCpsmApsmCpsmCpsmUpsmCp | | |
| | smUpsmCpsmU | | |
| 67 | mCpsmGpsmApsmCpsmCpsmApsmCpsmGpsmGpsm | B | B |
| | GpsmGpsmCpsmGpsmCpsmApsmCpsmCpsmUpsmCp | | |
| | smUpsmC | | |
| 68 | mGpsmApsmCpsmCpsmApsmCpsmGpsmGpsmGpsm | B | B |
| | GpsmCpsmGpsmCpsmApsmCpsmCpsmUpsmCpsmUp | | |
| | smCpsmU | | |
| 69 | mGpsmGpsmGpsmGpsmCpsmGpsmCpsmApsmCpsm | B | B |
| | CpsmUpsmCpsmUpsmCpsmUpsmUpsmUpsmApsmCp | | |
| | smGpsmCpsmG | | |
| 70 | mCpsln(5m)CpsmGpslnApsmCpsln(5m)CpsmApsln(5 | B | B |
| | m)CpsmGpslnGpsmGpslnGpsmCpslnGpsmCpslnApsm | | |
| | Cpsln(5m)CpsmUpsln(5m)CpsmUpsln(5m)C | | |
| 71 | mCpslnGpsmApsln(5m)CpsmCpslnApsmCpslnGpsmG | B | B |
| | pslnGpsmGpsln(5m)CpsmGpsln(5m)CpsmApsln(5m)C | | |
| | psmCpslnTpsmCpslnTpsmCpslnT | | |
| 72 | mGpslnApsmCpsln(5m)CpsmApsln(5m)CpsmGpslnGp | B | B |
| | smGpslnGpstnCpslnGpsmCpslnApsmCpsln(5m)Cpsm | | |
| | Upsln(5m)CpsmUpsln(5m)CpslnT | | |
| 73 | mGpslnApsmCpsln(5m)CpsmApsln(5m)CpsmGpslnGp | B | B |
| | smGpslnGpsmCpslnGpsmCpslnApsmCpsln(5m)Cpsm | | |
| | Upsln(5m)CpsmUpsln(5m)CpslnT | | |
| 74 | mGpslnGpsmGpslnGpsmCpslnGpsmCpslnApsmCpsln( | B | B |
| | 5m)CpsmUpsln(5m)CpsmUpsln(5m)CpsmUpslnTpsmU | | |
| | pslnApsmCpslnGpsmCpslnG | | |
| 75 | ln(5m)CpsmGpsln(5m)CpsmApsln(5m)CpsmCpslnTps | B | B |
| | mCpslnTpsmCpslnTpsmUpslnTpsmApsln(5m)CpsmG | | |
| 76 | lnGpsln(5m)CpslnApsln(5m)Cpsln(5m)CpslnTpsln(5m) | A | B |
| | CpslnTpsln(5m)CpslnTpslnTpslnTpslnApsln(5m)Cpsln | | |
| | G | | |
| 77 | lnGpsln(5m)CpslnApsln(5m)Cpsln(5m)CpslnTpsln(5m) | ND | ND |
| | CpslnTpsln(5m)CpslnTpslnTpslnTpslnApsln(5m)Cpsln | | |
| | Gpsln(5m)C | | |
| 78 | ln(5m)CpslnGpsln(5m)CpslnApsln(5m)Cpsln(5m)Cpsln | A | B |
| | Tpsln(5m)CpslnTpsln(5m)CpslnTpslnTpslnTpslnApsln( | | |
| | 5m)C | | |
| 79 | ln(5m)CpslnGpslnGpslnGpslnApsln(5m)CpslnGpslnTp | B | B |
| | sln(5m)Cpsln(5m)CpslnTpslnTpslnTpslnGpslnT | | |
| 80 | ln(5m)CpsmCpslnGpsmUpslnGpsmUpslnGpsmCpslnA | B | B |
| | psmCpslnTpsmUpsln(5m)CpsmGpsln(5m)C | | |
| 81 | ln(5m)CpsmCpslnTpsmCpslnTpsmCpslnTpsmUpslnTp | B | B |
| | smApsln(5m)CpsmGpsln(5m)CpsmGpslnG | | |
| 82 | lnApsmCpsln(5m)CpsmUpsln(5m)CpsmUpsln(5m)Cps | B | B |
| | mUpslnTpsmUpslnApsmCpslnGpsmCpslnG | | |
| 83 | ln(5m)CpsmApsln(5m)CpsmCpslnTpsmCpslnTpsmCps | B | B |
| | lnTpsmUpslnTpsmApsln(5m)CpsmGpsln(5m)C | | |
| 84 | ln(5m)CpsmApsln(5m)CpsmCpslnTpsmCpslnTpsmCps | B | B |
| | lnTpsmUpslnTpsmApsln(5m)CpsmGpsln(5m)CpsmGps | | |
| | LnG | | |
| 85 | lnGpsmCpslnApsmCpsln(5m)CpsmUpsln(5m)CpsmUp | B | B |
| | sln(5m)CpsmUpslnTpsmUpslnApsmCpslnG | | |
| 86 | ln(5m)CpsmGpsln(5m)CpsmApsln(5m)CpsmCpslnTps | B | B |
| | mCpslnTpsmCpslnTpsmUpslnTpsmApsln(5m)C | | |
| 87 | ln(5m)CpsmGpsln(5m)CpsmApsln(5m)CpsmCpslnTps | B | B |
| | mCpslnTpsmCpslnTpsmUpslnTpsmApsln(5m)CpsmGp | | |
| | sln(5m)C | | |
| 88 | ln(5m)CpsmGpsln(5m)CpsmApsln(5m)CpsmCpslnTps | A | A |
| | mCpslnTpsmCpslnTpsmUpslnTpsmApsln(5m)CpsmGp | | |
| | sln(5m)CpsmG | | |
| 89 | ln(5m)CpsmGpslnGpsmGpslnApsmCpslnGpsmUpsln(5 | B | B |
| | m)CpsmCpslnTpsmUpslnTpsmGpslnT | | |
| 90 | lnGpsmCpslnGpsmGpslnGpsmApsln(5m)CpsmGpslnT | B | B |
| | psmCpsln(5m)CpsmUpslnTpsmUpslnG | | |
| 91 | lnGpsmCpslnGpsmGpslnGpsmApsln(5m)CpsmGpslnT | A | B |
| | psmCpsln(5m)CpsmUpslnTpsmUpslnGpsmU | | |
| 92 | ln(5m)CpsmGpsln(5m)CpsmGpslnGpsmGpslnApsmCp | B | B |
| | slnGpsmUpsln(5m)CpsmCpslnTpsmUpslnTpsmGpslnT | | |
| 93 | lnTpsmGpsln(5m)CpsmCpslnApsmApsln(5m)CpsmUps | B | B |
| | lnGpsmGpslnApsmUpsln(5m)CpsmCpslnT | | |
| 94 | ln(5m)CpsmUpslnGpsmCpsln(5m)CpsmApslnApsmCp | B | B |
| | slnTpsmGpslnGpsmApslnTpsmCpsln(5m)C | | |
| 95 | ln(5m)CpsmUpslnGpsmCpsln(5m)CpsmApslnApsmCp | A | B |
| | slnTpsmGpslnGpsmApslnTpsmCpsln(5m)CpsmU | | |
| 96 | ln(5m)CpsmCpslnApsmUpslnApsmCpslnTpsmGpsln(5 | A | A |
| | m)CpsmGpslnGpsmApslnApsmCpslnT | | |
| 97 | lnTpsmCpsln(5m)CpsmApslnTpsmApsln(5m)CpsmUps | A | B |
| | lnGpsmCpslnGpsmGpslnApsmApsln(5m)CpsmU | | |
| 98 | lnApsmUpsln(5m)CpsmCpslnApsmUpslnApsmCpslnT | A | A |
| | psmGpsln(5m)CpsmGpslnGpsmApslnA | | |
| 99 | lnApsmUpsln(5m)CpsmCpslnApsmUpslnApsmCpslnT | B | B |
| | psmGpsln(5m)CpsmGpslnGpsmApslnApsmCpslnT | | |
| 100 | lnGpsmApslnTpsmCpsln(5m)CpsmApslnTpsmApsln(5 | A | B |
| | m)CpsmUpslnGpsmCpslnGpsmGpslnApsmA | | |
| 101 | ln(5m)CpsmGpslnApsmUpsln(5m)CpsmCpslnApsmUp | B | B |
| | slnApsmCpslnTpsmGpsln(5m)CpsmGpslnG | | |
| 102 | ln(5m)CpsmCpslnGpsmApslnTpsmCpsln(5m)CpsmAps | B | B |
| | lnTpsmApsln(5m)CpsmUpslnGpsmCpslnG | | |
| 103 | ln(5m)CpsmCpslnGpsmApslnTpsmCpsln(5m)CpsmAps | B | B |
| | lnTpsmApsln(5m)CpsmUpslnGpsmCpslnGpsmG | | |
| 104 | ln(5m)CpsmUpslnGpsmCpsln(5m)CpsmGpslnApsmUp | A | A |
| | sln(5m)CpsmCpslnApsmUpslnApsmCpslnT | | |
| 105 | ln(5m)CpsmUpslnGpsmCpsln(5m)CpsmGpslnApsmUp | A | A |
| | sln(5m)CpsmCpslnApsmUpslnApsmCpslnTpsmG | | |
| 106 | lnTpsmCpslnTpsmGpsln(5m)CpsmCpslnGpsmApslnTp | A | A |
| | smCpsln(5m)CpsmApslnTpsmApsln(5m)C | | |
| 107 | lnTpsmCpslnTpsmGpsln(5m)CpsmCpslnGpsmApslnTp | A | A |
| | smCpsln(5m)CpsmApslnTpsmApsln(5m)CpsmU | | |
| 108 | ln(5m)CpsmUpsln(5m)CpsmUpslnGpsmCpsln(5m)Cps | A | A |
| | mGpslnApsmUpsln(5m)CpsmCpslnApsmUpslnApsmC | | |
| 109 | ln(5m)CpsmUpsln(5m)CpsmUpslnGpsmCpsln(5m)Cps | A | A |
| | mGpslnApsmUpsln(5m)CpsmCpslnApsmUpslnApsmC | | |
| | pslnT | | |
| 110 | lnTpsmCpsln(5m)CpsmUpsln(5m)CpsmUpslnGpsmCps | A | A |
| | ln(5m)CpsmGpslnApsmUpsln(5m)CpsmCpslnA | | |
| 111 | lnTpsmCpsln(5m)CpsmUpsln(5m)CpsmUpslnGpsmCps | B | A |
| | ln(5m)CpsmGpslnApsmUpsln(5m)CpsmCpslnA | | |
| 112 | lnApsmGpslnTpsmGpslnTpsmUpslnTpsmGpsln(5m)Cp | A | B |
| | smUpslnGpsmApsln(5m)CpsmGpsln(5m)C | | |
| 113 | lnApsln(5m)Cpsln(5m)CpslnTpsln(5m)CpslnTpsln(5m) | B | B |
| | CpslnTpslnTpslnTpslnApsln(5m)CpslnGpsln(5m)Cpsln | | |
| | G | | |
| 114 | ln(5m)CpslnApsln(5m)Cpsln(5m)CpslnTpsln(5m)Cpsln | B | B |
| | Tpsln(5m)CpslnTpslnTpslnTpslnApsln(5m)CpslnGpsln | | |
| | (5m)C | | |
| 115 | lnApslnGpslnTpslnGpslnTpslnTpslnTpslnGpsln(5m)Cp | B | B |
| | slnTpslnGpslnApsln(5m)CpslnGpsln(5m)C | | |
| 116 | lnApsmCpsln(5m)CpsmUpsln(5m)CpsmUpsln(5m)Cps | A | B |
| | mUpslnTpsmUpslnApsmCpslnGpsmCpslnGpsmG | | |
| 117 | ln(5m)CpsmApsln(5m)CpsmCpslnTpsmCpslnTpsmCps | B | B |
| | lnTpsmUpslnTpsmApsln(5m)CpsmGpsln(5m)CpsmG | | |
| 118 | lnGpsmCpslnApsmCpsln(5m)CpsmUpsln(5m)CpsmUp | B | B |
| | sln(5m)CpsmUpslnTpsmUpslnApsmCpslnGpsmC | | |
| 119 | lnGpsmCpslnApsmCpsln(5m)CpsmUpsln(5m)CpsmUp | A | A |
| | sln(5m)CpsmUpslnTpsmUpslnApsmCpslnGpsmCpslnG | | |
| 120 | ln(5m)CpsmGpsln(5m)CpsmGpslnGpsmGpslnApsmCp | B | B |
| | slnGpsmUpsln(5m)CpsmCpslnTpsmUpslnTpsmG | | |
| 121 | ln(5m)CpsmGpslnApsmUpsln(5m)CpsmCpslnApsmUp | B | B |
| | slnApsmCpslnTpsmGpsln(5m)CpsmGpslnGpsmApslnA | | |
| 122 | lnTpsmGpsln(5m)CpsmCpslnGpsmApslnTpsmCpsln(5 | B | B |
| | m)CpsmApslnTpsmApsln(5m)CpsmUpslnG | | |
| 123 | lnTpsmCpslnTpsmGpsln(5m)CpsmCpslnGpsmApslnTp | B | B |
| | smCpsln(5m)CpsmApslnTpsmApsln(5m)CpsmUpslnG | | |
| 124 | ln(5m)CpsmCpslnTpsmCpslnTpsmGpsln(5m)CpsmCps | B | B |
| | lnGpsmApslnTpsmCpsln(5m)CpsmApslnT | | |
| 125 | ln(5m)CpsmUpsln(5m)CpsmCpslnTpsmCpslnTpsmGps | B | B |
| | ln(5m)CpsmCpslnGpsmApslnTpsmCpsln(5m)C | | |
| 126 | ln(5m)CpsmUpsln(5m)CpsmCpslnTpsmCpslnTpsmGps | B | B |
| | ln(5m)CpsmCpslnGpsmApslnTpsmCpsln(5m)CpsmA | | |
| 127 | lnGpslnApslnTpsln(5m)Cpsln(5m)CpslnApslnTpslnApsln(5 | A | B |
| | m)CpslnTpslnGpsln(5m)CpslnGpslnGpslnApslnA | | |
| 128 | mGpsmApsmUps mCpsmCpsmAps mUpsmApsmCps | A | B |
| | mUpsmGpsmCps mGpsmGpsmApsmA | | |
| 129 | mGpsmApsmUpsm(5m)Cpsm(5m)CpsmApsmUpsmApsm(5 | A | A |
| | m)CpsmUpsmGpsm(5m)CpsmGpsmGpsmApsmA | | |
| 130 | moeGpsmoeApsmoeTpsmoe(5m)Cpsmoe(5m)CpsmoeApsm | A | B |
| | oeTpsmoeApsmoe(5m)CpsmoeTpsmoeGpsmoe(5m)Cpsmoe | | |
| | GpsmoeGpsmoeApsmoeA | | |
| 131 | lnGpslnApslnTpsln(5m)Cpsm(5m)CpsmApsmUpsmApsm(5 | A | A |
| | m)CpsmUpsmGpsm(5m)CpslnGpslnGpslnApslnA | | |
| 132 | lnGpsApsTpsln(5m)Cps(5m)CpsApslnTpsAps(5m)CpslnTps | A | B |
| | Gps(5m)CpslnGpsGpsApslnA | | |
| 133 | mApsmGpsmUpsmGpsmUpsmUpsmUpsmGpsmCpsmUpsm | A | A |
| | GpsmApsmCpsmGpsmC | | |
| 134 | mApsmGpsmUpsmGpsmUpsmUpsmUpsmGpsm(5m)Cpsm | A | B |
| | UpsmGpsmApsm(5m)CpsmGpsm(5m)C | | |
| 135 | moeApsmoeGpsmoeTpsmoeGpsmoeTpsmoeTpsmoeTpsmoe | A | B |
| | Gpsmoe(5m)CpsmoeTpsmoeGpsmoeApsmoe(5m)CpsmoeG | | |
| | psmoe(5m)C | | |
| 136 | moeApsmGpsmoeTpsmGpsmoeTpsmUpsmoeTpsmGpsmoe( | B | B |
| | 5m)CpsmUpsmoeGpsmApsmoe(5m)CpsmGpsmoe(5m)C | | |
| 137 | lnApslnGpslnTpslnGpsmUpsmUpsmUpsmGpsm(5m)CpsmU | A | B |
| | psmGpslnApsln(5m)CpslnGpsln(5m)C | | |
| 138 | lnApsGpsTpslnGpsTpsTpslnTpsGps(5m)CpslnTpsGpsApsln | A | B |
| | (5m)CpsGpsln(5m)C | | |
| 139 | mApslnGpsmUps lnGpsmUpslnTps mUpslnGpsm(5m)Cps | A | B |
| | lnTpsmGpslnAps m(5m)CpslnGpsm(5m)C | | |
| 140 | ln(5m)Cpsm(5m)CpslnGpsmApsln(5m)Cpsm(5m)CpslnAps | A | B |
| | m(5m)CpslnGpsmGpslnGpsmGpsln(5m)CpsmGpsln(5m)Cps | | |
| | mA | | |
| 141 | ln(5m)CpsmGpslnApsm(5m)Cpsln(5m)CpsmApsln(5m)Cps | A | B |
| | mGpslnGpsmGpslnGpsm(5m)CpslnGpsm(5m)CpslnApsm(5 | | |
| | m)C | | |
| 142 | lnApsm(5m)CpslnGpsmGpslnGpsmGpsln(5m)CpsmGpsln(5 | A | B |
| | m)CpsmApsln(5m)Cpsm(5m)CpslnTpsm(5m)CpslnTpsm(5 | | |
| | m)C | | |
| 143 | lnGpsmApsln(5m)Cpsm(5m)CpslnApsm(5m)CpslnGpsmGps | A | B |
| | lnGpsmGpsln(5m)CpsmGpsln(5m)CpsmApsln(5m)Cpsm(5m) | | |
| | C | | |
| 144 | lnGpsmGpslnGpsmGpsln(5m)CpsmGpsln(5m)CpsmApsln(5 | A | B |
| | m)Cpsm(5m)CpslnTpsm(5m)CpslnTpsm(5m)CpslnTpsmU | | |
| 145 | lnGpsmGpslnGpsmGpsln(5m)CpsmGpsln(5m)CpsmApsln(5 | A | A |
| | m)Cpsm(5m)CpslnTpsm(5m)CpslnTpstn(5m)CpslnTpslnT | | |
| 146 | ln(5m)Cpsm(5m)CpslnGpsmApsln(5m)Cpsm(5m)CpslnAps | A | B |
| | m(5m)CpslnGpsmGpslnGpsmGpsln(5m)CpsmGpsln(5m)Cps | | |
| | mApsln(5m)Cpsm(5m)C | | |
| 147 | ln(5m)CpsmGpslnApsm(5m)Cpsln(5m)CpsmApsln(5m)Cps | A | B |
| | mGpslnGpsmGpslnGpsm(5m)CpslnGpsm(5m)CpslnApsm(5 | | |
| | m)Cpsln(5m)Cpsm(5m)CpslnT | | |
| 148 | ln(5m)Cpsm(5m)CpslnApsm(5m)CpslnGpsmGpslnGpsmGps | A | B |
| | ln(5m)CpsmGpsln(5m)CpsmApsln(5m)Cpsm(5m)CpslnTps | | |
| | m(5m)CpslnTpsm(5m)C | | |
| 149 | lnGpsmApsln(5m)Cpsm(5m)CpslnApsm(5m)CpslnGpsmGps | A | B |
| | lnGpsmGpsln(5m)CpsmGpsln(5m)CpsmApsln(5m)Cpsm(5m) | | |
| | Cpsln(5m)CpsmUpsln(5m)C | | |
| 150 | lnGpsmGpslnGpsmGpsln(5m)CpsmGpsln(5m)CpsmApsln(5 | A | B |
| | m)Cpsm(5m)CpslnTpsm(5m)CpslnTpsm(5m)CpslnTpsmUps | | |
| | lnTpsmA | | |
| 151 | lnGpsmGpslnGpsmGpsln(5m)CpsmGpsln(5m)CpsmApsln(5 | A | B |
| | m)Cpsm(5m)CpslnTpsm(5m)CpslnTpsm(5m)CpslnTpsmUps | | |
| | lnT | | |
| 152 | m(5m)CpsmGpsmGpsmGpsmApsm(5m)CpsmGpsmUpsm(5 | A | A |
| | m)Cpsm(5m)CpsmUpsmUpsmUpsmGpsmU | | |
| 153 | moe(5m)CpsmoeGpsmoeGpsmoeGpsmoeApsmoe(5m)Cpsm | A | A |
| | oeGpsmoeTpsmoe(5m)Cpsmoe(5m)CpsmoeTpsmoeTpsmoe | | |
| | TpsmoeGpsmoeT | | |
| 154 | ln(5m)CpsGpsGpslnGpsAps(5m)CpslnGpsTps(5m)Cpsln(5m) | A | B |
| | CpsTpsTpslnTpsGpslnT | | |
| 155 | GalNac4-ps2-p-mA-- | ND | ND |
| | lnGpsmApslnTpsmCpsln(5m)CpsmApslnTpsmApsln(5m)Cp | | |
| | smUpslnGpsmCpslnGpsmGpslnApsmA | | |
| 156 | ln(5m)CpslnGpslnGpslnGpsmApsm(5m)CpsmGpsmUpsm(5 | A | B |
| | m)Cpsm(5m)CpsmUpslnTpslnTpslnGpslnT | | |
| 157 | m(5m)CpslnGpsmGpslnGpsmApsln(5m)Cps | A | B |
| | mGpslnTpsm(5m)Cpslii(5m)CpsmUpslnTpsmUpslnGpsmU | | |
| 158 | mGpslnApsmUps ln(5m)Cpsm(5m)CpslnAps | A | B |
| | mUpslnApsm(5m)Cps lnTpsmGpsln(5m)Cps | | |
| | mGpslnGpsmAps lnA | | |
| 159 | moe(5m)CpsmoeGpsmoeGpsmoeGpsmApsm(5m)CpsmGps | A | B |
| | mUpsm(5m)Cpsm(5m)CpsmUpslnTpslnTpslnGpslnT | | |
| 160 | lnApsmGpslnTps mGpslnTpsmUps lnTpsmGpsln(5m)Cps | A | B |
| | mUpslnGpsmAps ln(5m)CpsmGpsln(5m)C | | |
| 161 | lnTpsmGpsln(5m)Cps m(5m)CpslnApsmAps | A | B |
| | ln(5m)CpsmUpslnGps mGpslnApsmUps | | |
| | ln(5m)Cpsm(5m)CpslnT | | |
| 162 | lnGpsm(5m)CpslnAps m(5m)Cpsln(5m)CpsmUps | A | B |
| | ln(5m)CpsmUpsln(5m)Cps mUpslnTpsmUps | | |
| | lnApsm(5m)CpslnG | | |
| 163 | lnGpsmApslnTps m(5m)Cpsln(5m)CpsmAps | A | B |
| | lnTpsmApsln(5m)Cps mUpslnGpsm(5m)Cps | | |
| | lnGpsmGpslnAps mA | | |
| 164 | ln(5m)CpsmUpslnGps m(5m)Cpsln(5m)CpsmAps | A | B |
| | lnApsm(5m)CpslnTps mGpslnGpsmAps lnTpsm(5m)Cpsln | | |
| | (5m)Cps mU | | |
| 165 | lnGpsm(5m)CpslnGps mGpslnGpsmAps | A | B |
| | ln(5m)CpsmGpslnTps m(5m)Cpsln(5m)CpsmUps LnTps | | |
| | mUpslnGps mU | | |
| 166 | lnGpsmGpslnTps m(5m)CpslnApsm(5m)Cps | B | B |
| | ln(5m)CpsmApslnTps mApslnTpsmUps | | |
| | ln(5m)CpsmUpslnTps mG | | |
| 167 | lnTpsm(5m)CpslnAps m(5m)Cpsln(5m)CpsmAps | A | A |
| | lnTpsmApslnTps mUpsln(5m)CpsmUps LnTpsmGpslnGps | | |
| | mG | | |
| 168 | ln(5m)CpsmApsln(5m)Cpsm(5m)CpslnApsmUpslnApsmUps | B | B |
| | lnTpsm(5m)CpslnTpsmUpslnGpsmGpslnGpsmA | | |
| 169 | ln(5m)Cpsm(5m)CpslnApsmUpslnApsmUpslnTpsm(5m)Cps | A | B |
| | lnTpsmUpslnGpsmGpslnGpsmApslnApsm(5m)C | | |
| 170 | lnApsmUpslnApsmUpslnTpsm(5m)CpslnTpsmUpslnGpsmG | B | B |
| | pslnGpsmApslnApsm(5m)CpslnApsmA | | |
| 171 | ln(5m)CpsmApslnApsmGpslnApsmApslnTpsmApslnTpsmG | A | B |
| | pslnGpsmUpslnGpsmApsln(5m)Cpsm(5m)C | | |
| 172 | ln(5m)Cpsm(5m)Cpsln(5m)CpsmApslnApsmGpslnApsmAps | A | B |
| | lnTpsmApslnTpsmGpslnGpsmUpslnGpsmA | | |
| 173 | lnTpsm(5m)Cpsln(5m)Cpsm(5m)CpslnApsmApslnGpsmAps | A | B |
| | lnApsmUpslnApsmUpslnGpsmGpslnTpsmG | | |
| 174 | lnGpsmUpslnTpsm(5m)Cpsln(5m)Cpsm(5m)CpslnApsmAps | A | B |
| | lnGpsmApslnApsmUpslnApsmUpslnGpsmG | | |
| 175 | lnTpsmUpslnGpsmUpslnTpsm(5m)Cpsln(5m)Cpsm(5m)Cps | A | B |
| | lnApsmApslnGpsniApslnApsmUpslnApsmU | | |
| 176 | LnTpsmUpslnGpsmGpslnGpsmGpslnTpsmGpslnGpsmApsln | A | A |
| | Gpsm(5m)Cpsln(5m)Cpsm(5m)CpslnTpsm(5m)C | | |
| 177 | lnTpsmUpslnGpsmGpslnGpsmGpslnTpsmGpslnGpsmApsln | A | A |
| | Gpsm(5m)Cpsln(5m)Cpsm(5m)CpslnTpsm(5m)CpslnA | | |
| 178 | lnTpsmGpslnGpsmGpslnGpsmUpslnGpsmGpslnApsmGpsln | A | A |
| | (5m)Cpsm(5m)Cpsln(5m)CpsmUpsln(5m)CpsmA | | |
| 179 | lnGpsmGpslnApsmGpsln(5m)Cpsm(5m)Cpsln(5m)CpsmUps | B | B |
| | ln(5m)CpsmApslnGpsmGpsln(5m)CpsmUpsln(5m)CpsmA | | |
| 180 | ln(5m)Cpsm(5m)Cpsln(5m)CpsmUpsln(5m)CpsmApslnGps | A | A |
| | mGpsln(5m)CpsmUpsln(5m)CpsmApslnGpsmGpslnGpsm(5 | | |
| | m)C | | |
| 181 | lnGpsmApslnGpsmGpslnGpsm(5m)CpslnTpsm(5m)Cpsln(5 | ND | B |
| | m)CpsmApsln(5m)Cpsm(5m)Cpsln(5m)Cpsm(5m)CpslnAps | | |
| | mA | | |
| 182 | lnTpsmGpslnApsmGpslnGpsmGpsln(5m)CpsmUpsln(5m)Cp | B | B |
| | sm(5m)CpslnApsm(5m)Cpsln(5m)Cpsm(5m)Cpsln(5m)Cps | | |
| | mApslnA | | |
| 183 | lnTpsmGpslnApsmGpslnGpsmGpsln(5m)CpsmUpsln(5m)Cp | A | B |
| | sm(5m)CpslnApsm(5m)Cpsln(5m)Cpsm(5m)Cpsln(5m)Cps | | |
| | mA | | |
| 184 | lnTpsmGpslnApsmGpsln(5m)Cpsm(5m)CpslnTpsmGpslnAp | A | B |
| | smGpslnGpsmGpsln(5m)CpsmUpsln(5m)Cpsm(5m)C | | |
| 185 | lnGpsm(5m)Cpsln(5m)Cpsm(5m)CpslnTpsmGpslnApsmGps | A | A |
| | ln(5m)Cpsm(5m)CpslnTpsmGpslnApsmGpslnGpsmG | | |
| 186 | mUpslnGpsmCps ln(5m)CpsmApslnAps mCpslnTpsmGps | A | B |
| | lnGpsmApslnTps mCpsln(5m)CpsmU | | |
| 187 | mUpsGpsln(5m)Cps m(5m)CpsApslnAps mCpsTpslnGps | A | A |
| | mGpsApslnTps mCps(5m)CpslnT | | |
| 188 | lnTpsGpsln(5m)Cps (5m)CpslnApsAps ln(5m)CpsTpslnGps | A | A |
| | GpslnApsTps ln(5m)Cps(5m)CpslnT | | |
| 189 | lnTpsGps(5m)Cps ln(5m)CpsApsAps ln(5m)CpsTpsGps | A | A |
| | lnGpsApsTps ln(5m)Cps(5m)CpslnT | | |
| 190 | lnTpsmGpsln(5m)Cps mCpslnApsmAps | A | B |
| | cp(5m)CpsmUpslnGps mGpslnApsmUps | | |
| | ln(5m)CpsmCpslnT | | |
| 191 | lnTpsmGpsln(5m)Cps m(5m)CpslnApsmAps | A | B |
| | cp(5m)CpsmUpscpGps mGpslnApsmUps | | |
| | ln(5m)Cpsm(5m)CpslnT | | |
| 192 | cpTpsmGpscp(5m)Cps m(5m)CpscpApsmAps | ND | B |
| | cp(5m)CpsmUpscpGps mGpscpApsmUps | | |
| | cp(5m)Cpsm(5m)CpscpT | | |
| 193 | lnTpsmGpsln(5m)Cps mCpslnApsmAps | A | B |
| | am(5m)CpsmUpslnGps mGpslnApsmUps | | |
| | ln(5m)CpsmCpslnT | | |
| 194 | lnTpsmGpsln(5m)Cps mCpslnApsmAps | ND | B |
| | am(5m)CpsmUpsamGps mGpslnApsmUps | | |
| | ln(5m)CpsmCpslnT | | |
| 195 | amTpsmGpsam(5m)Cps m(5m)CpsamApsmAps | A | B |
| | am(5m)CpsmUpsamGps mGpsamApsmUps | | |
| | am(5m)Cpsm(5m)CpsamT | | |
| 196 | mCpslnTpsmGps ln(5m)CpsmCpslnAps | ND | B |
| | mApsln(5m)CpsmUps LnGpsmGpslnAps | | |
| | mUpsln(5m)CpsmCps LnT | | |
| 161; | 5′- | A | B |
| 197 | lnTpsmGpsln(5m)Cpsm(5m)CpslnApsmApsln(5m)CpsmUps | | |
| | lnGpsmGpslnApsmUpsln(5m)Cpsm(5m)CpslnT-3′ (SEQ ID | | |
| | NO: 161) | | |
| | 3′-Aps(5m)CpsGps GpsTpsTps GpsAps(5m)Cps | | |
| | (5m)CpsTpsAps GpsGpsA-5′ (SEQ ID NO: 197) | | |
| 171; | 5′- | A | B |
| 198 | ln(5m)CpsmApslnApsmGpslnApsmApslnTpsmApslnTpsmG | | |
| | pslnGpsmUpslnGpsmApsln (5m)Cpsm(5m)C-3′ (SEQ ID | | |
| | NO: 171) | | |
| | 3′-GpsTpsTps (5m)CpsTpsTps ApsTpsAps | | |
| | (5m)Cps(5m)CpsAps(5m)CpsTpsGps G-5′ (SEQ ID NO: | | |
| | 198) | | |

A, C, (5m)C, G, T = Deoxy nucleoside
mA, mC, m(5m)C, mG, mU = 2′-O-methyl nucleoside;
lnA, lnG, ln(5m)C, lnT = locked nucleoside,;
ps = phosphorothioate linkage
lnX = locked nucleic acid (LNA) (e.g., lnG = locked nucleic acid (LNA) G);
amX = an amNA as disclosed in Table 4;
(5m)lnX = locked nucleic acid (LNA)-5 methyl nucleotide (e.g., (5m)lnC = LNA-5methyl C);
(5m)X = 5 methyl nucleotide (e.g., (5m)C = 5 methyl C);
mX = 2′-O-methoxy nucleotide (e.g., mA = 2′-O-methoxy A);
cpX = scpX = cyclopropyl nucleotide (e.g., cp(5m)C = scp(5m) C = cyclopropyl (5m) C);
moeX = 2′-O-methoxyethylribose nucleotide (e.g., moeG = 2′-O-methoxyethylribose G);
moe(5m)X = 2′-O-methoxyethylribose 5 methyl nucleotide (e.g., moe(5m)C = 2′-O-
methoxyethylribose 5 methyl C).
*For EC50: A < 1 μM, B ≥ 1 μM, ND = Not determined
**For CC50: A < 1 μM, B ≥ 1 μM, ND = Not determined

### EQUIVALENTS

The present technology has been described broadly and generically herein. Each of the narrower species and sub-generic groupings falling within the generic disclosure also form part of the present technology. This includes the generic description of the present technology with a proviso or negative limitation removing any subject matter from the genus, regardless of whether the excised material is specifically recited herein.

In addition, where features or aspects of the present technology are described in terms of Markush groups, those skilled in the art will recognize that the present technology is also thereby described in terms of any individual member or subgroup of members of the Markush group.

The practice of the present technology will employ, unless otherwise indicated, conventional techniques of organic chemistry, pharmacology, immunology, molecular biology, microbiology, cell biology and recombinant DNA, which are within the skill of the art. See, e.g., Sambrook, Fritsch and Maniatis, Molecular Cloning: A Laboratory Manual, 2nd edition (1989); Current Protocols In Molecular Biology (F. M. Ausubel, et al. eds., (1987)); the series Methods in Enzymology (Academic Press, Inc.): PCR 2: A Practical Approach (M. J. MacPherson, B. D. Hames and G. R. Taylor eds. (1995)), Harlow and Lane, eds. (1988) Antibodies, a Laboratory Manual, and Animal Cell Culture (R.I. Freshney, ed. (1987)).

Throughout this disclosure, various publications, patents, and published patent specifications may be referenced by an identifying citation or by an Arabic numeral. The full citation for the publications identified by an Arabic numeral is found immediately preceding the claims. All publications, patent applications, patents, and other references mentioned herein are expressly incorporated by reference in their entirety, to the same extent as if each were incorporated by reference individually. In case of conflict, the present specification, including definitions, will control.

Unless otherwise defined, all technical and scientific terms used herein have the same meaning as commonly understood by one of ordinary skill in the art to which this technology belongs.

Thus, it should be understood that the materials, methods, and examples provided here are representative of preferred aspects, are exemplary, and are not intended as limitations on the scope of the present technology.

Other aspects are set forth within the following claims.

1. An oligonucleotide comprising a nucleotide sequence comprising 5 to 40 nucleotides, wherein one or more of the 5 to 40 nucleotides is a modified nucleoside, wherein at least 5 consecutive nucleotides of the 5 to 40 nucleotides is identical, complementary, hybridizes or binds to a viral target sequence, wherein the viral target sequence is within (a) a relaxed circular DNA (rcDNA) form of a hepatitis B virus (HBV) genome; (b) a covalently closed circular DNA (cccDNA) of the HBV genome; or (c) an HBV transcript.

2. The oligonucleotide of claim 1, wherein the viral target sequence is in a gap region of the rcDNA, and wherein the gap region comprises positions 1 to 1600, 200 to 1600, 300 to 1600, 400 to 1600, 500 to 1600, 600 to 1600, 650 to 1600, 700 to 1600, 750 to 1600, 800 to 1600, 850 to 1600, 900 to 1600, 950 to 1600, 1000 to 1600, 1050 to 1600, 1100 to 1600, 1150 to 1600, 1200 to 1600, 1250 to 1600, 1300 to 1600, 1350 to 1600, 1400 to 1600, 1450 to 1600, 1500 to 1600, 1550 to 1600, or 1580 to 1600 of SEQ ID NO: 1 or a comparable region in SEQ ID NO: 2 or a sequence of any of HBV genotypes A-J.

3-4. (canceled)

5. The oligonucleotide of claim 1, wherein the viral target sequence is in a non-gap region of the rcDNA, and wherein the non-gap region comprises positions 1601 to 3215, 1601 to 3100, 1601 to 2900, 1601 to 2800, 1601 to 2700, 1601 to 2600, 1601 to 2500, 1601 to 2400, 1601 to 2300, 1601 to 2250, 1601 to 2200, 1601 to 2150, 1601 to 2100, 1601 to 2050, 1601 to 2000, 1601 to 1950, 1601 to 1900, 1601 to 1850, 1601 to 1800, 1601 to 1750, or 1601 to 1700 of SEQ ID NO: 1 or a comparable region in SEQ ID NO: 2 or a sequence of any of HBV genotypes A-J.

6-8. (canceled)

9. The oligonucleotide of claim 1, wherein the viral target sequence comprises 5 to 40 nucleotides within the cccDNA selected from positions 950-1050, 975-1025, 975-1010, 980-1010, 1150-1275, 1175-1275, 1180-1270, 1180-1250, 1180-1225, 1180-1210, 1225-1350, 1225-1325, 1225-1300, 1225-1290, 1250-1350, 1250-1325, 1250-1300, 1250-1290, 1375-1475, 1375-1450, 1375-1440, 1375-1430, 1390-1475, 1390-1450, 1390-1440, 1390-1430, 1500-1700, 1500-1650, 1500-1620, 1500-1595, 1510-1700, 1510-1650, 1510-1620, 1510-1595, 1515-1700, 1515-1650, 1515-1620, 1515-1590, and 2817-3050 of SEQ ID NO: 1 or a comparable region in SEQ ID NO: 2 or a sequence of any of HBV genotypes A-J.

10. (canceled)

11. The oligonucleotide of claim 1, wherein the nucleotide sequence is at least identical to 5 to 40 consecutive nucleotides starting at position 1181, 1255, 1256, 1257, 1258, 1259, 1260, 1261, 1263, 1264, 1265, 1266, 1267, 1268, 1393, 1394, 1410, 1411, 1412, 1515, 1516, 1517, 1523, 1527, 1528, 1529, 1530, 1531, 1577, or 2817 of SEQ ID NO: 1 or a comparable position in SEQ ID NO: 2 or a sequence of any of HBV genotypes A-J.

12-13. (canceled)

14. The oligonucleotide of claim 1, wherein the nucleotide sequence is at least complementary to 5 to 40 consecutive nucleotides within positions 950-1050, 975-1025, 975-1010, 980-1010, 1150-1275, 1175-1275, 1180-1270, 1180-1250, 1180-1225, 1180-1210, 1225-1350, 1225-1325, 1225-1300, 1225-1290, 1250-1350, 1250-1325, 1250-1300, 1250-1290, 1375-1475, 1375-1450, 1375-1440, 1375-1430, 1390-1475, 1390-1450, 1390-1440, 1390-1430, 1500-1700, 1500-1650, 1500-1620, 1500-1595, 1510-1700, 1510-1650, 1510-1620, 1510-1595, 1515-1700, 1515-1650, 1515-1620, 1515-1590, or 2817-3050 of SEQ ID NO: 1 or a comparable region in SEQ ID NO: 2 or a sequence of any of HBV genotypes A-J.

15. The oligonucleotide of claim 1, wherein the nucleotide sequence hybridizes under high stringency conditions to 5 to 40 consecutive nucleotides starting at position 1181, 1255, 1256, 1257, 1258, 1259, 1260, 1261, 1263, 1264, 1265, 1266, 1267, 1268, 1393, 1394, 1410, 1411, 1412, 1515, 1516, 1517, 1523, 1527, 1528, 1529, 1530, 1531, 1577, or 2817 of SEQ ID NO: 1 or a comparable region in SEQ ID NO: 2 or a sequence of any of HBV genotypes A-J.

16. The oligonucleotide of claim 1, wherein the nucleotide sequence hybridizes under high stringency conditions to 5 to 40 consecutive nucleotides within positions 950-1050, 975-1025, 975-1010, 980-1010, 1150-1275, 1175-1275, 1180-1270, 1180-1250, 1180-1225, 1180-1210, 1225-1350, 1225-1325, 1225-1300, 1225-1290, 1250-1350, 1250-1325, 1250-1300, 1250-1290, 1375-1475, 1375-1450, 1375-1440, 1375-1430, 1390-1475, 1390-1450, 1390-1440, 1390-1430, 1500-1700, 1500-1650, 1500-1620, 1500-1595, 1510-1700, 1510-1650, 1510-1620, 1510-1595, 1515-1700, 1515-1650, 1515-1620, 1515-1590, or 2817-3050 of SEQ ID NO: 1 or a comparable region in SEQ ID NO: 2 or a sequence of any of HBV genotypes A-J.

17-19. (canceled)

20. The oligonucleotide of claim 1, wherein the viral target sequence comprises 5 to 40 nucleotides within an X region of the rcDNA.

21. The oligonucleotide of claim 20, wherein the viral target sequence comprises 5 to 40 nucleotides within position 1374 to 1603, 1400 to 1603, 1450 to 1603, 1500 to 1603, or 1550 to 1603 of SEQ ID NO: 1 or a comparable region in SEQ ID NO: 2 or a sequence of any of HBV genotypes A-J.

22. (canceled)

23. The oligonucleotide of claim 1, wherein the viral target sequence comprises 5 to 40 nucleotides within an S region of the rcDNA.

24. The oligonucleotide of claim 23, wherein the viral target sequence comprises 5 to 40 nucleotides within position 155 to 1373, 200 to 1373, 300 to 1373, 400 to 1373, 500 to 1373, 600 to 1373, 650 to 1373, 700 to 1373, 750 to 1373, or 800 to 1373 of SEQ ID NO: 1 or a comparable region in SEQ ID NO: 2 or a sequence of any of HBV genotypes A-J.

25. The oligonucleotide of claim 1, wherein the viral target sequence is in the HBV transcript, wherein the HBV transcript is selected from pre-genomic RNA (pgRNA), pre-Core RNA, pre-S1 RNA, pre-S2 RNA and X RNA.

26-27. (canceled)

28. The oligonucleotide of claim 25, wherein the viral target sequence comprises 5 to 40 nucleotides within the pgRNA.

29. (canceled)

30. The oligonucleotide of claim 25, wherein the viral target sequence comprises 5 to 40 nucleotides within the pre-Core RNA.

31. (canceled)

32. The oligonucleotide of claim 25, wherein the viral target sequence comprises 5 to 40 nucleotides within the pre-S1 RNA.

33. (canceled)

34. The oligonucleotide of claim 25, wherein the viral target sequence comprises 5 to 40 nucleotides within the pre-S2 RNA.

35. (canceled)

36. The oligonucleotide of claim 25, wherein the viral target sequence comprises 5 to 40 nucleotides within the X RNA.

37. The oligonucleotide of claim 1, wherein the nucleotide sequence comprises 15 to 25 nucleotides.

38-85. (canceled)

86. The oligonucleotide of claim 1, wherein the nucleotide sequence comprises 15 or 16 nucleotides.

87. The oligonucleotide of claim 86, wherein the oligonucleotide comprises a nucleotide modification pattern of (XY)ₙ, wherein X represents a first class of modified nucleosides, and Y represents a second class of modified nucleosides, wherein X and Y are different, and n is a number between 1 to 8.

88. The oligonucleotide of claim 87, wherein the first class of modified nucleosides is selected from locked nucleosides and 2′-O-methyl nucleosides.

89. The oligonucleotide of claim 87, wherein the second class of modified nucleosides is selected from locked nucleosides and 2′-O-methyl nucleosides and 2′-MOE nucleosides.

90. The oligonucleotide of claim 87, wherein at least 2 consecutive nucleotides in the nucleotide modification pattern comprise at least 2 different nucleobases.

91. The oligonucleotide of claim 87, wherein at least 2 consecutive nucleotides in the nucleotide modification pattern comprise the same nucleobase.

92. The oligonucleotide of claim 1, wherein the nucleotide sequence comprises 20, 21, or 22 nucleotides.

93. The oligonucleotide of claim 92, wherein at least 50%, of the 20, 21, or 22 nucleotides are 2′-O-methyl nucleosides.

94. The oligonucleotide of claim 92, wherein at least 10 of the 20, 21, or 22 nucleotides are 2′-O-methyl nucleosides.

95. The oligonucleotide of claim 1, wherein the oligonucleotide has a melting temperature (Tm) for the complementary viral target sequence of between 50 to 90° C.

96. The oligonucleotide of claim 1, wherein at least 2 and fewer than 25 of the 5 to 40 nucleotides are linked by phosphorothioate linkages.

97. (canceled)

98. The oligonucleotide of claim 1, wherein at least 3% and less than or equal to 100% of the 5 to 40 nucleotides are linked by phosphorothioate linkages.

99. (canceled)

100. The oligonucleotide of claim 1, wherein the oligonucleotide further comprises a tissue targeting conjugate.

101-102. (canceled)

103. The oligonucleotide of claim 100, wherein the tissue targeting conjugate is a galactosamine selected from N-acetylgalactosamine (GalNAc) of Formula (I): 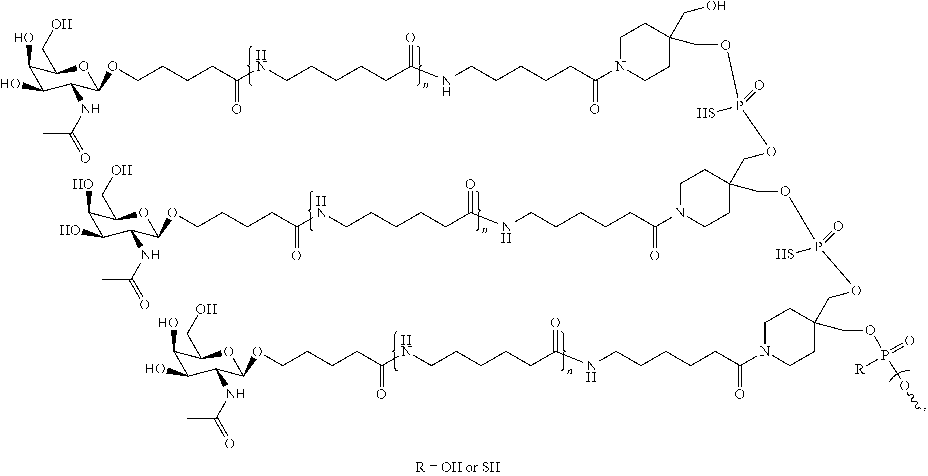
wherein each n is independently 1 or 2; and
N-acetylgalactosamine (GalNAc) of Formula (II): 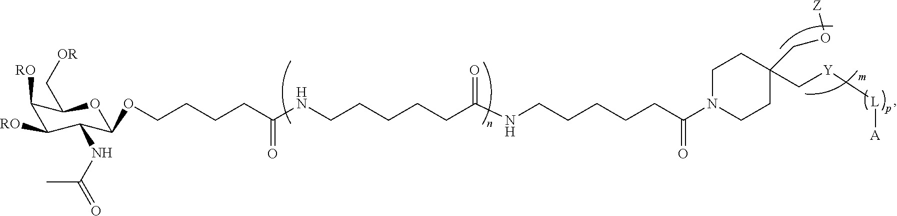
wherein
m is 1, 2, 3, 4, or 5;
each n is independently 1 or 2;
p is 0 or 1;
each R is independently H;
each Y is independently selected from —O—P(═O)(SH)—, —O—P(═O)(O)—, —O—P(═O)(OH)—, and —O—P(S)S—;
Z is H or a second protecting group;
either L is a linker or L and Y in combination are a linker; and
A is H, OH, a third protecting group, an activated group, or an oligonucleotide.

104. (canceled)

105. The oligonucleotide of claim 100, wherein the tissue targeting conjugate is attached to the 3′ end of the nucleotide sequence.

106. The oligonucleotide of claim 100, wherein the tissue targeting conjugate is attached to the 5′ end of the nucleotide sequence.

107. The oligonucleotide of claim 100, wherein the tissue targeting conjugate is attached to the nucleotide sequence via one or more linkages independently selected from a phosphodiester linkage, phosphorothioate linkage, or phosphorodithioate linkage.

108. The oligonucleotide of claim 100, wherein the tissue targeting conjugate is attached to the nucleotide sequence via a linker sequence, wherein the linker sequence comprises 1, 2, 3, 4, 5, 6, 7, 8, 9, 10, 11, 12, 13, 14, or 15 nucleotides.

109. (canceled)

110. The oligonucleotide of claim 109, wherein the tissue targeting conjugate is attached to the linker sequence via one or more linkages independently selected from a phosphodiester linkage, phosphorothioate linkage, or phosphorodithioate linkage.

111. (canceled)

112. The oligonucleotide claim 1, wherein the oligonucleotide does not result in cleavage of the viral target sequence.

113. The oligonucleotide of claim 1, wherein the oligonucleotide reduces conversion of the rcDNA to cccDNA.

114. The oligonucleotide of claim 1, wherein the oligonucleotide reduces the amount of cccDNA.

115. The oligonucleotide of claim 1, wherein the oligonucleotide results in degradation of cccDNA.

116. The oligonucleotide of claim 1, wherein the oligonucleotide reduces the viral titer.

117. The oligonucleotide of claim 1, wherein the oligonucleotide does not induce or activate RNAse H or RNA interference.

118. The oligonucleotide of claim 1, wherein the viral target sequence comprises at least a portion of the HBV genome of any one of HBV genotypes A-J.

119-120. (canceled)

121. The oligonucleotide of claim 1, wherein at least 10 of the 5 to 40 nucleotides is identical, complementary, hybridizes, or binds to the viral target sequence.

122-123. (canceled)

124. A composition comprising: (a) the oligonucleotide of claim 1; and (b) a pharmaceutically acceptable carrier, excipient, diluent, or adjuvant.

125. (canceled)

126. A composition comprising two or more oligonucleotides of claim 1, wherein the two or more oligonucleotides differ by at least one nucleotide.

127. A composition comprising: (a) the oligonucleotide of claim 1; and (b) an anti-HBV drug.

128. (canceled)

129. A composition comprising (a) two or more oligonucleotides of claim 1, wherein the two or more oligonucleotides differ by at least one nucleotide; and (b) an anti-HBV drug.

130-133. (canceled)

134. A kit comprising the oligonucleotide of claim 1.

135. A plasmid comprising the oligonucleotide of claim 1.

136. A viral vector comprising the oligonucleotide of claim 1.

137. A particle comprising the oligonucleotide of claim 1.

138. A method of reducing conversion of hepatitis B virus (HBV) relaxed circular DNA (rcDNA) to covalently closed circular DNA (cccDNA) conversion, comprising contacting a cell with the oligonucleotide of claim 1.

139. A method of targeting hepatitis B virus (HBV) covalently closed circular DNA (cccDNA) for degradation, comprising contacting a cell with the oligonucleotide of claim 1.

140. A method of reducing the amount of hepatitis B virus (HBV) covalently closed circular DNA (cccDNA) in a cell, comprising contacting the cell with the oligonucleotide of claim 1.

141-155. (canceled)

156. A method of treating a hepatitis B virus infection in a subject in need thereof, comprising administering to the subject the oligonucleotide of claim 1.

157-182. (canceled)


SEQUENCE LISTING (271 sequences)

SEQ ID NO: 1 - DNA, 3215 bp, Hepatitis B virus
ctccaccactttccaccaaactcttcaagatcccagagtcagggccctgtactttcctgctggtggctcaagttccggaacagtaaaccctgctccgactactgcctctcccatatcgtcaatcttctcgaggactggggaccctgtaccgaatatggagagcaccacatcaggattcctaggacccctgctcgtgttacaggcggggtttttcttgttgacaagaatcctcacaataccacagagtctagactcgtggtggacttctctcaattttctagggggagcacccacgtgtcctggccaaaatttgcagtccccaacctccaatcactcaccaacctcttgtcctccaatttgtcctggttatcgctggatgtgtctgcggcgttttatcatcttcctcttcatcctgctgctatgcctcatcttcttgttggttcttctggactaccaaggtatgttgcccgtttgtcctctacttccaggaacatcaactaccagcaccggaccatgcaaaacctgcacaactactgctcaagggacctctatgtttccctcatgttgctgtacaaaacctacggacggaaactgcacctgtattcccatcccatcatcttgggctttcgcaaaatacctatgggagtgggcctcagtccgtttctcttggctcagtttactagtgccatttgttcagtggttcgtagggctttcccccactgtctggctttcagttatatggatgatgtggttttgggggccaagtctgtacaacatcttgagtccctttataccgctgttaccaattttcttttatctttgggtatacatttaaaccctcacaaaacaaaaagatggggatattcccttaacttcatgggatatgtaattgggagttggggcactttgcctcaggaacatattgtacaaaaaatcaagcaatgttttaggaaacttcctgtaaacaggcctattgattggaaagtatgtcaacraattgtgggtcttttggggtttgccgcccctttcacgcaatgtggatatcctgctttaatgcctttatatgcatgtatacaagctaagcaggcttttactttctcgccaacttacaaggcctttctgtgtaaacaatatctgaacctttaccccgttgctcggcaacggtcaggtctttgccaagtgtttgctgacgcaacccccactggttggggcttggccataggccatcagcgcatgcgtggaacctttgtggctcctctgccgatccatactgcggaactcctagcagcttgttttgctcgcagccggtctggagcaaaacttatcggcaccgacaactctgttgtcctctctcggaaatacacctcctttccatggctgctaggatgtgctgccaactggatcctgcgcgggacgtcctttgtctacgtcccgtcggcgctgaatcccgcggacgacccatctcggggccgtttgggactctaccgtccccttctgcgtctgccgttccgcccgaccacggggcgcacctctctttacgcggtctccccgtctgtgccttctcatctgccggaccgtgtgcacttcgcttcacctctgcacgtcgcatggagaccaccgtgaacgcccacgggaacctgcccaaggtcttgcataagaggactcttggactttcagcaatgtcaacgaccgaccttgaggcatacttcaaagactgtgtgtttactgagtgggaggagttgggggaggaggttaggttaaaggtctttgtactaggaggctgtaggcataaattggtgtgttcaccagcaccatgcaactttttcacctctgcctaatcatctcatgttcatgtcctactgttcaagcctccaagctgtgccttgggtggctttggggcatggacattgacccgtataaagaatttggagcttctgtggagttactctcttttttgccttctgacttctttccttctattcgagatctcctcgacaccgcctctgctctgtatcgggaggccttagagtctccggaacattgttcacctcaccatacggcactcaggcaagcaattctgtgttggggtgagttaatgaatctagccacctgggtgggaagtaatttggaagatccagcatccagggaattagtagtcagctatgtcaacgttaatatgggcctaaaaatcagacaactattgtggtttcacatttcctgtcttacttttgggagagaaactgttcttgaatatttggtgtcttttggagtgtggattcgcactcctcctgcatatagaccacaaaatgcccctatcttatcaacacttccggaaactactgttgttagacgaagaggcaggtcccctagaagaagaactccctcgcctcgcagacgaaggtctcaatcgccgcgtcgcagaagatctcaatctcgggaatctcaatgttagtattccttggacacataaggtgggaaactttacggggctttattcttctacggtaccttgctttaatcctaaatggcaaactccttcttttcctgacattcatttgcaggaggacattgttgatagatgtaagcaatttgtggggccccttacagtaaatgaaaacaggagacttaaattaattatgcctgctaggttttatcccaatgttactaaatatttgcccttagataaagggatcaaaccgtattatccagagtatgtagttaatcattacttccagacgcgacattatttacacactctttggaaggcggggatcttatataaaagagagtccacacgtagcgcctcattttgcgggtcaccatattcttgggaacaagatctacagcatgggaggttggtcttccaaacctcgaaaaggcatggggacaaatctttctgtccccaatcccctgggattcttccccgatcatcagttggaccctgcattcaaagccaactcagaaaatccagattgggacctcaacccacacaaggacaactggccggacgccaacaaggtgggagtgggagcattcgggccagggttcacccctcctcatgggggactgttggggtggagccctcaggctcagggcatattcacaacagtgccagcagctcctcctcctgcctccaccaatcggcagtcaggaaggcagcctactcccttctctccacctctaagagacactcatcctcaggccatgcagtggaa

SEQ ID NO: 2 - DNA, 3221 bp, Hepatitis B virus
ttccactgccttccaccaagctctgcaggatcccaaagtcaggggtctgtattttcctgctggtggctccagttcaggaacagtaaaccctgctccgaatattgcctctcacatctcgtcaatctccgcgaggactggggaccctgtgacgaatatggagaacatcacatcaggattcctaggacccctgctcgtgttacaggcggggtttttcttgttgacaagaatcctcacaataccgcagagtctagactcgtggtggacttctctcaattttctagggggatcacccgtgtgtcttggccaaaattcgcagtccccaacctccaatcactcaccaacctcctgtcctccaatttgtcctggttatcgctggatgtgtctgcggcgttttatcatattcctcttcatcctgctgctatgcctcatcttcttgttggttcttctggattatcaaggtatgttgcccgtttgtcctctaattccaggaacaacaacaaccagtacgggaccatgcaaaacctgcacgactcctgctcaaggcaactctatgtttccctcatgttgctgtacaaaaccttcggatggaaattgcacctgtattcccatcccatcgtcttgggctttcgcaaaatacctatgggagtgggcctcagtccgtttctcttggctcagtttactagtgccatttgttcagtggttcgtagggctttcccccactgtttggctttcagctatatggatgatgtggtattgggggccaagtctgtacagcatcgtgagtccctttataccgctgttaccaattttcttttgtctctgggtatacatttaaaccctaacaaaacaaaaagatggggttattccctaaacttcatgggttacataattggaagttggggaacgttgccacaggatcatattgtacaaaagatcaaacactgttttagaaaacttcctgttaacaggcctattgattggaaagtatgtcaaagaattgtgggtcttttgggctttgctgctccatttacacaatgtggatatcctgccttaatgcctttgtatgcctgtatacaagctaaacaggctttcactttctcgccaacttacaaggcctttctaagtaaacagtacatgaacctttaccccgttgctcggcaacggcctggtctgtgccaagtgtttgctgacgcaacccccactggctggggcttggccataggccatcagcgcatgcgtggaacctttgtggctcctctgccgatccatactgcggaactcctagccgcttgttttgctcgcagccggtctggggcaaagctcatcggaactgacaattctgtcgtcctctcgcggaaatatacatcgtttccatggctgctaggttgtactgccaactggatccttcgcgggacgtcctttgtttacgtcccgtcggcgctgaatcccgcggacgacccctctcggggccgcttgggactctctcgtccccttctccgtctgccgttccagccgaccacggggcgcacctctctttacgcggtctccccgtctgtgccttctcatctgccggtccgtgtgcacttcgcttcacctctgcacgttgcatggagaccaccgtgaacgcccatcagatcctgcccaaggtcttacataagaggactcttggactcccagcaatgtcaacgaccgaccttgaggcctacttcaaagactgtgtgtttaaggactgggaggagctgggggaggagattaggttaaaggtctttgtattaggaggctgtaggcataaattggtctgcgcaccagcaccatgcaactttttcacctctgcctaatcatctcttgtacatgtcccactgttcaagcctccaagctgtgccttgggtggctttggggcatggacattgacccttataaagaatttggagctactgtggagttactctcgtttttgccttctgacttttttccttccgtcagagatctcctagacaccgcctcagctctgtatcgggaagccttagagtctcctgagcattgctcacctcaccatactgcactcaggcaagcaattctctgctggggggaattgatgactctagctacctgggtgggtaataatttggaagatccagcatccagggatctagtagtcaattatgttaatactaacatgggtttaaagatcaggcaactattgtggtttcatatatcttgccttacttttggaagagagactgtacttgaatatttggtctctttcggagtgtggattcgcactcctccagcctatagaccaccaaatgcccctatcttatcaacacttccggaaactactgttgttagacgacgggaccgaggcaggtcccctagaagaagaactccctcgcctcgcagacgcagatctcaatcgccgcgtcgcagaagatctcaatctcgggaatctcaatgttagtattccttggactcataaggtgggaaactttactgggctttattcctctacagtacctatctttaatcctgaatggcaaactccttcctttcctaagattcatttacaagaggacattattaataggtgtcaacaatttgtgggccctctcactgtaaatgaaaagagaagattgaaattaattatgcctgctagattctatcctacccacactaaatatttgcccttagacaaaggaattaaaccttattatccagatcaggtagttaatcattacttcaaaaccagacattatttacatactctttggaaggctggtattctatataagagggaaaccacacgtagcgcatcattttgcgggtcaccatattcttgggaacaagagctacagcatgggaggttggtcatcgaaacctcgcaaaggcatggggacgaatctttctgttcccaaccctctgggattctttcccgatcatcagttggaccctgcattcggagccaactcaaacaatccagattgggacttcaaccccatcaaggaccactggccagcagccaaccaggtaggagtgggagcattcgggccagggttcacccctccacacggcggtgttttggggtggagccctcaggctcagggcatattgaccacagtgtcaacaattcctcctcctgcctccaccaatcggcagtcaggaaggcagcctactcccatctctccacctctaagagacagtcatcctcaggccatgcagtggaa

SEQ ID NO: 3 - RNA, 22 nt, Artificial Sequence
  Description of Artificial Sequence Synthetic oligonucleotide
ccgaccacggggcgcaccucuc

SEQ ID NO: 4 - RNA, 22 nt, Artificial Sequence
  Description of Artificial Sequence Synthetic oligonucleotide
cgaccacggggcgcaccucucu

SEQ ID NO: 5 - RNA, 21 nt, Artificial Sequence
  Description of Artificial Sequence Synthetic oligonucleotide
cgaccacggggcgcaccucuc

SEQ ID NO: 6 - RNA, 21 nt, Artificial Sequence
  Description of Artificial Sequence Synthetic oligonucleotide
gaccacggggcgcaccucucu

SEQ ID NO: 7 - RNA, 22 nt, Artificial Sequence
  Description of Artificial Sequence Synthetic oligonucleotide
ggggcgcaccucucuuuacgcg

SEQ ID NO: 8 - RNA, 22 nt, Artificial Sequence
  Description of Artificial Sequence Synthetic oligonucleotide
ccgaccacggggcgcaccucuc

SEQ ID NO: 9 - RNA, 22 nt, Artificial Sequence
  Description of Artificial Sequence Synthetic oligonucleotide
cgaccacggggcgcaccucucu

SEQ ID NO: 10 - RNA, 21 nt, Artificial Sequence
  Description of Artificial Sequence Synthetic oligonucleotide
cgaccacggggcgcaccucuc

SEQ ID NO: 11 - RNA, 21 nt, Artificial Sequence
  Description of Artificial Sequence Synthetic oligonucleotide
gaccacggggcgcaccucucu

SEQ ID NO: 12 - RNA, 22 nt, Artificial Sequence
  Description of Artificial Sequence Synthetic oligonucleotide
ggggcgcaccucucuuuacgcg

SEQ ID NO: 13 - DNA, 16 bp, Artificial Sequence
  Description of Artificial Sequence Synthetic oligonucleotide
cgcacctctctttacg

SEQ ID NO: 14 - DNA, 15 bp, Artificial Sequence
  Description of Artificial Sequence Synthetic oligonucleotide
gcacctctctttacg

SEQ ID NO: 15 - DNA, 16 bp, Artificial Sequence
  Description of Artificial Sequence Synthetic oligonucleotide
gcacctctctttacgc

SEQ ID NO: 16 - DNA, 15 bp, Artificial Sequence
  Description of Artificial Sequence Synthetic oligonucleotide
cgcacctctctttac

SEQ ID NO: 17 - DNA, 15 bp, Artificial Sequence
  Description of Artificial Sequence Synthetic oligonucleotide
cgggacgtcctttgt

SEQ ID NO: 18 - DNA, 15 bp, Artificial Sequence
  Description of Artificial Sequence Synthetic oligonucleotide
ccgtgtgcacttcgc

SEQ ID NO: 19 - DNA, 15 bp, Artificial Sequence
  Description of Artificial Sequence Synthetic oligonucleotide
cctctctttacgcgg

SEQ ID NO: 20 - DNA, 15 bp, Artificial Sequence
  Description of Artificial Sequence Synthetic oligonucleotide
acctctctttacgcg

SEQ ID NO: 21 - DNA, 15 bp, Artificial Sequence
  Description of Artificial Sequence Synthetic oligonucleotide
cacctctctttacgc

SEQ ID NO: 22 - DNA, 17 bp, Artificial Sequence
  Description of Artificial Sequence Synthetic oligonucleotide
cacctctctttacgcgg

SEQ ID NO: 23 - DNA, 15 bp, Artificial Sequence
  Description of Artificial Sequence Synthetic oligonucleotide
gcacctctctttacg

SEQ ID NO: 24 - DNA, 15 bp, Artificial Sequence
  Description of Artificial Sequence Synthetic oligonucleotide
cgcacctctctttac

SEQ ID NO: 25 - DNA, 17 bp, Artificial Sequence
  Description of Artificial Sequence Synthetic oligonucleotide
cgcacctctctttacgc

SEQ ID NO: 26 - DNA, 18 bp, Artificial Sequence
  Description of Artificial Sequence Synthetic oligonucleotide
cgcacctctctttacgcg

SEQ ID NO: 27 - DNA, 15 bp, Artificial Sequence
  Description of Artificial Sequence Synthetic oligonucleotide
cgggacgtcctttgt

SEQ ID NO: 28 - DNA, 15 bp, Artificial Sequence
  Description of Artificial Sequence Synthetic oligonucleotide
gcgggacgtcctttg

SEQ ID NO: 29 - DNA, 16 bp, Artificial Sequence
  Description of Artificial Sequence Synthetic oligonucleotide
gcgggacgtcctttgt

SEQ ID NO: 30 - DNA, 17 bp, Artificial Sequence
  Description of Artificial Sequence Synthetic oligonucleotide
cgcgggacgtcctttgt

SEQ ID NO: 31 - DNA, 15 bp, Artificial Sequence
  Description of Artificial Sequence Synthetic oligonucleotide
tgccaactggatcct

SEQ ID NO: 32 - DNA, 15 bp, Artificial Sequence
  Description of Artificial Sequence Synthetic oligonucleotide
ctgccaactggatcc

SEQ ID NO: 33 - DNA, 16 bp, Artificial Sequence
  Description of Artificial Sequence Synthetic oligonucleotide
ctgccaactggatcct

SEQ ID NO: 34 - DNA, 15 bp, Artificial Sequence
  Description of Artificial Sequence Synthetic oligonucleotide
ccatactgcggaact

SEQ ID NO: 35 - DNA, 16 bp, Artificial Sequence
  Description of Artificial Sequence Synthetic oligonucleotide
tccatactgcggaact

SEQ ID NO: 36 - DNA, 15 bp, Artificial Sequence
  Description of Artificial Sequence Synthetic oligonucleotide
atccatactgcggaa

SEQ ID NO: 37 - DNA, 17 bp, Artificial Sequence
  Description of Artificial Sequence Synthetic oligonucleotide
atccatactgcggaact

SEQ ID NO: 38 - DNA, 16 bp, Artificial Sequence
  Description of Artificial Sequence Synthetic oligonucleotide
gatccatactgcggaa

SEQ ID NO: 39 - DNA, 15 bp, Artificial Sequence
  Description of Artificial Sequence Synthetic oligonucleotide
cgatccatactgcgg

SEQ ID NO: 40 - DNA, 15 bp, Artificial Sequence
  Description of Artificial Sequence Synthetic oligonucleotide
ccgatccatactgcg

SEQ ID NO: 41 - DNA, 16 bp, Artificial Sequence
  Description of Artificial Sequence Synthetic oligonucleotide
ccgatccatactgcgg

SEQ ID NO: 42 - DNA, 15 bp, Artificial Sequence
  Description of Artificial Sequence Synthetic oligonucleotide
ctgccgatccatact

SEQ ID NO: 43 - DNA, 16 bp, Artificial Sequence
  Description of Artificial Sequence Synthetic oligonucleotide
ctgccgatccatactg

SEQ ID NO: 44 - DNA, 15 bp, Artificial Sequence
  Description of Artificial Sequence Synthetic oligonucleotide
tctgccgatccatac

SEQ ID NO: 45 - DNA, 16 bp, Artificial Sequence
  Description of Artificial Sequence Synthetic oligonucleotide
tctgccgatccatact

SEQ ID NO: 46 - DNA, 16 bp, Artificial Sequence
  Description of Artificial Sequence Synthetic oligonucleotide
ctctgccgatccatac

SEQ ID NO: 47 - DNA, 17 bp, Artificial Sequence
  Description of Artificial Sequence Synthetic oligonucleotide
ctctgccgatccatact

SEQ ID NO: 48 - DNA, 15 bp, Artificial Sequence
  Description of Artificial Sequence Synthetic oligonucleotide
tcctctgccgatcca

SEQ ID NO: 49 - DNA, 16 bp, Artificial Sequence
  Description of Artificial Sequence Synthetic oligonucleotide
tcctctgccgatccat

SEQ ID NO: 50 - DNA, 15 bp, Artificial Sequence
  Description of Artificial Sequence Synthetic oligonucleotide
agtgtttgctgacgc

SEQ ID NO: 51 - DNA, 15 bp, Artificial Sequence
  Description of Artificial Sequence Synthetic oligonucleotide
acctctctttacgcg

SEQ ID NO: 52 - DNA, 15 bp, Artificial Sequence
  Description of Artificial Sequence Synthetic oligonucleotide
cacctctctttacgc

SEQ ID NO: 53 - DNA, 15 bp, Artificial Sequence
  Description of Artificial Sequence Synthetic oligonucleotide
agtgtttgctgacgc

SEQ ID NO: 54 - DNA, 16 bp, Artificial Sequence
  Description of Artificial Sequence Synthetic oligonucleotide
acctctctttacgcgg

SEQ ID NO: 55 - DNA, 16 bp, Artificial Sequence
  Description of Artificial Sequence Synthetic oligonucleotide
cacctctctttacgcg

SEQ ID NO: 56 - DNA, 16 bp, Artificial Sequence
  Description of Artificial Sequence Synthetic oligonucleotide
gcacctctctttacgc

SEQ ID NO: 57 - DNA, 17 bp, Artificial Sequence
  Description of Artificial Sequence Synthetic oligonucleotide
gcacctctctttacgcg

SEQ ID NO: 58 - DNA, 16 bp, Artificial Sequence
  Description of Artificial Sequence Synthetic oligonucleotide
cgcgggacgtcctttg

SEQ ID NO: 59 - DNA, 17 bp, Artificial Sequence
  Description of Artificial Sequence Synthetic oligonucleotide
cgatccatactgcggaa

SEQ ID NO: 60 - DNA, 15 bp, Artificial Sequence
  Description of Artificial Sequence Synthetic oligonucleotide
tgccgatccatactg

SEQ ID NO: 61 - DNA, 17 bp, Artificial Sequence
  Description of Artificial Sequence Synthetic oligonucleotide
tctgccgatccatactg

SEQ ID NO: 62 - DNA, 15 bp, Artificial Sequence
  Description of Artificial Sequence Synthetic oligonucleotide
cctctgccgatccat

SEQ ID NO: 63 - DNA, 15 bp, Artificial Sequence
  Description of Artificial Sequence Synthetic oligonucleotide
ctcctctgccgatcc

SEQ ID NO: 64 - DNA, 16 bp, Artificial Sequence
  Description of Artificial Sequence Synthetic oligonucleotide
ctcctctgccgatcca

SEQ ID NO: 65 - RNA, 22 nt, Artificial Sequence
  Description of Artificial Sequence Synthetic oligonucleotide
ccgaccacggggcgcaccucuc

SEQ ID NO: 66 - RNA, 22 nt, Artificial Sequence
  Description of Artificial Sequence Synthetic oligonucleotide
cgaccacggggcgcaccucucu

SEQ ID NO: 67 - RNA, 21 nt, Artificial Sequence
  Description of Artificial Sequence Synthetic oligonucleotide
cgaccacggggcgcaccucuc

SEQ ID NO: 68 - RNA, 21 nt, Artificial Sequence
  Description of Artificial Sequence Synthetic oligonucleotide
gaccacggggcgcaccucucu

SEQ ID NO: 69 - RNA, 22 nt, Artificial Sequence
  Description of Artificial Sequence Synthetic oligonucleotide
ggggcgcaccucucuuuacgcg

SEQ ID NO: 70 - RNA, 22 nt, Artificial Sequence
  Description of Artificial Sequence Synthetic oligonucleotide
ccgaccacggggcgcaccucuc

SEQ ID NO: 71 - DNA, 22 bp, Artificial Sequence
  Description of Artificial Sequence Synthetic oligonucleotide
  Description of Combined DNA/RNA Molecule Synthetic oligonucleotide
cgaccacggggcgcacctctct

SEQ ID NO: 72 - DNA, 21 bp, Artificial Sequence
  Description of Artificial Sequence Synthetic oligonucleotide
  Description of Combined DNA/RNA Molecule Synthetic oligonucleotide
gaccacggggcgcaccucuct

SEQ ID NO: 73 - DNA, 21 bp, Artificial Sequence
  Description of Artificial Sequence Synthetic oligonucleotide
  Description of Combined DNA/RNA Molecule Synthetic oligonucleotide
gaccacggggcgcaccucuct

SEQ ID NO: 74 - DNA, 22 bp, Artificial Sequence
  Description of Artificial Sequence Synthetic oligonucleotide
  Description of Combined DNA/RNA Molecule Synthetic oligonucleotide
ggggcgcaccucucutuacgcg

SEQ ID NO: 75 - DNA, 16 bp, Artificial Sequence
  Description of Artificial Sequence Synthetic oligonucleotide
  Description of Combined DNA/RNA Molecule Synthetic oligonucleotide
cgcacctctctutacg

SEQ ID NO: 76 - DNA, 15 bp, Artificial Sequence
  Description of Artificial Sequence Synthetic oligonucleotide
  Description of Combined DNA/RNA Molecule Synthetic oligonucleotide
gcacctctctttacg

SEQ ID NO: 77 - DNA, 16 bp, Artificial Sequence
  Description of Artificial Sequence Synthetic oligonucleotide
  Description of Combined DNA/RNA Molecule Synthetic oligonucleotide
gcacctctctttacgc

SEQ ID NO: 78 - DNA, 15 bp, Artificial Sequence
  Description of Artificial Sequence Synthetic oligonucleotide
  Description of Combined DNA/RNA Molecule Synthetic oligonucleotide
cgcacctctctttac

SEQ ID NO: 79 - DNA, 15 bp, Artificial Sequence
  Description of Artificial Sequence Synthetic oligonucleotide
  Description of Combined DNA/RNA Molecule Synthetic oligonucleotide
cgggacgtcctttgt

SEQ ID NO: 80 - DNA, 15 bp, Artificial Sequence
  Description of Artificial Sequence Synthetic oligonucleotide
  Description of Combined DNA/RNA Molecule Synthetic oligonucleotide
ccgugugcactucgc

SEQ ID NO: 81 - DNA, 15 bp, Artificial Sequence
  Description of Artificial Sequence Synthetic oligonucleotide
  Description of Combined DNA/RNA Molecule Synthetic oligonucleotide
cctctctutacgcgg

SEQ ID NO: 82 - DNA, 15 bp, Artificial Sequence
  Description of Artificial Sequence Synthetic oligonucleotide
  Description of Combined DNA/RNA Molecule Synthetic oligonucleotide
accucucutuacgcg

SEQ ID NO: 83 - DNA, 15 bp, Artificial Sequence
  Description of Artificial Sequence Synthetic oligonucleotide
  Description of Combined DNA/RNA Molecule Synthetic oligonucleotide
cacctctctutacgc

SEQ ID NO: 84 - DNA, 17 bp, Artificial Sequence
  Description of Artificial Sequence Synthetic oligonucleotide
  Description of Combined DNA/RNA Molecule Synthetic oligonucleotide
cacctctctutacgcgg

SEQ ID NO: 85 - DNA, 15 bp, Artificial Sequence
  Description of Artificial Sequence Synthetic oligonucleotide
  Description of Combined DNA/RNA Molecule Synthetic oligonucleotide
gcaccucucutuacg

SEQ ID NO: 86 - DNA, 15 bp, Artificial Sequence
  Description of Artificial Sequence Synthetic oligonucleotide
  Description of Combined DNA/RNA Molecule Synthetic oligonucleotide
cgcacctctctutac

SEQ ID NO: 87 - DNA, 17 bp, Artificial Sequence
  Description of Artificial Sequence Synthetic oligonucleotide
  Description of Combined DNA/RNA Molecule Synthetic oligonucleotide
cgcacctctctutacgc

SEQ ID NO: 88 - DNA, 18 bp, Artificial Sequence
  Description of Artificial Sequence Synthetic oligonucleotide
  Description of Combined DNA/RNA Molecule Synthetic oligonucleotide
cgcacctctctutacgcg

SEQ ID NO: 89 - DNA, 15 bp, Artificial Sequence
  Description of Artificial Sequence Synthetic oligonucleotide
  Description of Combined DNA/RNA Molecule Synthetic oligonucleotide
cgggacgucctutgt

SEQ ID NO: 90 - DNA, 15 bp, Artificial Sequence
  Description of Artificial Sequence Synthetic oligonucleotide
  Description of Combined DNA/RNA Molecule Synthetic oligonucleotide
gcgggacgtccutug

SEQ ID NO: 91 - DNA, 16 bp, Artificial Sequence
  Description of Artificial Sequence Synthetic oligonucleotide
  Description of Combined DNA/RNA Molecule Synthetic oligonucleotide
gcgggacgtccutugu

SEQ ID NO: 92 - DNA, 17 bp, Artificial Sequence
  Description of Artificial Sequence Synthetic oligonucleotide
  Description of Combined DNA/RNA Molecule Synthetic oligonucleotide
cgcgggacgucctutgt

SEQ ID NO: 93 - DNA, 15 bp, Artificial Sequence
  Description of Artificial Sequence Synthetic oligonucleotide
  Description of Combined DNA/RNA Molecule Synthetic oligonucleotide
tgccaacuggaucct

SEQ ID NO: 94 - DNA, 15 bp, Artificial Sequence
  Description of Artificial Sequence Synthetic oligonucleotide
  Description of Combined DNA/RNA Molecule Synthetic oligonucleotide
cugccaactggatcc

SEQ ID NO: 95 - DNA, 16 bp, Artificial Sequence
  Description of Artificial Sequence Synthetic oligonucleotide
  Description of Combined DNA/RNA Molecule Synthetic oligonucleotide
cugccaactggatccu

SEQ ID NO: 96 - DNA, 15 bp, Artificial Sequence
  Description of Artificial Sequence Synthetic oligonucleotide
  Description of Combined DNA/RNA Molecule Synthetic oligonucleotide
ccauactgcggaact

SEQ ID NO: 97 - DNA, 16 bp, Artificial Sequence
  Description of Artificial Sequence Synthetic oligonucleotide
  Description of Combined DNA/RNA Molecule Synthetic oligonucleotide
tccatacugcggaacu

SEQ ID NO: 98 - DNA, 15 bp, Artificial Sequence
  Description of Artificial Sequence Synthetic oligonucleotide
  Description of Combined DNA/RNA Molecule Synthetic oligonucleotide
auccauactgcggaa

SEQ ID NO: 99 - DNA, 17 bp, Artificial Sequence
  Description of Artificial Sequence Synthetic oligonucleotide
  Description of Combined DNA/RNA Molecule Synthetic oligonucleotide
auccauactgcggaact

SEQ ID NO: 100 - DNA, 16 bp, Artificial Sequence
  Description of Artificial Sequence Synthetic oligonucleotide
  Description of Combined DNA/RNA Molecule Synthetic oligonucleotide
gatccatacugcggaa

SEQ ID NO: 101 - DNA, 15 bp, Artificial Sequence
  Description of Artificial Sequence Synthetic oligonucleotide
  Description of Combined DNA/RNA Molecule Synthetic oligonucleotide
cgauccauactgcgg

SEQ ID NO: 102 - DNA, 15 bp, Artificial Sequence
  Description of Artificial Sequence Synthetic oligonucleotide
  Description of Combined DNA/RNA Molecule Synthetic oligonucleotide
ccgatccatacugcg

SEQ ID NO: 103 - DNA, 16 bp, Artificial Sequence
  Description of Artificial Sequence Synthetic oligonucleotide
  Description of Combined DNA/RNA Molecule Synthetic oligonucleotide
ccgatccatacugcgg

SEQ ID NO: 104 - DNA, 15 bp, Artificial Sequence
  Description of Artificial Sequence Synthetic oligonucleotide
  Description of Combined DNA/RNA Molecule Synthetic oligonucleotide
cugccgauccauact

SEQ ID NO: 105 - DNA, 16 bp, Artificial Sequence
  Description of Artificial Sequence Synthetic oligonucleotide
  Description of Combined DNA/RNA Molecule Synthetic oligonucleotide
cugccgauccauactg

SEQ ID NO: 106 - DNA, 15 bp, Artificial Sequence
  Description of Artificial Sequence Synthetic oligonucleotide
  Description of Combined DNA/RNA Molecule Synthetic oligonucleotide
tctgccgatccatac

SEQ ID NO: 107 - DNA, 16 bp, Artificial Sequence
  Description of Artificial Sequence Synthetic oligonucleotide
  Description of Combined DNA/RNA Molecule Synthetic oligonucleotide
tctgccgatccatacu

SEQ ID NO: 108 - RNA, 16 nt, Artificial Sequence
  Description of Artificial Sequence Synthetic oligonucleotide
cucugccgauccauac

SEQ ID NO: 109 - DNA, 17 bp, Artificial Sequence
  Description of Artificial Sequence Synthetic oligonucleotide
  Description of Combined DNA/RNA Molecule Synthetic oligonucleotide
cucugccgauccauact

SEQ ID NO: 110 - DNA, 15 bp, Artificial Sequence
  Description of Artificial Sequence Synthetic oligonucleotide
  Description of Combined DNA/RNA Molecule Synthetic oligonucleotide
tccucugccgaucca

SEQ ID NO: 111 - DNA, 16 bp, Artificial Sequence
  Description of Artificial Sequence Synthetic oligonucleotide
  Description of Combined DNA/RNA Molecule Synthetic oligonucleotide
tccucugccgauccau

SEQ ID NO: 112 - DNA, 15 bp, Artificial Sequence
  Description of Artificial Sequence Synthetic oligonucleotide
  Description of Combined DNA/RNA Molecule Synthetic oligonucleotide
agtgtutgcugacgc

SEQ ID NO: 113 - DNA, 15 bp, Artificial Sequence
  Description of Artificial Sequence Synthetic oligonucleotide
  Description of Combined DNA/RNA Molecule Synthetic oligonucleotide
acctctctttacgcg

SEQ ID NO: 114 - DNA, 15 bp, Artificial Sequence
  Description of Artificial Sequence Synthetic oligonucleotide
  Description of Combined DNA/RNA Molecule Synthetic oligonucleotide
cacctctctttacgc

SEQ ID NO: 115 - DNA, 15 bp, Artificial Sequence
  Description of Artificial Sequence Synthetic oligonucleotide
  Description of Combined DNA/RNA Molecule Synthetic oligonucleotide
agtgtttgctgacgc

SEQ ID NO: 116 - DNA, 16 bp, Artificial Sequence
  Description of Artificial Sequence Synthetic oligonucleotide
  Description of Combined DNA/RNA Molecule Synthetic oligonucleotide
accucucutuacgcgg

SEQ ID NO: 117 - DNA, 16 bp, Artificial Sequence
  Description of Artificial Sequence Synthetic oligonucleotide
  Description of Combined DNA/RNA Molecule Synthetic oligonucleotide
cacctctctutacgcg

SEQ ID NO: 118 - DNA, 16 bp, Artificial Sequence
  Description of Artificial Sequence Synthetic oligonucleotide
  Description of Combined DNA/RNA Molecule Synthetic oligonucleotide
gcaccucucutuacgc

SEQ ID NO: 119 - DNA, 17 bp, Artificial Sequence
  Description of Artificial Sequence Synthetic oligonucleotide
  Description of Combined DNA/RNA Molecule Synthetic oligonucleotide
gcaccucucutuacgcg

SEQ ID NO: 120 - DNA, 16 bp, Artificial Sequence
  Description of Artificial Sequence Synthetic oligonucleotide
  Description of Combined DNA/RNA Molecule Synthetic oligonucleotide
cgcgggacgucctutg

SEQ ID NO: 121 - DNA, 17 bp, Artificial Sequence
  Description of Artificial Sequence Synthetic oligonucleotide
  Description of Combined DNA/RNA Molecule Synthetic oligonucleotide
cgauccauactgcggaa

SEQ ID NO: 122 - DNA, 15 bp, Artificial Sequence
  Description of Artificial Sequence Synthetic oligonucleotide
  Description of Combined DNA/RNA Molecule Synthetic oligonucleotide
tgccgatccatacug

SEQ ID NO: 123 - DNA, 17 bp, Artificial Sequence
  Description of Artificial Sequence Synthetic oligonucleotide
  Description of Combined DNA/RNA Molecule Synthetic oligonucleotide
tctgccgatccatacug

SEQ ID NO: 124 - DNA, 15 bp, Artificial Sequence
  Description of Artificial Sequence Synthetic oligonucleotide
  Description of Combined DNA/RNA Molecule Synthetic oligonucleotide
cctctgccgatccat

SEQ ID NO: 125 - DNA, 15 bp, Artificial Sequence
  Description of Artificial Sequence Synthetic oligonucleotide
  Description of Combined DNA/RNA Molecule Synthetic oligonucleotide
cucctctgccgatcc

SEQ ID NO: 126 - DNA, 16 bp, Artificial Sequence
  Description of Artificial Sequence Synthetic oligonucleotide
  Description of Combined DNA/RNA Molecule Synthetic oligonucleotide
cucctctgccgatcca

SEQ ID NO: 127 - DNA, 16 bp, Artificial Sequence
  Description of Artificial Sequence Synthetic oligonucleotide
  Description of Combined DNA/RNA Molecule Synthetic oligonucleotide
gatccatactgcggaa

SEQ ID NO: 128 - RNA, 16 nt, Artificial Sequence
  Description of Artificial Sequence Synthetic oligonucleotide
gauccauacugcggaa

SEQ ID NO: 129 - RNA, 16 nt, Artificial Sequence
  Description of Artificial Sequence Synthetic oligonucleotide
gauccauacugcggaa

SEQ ID NO: 130 - DNA, 16 bp, Artificial Sequence
  Description of Artificial Sequence Synthetic oligonucleotide
  Description of Combined DNA/RNA Molecule Synthetic oligonucleotide
gatccatactgcggaa

SEQ ID NO: 131 - DNA, 16 bp, Artificial Sequence
  Description of Artificial Sequence Synthetic oligonucleotide
  Description of Combined DNA/RNA Molecule Synthetic oligonucleotide
gatccauacugcggaa

SEQ ID NO: 132 - DNA, 16 bp, Artificial Sequence
  Description of Artificial Sequence Synthetic oligonucleotide
  Description of Combined DNA/RNA Molecule Synthetic oligonucleotide
gatccatactgcggaa

SEQ ID NO: 133 - RNA, 15 nt, Artificial Sequence
  Description of Artificial Sequence Synthetic oligonucleotide
aguguuugcugacgc

SEQ ID NO: 134 - RNA, 15 nt, Artificial Sequence
  Description of Artificial Sequence Synthetic oligonucleotide
aguguuugcugacgc

SEQ ID NO: 135 - DNA, 15 bp, Artificial Sequence
  Description of Artificial Sequence Synthetic oligonucleotide
  Description of Combined DNA/RNA Molecule Synthetic oligonucleotide
agtgtttgctgacgc

SEQ ID NO: 136 - DNA, 15 bp, Artificial Sequence
  Description of Artificial Sequence Synthetic oligonucleotide
  Description of Combined DNA/RNA Molecule Synthetic oligonucleotide
agtgtutgcugacgc

SEQ ID NO: 137 - DNA, 15 bp, Artificial Sequence
  Description of Artificial Sequence Synthetic oligonucleotide
  Description of Combined DNA/RNA Molecule Synthetic oligonucleotide
agtguuugcugacgc

SEQ ID NO: 138 - DNA, 15 bp, Artificial Sequence
  Description of Artificial Sequence Synthetic oligonucleotide
  Description of Combined DNA/RNA Molecule Synthetic oligonucleotide
agtgtttgctgacgc

SEQ ID NO: 139 - DNA, 15 bp, Artificial Sequence
  Description of Artificial Sequence Synthetic oligonucleotide
  Description of Combined DNA/RNA Molecule Synthetic oligonucleotide
agugutugctgacgc

SEQ ID NO: 140 - RNA, 16 nt, Artificial Sequence
  Description of Artificial Sequence Synthetic oligonucleotide
ccgaccacggggcgca

SEQ ID NO: 141 - RNA, 16 nt, Artificial Sequence
  Description of Artificial Sequence Synthetic oligonucleotide
cgaccacggggcgcac

SEQ ID NO: 142 - DNA, 16 bp, Artificial Sequence
  Description of Artificial Sequence Synthetic oligonucleotide
  Description of Combined DNA/RNA Molecule Synthetic oligonucleotide
acggggcgcacctctc

SEQ ID NO: 143 - RNA, 16 nt, Artificial Sequence
  Description of Artificial Sequence Synthetic oligonucleotide
gaccacggggcgcacc

SEQ ID NO: 144 - DNA, 16 bp, Artificial Sequence
  Description of Artificial Sequence Synthetic oligonucleotide
  Description of Combined DNA/RNA Molecule Synthetic oligonucleotide
ggggcgcacctctctu

SEQ ID NO: 145 - DNA, 16 bp, Artificial Sequence
  Description of Artificial Sequence Synthetic oligonucleotide
  Description of Combined DNA/RNA Molecule Synthetic oligonucleotide
ggggcgcacctctctt

SEQ ID NO: 146 - RNA, 18 nt, Artificial Sequence
  Description of Artificial Sequence Synthetic oligonucleotide
ccgaccacggggcgcacc

SEQ ID NO: 147 - DNA, 19 bp, Artificial Sequence
  Description of Artificial Sequence Synthetic oligonucleotide
  Description of Combined DNA/RNA Molecule Synthetic oligonucleotide
cgaccacggggcgcaccct

SEQ ID NO: 148 - DNA, 18 bp, Artificial Sequence
  Description of Artificial Sequence Synthetic oligonucleotide
  Description of Combined DNA/RNA Molecule Synthetic oligonucleotide
ccacggggcgcacctctc

SEQ ID NO: 149 - RNA, 19 nt, Artificial Sequence
  Description of Artificial Sequence Synthetic oligonucleotide
gaccacggggcgcacccuc

SEQ ID NO: 150 - DNA, 18 bp, Artificial Sequence
  Description of Artificial Sequence Synthetic oligonucleotide
  Description of Combined DNA/RNA Molecule Synthetic oligonucleotide
ggggcgcacctctctuta

SEQ ID NO: 151 - DNA, 17 bp, Artificial Sequence
  Description of Artificial Sequence Synthetic oligonucleotide
  Description of Combined DNA/RNA Molecule Synthetic oligonucleotide
ggggcgcacctctctut

SEQ ID NO: 152 - RNA, 15 nt, Artificial Sequence
  Description of Artificial Sequence Synthetic oligonucleotide
cgggacguccuuugu

SEQ ID NO: 153 - DNA, 15 bp, Artificial Sequence
  Description of Artificial Sequence Synthetic oligonucleotide
  Description of Combined DNA/RNA Molecule Synthetic oligonucleotide
cgggacgtcctttgt

SEQ ID NO: 154 - DNA, 15 bp, Artificial Sequence
  Description of Artificial Sequence Synthetic oligonucleotide
  Description of Combined DNA/RNA Molecule Synthetic oligonucleotide
cgggacgtcctttgt

SEQ ID NO: 155 - DNA, 17 bp, Artificial Sequence
  Description of Artificial Sequence Synthetic oligonucleotide
  Description of Combined DNA/RNA Molecule Synthetic oligonucleotide
agatccatacugcggaa

SEQ ID NO: 156 - DNA, 15 bp, Artificial Sequence
  Description of Artificial Sequence Synthetic oligonucleotide
  Description of Combined DNA/RNA Molecule Synthetic oligonucleotide
cgggacguccuttgt

SEQ ID NO: 157 - DNA, 15 bp, Artificial Sequence
  Description of Artificial Sequence Synthetic oligonucleotide
  Description of Combined DNA/RNA Molecule Synthetic oligonucleotide
cgggacgtccutugu

SEQ ID NO: 158 - DNA, 16 bp, Artificial Sequence
  Description of Artificial Sequence Synthetic oligonucleotide
  Description of Combined DNA/RNA Molecule Synthetic oligonucleotide
gauccauactgcggaa

SEQ ID NO: 159 - DNA, 15 bp, Artificial Sequence
  Description of Artificial Sequence Synthetic oligonucleotide
  Description of Combined DNA/RNA Molecule Synthetic oligonucleotide
cgggacguccuttgt

SEQ ID NO: 160 - DNA, 15 bp, Artificial Sequence
  Description of Artificial Sequence Synthetic oligonucleotide
  Description of Combined DNA/RNA Molecule Synthetic oligonucleotide
agtgtutgcugacgc

SEQ ID NO: 161 - DNA, 15 bp, Artificial Sequence
  Description of Artificial Sequence Synthetic oligonucleotide
  Description of Combined DNA/RNA Molecule Synthetic oligonucleotide
tgccaacuggaucct

SEQ ID NO: 162 - DNA, 15 bp, Artificial Sequence
  Description of Artificial Sequence Synthetic oligonucleotide
  Description of Combined DNA/RNA Molecule Synthetic oligonucleotide
gcaccucucutuacg

SEQ ID NO: 163 - DNA, 16 bp, Artificial Sequence
  Description of Artificial Sequence Synthetic oligonucleotide
  Description of Combined DNA/RNA Molecule Synthetic oligonucleotide
gatccatacugcggaa

SEQ ID NO: 164 - DNA, 16 bp, Artificial Sequence
  Description of Artificial Sequence Synthetic oligonucleotide
  Description of Combined DNA/RNA Molecule Synthetic oligonucleotide
cugccaactggatccu

SEQ ID NO: 165 - DNA, 16 bp, Artificial Sequence
  Description of Artificial Sequence Synthetic oligonucleotide
  Description of Combined DNA/RNA Molecule Synthetic oligonucleotide
gcgggacgtccutugu

SEQ ID NO: 166 - DNA, 16 bp, Artificial Sequence
  Description of Artificial Sequence Synthetic oligonucleotide
  Description of Combined DNA/RNA Molecule Synthetic oligonucleotide
ggtcaccatatucutg

SEQ ID NO: 167 - DNA, 16 bp, Artificial Sequence
  Description of Artificial Sequence Synthetic oligonucleotide
  Description of Combined DNA/RNA Molecule Synthetic oligonucleotide
tcaccatatucutggg

SEQ ID NO: 168 - DNA, 16 bp, Artificial Sequence
  Description of Artificial Sequence Synthetic oligonucleotide
  Description of Combined DNA/RNA Molecule Synthetic oligonucleotide
caccauautctuggga

SEQ ID NO: 169 - DNA, 16 bp, Artificial Sequence
  Description of Artificial Sequence Synthetic oligonucleotide
  Description of Combined DNA/RNA Molecule Synthetic oligonucleotide
ccauautctugggaac

SEQ ID NO: 170 - DNA, 16 bp, Artificial Sequence
  Description of Artificial Sequence Synthetic oligonucleotide
  Description of Combined DNA/RNA Molecule Synthetic oligonucleotide
auautctugggaacaa

SEQ ID NO: 171 - DNA, 16 bp, Artificial Sequence
  Description of Artificial Sequence Synthetic oligonucleotide
  Description of Combined DNA/RNA Molecule Synthetic oligonucleotide
caagaatatggugacc

SEQ ID NO: 172 - DNA, 16 bp, Artificial Sequence
  Description of Artificial Sequence Synthetic oligonucleotide
  Description of Combined DNA/RNA Molecule Synthetic oligonucleotide
cccaagaatatgguga

SEQ ID NO: 173 - DNA, 16 bp, Artificial Sequence
  Description of Artificial Sequence Synthetic oligonucleotide
  Description of Combined DNA/RNA Molecule Synthetic oligonucleotide
tcccaagaauauggtg

SEQ ID NO: 174 - DNA, 16 bp, Artificial Sequence
  Description of Artificial Sequence Synthetic oligonucleotide
  Description of Combined DNA/RNA Molecule Synthetic oligonucleotide
gutcccaagaauaugg

SEQ ID NO: 175 - DNA, 16 bp, Artificial Sequence
  Description of Artificial Sequence Synthetic oligonucleotide
  Description of Combined DNA/RNA Molecule Synthetic oligonucleotide
tugutcccaagaauau

SEQ ID NO: 176 - DNA, 16 bp, Artificial Sequence
  Description of Artificial Sequence Synthetic oligonucleotide
  Description of Combined DNA/RNA Molecule Synthetic oligonucleotide
tuggggtggagccctc

SEQ ID NO: 177 - DNA, 17 bp, Artificial Sequence
  Description of Artificial Sequence Synthetic oligonucleotide
  Description of Combined DNA/RNA Molecule Synthetic oligonucleotide
tuggggtggagccctca

SEQ ID NO: 178 - DNA, 16 bp, Artificial Sequence
  Description of Artificial Sequence Synthetic oligonucleotide
  Description of Combined DNA/RNA Molecule Synthetic oligonucleotide
tgggguggagcccuca

SEQ ID NO: 179 - RNA, 16 nt, Artificial Sequence
  Description of Artificial Sequence Synthetic oligonucleotide
ggagcccucaggcuca

SEQ ID NO: 180 - RNA, 16 nt, Artificial Sequence
  Description of Artificial Sequence Synthetic oligonucleotide
cccucaggcucagggc

SEQ ID NO: 181 - DNA, 16 bp, Artificial Sequence
  Description of Artificial Sequence Synthetic oligonucleotide
  Description of Combined DNA/RNA Molecule Synthetic oligonucleotide
gagggctccaccccaa

SEQ ID NO: 182 - DNA, 17 bp, Artificial Sequence
  Description of Artificial Sequence Synthetic oligonucleotide
  Description of Combined DNA/RNA Molecule Synthetic oligonucleotide
tgagggcuccaccccaa

SEQ ID NO: 183 - DNA, 16 bp, Artificial Sequence
  Description of Artificial Sequence Synthetic oligonucleotide
  Description of Combined DNA/RNA Molecule Synthetic oligonucleotide
tgagggcuccacccca

SEQ ID NO: 184 - DNA, 16 bp, Artificial Sequence
  Description of Artificial Sequence Synthetic oligonucleotide
  Description of Combined DNA/RNA Molecule Synthetic oligonucleotide
tgagcctgagggcucc

SEQ ID NO: 185 - DNA, 16 bp, Artificial Sequence
  Description of Artificial Sequence Synthetic oligonucleotide
  Description of Combined DNA/RNA Molecule Synthetic oligonucleotide
gccctgagcctgaggg

SEQ ID NO: 186 - DNA, 15 bp, Artificial Sequence
  Description of Artificial Sequence Synthetic oligonucleotide
  Description of Combined DNA/RNA Molecule Synthetic oligonucleotide
ugccaactggatccu

SEQ ID NO: 187 - DNA, 15 bp, Artificial Sequence
  Description of Artificial Sequence Synthetic oligonucleotide
  Description of Combined DNA/RNA Molecule Synthetic oligonucleotide
ugccaactggatcct

SEQ ID NO: 188 - DNA, 15 bp, Artificial Sequence
  Description of Artificial Sequence Synthetic oligonucleotide
  Description of Combined DNA/RNA Molecule Synthetic oligonucleotide
tgccaactggatcct

SEQ ID NO: 189 - DNA, 15 bp, Artificial Sequence
  Description of Artificial Sequence Synthetic oligonucleotide
  Description of Combined DNA/RNA Molecule Synthetic oligonucleotide
tgccaactggatcct

SEQ ID NO: 190 - DNA, 15 bp, Artificial Sequence
  Description of Artificial Sequence Synthetic oligonucleotide
  Description of Combined DNA/RNA Molecule Synthetic oligonucleotide
tgccaacuggaucct

SEQ ID NO: 191 - DNA, 15 bp, Artificial Sequence
  Description of Artificial Sequence Synthetic oligonucleotide
  Description of Combined DNA/RNA Molecule Synthetic oligonucleotide
tgccaacuggaucct

SEQ ID NO: 192 - DNA, 15 bp, Artificial Sequence
  Description of Artificial Sequence Synthetic oligonucleotide
  Description of Combined DNA/RNA Molecule Synthetic oligonucleotide
tgccaacuggaucct

SEQ ID NO: 193 - DNA, 15 bp, Artificial Sequence
  Description of Artificial Sequence Synthetic oligonucleotide
  Description of Combined DNA/RNA Molecule Synthetic oligonucleotide
tgccaacuggaucct

SEQ ID NO: 194 - DNA, 15 bp, Artificial Sequence
  Description of Artificial Sequence Synthetic oligonucleotide
  Description of Combined DNA/RNA Molecule Synthetic oligonucleotide
tgccaacuggaucct

SEQ ID NO: 195 - DNA, 15 bp, Artificial Sequence
  Description of Artificial Sequence Synthetic oligonucleotide
  Description of Combined DNA/RNA Molecule Synthetic oligonucleotide
tgccaacuggaucct

SEQ ID NO: 196 - DNA, 16 bp, Artificial Sequence
  Description of Artificial Sequence Synthetic oligonucleotide
  Description of Combined DNA/RNA Molecule Synthetic oligonucleotide
ctgccaacuggaucct

SEQ ID NO: 197 - DNA, 15 bp, Artificial Sequence
  Description of Artificial Sequence Synthetic oligonucleotide
aggatccagttggca

SEQ ID NO: 198 - DNA, 16 bp, Artificial Sequence
  Description of Artificial Sequence Synthetic oligonucleotide
ggtcaccatattcttg

SEQ ID NO: 199 - DNA, 16 bp, Artificial Sequence
  Description of Artificial Sequence Synthetic oligonucleotide
gatccatactgcggaa

SEQ ID NO: 200 - RNA, 16 nt, Artificial Sequence
  Description of Artificial Sequence Synthetic oligonucleotide
gauccauacugcggaa

SEQ ID NO: 201 - RNA, 16 nt, Artificial Sequence
  Description of Artificial Sequence Synthetic oligonucleotide
gauccauacugcggaa

SEQ ID NO: 202 - DNA, 16 bp, Artificial Sequence
  Description of Artificial Sequence Synthetic oligonucleotide
gatccatactgcggaa

SEQ ID NO: 203 - DNA, 16 bp, Artificial Sequence
  Description of Artificial Sequence Synthetic oligonucleotide
  Description of Combined DNA/RNA Molecule Synthetic oligonucleotide
gatccauacugcggaa

SEQ ID NO: 204 - DNA, 16 bp, Artificial Sequence
  Description of Artificial Sequence Synthetic oligonucleotide
gatccatactgcggaa

SEQ ID NO: 205 - RNA, 15 nt, Artificial Sequence
  Description of Artificial Sequence Synthetic oligonucleotide
aguguuugcugacgc

SEQ ID NO: 206 - RNA, 15 nt, Artificial Sequence
  Description of Artificial Sequence Synthetic oligonucleotide
aguguuugcugacgc

SEQ ID NO: 207 - DNA, 15 bp, Artificial Sequence
  Description of Artificial Sequence Synthetic oligonucleotide
agtgtttgctgacgc

SEQ ID NO: 208 - DNA, 15 bp, Artificial Sequence
  Description of Artificial Sequence Synthetic oligonucleotide
  Description of Combined DNA/RNA Molecule Synthetic oligonucleotide
agtgtutgcugacgc

SEQ ID NO: 209 - DNA, 15 bp, Artificial Sequence
  Description of Artificial Sequence Synthetic oligonucleotide
  Description of Combined DNA/RNA Molecule Synthetic oligonucleotide
agtguuugcugacgc

SEQ ID NO: 210 - DNA, 15 bp, Artificial Sequence
  Description of Artificial Sequence Synthetic oligonucleotide
agtgtttgctgacgc

SEQ ID NO: 211 - DNA, 15 bp, Artificial Sequence
  Description of Artificial Sequence Synthetic oligonucleotide
  Description of Combined DNA/RNA Molecule Synthetic oligonucleotide
agugutugctgacgc

SEQ ID NO: 212 - DNA, 16 bp, Artificial Sequence
  Description of Artificial Sequence Synthetic oligonucleotide
ccgaccacggggcgca

SEQ ID NO: 213 - DNA, 16 bp, Artificial Sequence
  Description of Artificial Sequence Synthetic oligonucleotide
cgaccacggggcgcac

SEQ ID NO: 214 - DNA, 16 bp, Artificial Sequence
  Description of Artificial Sequence Synthetic oligonucleotide
acggggcgcacctctc

SEQ ID NO: 215 - DNA, 16 bp, Artificial Sequence
  Description of Artificial Sequence Synthetic oligonucleotide
gaccacggggcgcacc

SEQ ID NO: 216 - DNA, 16 bp, Artificial Sequence
  Description of Artificial Sequence Synthetic oligonucleotide
  Description of Combined DNA/RNA Molecule Synthetic oligonucleotide
ggggcgcacctctctu

SEQ ID NO: 217 - DNA, 16 bp, Artificial Sequence
  Description of Artificial Sequence Synthetic oligonucleotide
ggggcgcacctctctt

SEQ ID NO: 218 - DNA, 18 bp, Artificial Sequence
  Description of Artificial Sequence Synthetic oligonucleotide
ccgaccacggggcgcacc

SEQ ID NO: 219 - DNA, 19 bp, Artificial Sequence
  Description of Artificial Sequence Synthetic oligonucleotide
cgaccacggggcgcaccct

SEQ ID NO: 220 - DNA, 18 bp, Artificial Sequence
  Description of Artificial Sequence Synthetic oligonucleotide
ccacggggcgcacctctc

SEQ ID NO: 221 - RNA, 19 nt, Artificial Sequence
  Description of Artificial Sequence Synthetic oligonucleotide
gaccacggggcgcacccuc

SEQ ID NO: 222 - DNA, 18 bp, Artificial Sequence
  Description of Artificial Sequence Synthetic oligonucleotide
  Description of Combined DNA/RNA Molecule Synthetic oligonucleotide
ggggcgcacctctctuta

SEQ ID NO: 223 - DNA, 17 bp, Artificial Sequence
  Description of Artificial Sequence Synthetic oligonucleotide
  Description of Combined DNA/RNA Molecule Synthetic oligonucleotide
ggggcgcacctctctut

SEQ ID NO: 224 - RNA, 15 nt, Artificial Sequence
  Description of Artificial Sequence Synthetic oligonucleotide
cgggacguccuuugu

SEQ ID NO: 225 - DNA, 15 bp, Artificial Sequence
  Description of Artificial Sequence Synthetic oligonucleotide
cgggacgtcctttgt

SEQ ID NO: 226 - DNA, 15 bp, Artificial Sequence
  Description of Artificial Sequence Synthetic oligonucleotide
cgggacgtcctttgt

SEQ ID NO: 227 - DNA, 17 bp, Artificial Sequence
  Description of Artificial Sequence Synthetic oligonucleotide
  Description of Combined DNA/RNA Molecule Synthetic oligonucleotide
agatccatacugcggaa

SEQ ID NO: 228 - DNA, 15 bp, Artificial Sequence
  Description of Artificial Sequence Synthetic oligonucleotide
  Description of Combined DNA/RNA Molecule Synthetic oligonucleotide
cgggacguccuttgt

SEQ ID NO: 229 - DNA, 15 bp, Artificial Sequence
  Description of Artificial Sequence Synthetic oligonucleotide
  Description of Combined DNA/RNA Molecule Synthetic oligonucleotide
cgggacgtccutugu

SEQ ID NO: 230 - DNA, 16 bp, Artificial Sequence
  Description of Artificial Sequence Synthetic oligonucleotide
  Description of Combined DNA/RNA Molecule Synthetic oligonucleotide
gauccauactgcggaa

SEQ ID NO: 231 - DNA, 15 bp, Artificial Sequence
  Description of Artificial Sequence Synthetic oligonucleotide
  Description of Combined DNA/RNA Molecule Synthetic oligonucleotide
cgggacguccuttgt

SEQ ID NO: 232 - DNA, 15 bp, Artificial Sequence
  Description of Artificial Sequence Synthetic oligonucleotide
  Description of Combined DNA/RNA Molecule Synthetic oligonucleotide
agtgtutgcugacgc

SEQ ID NO: 233 - DNA, 15 bp, Artificial Sequence
  Description of Artificial Sequence Synthetic oligonucleotide
  Description of Combined DNA/RNA Molecule Synthetic oligonucleotide
tgccaacuggaucct

SEQ ID NO: 234 - DNA, 15 bp, Artificial Sequence
  Description of Artificial Sequence Synthetic oligonucleotide
  Description of Combined DNA/RNA Molecule Synthetic oligonucleotide
gcaccucucutuacg

SEQ ID NO: 235 - DNA, 16 bp, Artificial Sequence
  Description of Artificial Sequence Synthetic oligonucleotide
  Description of Combined DNA/RNA Molecule Synthetic oligonucleotide
gatccatacugcggaa

SEQ ID NO: 236 - DNA, 16 bp, Artificial Sequence
  Description of Artificial Sequence Synthetic oligonucleotide
  Description of Combined DNA/RNA Molecule Synthetic oligonucleotide
cugccaactggatccu

SEQ ID NO: 237 - DNA, 16 bp, Artificial Sequence
  Description of Artificial Sequence Synthetic oligonucleotide
  Description of Combined DNA/RNA Molecule Synthetic oligonucleotide
gcgggacgtccutugu

SEQ ID NO: 238 - DNA, 16 bp, Artificial Sequence
  Description of Artificial Sequence Synthetic oligonucleotide
  Description of Combined DNA/RNA Molecule Synthetic oligonucleotide
ggtcaccatatucutg

SEQ ID NO: 239 - DNA, 16 bp, Artificial Sequence
  Description of Artificial Sequence Synthetic oligonucleotide
  Description of Combined DNA/RNA Molecule Synthetic oligonucleotide
tcaccatatucutggg

SEQ ID NO: 240 - DNA, 16 bp, Artificial Sequence
  Description of Artificial Sequence Synthetic oligonucleotide
  Description of Combined DNA/RNA Molecule Synthetic oligonucleotide
caccauautctuggga

SEQ ID NO: 241 - DNA, 16 bp, Artificial Sequence
  Description of Artificial Sequence Synthetic oligonucleotide
  Description of Combined DNA/RNA Molecule Synthetic oligonucleotide
ccauautctugggaac

SEQ ID NO: 242 - DNA, 16 bp, Artificial Sequence
  Description of Artificial Sequence Synthetic oligonucleotide
  Description of Combined DNA/RNA Molecule Synthetic oligonucleotide
auautctugggaacaa

SEQ ID NO: 243 - DNA, 16 bp, Artificial Sequence
  Description of Artificial Sequence Synthetic oligonucleotide
  Description of Combined DNA/RNA Molecule Synthetic oligonucleotide
caagaatatggugacc

SEQ ID NO: 244 - DNA, 16 bp, Artificial Sequence
  Description of Artificial Sequence Synthetic oligonucleotide
  Description of Combined DNA/RNA Molecule Synthetic oligonucleotide
cccaagaatatgguga

SEQ ID NO: 245 - DNA, 16 bp, Artificial Sequence
  Description of Artificial Sequence Synthetic oligonucleotide
  Description of Combined DNA/RNA Molecule Synthetic oligonucleotide
tcccaagaauauggtg

SEQ ID NO: 246 - DNA, 16 bp, Artificial Sequence
  Description of Artificial Sequence Synthetic oligonucleotide
  Description of Combined DNA/RNA Molecule Synthetic oligonucleotide
gutcccaagaauaugg

SEQ ID NO: 247 - DNA, 16 bp, Artificial Sequence
  Description of Artificial Sequence Synthetic oligonucleotide
  Description of Combined DNA/RNA Molecule Synthetic oligonucleotide
tugutcccaagaauau

SEQ ID NO: 248 - DNA, 16 bp, Artificial Sequence
  Description of Artificial Sequence Synthetic oligonucleotide
  Description of Combined DNA/RNA Molecule Synthetic oligonucleotide
tuggggtggagccctc

SEQ ID NO: 249 - DNA, 17 bp, Artificial Sequence
  Description of Artificial Sequence Synthetic oligonucleotide
  Description of Combined DNA/RNA Molecule Synthetic oligonucleotide
tuggggtggagccctca

SEQ ID NO: 250 - DNA, 16 bp, Artificial Sequence
  Description of Artificial Sequence Synthetic oligonucleotide
  Description of Combined DNA/RNA Molecule Synthetic oligonucleotide
tgggguggagcccuca

SEQ ID NO: 251 - RNA, 16 nt, Artificial Sequence
  Description of Artificial Sequence Synthetic oligonucleotide
ggagcccucaggcuca

SEQ ID NO: 252 - RNA, 16 nt, Artificial Sequence
  Description of Artificial Sequence Synthetic oligonucleotide
cccucaggcucagggc

SEQ ID NO: 253 - DNA, 16 bp, Artificial Sequence
  Description of Artificial Sequence Synthetic oligonucleotide
gagggctccaccccaa

SEQ ID NO: 254 - DNA, 17 bp, Artificial Sequence
  Description of Artificial Sequence Synthetic oligonucleotide
  Description of Combined DNA/RNA Molecule Synthetic oligonucleotide
tgagggcuccaccccaa

SEQ ID NO: 255 - DNA, 16 bp, Artificial Sequence
  Description of Artificial Sequence Synthetic oligonucleotide
  Description of Combined DNA/RNA Molecule Synthetic oligonucleotide
tgagggcuccacccca

SEQ ID NO: 256 - DNA, 16 bp, Artificial Sequence
  Description of Artificial Sequence Synthetic oligonucleotide
  Description of Combined DNA/RNA Molecule Synthetic oligonucleotide
tgagcctgagggcucc

SEQ ID NO: 257 - DNA, 16 bp, Artificial Sequence
  Description of Artificial Sequence Synthetic oligonucleotide
gccctgagcctgaggg

SEQ ID NO: 258 - DNA, 15 bp, Artificial Sequence
  Description of Artificial Sequence Synthetic oligonucleotide
  Description of Combined DNA/RNA Molecule Synthetic oligonucleotide
ugccaactggatccu

SEQ ID NO: 259 - DNA, 15 bp, Artificial Sequence
  Description of Artificial Sequence Synthetic oligonucleotide
  Description of Combined DNA/RNA Molecule Synthetic oligonucleotide
ugccaactggatcct

SEQ ID NO: 260 - DNA, 15 bp, Artificial Sequence
  Description of Artificial Sequence Synthetic oligonucleotide
tgccaactggatcct

SEQ ID NO: 261 - DNA, 15 bp, Artificial Sequence
  Description of Artificial Sequence Synthetic oligonucleotide
tgccaactggatcct

SEQ ID NO: 262 - DNA, 15 bp, Artificial Sequence
  Description of Artificial Sequence Synthetic oligonucleotide
  Description of Combined DNA/RNA Molecule Synthetic oligonucleotide
tgccaacuggaucct

SEQ ID NO: 263 - DNA, 15 bp, Artificial Sequence
  Description of Artificial Sequence Synthetic oligonucleotide
  Description of Combined DNA/RNA Molecule Synthetic oligonucleotide
tgccaacuggaucct

SEQ ID NO: 264 - DNA, 15 bp, Artificial Sequence
  Description of Artificial Sequence Synthetic oligonucleotide
  Description of Combined DNA/RNA Molecule Synthetic oligonucleotide
tgccaacuggaucct

SEQ ID NO: 265 - DNA, 15 bp, Artificial Sequence
  Description of Artificial Sequence Synthetic oligonucleotide
  Description of Combined DNA/RNA Molecule Synthetic oligonucleotide
tgccaacuggaucct

SEQ ID NO: 266 - DNA, 15 bp, Artificial Sequence
  Description of Artificial Sequence Synthetic oligonucleotide
  Description of Combined DNA/RNA Molecule Synthetic oligonucleotide
tgccaacuggaucct

SEQ ID NO: 267 - DNA, 15 bp, Artificial Sequence
  Description of Artificial Sequence Synthetic oligonucleotide
  Description of Combined DNA/RNA Molecule Synthetic oligonucleotide
tgccaacuggaucct

SEQ ID NO: 268 - DNA, 16 bp, Artificial Sequence
  Description of Artificial Sequence Synthetic oligonucleotide
  Description of Combined DNA/RNA Molecule Synthetic oligonucleotide
ctgccaacuggaucct

SEQ ID NO: 269 - DNA, 15 bp, Artificial Sequence
  Description of Artificial Sequence Synthetic oligonucleotide
aggatccagttggca

SEQ ID NO: 270 - DNA, 16 bp, Artificial Sequence
  Description of Artificial Sequence Synthetic oligonucleotide
ggtcaccatattcttg

SEQ ID NO: 271 - protein, 6 aa, Artificial Sequence
  Description of Artificial Sequence Synthetic 6xHis tag
HHHHHH